(19) United States
(12) Patent Application Publication
(10) Pub. No.: US 2022/0263031 A1
(43) Pub. Date: Aug. 18, 2022

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Ji-Won UM, Gyeonggi-do (KR); Hong-Se OH, Gyeonggi-do (KR); Jeong-Hwan JEON, Gyeonggi-do (KR); Mi-Ran SEO, Gyeonggi-do (KR); Sang-Hee CHO, Gyeonggi-do (KR); Dong-Hyung LEE, Gyeonggi-do (KR); Tae-Jun HAN, Gyeonggi-do (KR)

(21) Appl. No.: 17/584,791

(22) Filed: Jan. 26, 2022

(30) Foreign Application Priority Data
Feb. 2, 2021 (KR) 10-2021-0014865
Jun. 28, 2021 (KR) 10-2021-0083798

(51) Int. Cl. H01L 51/00; C07D 403/10; C09K 11/02; C07D 403/04; C07D 209/58; C07D 405/04

(52) U.S. Cl. CPC H01L 51/0072; C07D 403/10; C09K 11/02; H01L 51/0067; H01L 51/5016; C07D 209/58; H01L 51/0061; C07D 405/04; H01L 51/0073; C07D 403/04

(57) ABSTRACT

The present disclosure relates to an organic electroluminescent compound, a plurality of host materials, and an organic electroluminescent device comprising the same. By comprising an organic electroluminescent compound and/or a plurality of host materials according to the present disclosure, an organic electroluminescent device having low driving voltage and/or high luminous efficiency and/or long lifespan can be provided.

### TECHNICAL FIELD

The present disclosure relates to an organic electroluminescent compound, a plurality of host materials, and an organic electroluminescent device comprising the same.

### BACKGROUND ART

An electroluminescent device (EL device) is a self-light-emitting display device which has advantages in that it provides a wider viewing angle, a greater contrast ratio, and a faster response time. An organic electroluminescent device (OLED) was first developed by Eastman Kodak in 1987, by using small aromatic diamine molecules and aluminum complexes as materials for forming a light-emitting layer [Appl. Phys. Lett. 51, 913, 1987].

The most important factor determining luminous efficiency in an OLED is the light-emitting materials used. The light-emitting material is classified into a host material and a dopant material in a functional aspect. A light-emitting material can be used as a combination of a host and a dopant to improve color purity, luminous efficiency, and stability. Generally, a device having excellent electroluminescent (EL) characteristics has a structure comprising a light-emitting layer formed by doping a dopant to a host. When using such a dopant/host material system as a light-emitting material, their selection is important since host materials greatly influence the efficiency and lifespan of the light-emitting device.

Recently, an urgent task is the development of an OLED having high efficiency and long lifespan property. In particular, the development of highly excellent light-emitting material over conventional light-emitting materials is urgently required, considering the EL properties necessary for medium and large-sized OLED panels.

US 2013/0175519 A1 discloses an organic light-emitting element using a fused polycyclic compound, specifically a fused triphenylene-based compound as a host. However, there is still a need for development of a light-emitting material having improved performances, such as improved driving voltage, luminous efficiency, and/or lifespan property.

### DISCLOSURE OF THE INVENTION

### Problems to be Solved

The object of the present disclosure is firstly, to provide an organic electroluminescent compound which is able to produce an organic electroluminescent device having low driving voltage and/or high luminous efficiency and/or long lifespan property, and/or a plurality of host materials comprising the same, and secondly, to provide an organic electroluminescent device comprising the organic electroluminescent compound and/or the plurality of host materials.

### Solution to Problems

As a result of intensive studies to solve the technical problem above, the present inventors found that the aforementioned objective can be achieved by an organic electroluminescent compound represented by the following formula 1, and a plurality of host materials comprising a compound represented by the following formula 1 as a first host material and a compound represented by the following formula 2 as a second host material, so that the present invention was completed.

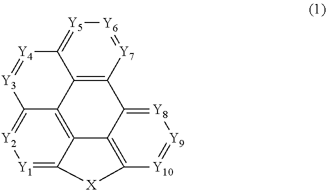

In formula 1,

X represents N-L₁-Ar₁, O, S, or CR₁R₂;

Y₁ to Y₁₀ each independently represent, CR₃ or N;

R₁ and R₂ each independently represent, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to each other to form a ring(s);

R₃ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or -L₂-Ar₂; or the adjacent R₃'s may be linked to each other to form a ring(s);

L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁ and Ar₂ represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted fused ring of a (C3-C30) aliphatic ring and a (C6-C30) aromatic ring. —Si—(R′₁)(R′₂) or —N—(R′₃)(R′₄); and

R′₁ to R′₄ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

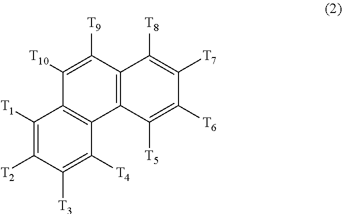

In formula 2,

T₅ and T₆ are linked to each other to form a ring of the following formula 3; or

T₇ and T₈ are linked to each other to form a ring of the following formula 3; or

T₅ and T₆ are linked to each other to form a ring of the following formula 3 and T₇ and T₈ are linked to each other to form a ring of the following formula 3;

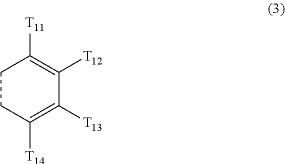

in formulas 2 and 3,

T₁ to T₄, T₉ to T₁₄, and T₅ to T₈ that do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂; provided that at least one of T₁ to T₁₄ is -L₂-Ar₂:

L₂ represents a single bond, a substituted or unsubstituted (C6-C30)arylene, or a substituted or unsubstituted (3- to 30-membered)heteroarylene;

Ar₂ represents a substituted or unsubstituted (C6-C30)aryl or a substituted or unsubstituted (3- to 30-membered)heteroaryl;

[symbol] represents a site fused to the formula 2; and

the heteroaryl comprises at least one hetero atom selected from B, N, O, S, Si and P.

### Advantageous Effects of Invention

By comprising an organic electroluminescent compound and/or a plurality of host materials according to the present disclosure, an organic electroluminescent device having low driving voltage and/or high luminous efficiency and/or long lifespan property can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to an organic electroluminescent compound represented by the formula 1, an organic electroluminescent material comprising the organic electroluminescent compound, and an organic electroluminescent device comprising the organic electroluminescent compound and/or the organic electroluminescent material.

The present disclosure relates to a plurality of host materials comprising a first host material including a compound represented by formula 1 and a second host material including a compound represented by formula 2, and an organic electroluminescent device comprising the host materials.

The term “organic electroluminescent compound” in the present disclosure means a compound that may be used in an organic electroluminescent device, and may be comprised in any material layer constituting an organic electroluminescent device, as necessary.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such of the at least two compounds may be comprised in the same layer or different layers, and may be mixture-evaporated or co-evaporated, or may be individually evaporated.

Herein, “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two host materials are comprised in one layer, the at least two host materials may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xytyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-8-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 5 to 25. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetriazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acrdinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrmidinyl, 5-pyrmidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrmidinyl, 7-benzofuro[3,2-d]pyrimidinyl, 8-benzofuro[3,2-d]pyrimidinyl, 9-benzofuro[3,2-d]pyrmidinyl, 2-benzothio[3,2-d]pyrimidinyl, 6-benzothio[3,2-d]pyrmidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatoms selected from B, N, O, S, Si and P, preferably at least one heteroatom selected from N, O and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si and P, preferably, N. O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. The substituents of the substituted alkyl(ene), the substituted alkenyl, the substituted aryl(ene), the substituted heteroaryl(ene), the substituted cycloalkyl(ene), the substituted alkoxy, and the substituted fused ring of aliphatic ring and aromatic ring in the formulas of the present disclosure, each independently represent, at least one selected from the group consisting of deuterium, halogen, cyano, carboxyl, nitro, hydroxyl, phosphine oxide, (C1-C30)alkyl, halo (C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (3- to 30-membered)heteroaryl unsubstituted or substituted with at least one of deuterium and (C6-C30)aryl, (C6-C30)aryl unsubstituted or substituted with at least one of deuterium and (3- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, a fused ring of (C3-C30) aliphatic ring and (C6-C30) aromatic ring, amino, mono- or di-(C1-C30)alkylamino, mono- or di-(C2-C30)alkenylamino, (C1-C30)alkyl(C2-C30)alkenylamino, a substituted or unsubstituted mono- or di-(C6-C30)arylamino, (C1-C30)alkyl(C6-C30)arylamino, mono- or di-(3- to 30-membered)heteroarylamino, (C1-C30)alkyl(3- to 30-membered)heteroarylamino, (C2-C30)alkenyl(C6-C30)arylamino, (C2-C30)alkenyl(3- to 30-membered)heteroarylamino, (C6-C30)aryl(3- to 30-membered)heteroarylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl.

Hereinafter, the organic electroluminescent compound according to one embodiment will be described.

The organic electroluminescent compound according to one embodiment is represented by the following formula 1.

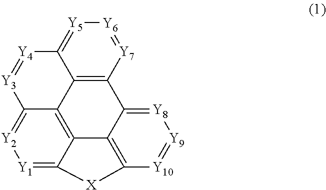

In formula 1,

X represents N-L₁-Ar₁, O, S, or CR₁R₂;

Y₁ to Y₁₀ each independently represent, CR₃ or N;

R₁ and R₂ each independently represent, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to each other to form a ring(s);

R₃ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or -L₂-Ar₂; or the adjacent R₃'s may be linked to each other to form a ring(s);

L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁ and Ar₂ each independently represent, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted fused ring of a (C3-C30) aliphatic ring and a (C6-C30) aromatic ring, —Si—(R′₁)(R′₂) or —N—(R′₃)(R′₄); and

R′₁ to R′₄ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

In one embodiment, X may be N-L₁-Ar₁.

In one embodiment, L₁ may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₁ may be a single bond, a substituted or unsubstituted phenylene, a substituted or unsubstituted o-biphenylene, a substituted or unsubstituted m-biphenylene, a substituted or unsubstituted p-biphenylene, a substituted or unsubstituted naphthylene, or a substituted or unsubstituted pyridylene. For example, the substituent of the substituted groups may be deuterium.

In one embodiment, Ar₁ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₁ may be a substituted or unsubstituted phenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted pyridyl, a substituted or unsubstituted pyrimidyl, a substituted or unsubstituted triazinyl, a substituted or unsubstituted quinazolinyl, a substituted or unsubstituted quinoxalinyl, a substituted or unsubstituted dibenzofuranyl, a substituted or unsubstituted benzoquinoxalinyl, or a substituted or unsubstituted dibenzoquinoxalinyl. For example, the substituent of the substituted groups may be at least one selected from the group consisting of deuterium, methyl, phenyl unsubstituted or substituted with deuterium, biphenyl, naphthyl, fluorenyl, pyridyl unsubstituted or substituted with phenyl, dibenzofuranyl, and dibenzothiophenyl.

In one embodiment, Ar₁ may be —N—(R′₃)(R′₄). Wherein, R′₃ and R′₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R′₃ and R′₄ each independently may be a substituted or unsubstituted phenyl, a substituted or unsubstituted p-biphenyl, a substituted or unsubstituted m-biphenyl, a substituted or unsubstituted fluorenyl, or a substituted or unsubstituted dibenzofuranyl. For example, the substituent of the substituted groups may be at least one selected from the group consisting of deuterium, methyl, and naphthyl.

In one embodiment, X may be O, S, or CR₁R₂.

In one embodiment, R₁ and R₂ each independently may be a substituted or unsubstituted (C1-C30)alkyl, a substituted or unsubstituted (C6-C30)aryl, or a substituted or unsubstituted (5- to 30-membered)heteroaryl, preferably a substituted or unsubstituted (C1-C10)alkyl, a substituted or unsubstituted (C6-C25)aryl, or a substituted or unsubstituted (5- to 25-membered)heteroaryl, more preferably a substituted or unsubstituted (C1-C4)alkyl, a substituted or unsubstituted (C6-C18)aryl, or a substituted or unsubstituted (5- to 18-membered)heteroaryl. For example, R₁ and R₂ each independently may be methyl, a substituted or unsubstituted phenyl, a substituted or unsubstituted pyridyl, a substituted or unsubstituted pyrimidyl, a substituted or unsubstituted triazinyl, a substituted or unsubstituted dibenzofuranyl, or a substituted or unsubstituted dibenzothiophenyl. For example, the substituent of the substituted groups may be at least one selected from the group consisting of phenyl, p-biphenyl, m-biphenyl, naphthyl, dimethylfluorenyl, pyridyl unsubstituted or substituted with phenyl, dibenzofuranyl, and dibenzothiophenyl.

In one embodiment, Y₁ to Y₁₀ each independently may be CR₃ or N, for example, all of Y₁ to Y₁₀ may be CR₃.

In one embodiment, R₃ may be hydrogen, a substituted or unsubstituted (C6-C30)aryl, or a substituted or unsubstituted (5- to 30-membered)heteroaryl; or the adjacent R₃'s may be linked to each other to form a ring(s), preferably hydrogen, a substituted or unsubstituted (C6-C25)aryl, or a substituted or unsubstituted (5- to 25-membered)heteroaryl; or the adjacent R₃'s may be linked to each other to form a substituted or unsubstituted (5- to 30-membered) monocyclic ring or polycyclic, alicyclic or aromatic ring, or a combination thereof, more preferably hydrogen, a substituted or unsubstituted (C6-C18)aryl, or a substituted or unsubstituted (5- to 18-membered)heteroaryl; or the adjacent R₃'s may be linked to each other to form a substituted or unsubstituted (5- to 25-membered)monocyclic or polycyclic aromatic ring. For example, R₃ may be hydrogen, a substituted or unsubstituted phenyl, a substituted or unsubstituted pyridyl, or a substituted or unsubstituted triazinyl; or the adjacent R₃'s may be linked or fused to each other to form a substituted or unsubstituted indole ring or a substituted or unsubstituted benzofuran ring. For example, the substituent of the substituted groups may be phenyl or a substituted or unsubstituted triazinyl.

In one embodiment, R₃ may be -L₂-Ar₂.

In one embodiment, L₂ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₂ may be a single bond, a substituted or unsubstituted phenylene, a substituted or unsubstituted biphenylene, a substituted or unsubstituted anthracenylene. For example, the substituent of the substituted groups may be deuterium.

In one embodiment, Ar₂ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₂ may be a substituted or unsubstituted phenyl, a substituted or unsubstituted p-terphenyl, a substituted or unsubstituted pyridyl, a substituted or unsubstituted triazinyl, a substituted or unsubstituted quinazolinyl, a substituted or unsubstituted quinoxalinyl, or a substituted or unsubstituted benzoquinoxalinyl. For example, the substituent of the substituted groups may be at least one selected from the group consisting of deuterium, methyl, phenyl unsubstituted or substituted with deuterium or naphthyl, biphenyl unsubstituted or substituted with deuterium, naphthyl, fluorenyl, pyridyl unsubstituted or substituted with phenyl, dibenzofuranyl, and dibenzothiophenyl.

In one embodiment, Ar₂ may be —N—(R′₃)(R′₄). Wherein, R′₃ and R′₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R′₃ and R′₄ each independently may be a substituted or unsubstituted phenyl, a substituted or unsubstituted p-biphenyl, a substituted or unsubstituted m-biphenyl, a substituted or unsubstituted fluorenyl, or a substituted or unsubstituted dibenzofuranyl. For example, the substituent of the substituted groups may be at least one selected from the group consisting of deuterium, methyl, and naphthyl.

The organic electroluminescent compound represented by formula I according to one embodiment may be represented by any one of the following formulas 1-1 to 1-8.

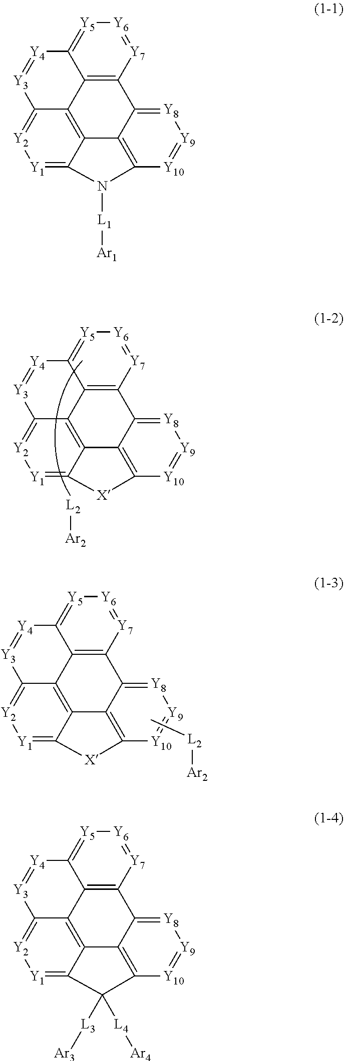

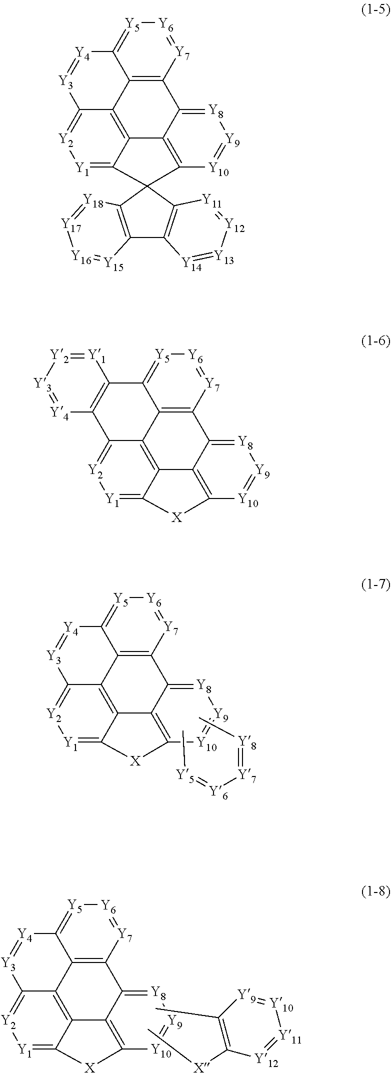

In formulas 1-1 to 1-8,

X, Y₁ to Y₁₀, L₁, L₂, Ar₁ and Ar₂ are as defined in formula 1;

Y₁₁ to Y₁₈ and Y′₁ to Y′₁₂ are as defined as Y₁ in formula 1:

L₃ and L₄ are as defined as L₁ in formula 1;

Ar₃ and Ar₄ are as defined as Ar₁ in formula 1;

X′ represents O or S;

X″ represents O, S, CR₁₁R₁₂, or NR₁₃; and

R₁₁ to R₁₃ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to the adjacent substituent to form a ring(s).

In one embodiment, L₃ and L₄ in formula 1-4 each independently may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₃ and L₄ each independently may be a single bond, a substituted or unsubstituted phenylene, or a substituted or unsubstituted dibenzofuranylene.

In one embodiment, Ar₃ and Ar₄ in formula 1-4 each independently may be a substituted or unsubstituted (C1-C30)alkyl, a substituted or unsubstituted (C6-C30)aryl, or a substituted or unsubstituted (5- to 30-membered)heteroaryl, preferably a substituted or unsubstituted (C1-C10)alkyl, a substituted or unsubstituted (C6-C25)aryl, or a substituted or unsubstituted (5- to 25-membered)heteroaryl, more preferably a substituted or unsubstituted (C1-C4)alkyl, a substituted or unsubstituted (C6-C18)aryl, or a substituted or unsubstituted (5- to 18-membered)heteroaryl. For example, Ar₃ and Ar₄ each independently may be methyl, a substituted or unsubstituted phenyl, a substituted or unsubstituted pyridyl, a substituted or unsubstituted pyrimidyl, a substituted or unsubstituted triazinyl, a substituted or unsubstituted dibenzofuranyl, or a substituted or unsubstituted dibenzothiophenyl. For example, the substituent of the substituted groups may be at least one selected from the group consisting of phenyl, p-biphenyl, m-biphenyl, naphthyl, dimethylfluorenyl, pyridyl unsubstituted or unsubstituted with phenyl, and dibenzofuranyl.

In one embodiment, Y₁₁ to Y₁₈ in formula 1-5 each independently may be CR₃ or N, preferably all of Y₁₁ to Y₁₈ may be CR₃. Wherein, R₃ may be hydrogen, a substituted or unsubstituted (C6-C30)aryl, or a substituted or unsubstituted (5- to 30-membered)heteroaryl, preferably hydrogen, a substituted or unsubstituted (C6-C25)aryl, or a substituted or unsubstituted (5- to 25-membered)heteroaryl, more preferably hydrogen, a substituted or unsubstituted (C6-C18)aryl, or a substituted or unsubstituted (5- to 18-membered)heteroaryl. For example, R₃ may be a substituted or unsubstituted biphenyl, a substituted or unsubstituted pyridyl, or a substituted or unsubstituted triazinyl. For example, the substituent of the substituted groups may be at least one selected from the group consisting of phenyl, p-biphenyl, m-biphenyl, and naphthyl.

In addition, R₃ in formula 1-5 may be -L₂-Ar₂.

In one embodiment. L₂ in formula 1-5 may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₂ may be a single bond or phenylene.

In one embodiment, Ar₂ in formula 1-5 may be —N—(R′₃)(R′₄). Wherein, R′₃ and R′₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R′₃ and R′₄ each independently may be a substituted or unsubstituted phenyl, a substituted or unsubstituted p-biphenyl, a substituted or unsubstituted m-biphenyl, a substituted or unsubstituted fluorenyl, a substituted or unsubstituted dibenzofuranyl. For example, the substituent of the substituted groups may be methyl.

In one embodiment, X in formula 1-6 may be N-L₁-Ar₁.

In one embodiment, L₁ in formula 1-6 may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁ may be a single bond, a substituted or unsubstituted phenylene, a substituted or unsubstituted biphenylene, or a substituted or unsubstituted naphthylene. For example, the substituent of the substituted groups may be deuterium.

In one embodiment, Ar₁ in formula 1-6 may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₁ may be a substituted or unsubstituted phenyl, a substituted or unsubstituted o-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted pyridyl, a substituted or unsubstituted pyrimidyl, a substituted or unsubstituted triazinyl, a substituted or unsubstituted quinazolinyl, a substituted or unsubstituted quinoxalinyl, a substituted or unsubstituted benzoquinoxalinyl, or a substituted or unsubstituted dibenzoquinoxalinyl. For example, the substituent of the substituted groups may be at least one selected from the group consisting of phenyl unsubstituted or substituted with deuterium, biphenyl unsubstituted or substituted with deuterium, naphthyl, dimethylfluorenyl, pyridyl unsubstituted or substituted with phenyl, and dibenzofuranyl.

In one embodiment, Ar₁ in formula 1-6 may be —N—(R′₃)(R′₄). Wherein, R′₃ and R′₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R′₃ and R′₄ each independently may be a substituted or unsubstituted phenyl, a substituted or unsubstituted p-biphenyl, a substituted or unsubstituted m-biphenyl, a substituted or unsubstituted fluorenyl, or a substituted or unsubstituted dibenzofuranyl. For example, the substituent of the substituted groups may be methyl.

In one embodiment, Y's to Y's in formula 1-7 each independently may be CR₃ or N, wherein R₃ may be hydrogen.

In one embodiment, X″ in formula 1-8 may be O or NR₁₃, wherein R₁₃ may be phenyl.

In one embodiment, Y′₉ to Y′₁₂ in formula 1-8 each independently may be CR₃, wherein R₃ may be hydrogen.

According to one embodiment, the organic electroluminescent compound represented by formula 1 may be more specifically illustrated by the following compounds, but is not limited thereto.

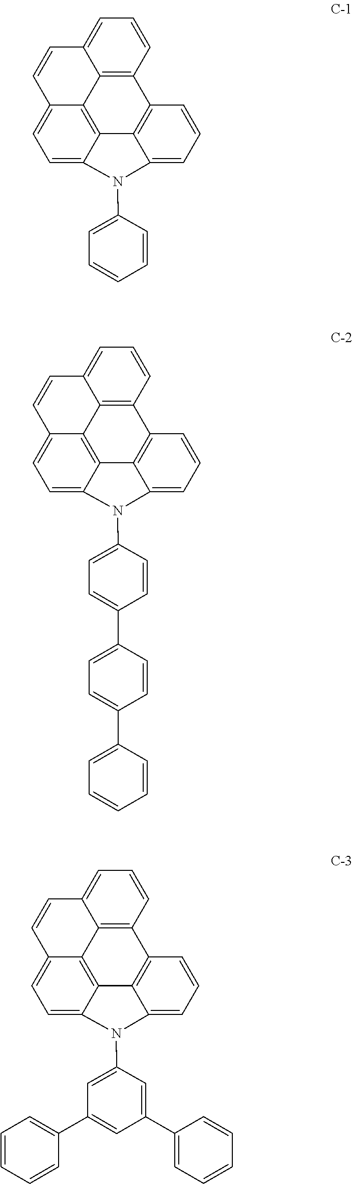 [SMILES: c1ccc(-n2c3cccc4c5cccc6ccc7ccc2c(c7c65)c43)cc1 ; c1ccc(-c2ccc(-c3ccc(-n4c5cccc6c7cccc8ccc9ccc4c(c9c87)c65)cc3)cc2)cc1 ; c1ccc(-c2cc(-c3ccccc3)cc(-n3c4cccc5c6cccc7ccc8ccc3c(c8c76)c54)c2)cc1]

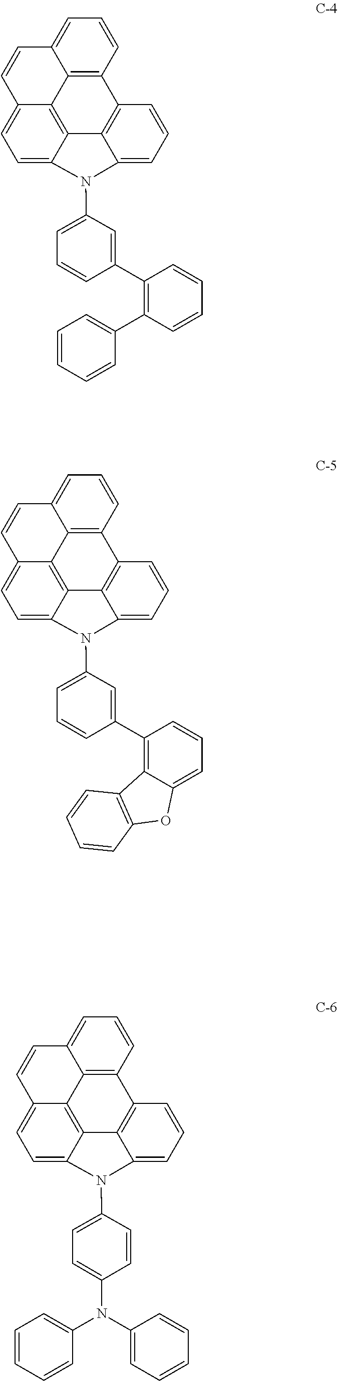 [SMILES: c1ccc(-c2ccccc2-c2cccc(-n3c4cccc5c6cccc7ccc8ccc3c(c8c76)c54)c2)cc1 ; c1cc(-c2cccc3oc4ccccc4c23)cc(-n2c3cccc4c5cccc6ccc7ccc2c(c7c65)c43)c1 ; c1ccc(N(c2ccccc2)c2ccc(-n3c4cccc5c6cccc7ccc8ccc3c(c8c76)c54)cc2)cc1]

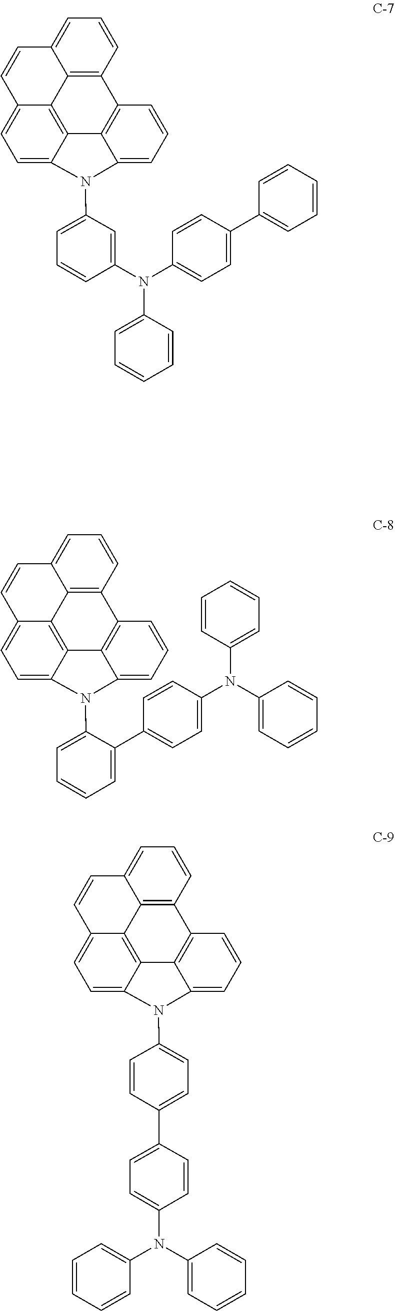 [SMILES: c1ccc(-c2ccc(N(c3ccccc3)c3cccc(-n4c5cccc6c7cccc8ccc9ccc4c(c9c87)c65)c3)cc2)cc1 ; c1ccc(N(c2ccccc2)c2ccc(-c3ccccc3-n3c4cccc5c6cccc7ccc8ccc3c(c8c76)c54)cc2)cc1 ; c1ccc(N(c2ccccc2)c2ccc(-c3ccc(-n4c5cccc6c7cccc8ccc9ccc4c(c9c87)c65)cc3)cc2)cc1]

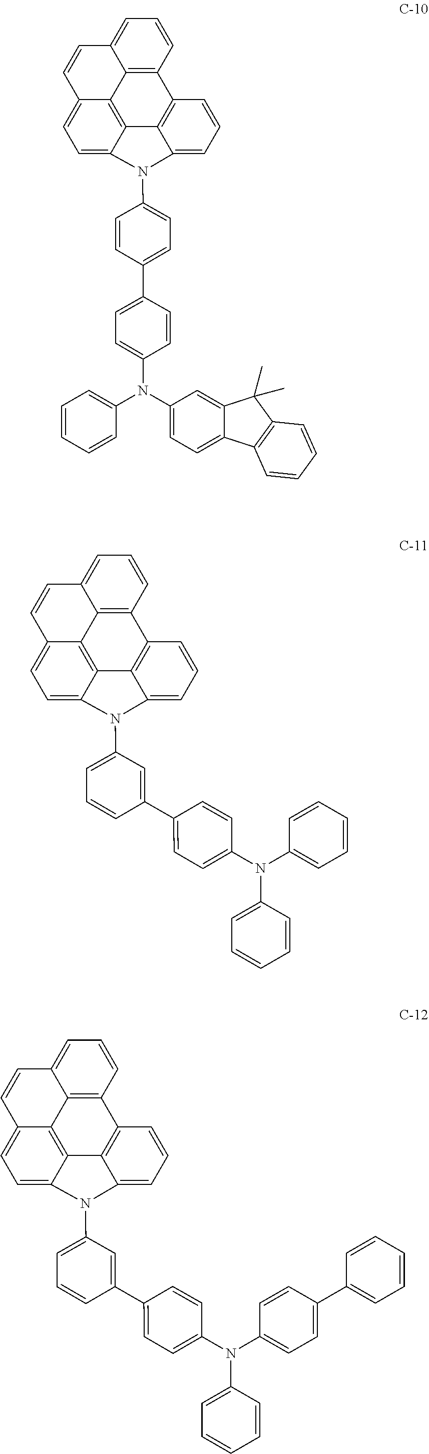 [SMILES: CC1(C)c2ccccc2-c2ccc(N(c3ccccc3)c3ccc(-c4ccc(-n5c6cccc7c8cccc9ccc%10ccc5c(c%10c98)c76)cc4)cc3)cc21 ; c1ccc(N(c2ccccc2)c2ccc(-c3cccc(-n4c5cccc6c7cccc8ccc9ccc4c(c9c87)c65)c3)cc2)cc1 ; c1ccc(-c2ccc(N(c3ccccc3)c3ccc(-c4cccc(-n5c6cccc7c8cccc9ccc%10ccc5c(c%10c98)c76)c4)cc3)cc2)cc1]

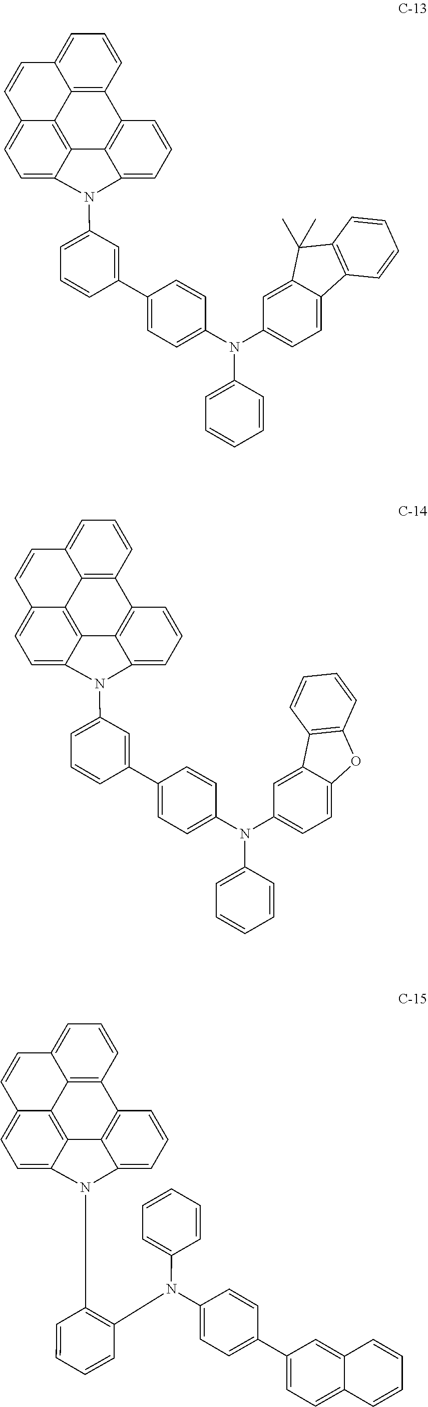 [SMILES: CC1(C)c2ccccc2-c2ccc(N(c3ccccc3)c3ccc(-c4cccc(-n5c6cccc7c8cccc9ccc%10ccc5c(c%10c98)c76)c4)cc3)cc21 ; c1ccc(N(c2ccc(-c3cccc(-n4c5cccc6c7cccc8ccc9ccc4c(c9c87)c65)c3)cc2)c2ccc3oc4ccccc4c3c2)cc1 ; c1ccc(N(c2ccc(-c3ccc4ccccc4c3)cc2)c2ccccc2-n2c3cccc4c5cccc6ccc7ccc2c(c7c65)c43)cc1]

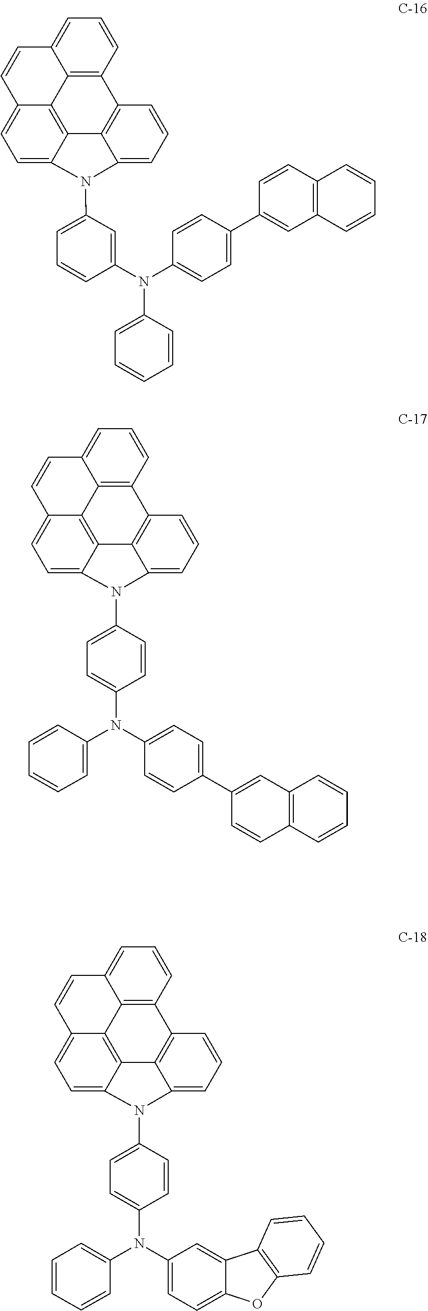 [SMILES: c1ccc(N(c2ccc(-c3ccc4ccccc4c3)cc2)c2cccc(-n3c4cccc5c6cccc7ccc8ccc3c(c8c76)c54)c2)cc1 ; c1ccc(N(c2ccc(-c3ccc4ccccc4c3)cc2)c2ccc(-n3c4cccc5c6cccc7ccc8ccc3c(c8c76)c54)cc2)cc1 ; c1ccc(N(c2ccc(-n3c4cccc5c6cccc7ccc8ccc3c(c8c76)c54)cc2)c2ccc3oc4ccccc4c3c2)cc1]

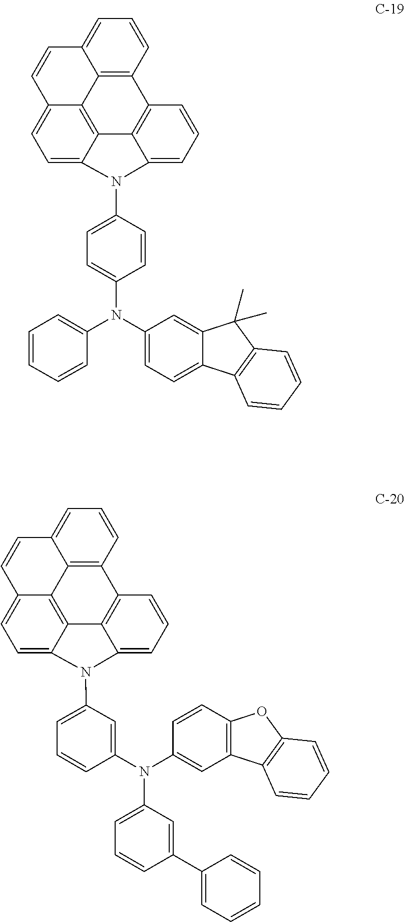 [SMILES: CC1(C)c2ccccc2-c2ccc(N(c3ccccc3)c3ccc(-n4c5cccc6c7cccc8ccc9ccc4c(c9c87)c65)cc3)cc21 ; c1ccc(-c2cccc(N(c3cccc(-n4c5cccc6c7cccc8ccc9ccc4c(c9c87)c65)c3)c3ccc4oc5ccccc5c4c3)c2)cc1]

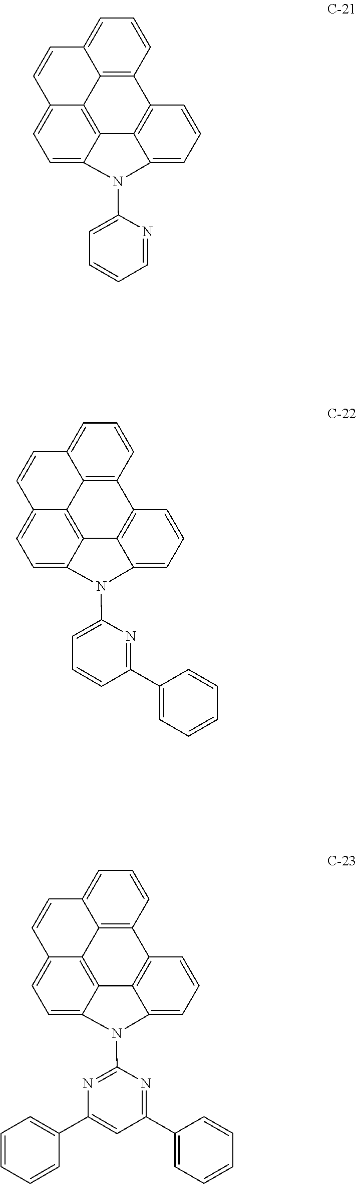 [SMILES: c1ccc(-n2c3cccc4c5cccc6ccc7ccc2c(c7c65)c43)nc1 ; c1ccc(-c2cccc(-n3c4cccc5c6cccc7ccc8ccc3c(c8c76)c54)n2)cc1 ; c1ccc(-c2cc(-c3ccccc3)nc(-n3c4cccc5c6cccc7ccc8ccc3c(c8c76)c54)n2)cc1]

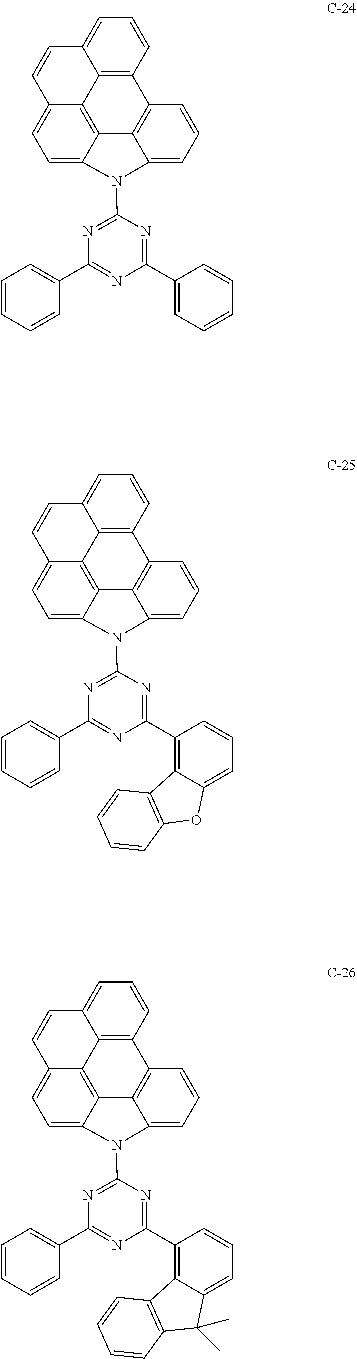 [SMILES: c1ccc(-c2nc(-c3ccccc3)nc(-n3c4cccc5c6cccc7ccc8ccc3c(c8c76)c54)n2)cc1 ; c1ccc(-c2nc(-c3cccc4oc5ccccc5c34)nc(-n3c4cccc5c6cccc7ccc8ccc3c(c8c76)c54)n2)cc1 ; CC1(C)c2ccccc2-c2c(-c3nc(-c4ccccc4)nc(-n4c5cccc6c7cccc8ccc9ccc4c(c9c87)c65)n3)cccc21]

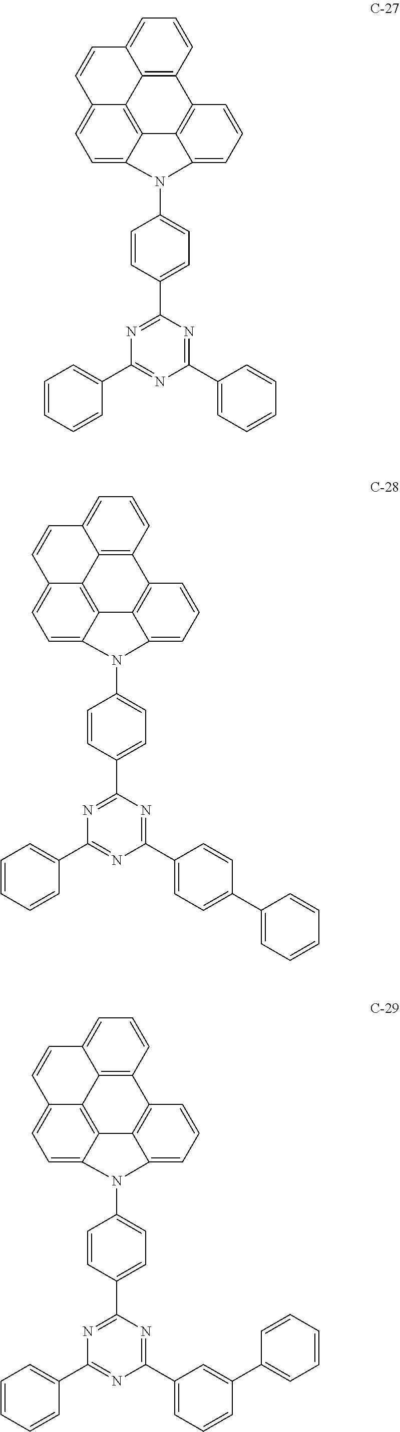 [SMILES: c1ccc(-c2nc(-c3ccccc3)nc(-c3ccc(-n4c5cccc6c7cccc8ccc9ccc4c(c9c87)c65)cc3)n2)cc1 ; c1ccc(-c2ccc(-c3nc(-c4ccccc4)nc(-c4ccc(-n5c6cccc7c8cccc9ccc%10ccc5c(c%10c98)c76)cc4)n3)cc2)cc1 ; c1ccc(-c2cccc(-c3nc(-c4ccccc4)nc(-c4ccc(-n5c6cccc7c8cccc9ccc%10ccc5c(c%10c98)c76)cc4)n3)c2)cc1]

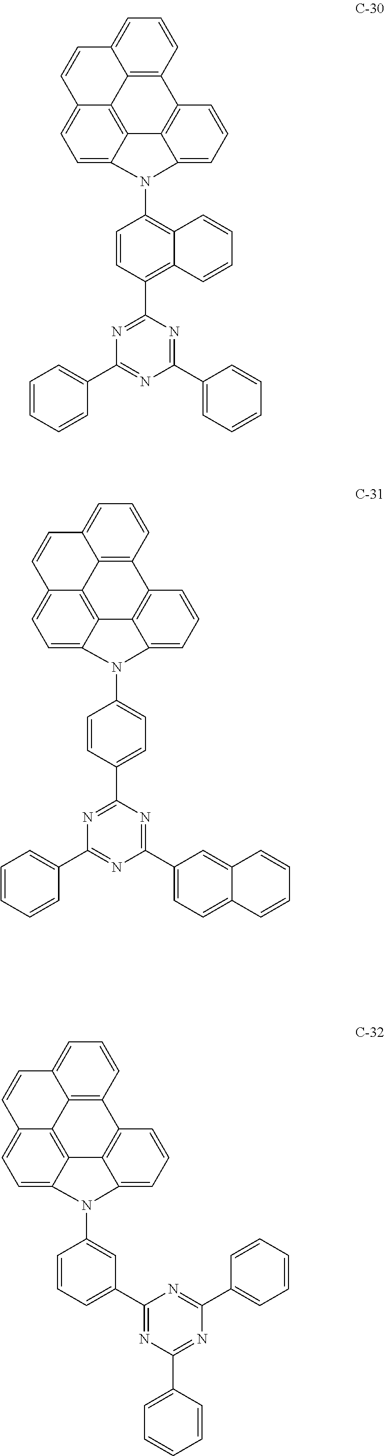 [SMILES: c1ccc(-c2nc(-c3ccccc3)nc(-c3ccc(-n4c5cccc6c7cccc8ccc9ccc4c(c9c87)c65)c4ccccc34)n2)cc1 ; c1ccc(-c2nc(-c3ccc(-n4c5cccc6c7cccc8ccc9ccc4c(c9c87)c65)cc3)nc(-c3ccc4ccccc4c3)n2)cc1 ; c1ccc(-c2nc(-c3ccccc3)nc(-c3cccc(-n4c5cccc6c7cccc8ccc9ccc4c(c9c87)c65)c3)n2)cc1]

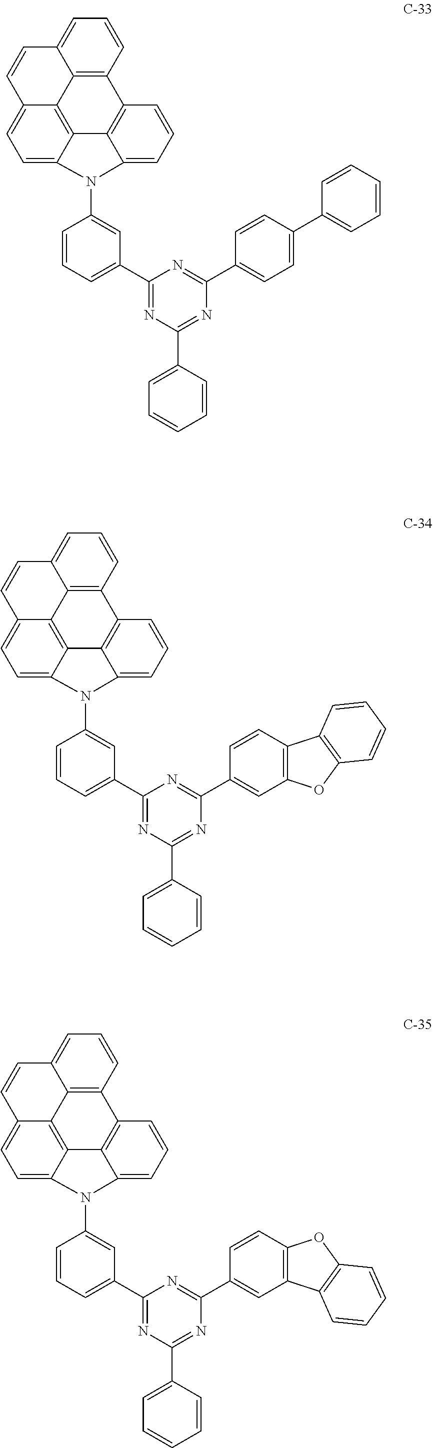 [SMILES: c1ccc(-c2ccc(-c3nc(-c4ccccc4)nc(-c4cccc(-n5c6cccc7c8cccc9ccc%10ccc5c(c%10c98)c76)c4)n3)cc2)cc1 ; c1ccc(-c2nc(-c3cccc(-n4c5cccc6c7cccc8ccc9ccc4c(c9c87)c65)c3)nc(-c3ccc4c(c3)oc3ccccc34)n2)cc1 ; c1ccc(-c2nc(-c3cccc(-n4c5cccc6c7cccc8ccc9ccc4c(c9c87)c65)c3)nc(-c3ccc4oc5ccccc5c4c3)n2)cc1]

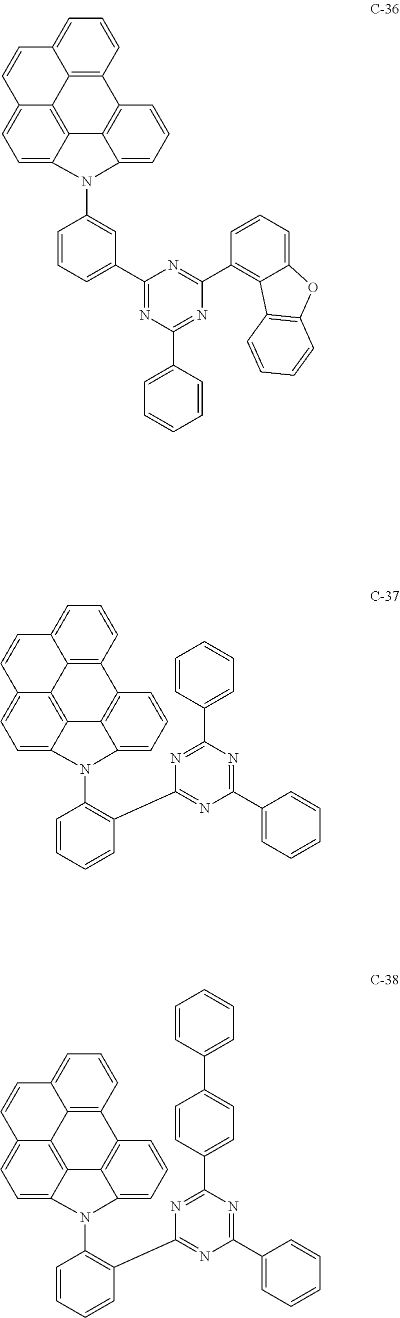 [SMILES: c1ccc(-c2nc(-c3cccc(-n4c5cccc6c7cccc8ccc9ccc4c(c9c87)c65)c3)nc(-c3cccc4oc5ccccc5c34)n2)cc1 ; c1ccc(-c2nc(-c3ccccc3)nc(-c3ccccc3-n3c4cccc5c6cccc7ccc8ccc3c(c8c76)c54)n2)cc1 ; c1ccc(-c2ccc(-c3nc(-c4ccccc4)nc(-c4ccccc4-n4c5cccc6c7cccc8ccc9ccc4c(c9c87)c65)n3)cc2)cc1]

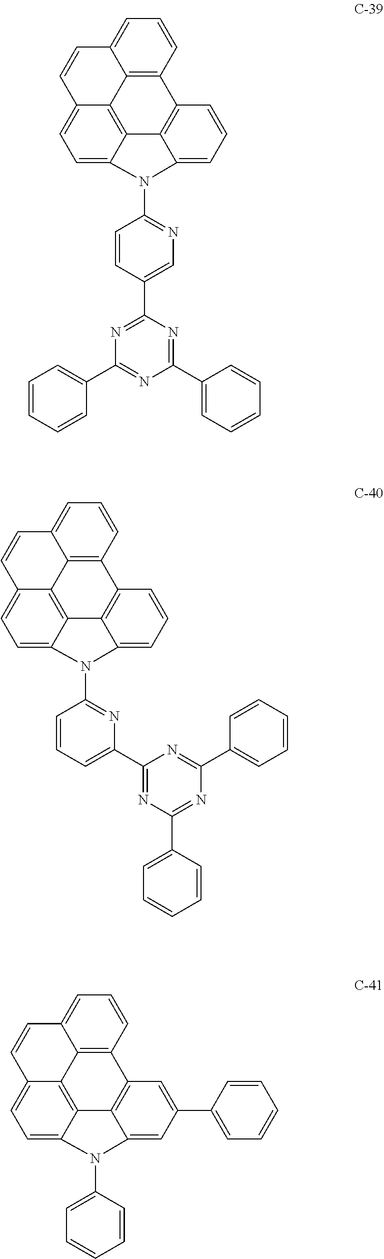 [SMILES: c1ccc(-c2nc(-c3ccccc3)nc(-c3ccc(-n4c5cccc6c7cccc8ccc9ccc4c(c9c87)c65)nc3)n2)cc1 ; c1ccc(-c2nc(-c3ccccc3)nc(-c3cccc(-n4c5cccc6c7cccc8ccc9ccc4c(c9c87)c65)n3)n2)cc1 ; c1ccc(-c2cc3c4cccc5ccc6ccc7c(c6c54)c3c(c2)n7-c2ccccc2)cc1]

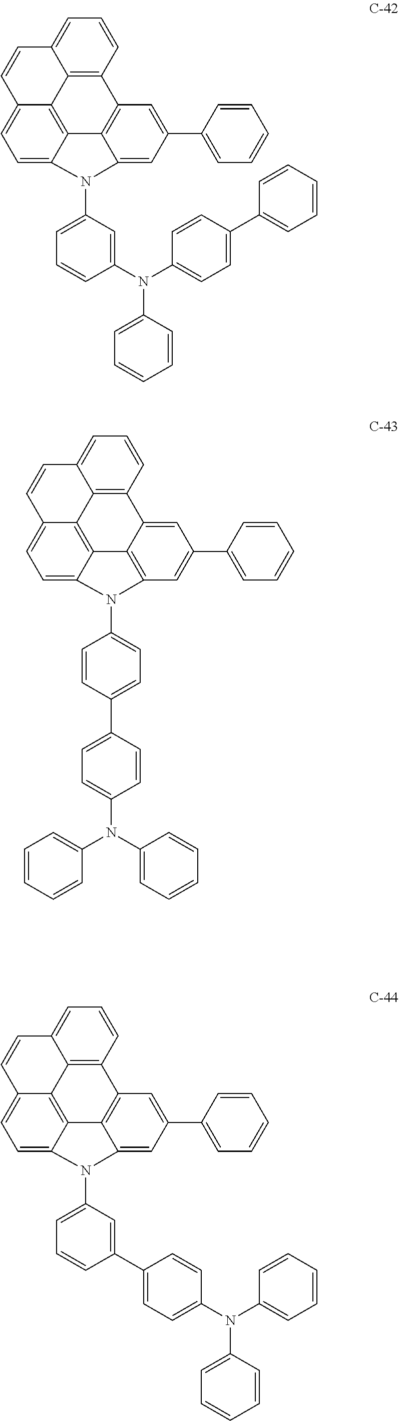 [SMILES: c1ccc(-c2ccc(N(c3ccccc3)c3cccc(-n4c5cc(-c6ccccc6)cc6c7cccc8ccc9ccc4c(c9c87)c65)c3)cc2)cc1 ; c1ccc(-c2cc3c4cccc5ccc6ccc7c(c6c54)c3c(c2)n7-c2ccc(-c3ccc(N(c4ccccc4)c4ccccc4)cc3)cc2)cc1 ; c1ccc(-c2cc3c4cccc5ccc6ccc7c(c6c54)c3c(c2)n7-c2cccc(-c3ccc(N(c4ccccc4)c4ccccc4)cc3)c2)cc1]

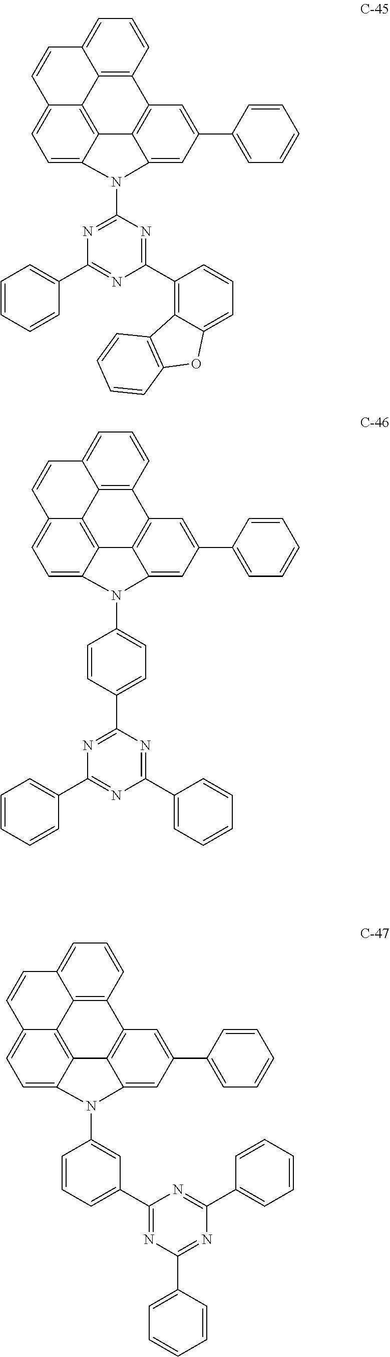

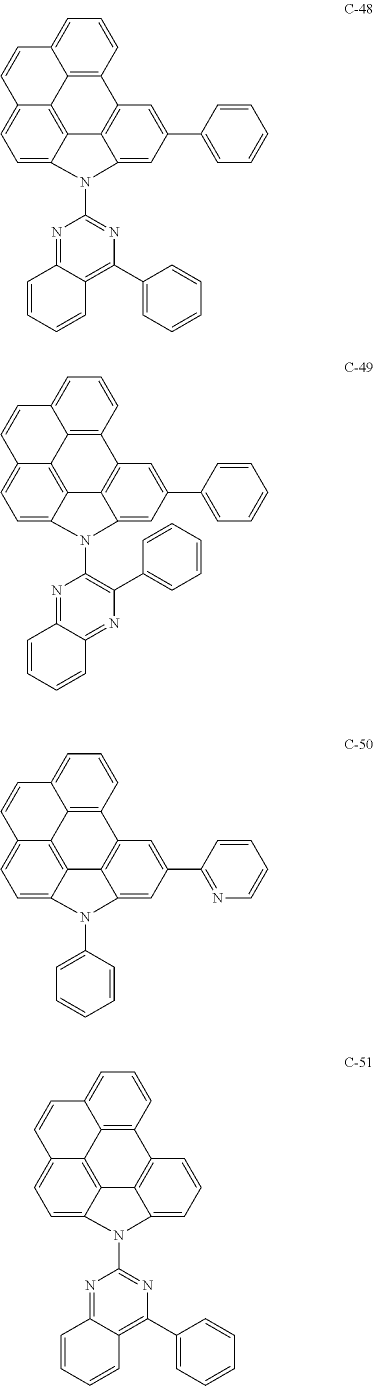

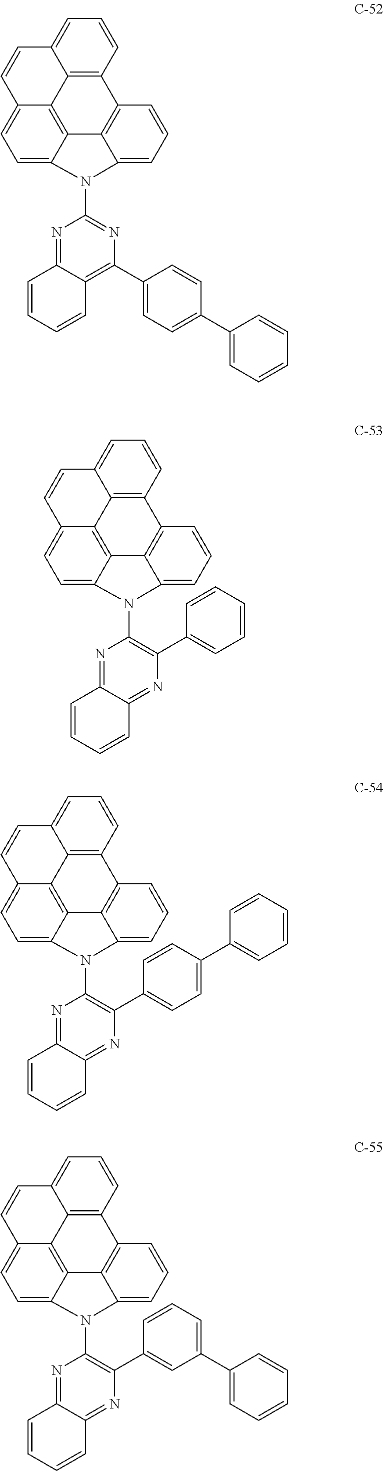

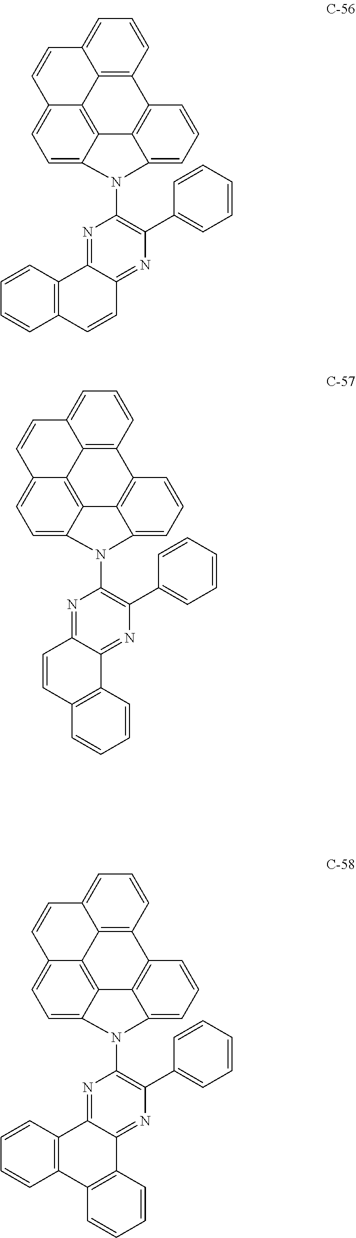 [SMILES: c1ccc(-c2nc3ccc4ccccc4c3nc2-n2c3cccc4c5cccc6ccc7ccc2c(c7c65)c43)cc1 ; c1ccc(-c2nc3c(ccc4ccccc43)nc2-n2c3cccc4c5cccc6ccc7ccc2c(c7c65)c43)cc1 ; c1ccc(-c2nc3c4ccccc4c4ccccc4c3nc2-n2c3cccc4c5cccc6ccc7ccc2c(c7c65)c43)cc1]

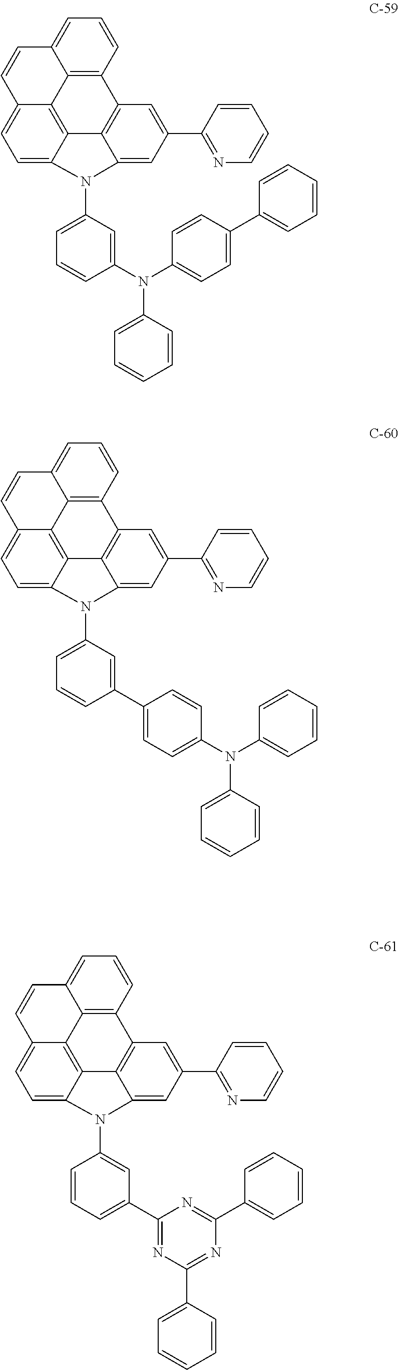 [SMILES: c1ccc(-c2ccc(N(c3ccccc3)c3cccc(-n4c5cc(-c6ccccn6)cc6c7cccc8ccc9ccc4c(c9c87)c65)c3)cc2)cc1 ; c1ccc(N(c2ccccc2)c2ccc(-c3cccc(-n4c5cc(-c6ccccn6)cc6c7cccc8ccc9ccc4c(c9c87)c65)c3)cc2)cc1 ; c1ccc(-c2nc(-c3ccccc3)nc(-c3cccc(-n4c5cc(-c6ccccn6)cc6c7cccc8ccc9ccc4c(c9c87)c65)c3)n2)cc1]

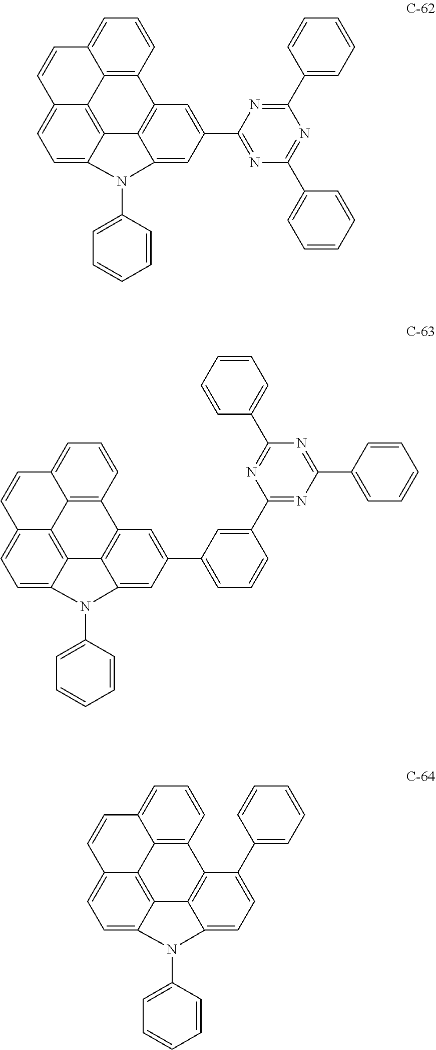 [SMILES: c1ccc(-c2nc(-c3ccccc3)nc(-c3cc4c5cccc6ccc7ccc8c(c7c65)c4c(c3)n8-c3ccccc3)n2)cc1 ; c1ccc(-c2nc(-c3ccccc3)nc(-c3cccc(-c4cc5c6cccc7ccc8ccc9c(c8c76)c5c(c4)n9-c4ccccc4)c3)n2)cc1 ; c1ccc(-c2ccc3c4c2c2cccc5ccc6ccc(c4c6c52)n3-c2ccccc2)cc1]

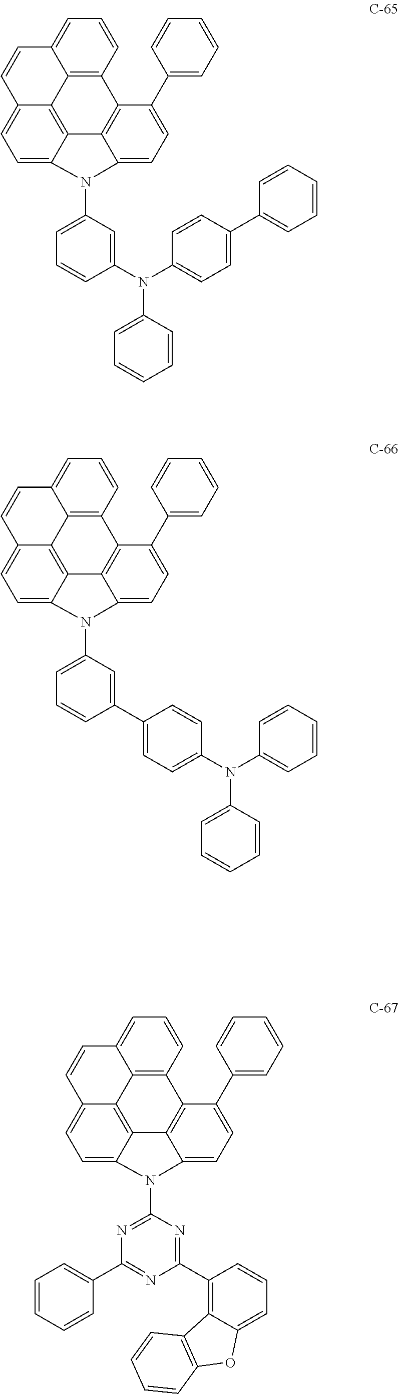 [SMILES: c1ccc(-c2ccc(N(c3ccccc3)c3cccc(-n4c5ccc(-c6ccccc6)c6c7cccc8ccc9ccc4c(c9c87)c65)c3)cc2)cc1 ; c1ccc(-c2ccc3c4c2c2cccc5ccc6ccc(c4c6c52)n3-c2cccc(-c3ccc(N(c4ccccc4)c4ccccc4)cc3)c2)cc1 ; c1ccc(-c2nc(-c3cccc4oc5ccccc5c34)nc(-n3c4ccc(-c5ccccc5)c5c6cccc7ccc8ccc3c(c8c76)c54)n2)cc1]

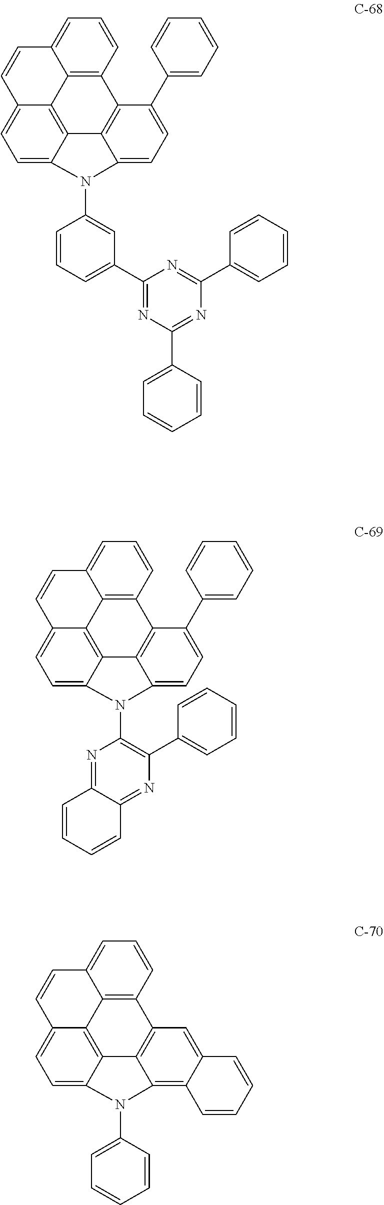 [SMILES: c1ccc(-c2nc(-c3ccccc3)nc(-c3cccc(-n4c5ccc(-c6ccccc6)c6c7cccc8ccc9ccc4c(c9c87)c65)c3)n2)cc1 ; c1ccc(-c2nc3ccccc3nc2-n2c3ccc(-c4ccccc4)c4c5cccc6ccc7ccc2c(c7c65)c43)cc1 ; c1ccc(-n2c3ccc4ccc5cccc6c7cc8ccccc8c2c7c3c4c56)cc1]

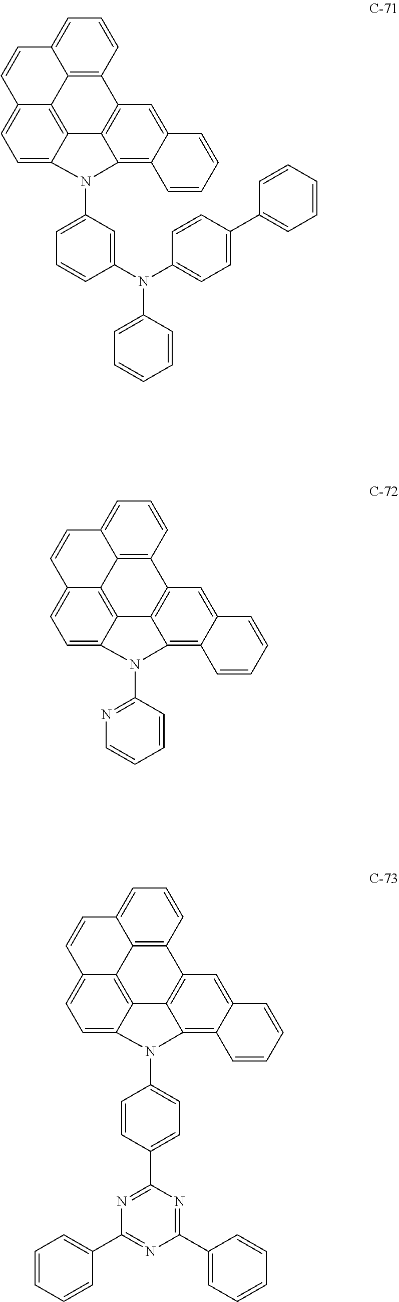 [SMILES: c1ccc(-c2ccc(N(c3ccccc3)c3cccc(-n4c5ccc6ccc7cccc8c9cc%10ccccc%10c4c9c5c6c78)c3)cc2)cc1 ; c1ccc(-n2c3ccc4ccc5cccc6c7cc8ccccc8c2c7c3c4c56)nc1 ; c1ccc(-c2nc(-c3ccccc3)nc(-c3ccc(-n4c5ccc6ccc7cccc8c9cc%10ccccc%10c4c9c5c6c78)cc3)n2)cc1]

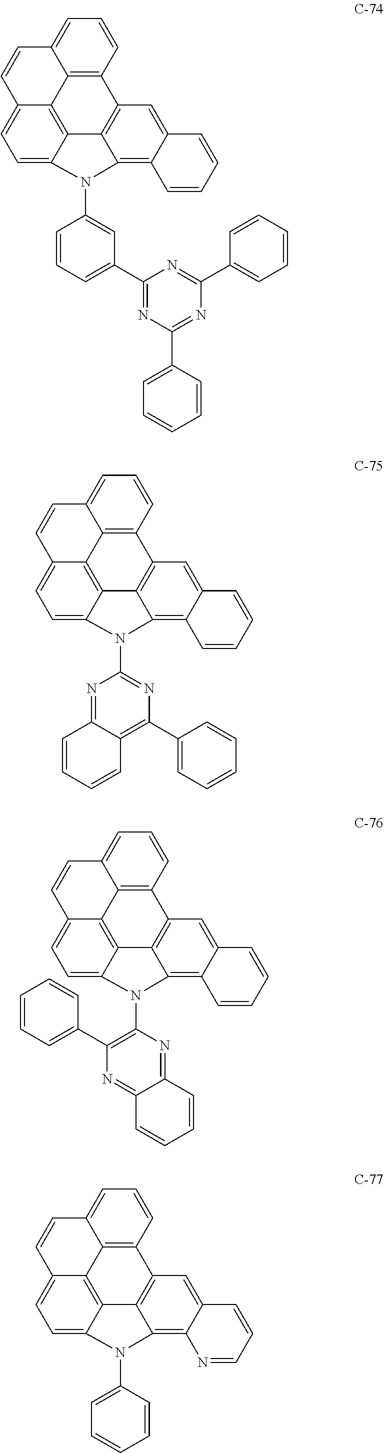 [SMILES: c1ccc(-c2nc(-c3ccccc3)nc(-c3cccc(-n4c5ccc6ccc7cccc8c9cc%10ccccc%10c4c9c5c6c78)c3)n2)cc1 ; c1ccc(-c2nc(-n3c4ccc5ccc6cccc7c8cc9ccccc9c3c8c4c5c67)nc3ccccc23)cc1 ; c1ccc(-c2nc3ccccc3nc2-n2c3ccc4ccc5cccc6c7cc8ccccc8c2c7c3c4c56)cc1 ; c1ccc(-n2c3ccc4ccc5cccc6c7cc8cccnc8c2c7c3c4c56)cc1]

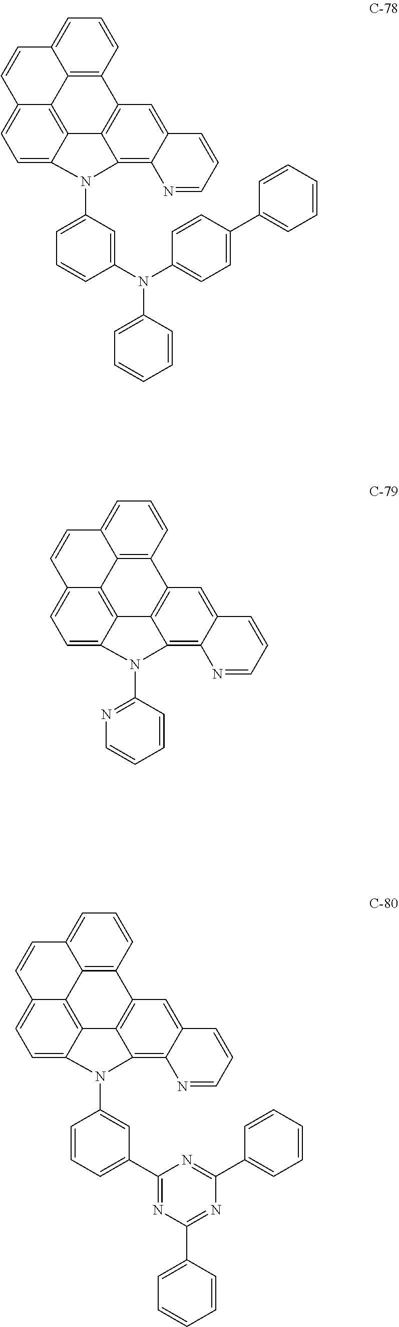 [SMILES: c1ccc(-c2ccc(N(c3ccccc3)c3cccc(-n4c5ccc6ccc7cccc8c9cc%10cccnc%10c4c9c5c6c78)c3)cc2)cc1 ; c1ccc(-n2c3ccc4ccc5cccc6c7cc8cccnc8c2c7c3c4c56)nc1 ; c1ccc(-c2nc(-c3ccccc3)nc(-c3cccc(-n4c5ccc6ccc7cccc8c9cc%10cccnc%10c4c9c5c6c78)c3)n2)cc1]

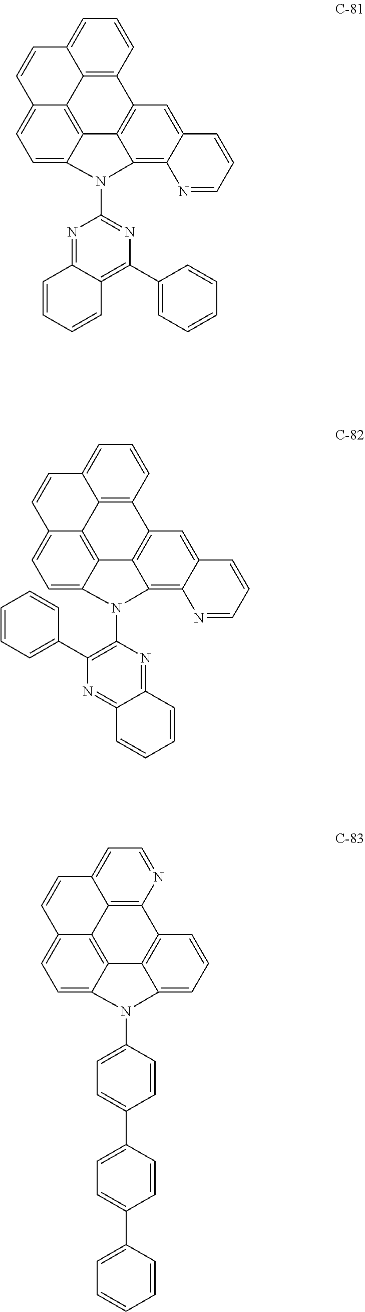 [SMILES: c1ccc(-c2nc(-n3c4ccc5ccc6cccc7c8cc9cccnc9c3c8c4c5c67)nc3ccccc23)cc1 ; c1ccc(-c2nc3ccccc3nc2-n2c3ccc4ccc5cccc6c7cc8cccnc8c2c7c3c4c56)cc1 ; c1ccc(-c2ccc(-c3ccc(-n4c5cccc6c7nccc8ccc9ccc4c(c9c87)c65)cc3)cc2)cc1]

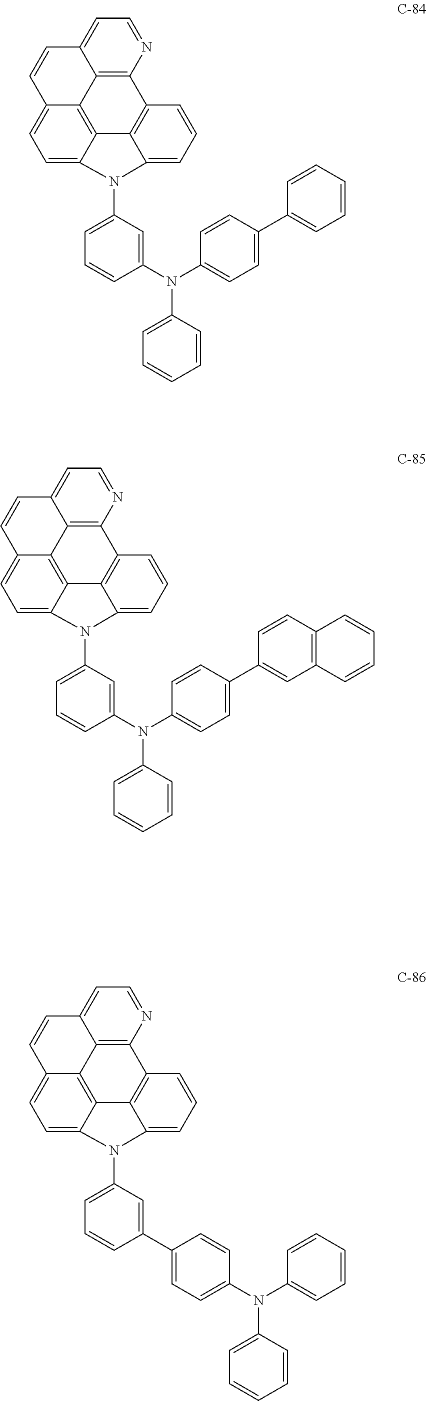 [SMILES: c1ccc(-c2ccc(N(c3ccccc3)c3cccc(-n4c5cccc6c7nccc8ccc9ccc4c(c9c87)c65)c3)cc2)cc1 ; c1ccc(N(c2ccc(-c3ccc4ccccc4c3)cc2)c2cccc(-n3c4cccc5c6nccc7ccc8ccc3c(c8c76)c54)c2)cc1 ; c1ccc(N(c2ccccc2)c2ccc(-c3cccc(-n4c5cccc6c7nccc8ccc9ccc4c(c9c87)c65)c3)cc2)cc1]

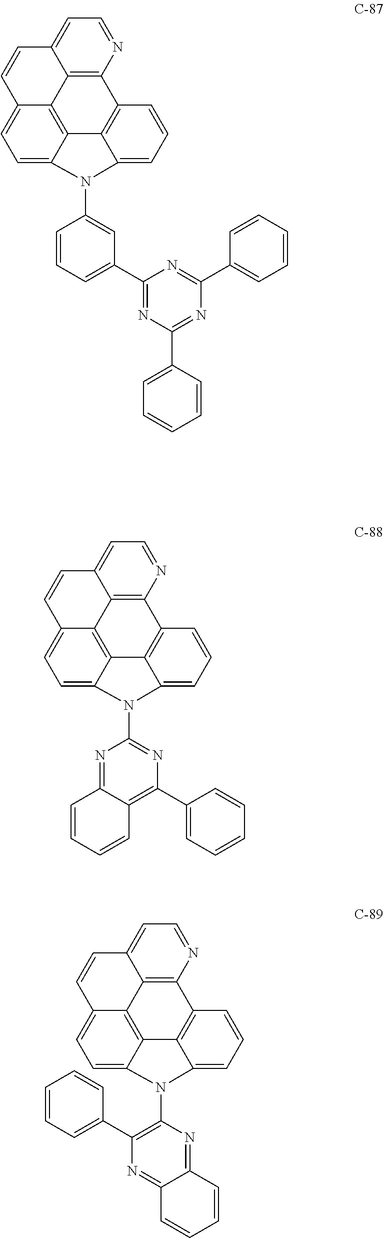 [SMILES: c1ccc(-c2nc(-c3ccccc3)nc(-c3cccc(-n4c5cccc6c7nccc8ccc9ccc4c(c9c87)c65)c3)n2)cc1 ; c1ccc(-c2nc(-n3c4cccc5c6nccc7ccc8ccc3c(c8c76)c54)nc3ccccc23)cc1 ; c1ccc(-c2nc3ccccc3nc2-n2c3cccc4c5nccc6ccc7ccc2c(c7c65)c43)cc1]

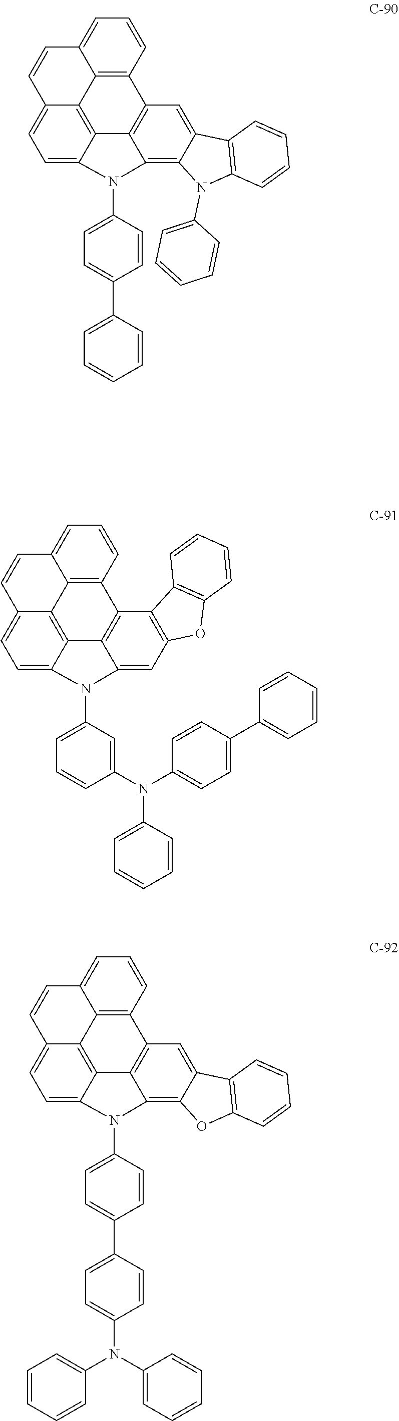 [SMILES: c1ccc(-c2ccc(-n3c4ccc5ccc6cccc7c8cc9c%10ccccc%10n(-c%10ccccc%10)c9c3c8c4c5c67)cc2)cc1 ; c1ccc(-c2ccc(N(c3ccccc3)c3cccc(-n4c5ccc6ccc7cccc8c7c6c5c5c8c6c(cc54)oc4ccccc46)c3)cc2)cc1 ; c1ccc(N(c2ccccc2)c2ccc(-c3ccc(-n4c5ccc6ccc7cccc8c9cc%10c%11ccccc%11oc%10c4c9c5c6c78)cc3)cc2)cc1]

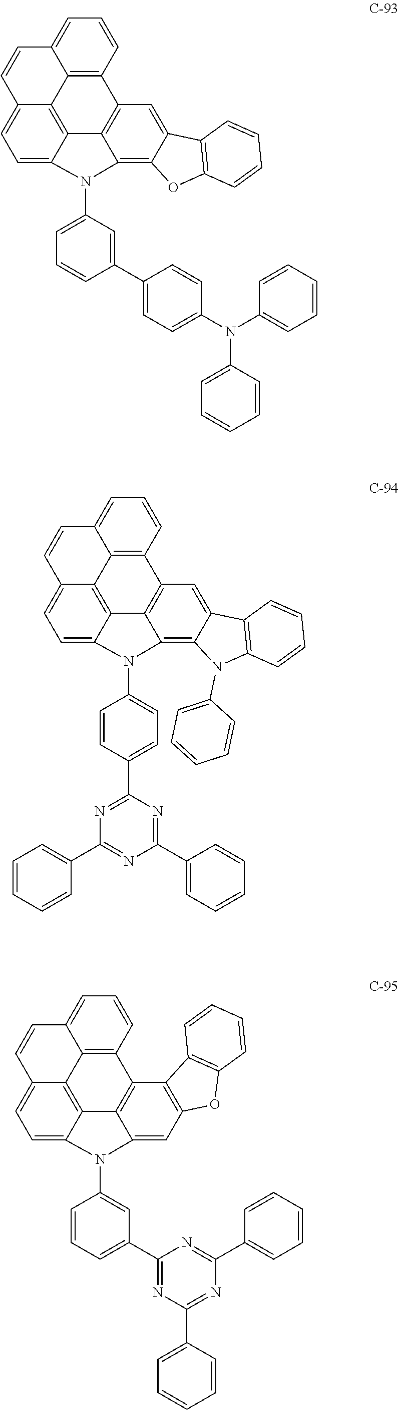 [SMILES: c1ccc(N(c2ccccc2)c2ccc(-c3cccc(-n4c5ccc6ccc7cccc8c9cc%10c%11ccccc%11oc%10c4c9c5c6c78)c3)cc2)cc1 ; c1ccc(-c2nc(-c3ccccc3)nc(-c3ccc(-n4c5ccc6ccc7cccc8c9cc%10c%11ccccc%11n(-c%11ccccc%11)c%10c4c9c5c6c78)cc3)n2)cc1 ; c1ccc(-c2nc(-c3ccccc3)nc(-c3cccc(-n4c5ccc6ccc7cccc8c7c6c5c5c8c6c(cc54)oc4ccccc46)c3)n2)cc1]

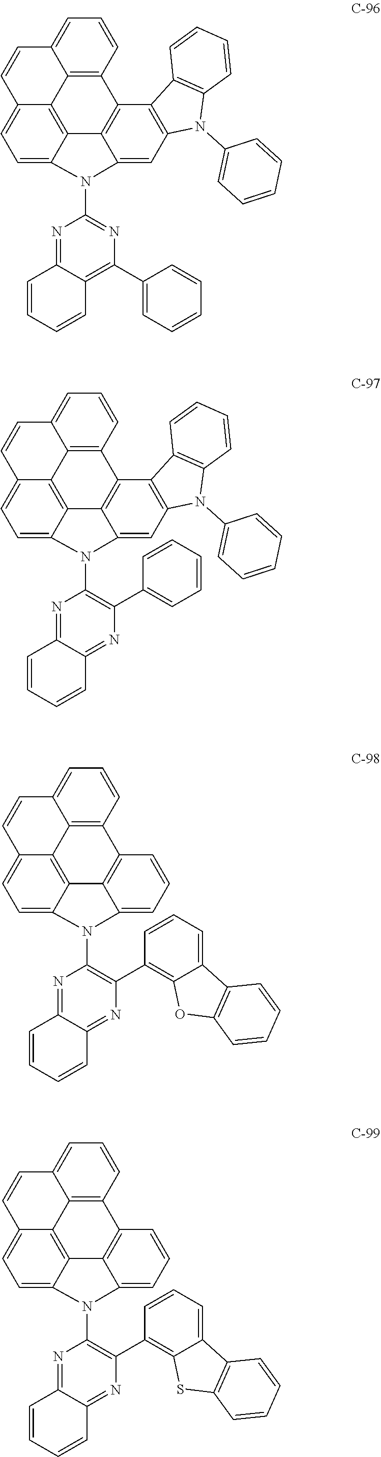 [SMILES: c1ccc(-c2nc(-n3c4ccc5ccc6cccc7c6c5c4c4c7c5c6ccccc6n(-c6ccccc6)c5cc43)nc3ccccc23)cc1 ; c1ccc(-c2nc3ccccc3nc2-n2c3ccc4ccc5cccc6c5c4c3c3c6c4c5ccccc5n(-c5ccccc5)c4cc32)cc1 ; c1ccc2nc(-n3c4cccc5c6cccc7ccc8ccc3c(c8c76)c54)c(-c3cccc4c3oc3ccccc34)nc2c1 ; c1ccc2nc(-n3c4cccc5c6cccc7ccc8ccc3c(c8c76)c54)c(-c3cccc4c3sc3ccccc34)nc2c1]

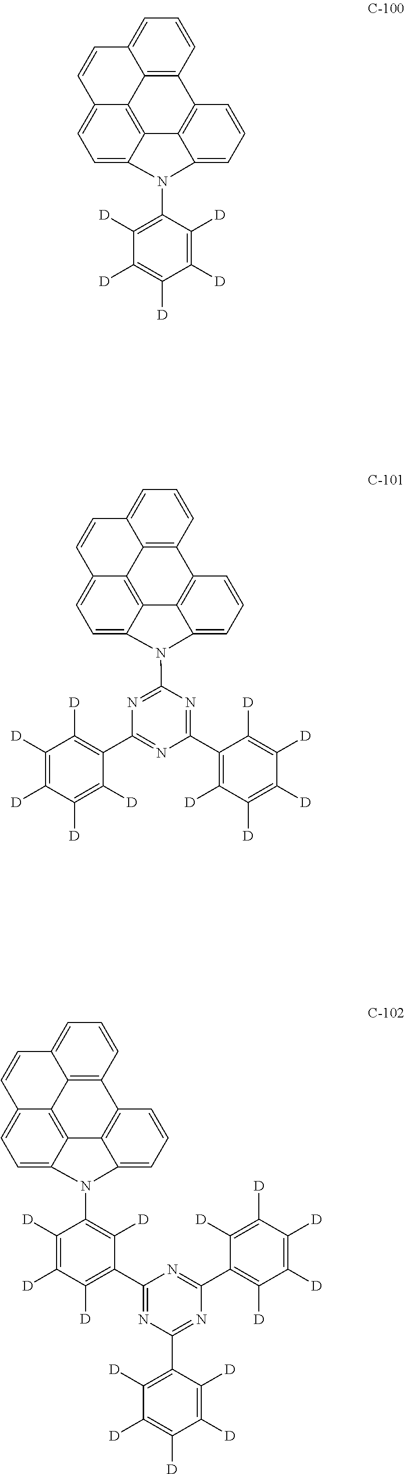 [SMILES: [2H]c1c([2H])c([2H])c(-n2c3cccc4c5cccc6ccc7ccc2c(c7c65)c43)c([2H])c1[2H] ; [2H]c1c([2H])c([2H])c(-c2nc(-c3c([2H])c([2H])c([2H])c([2H])c3[2H])nc(-n3c4cccc5c6cccc7ccc8ccc3c(c8c76)c54)n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c(-n4c5cccc6c7cccc8ccc9ccc4c(c9c87)c65)c3[2H])n2)c([2H])c1[2H]]

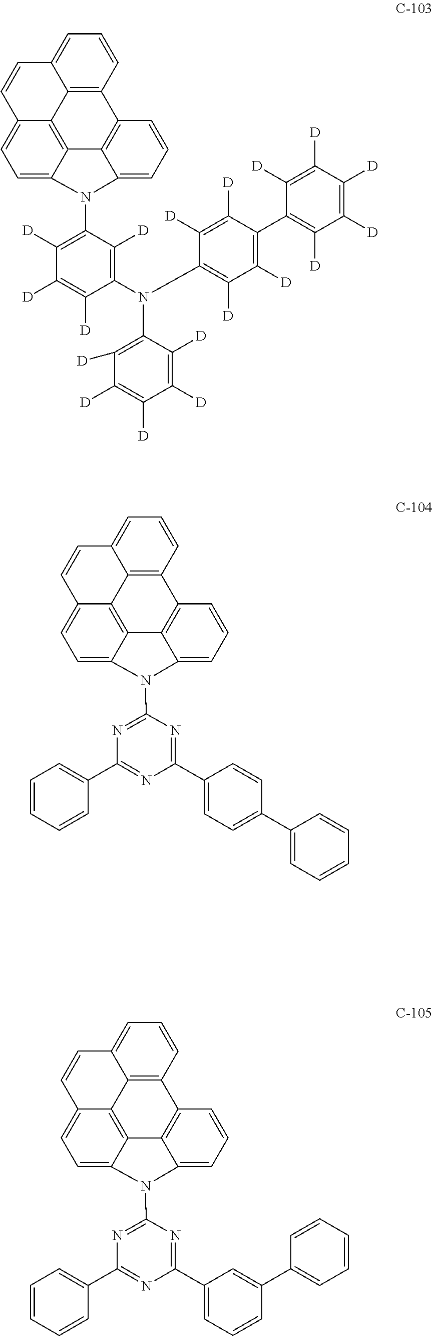 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-n4c5cccc6c7cccc8ccc9ccc4c(c9c87)c65)c3[2H])c([2H])c2[2H])c([2H])c1[2H] ; c1ccc(-c2ccc(-c3nc(-c4ccccc4)nc(-n4c5cccc6c7cccc8ccc9ccc4c(c9c87)c65)n3)cc2)cc1 ; c1ccc(-c2cccc(-c3nc(-c4ccccc4)nc(-n4c5cccc6c7cccc8ccc9ccc4c(c9c87)c65)n3)c2)cc1]

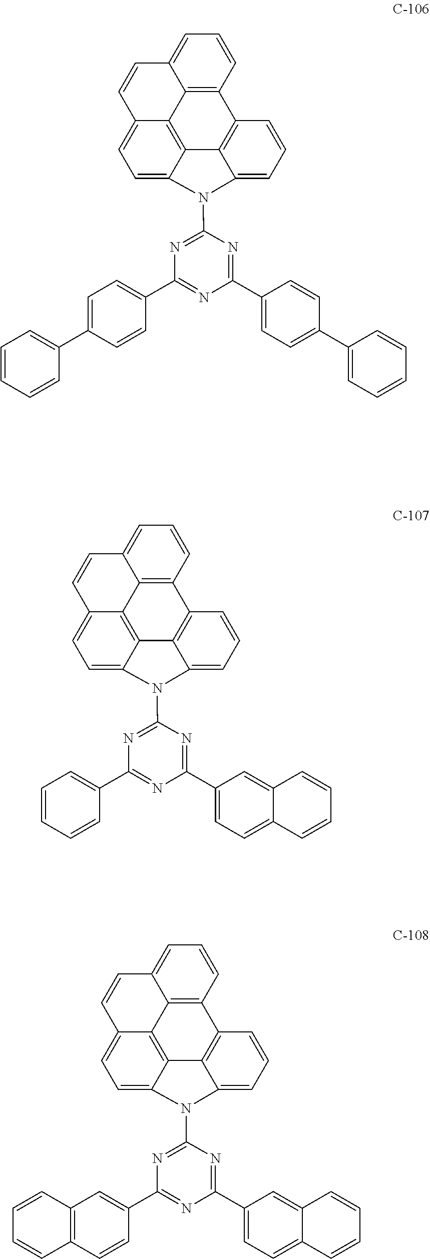 [SMILES: c1ccc(-c2ccc(-c3nc(-c4ccc(-c5ccccc5)cc4)nc(-n4c5cccc6c7cccc8ccc9ccc4c(c9c87)c65)n3)cc2)cc1 ; c1ccc(-c2nc(-c3ccc4ccccc4c3)nc(-n3c4cccc5c6cccc7ccc8ccc3c(c8c76)c54)n2)cc1 ; c1ccc2cc(-c3nc(-c4ccc5ccccc5c4)nc(-n4c5cccc6c7cccc8ccc9ccc4c(c9c87)c65)n3)ccc2c1]

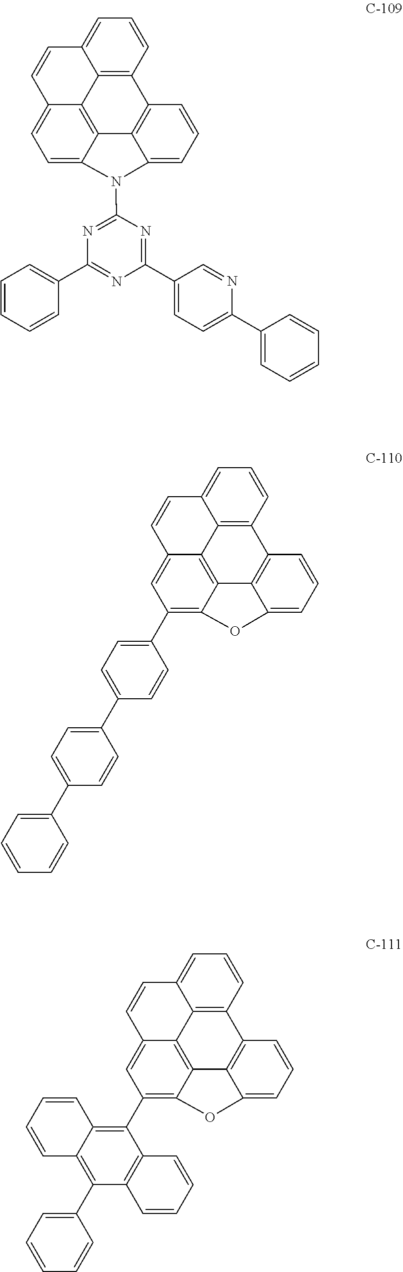 [SMILES: c1ccc(-c2ccc(-c3nc(-c4ccccc4)nc(-n4c5cccc6c7cccc8ccc9ccc4c(c9c87)c65)n3)cn2)cc1 ; c1ccc(-c2ccc(-c3ccc(-c4cc5ccc6cccc7c8cccc9oc4c(c98)c5c67)cc3)cc2)cc1 ; c1ccc(-c2c3ccccc3c(-c3cc4ccc5cccc6c7cccc8oc3c(c87)c4c56)c3ccccc23)cc1]

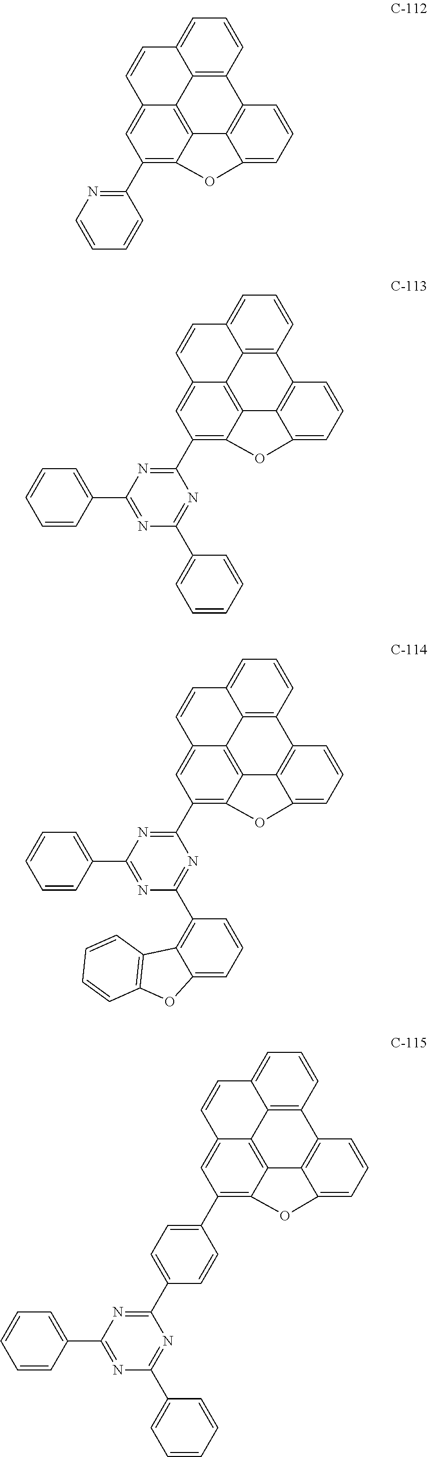 [SMILES: c1ccc(-c2cc3ccc4cccc5c6cccc7oc2c(c76)c3c45)nc1 ; c1ccc(-c2nc(-c3ccccc3)nc(-c3cc4ccc5cccc6c7cccc8oc3c(c87)c4c56)n2)cc1 ; c1ccc(-c2nc(-c3cc4ccc5cccc6c7cccc8oc3c(c87)c4c56)nc(-c3cccc4oc5ccccc5c34)n2)cc1 ; c1ccc(-c2nc(-c3ccccc3)nc(-c3ccc(-c4cc5ccc6cccc7c8cccc9oc4c(c98)c5c67)cc3)n2)cc1]

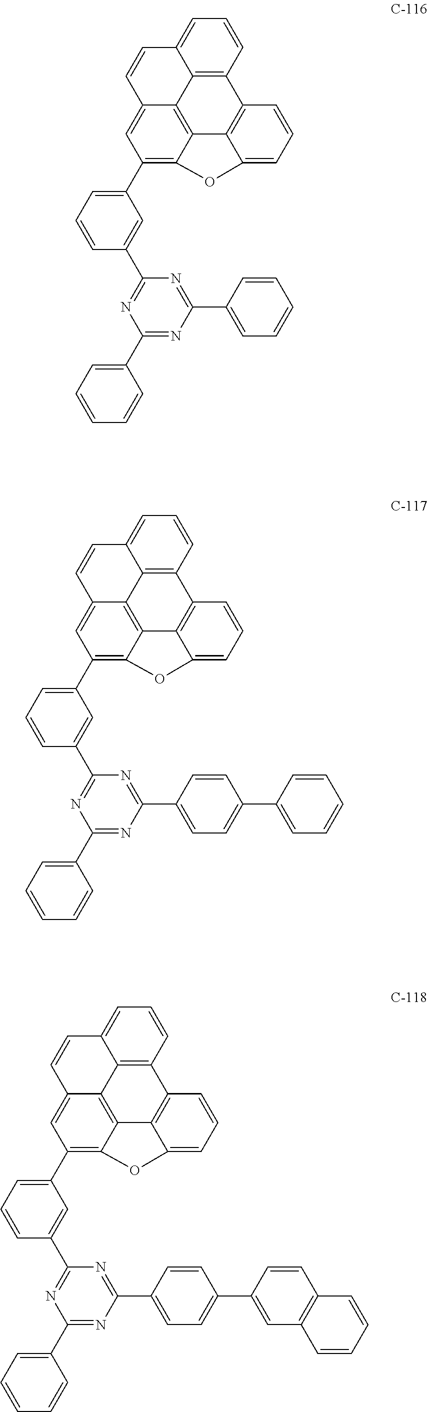 [SMILES: c1ccc(-c2nc(-c3ccccc3)nc(-c3cccc(-c4cc5ccc6cccc7c8cccc9oc4c(c98)c5c67)c3)n2)cc1 ; c1ccc(-c2ccc(-c3nc(-c4ccccc4)nc(-c4cccc(-c5cc6ccc7cccc8c9cccc%10oc5c(c%109)c6c78)c4)n3)cc2)cc1 ; c1ccc(-c2nc(-c3ccc(-c4ccc5ccccc5c4)cc3)nc(-c3cccc(-c4cc5ccc6cccc7c8cccc9oc4c(c98)c5c67)c3)n2)cc1]

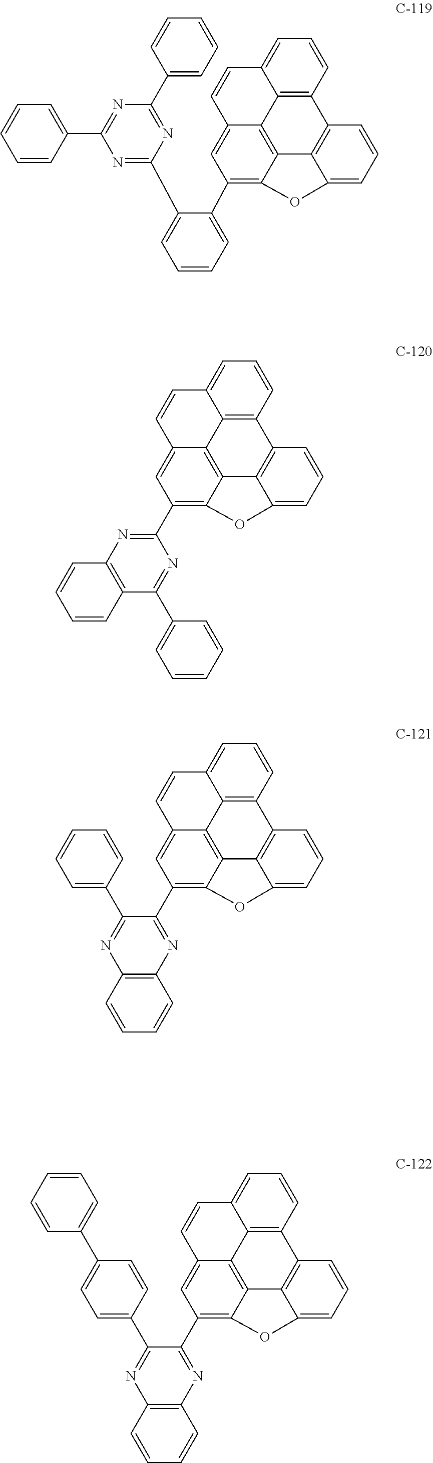 [SMILES: c1ccc(-c2nc(-c3ccccc3)nc(-c3ccccc3-c3cc4ccc5cccc6c7cccc8oc3c(c87)c4c56)n2)cc1 ; c1ccc(-c2nc(-c3cc4ccc5cccc6c7cccc8oc3c(c87)c4c56)nc3ccccc23)cc1 ; c1ccc(-c2nc3ccccc3nc2-c2cc3ccc4cccc5c6cccc7oc2c(c76)c3c45)cc1 ; c1ccc(-c2ccc(-c3nc4ccccc4nc3-c3cc4ccc5cccc6c7cccc8oc3c(c87)c4c56)cc2)cc1]

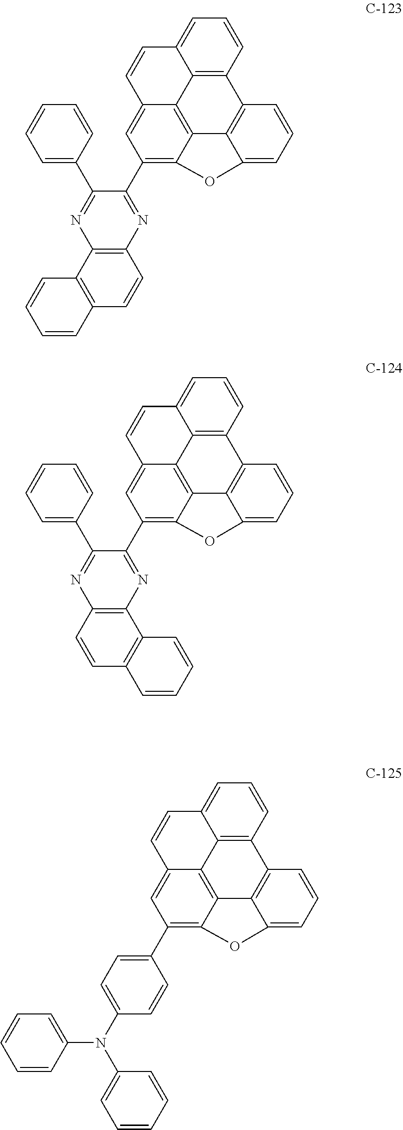 [SMILES: c1ccc(-c2nc3c(ccc4ccccc43)nc2-c2cc3ccc4cccc5c6cccc7oc2c(c76)c3c45)cc1 ; c1ccc(-c2nc3ccc4ccccc4c3nc2-c2cc3ccc4cccc5c6cccc7oc2c(c76)c3c45)cc1 ; c1ccc(N(c2ccccc2)c2ccc(-c3cc4ccc5cccc6c7cccc8oc3c(c87)c4c56)cc2)cc1]

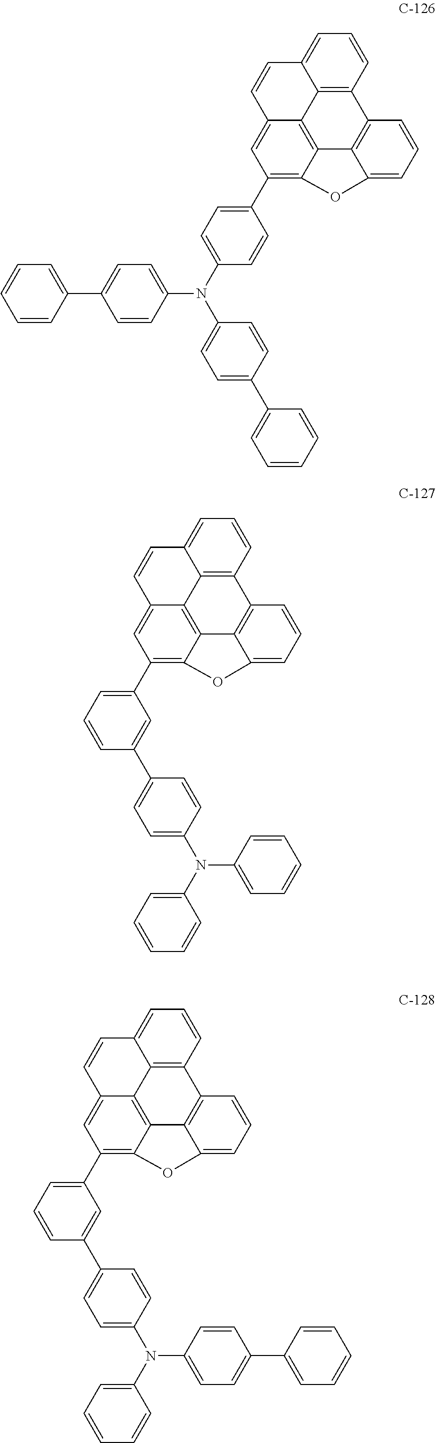 [SMILES: c1ccc(-c2ccc(N(c3ccc(-c4ccccc4)cc3)c3ccc(-c4cc5ccc6cccc7c8cccc9oc4c(c98)c5c67)cc3)cc2)cc1 ; c1ccc(N(c2ccccc2)c2ccc(-c3cccc(-c4cc5ccc6cccc7c8cccc9oc4c(c98)c5c67)c3)cc2)cc1 ; c1ccc(-c2ccc(N(c3ccccc3)c3ccc(-c4cccc(-c5cc6ccc7cccc8c9cccc%10oc5c(c%109)c6c78)c4)cc3)cc2)cc1]

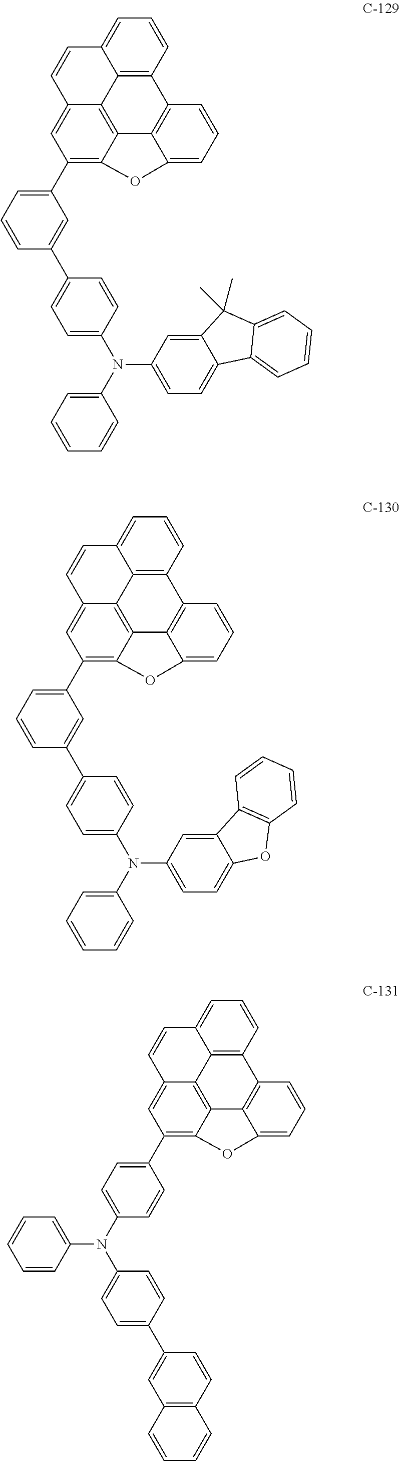 [SMILES: CC1(C)c2ccccc2-c2ccc(N(c3ccccc3)c3ccc(-c4cccc(-c5cc6ccc7cccc8c9cccc%10oc5c(c%109)c6c78)c4)cc3)cc21 ; c1ccc(N(c2ccc(-c3cccc(-c4cc5ccc6cccc7c8cccc9oc4c(c98)c5c67)c3)cc2)c2ccc3oc4ccccc4c3c2)cc1 ; c1ccc(N(c2ccc(-c3ccc4ccccc4c3)cc2)c2ccc(-c3cc4ccc5cccc6c7cccc8oc3c(c87)c4c56)cc2)cc1]

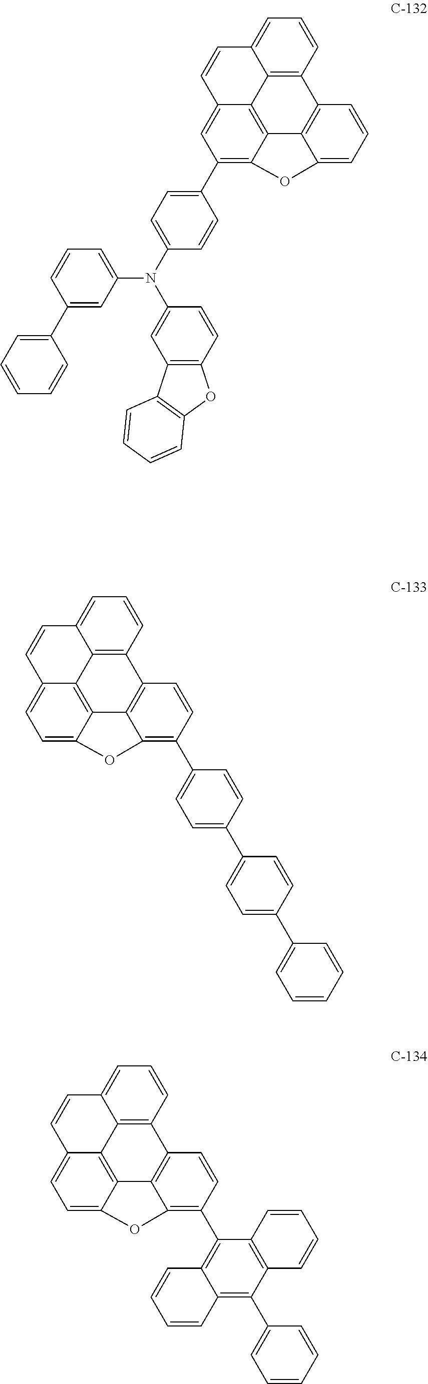 [SMILES: c1ccc(-c2cccc(N(c3ccc(-c4cc5ccc6cccc7c8cccc9oc4c(c98)c5c67)cc3)c3ccc4oc5ccccc5c4c3)c2)cc1 ; c1ccc(-c2ccc(-c3ccc(-c4ccc5c6cccc7ccc8ccc9oc4c5c9c8c76)cc3)cc2)cc1 ; c1ccc(-c2c3ccccc3c(-c3ccc4c5cccc6ccc7ccc8oc3c4c8c7c65)c3ccccc23)cc1]

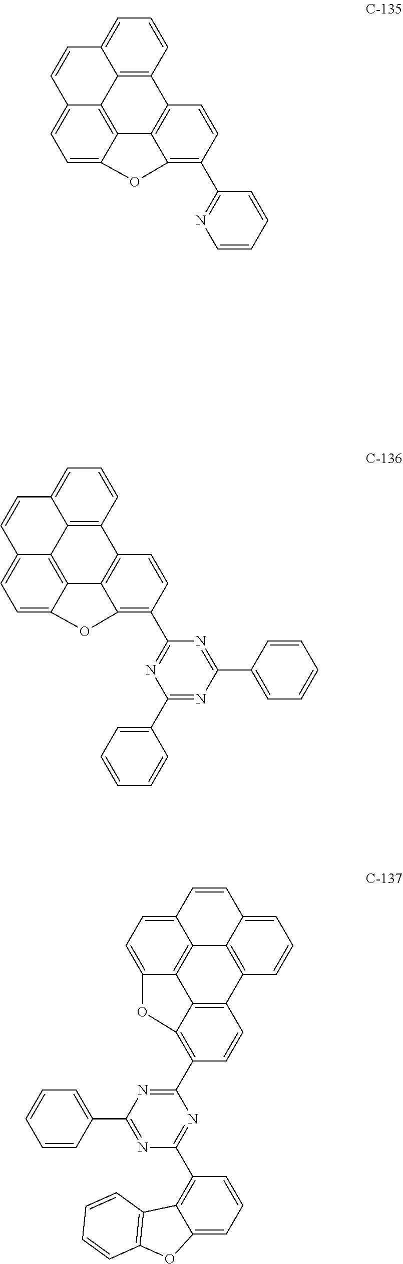 [SMILES: c1ccc(-c2ccc3c4cccc5ccc6ccc7oc2c3c7c6c54)nc1 ; c1ccc(-c2nc(-c3ccccc3)nc(-c3ccc4c5cccc6ccc7ccc8oc3c4c8c7c65)n2)cc1 ; c1ccc(-c2nc(-c3ccc4c5cccc6ccc7ccc8oc3c4c8c7c65)nc(-c3cccc4oc5ccccc5c34)n2)cc1]

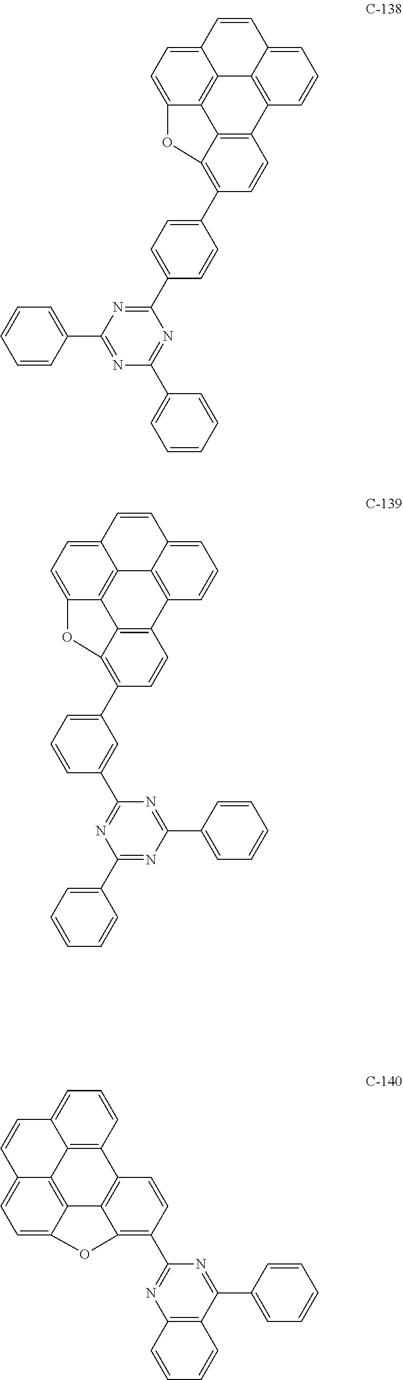 [SMILES: c1ccc(-c2nc(-c3ccccc3)nc(-c3ccc(-c4ccc5c6cccc7ccc8ccc9oc4c5c9c8c76)cc3)n2)cc1 ; c1ccc(-c2nc(-c3ccccc3)nc(-c3cccc(-c4ccc5c6cccc7ccc8ccc9oc4c5c9c8c76)c3)n2)cc1 ; c1ccc(-c2nc(-c3ccc4c5cccc6ccc7ccc8oc3c4c8c7c65)nc3ccccc23)cc1]

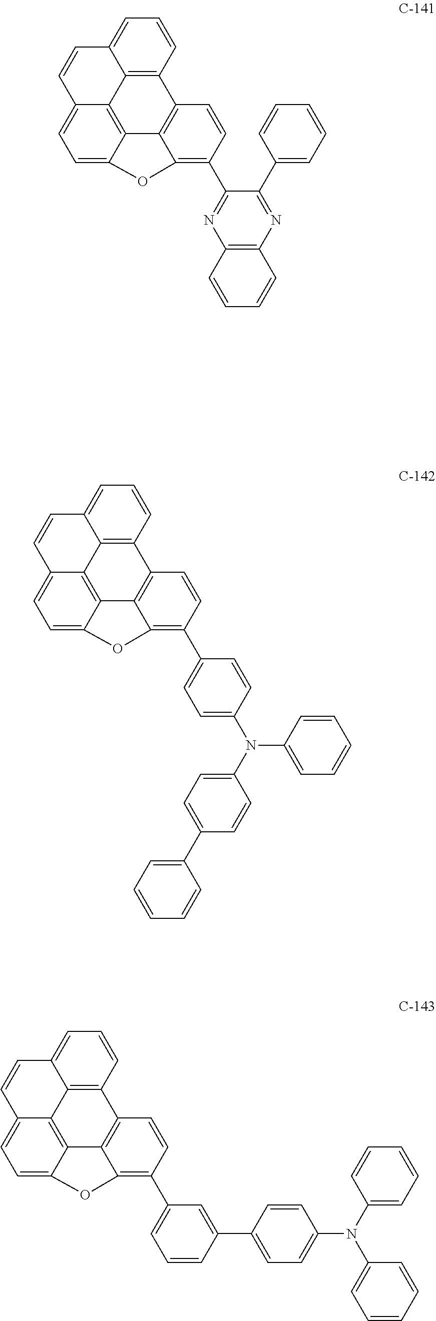 [SMILES: c1ccc(-c2nc3ccccc3nc2-c2ccc3c4cccc5ccc6ccc7oc2c3c7c6c54)cc1 ; c1ccc(-c2ccc(N(c3ccccc3)c3ccc(-c4ccc5c6cccc7ccc8ccc9oc4c5c9c8c76)cc3)cc2)cc1 ; c1ccc(N(c2ccccc2)c2ccc(-c3cccc(-c4ccc5c6cccc7ccc8ccc9oc4c5c9c8c76)c3)cc2)cc1]

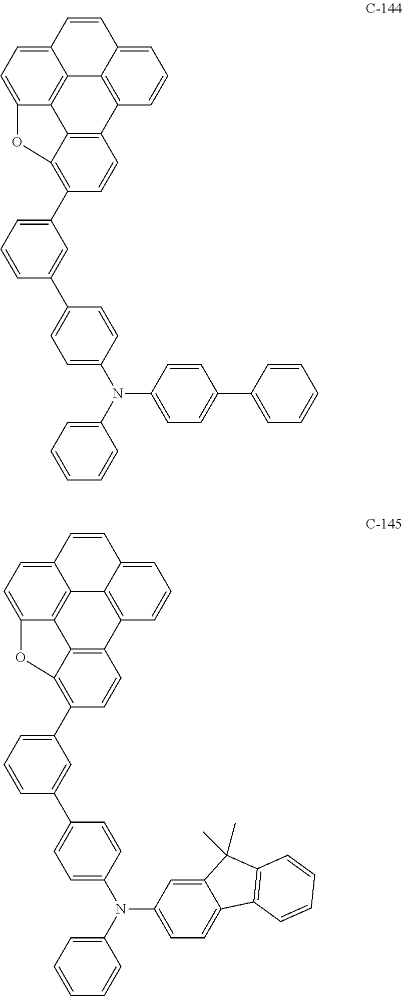 [SMILES: c1ccc(-c2ccc(N(c3ccccc3)c3ccc(-c4cccc(-c5ccc6c7cccc8ccc9ccc%10oc5c6c%10c9c87)c4)cc3)cc2)cc1 ; CC1(C)c2ccccc2-c2ccc(N(c3ccccc3)c3ccc(-c4cccc(-c5ccc6c7cccc8ccc9ccc%10oc5c6c%10c9c87)c4)cc3)cc21]

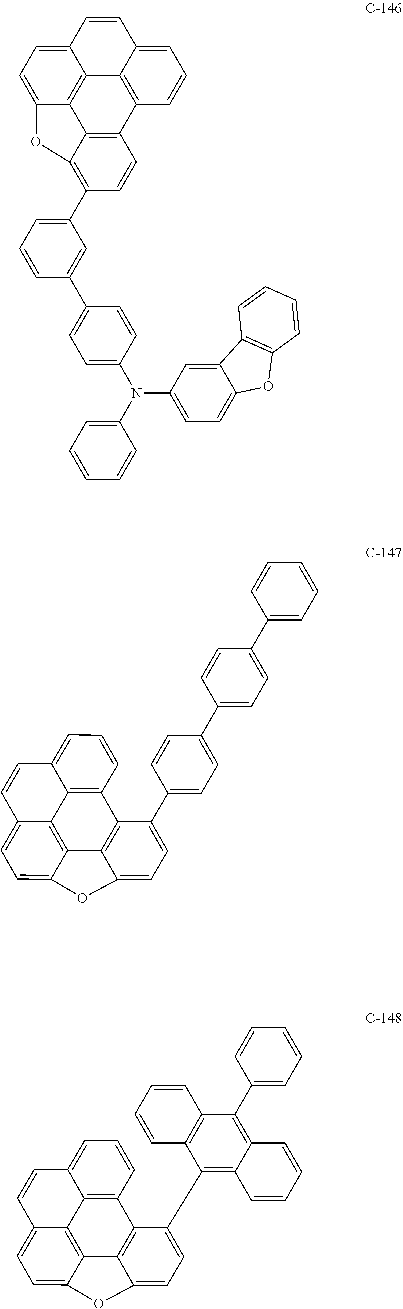 [SMILES: c1ccc(N(c2ccc(-c3cccc(-c4ccc5c6cccc7ccc8ccc9oc4c5c9c8c76)c3)cc2)c2ccc3oc4ccccc4c3c2)cc1 ; c1ccc(-c2ccc(-c3ccc(-c4ccc5oc6ccc7ccc8cccc9c4c5c6c7c89)cc3)cc2)cc1 ; c1ccc(-c2c3ccccc3c(-c3ccc4oc5ccc6ccc7cccc8c3c4c5c6c78)c3ccccc23)cc1]

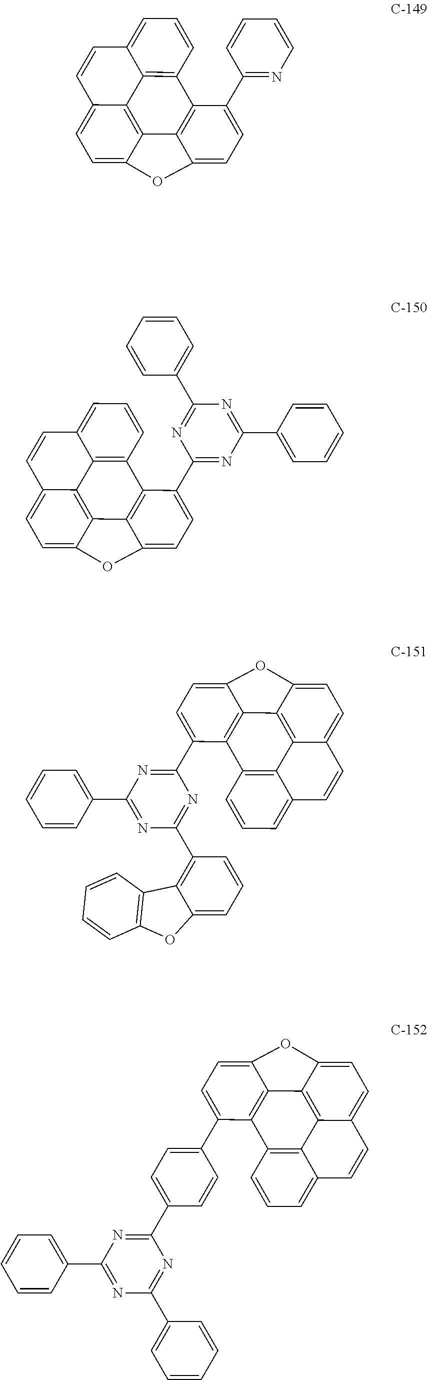 [SMILES: c1ccc(-c2ccc3oc4ccc5ccc6cccc7c2c3c4c5c67)nc1 ; c1ccc(-c2nc(-c3ccccc3)nc(-c3ccc4oc5ccc6ccc7cccc8c3c4c5c6c78)n2)cc1 ; c1ccc(-c2nc(-c3cccc4oc5ccccc5c34)nc(-c3ccc4oc5ccc6ccc7cccc8c3c4c5c6c78)n2)cc1 ; c1ccc(-c2nc(-c3ccccc3)nc(-c3ccc(-c4ccc5oc6ccc7ccc8cccc9c4c5c6c7c89)cc3)n2)cc1]

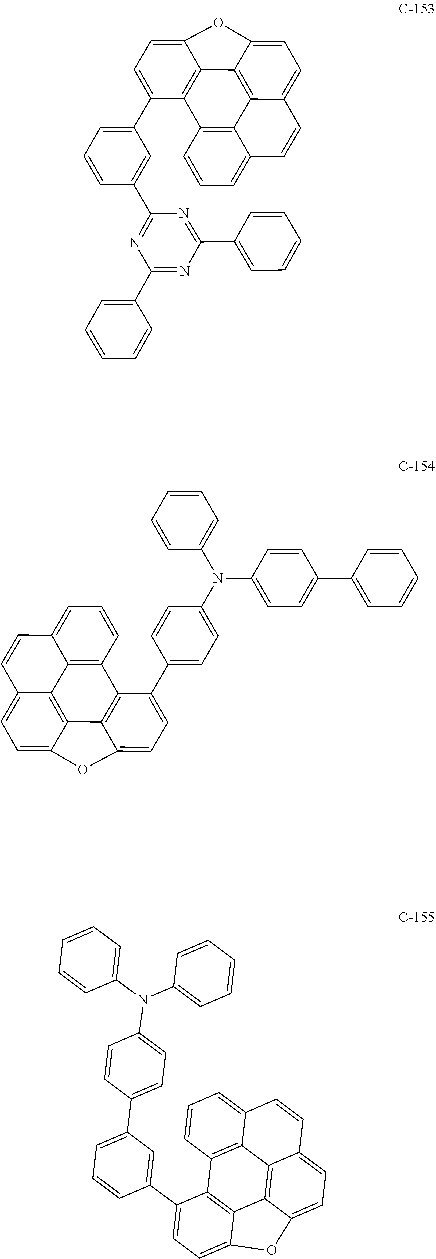 [SMILES: c1ccc(-c2nc(-c3ccccc3)nc(-c3cccc(-c4ccc5oc6ccc7ccc8cccc9c4c5c6c7c89)c3)n2)cc1 ; c1ccc(-c2ccc(N(c3ccccc3)c3ccc(-c4ccc5oc6ccc7ccc8cccc9c4c5c6c7c89)cc3)cc2)cc1 ; c1ccc(N(c2ccccc2)c2ccc(-c3cccc(-c4ccc5oc6ccc7ccc8cccc9c4c5c6c7c89)c3)cc2)cc1]

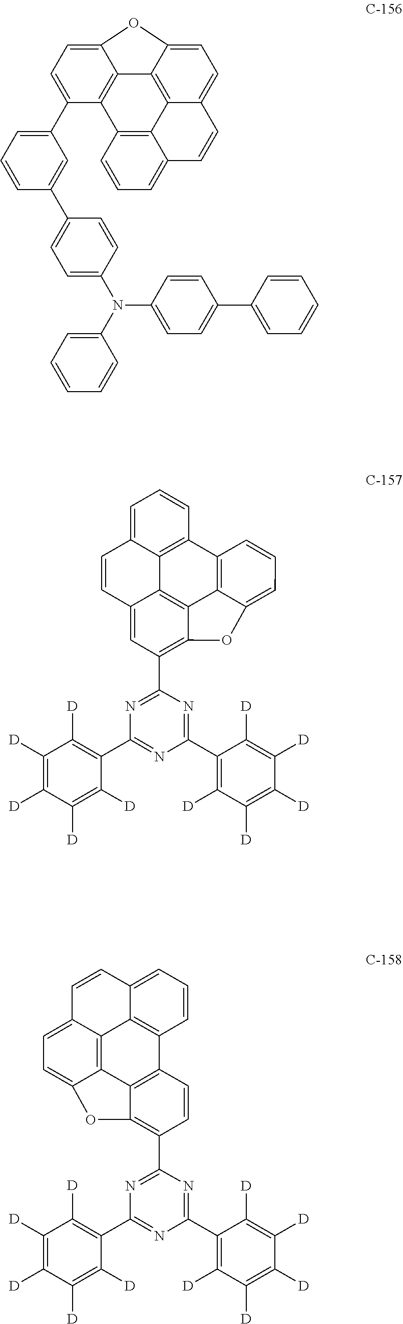 [SMILES: c1ccc(-c2ccc(N(c3ccccc3)c3ccc(-c4cccc(-c5ccc6oc7ccc8ccc9cccc%10c5c6c7c8c9%10)c4)cc3)cc2)cc1 ; [2H]c1c([2H])c([2H])c(-c2nc(-c3c([2H])c([2H])c([2H])c([2H])c3[2H])nc(-c3cc4ccc5cccc6c7cccc8oc3c(c87)c4c56)n2)c([2H])c1[2H] ; [2H]c1c([2H])c([2H])c(-c2nc(-c3c([2H])c([2H])c([2H])c([2H])c3[2H])nc(-c3ccc4c5cccc6ccc7ccc8oc3c4c8c7c65)n2)c([2H])c1[2H]]

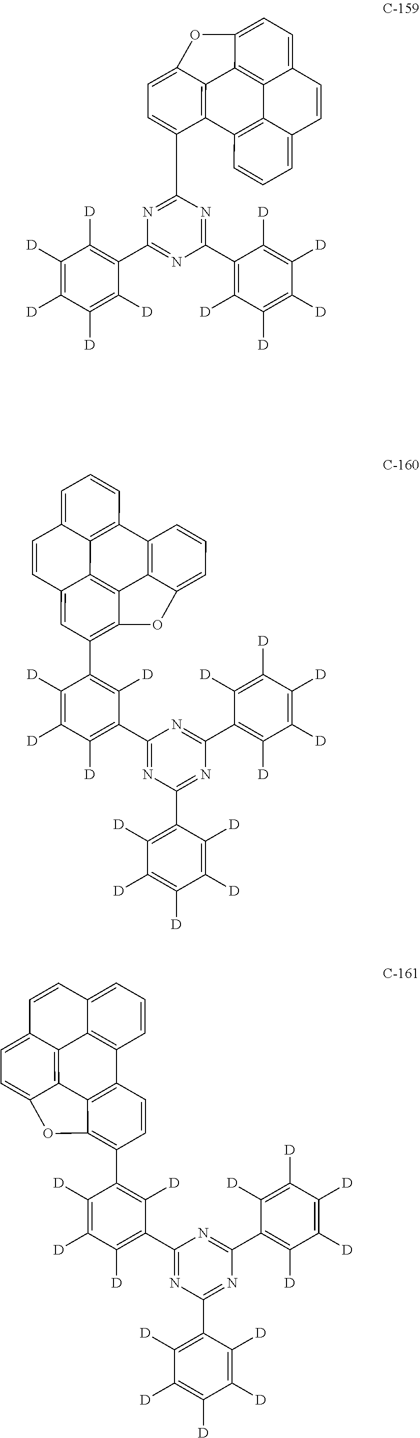 [SMILES: [2H]c1c([2H])c([2H])c(-c2nc(-c3c([2H])c([2H])c([2H])c([2H])c3[2H])nc(-c3ccc4oc5ccc6ccc7cccc8c3c4c5c6c78)n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c(-c4cc5ccc6cccc7c8cccc9oc4c(c98)c5c67)c3[2H])n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c(-c4ccc5c6cccc7ccc8ccc9oc4c5c9c8c76)c3[2H])n2)c([2H])c1[2H]]

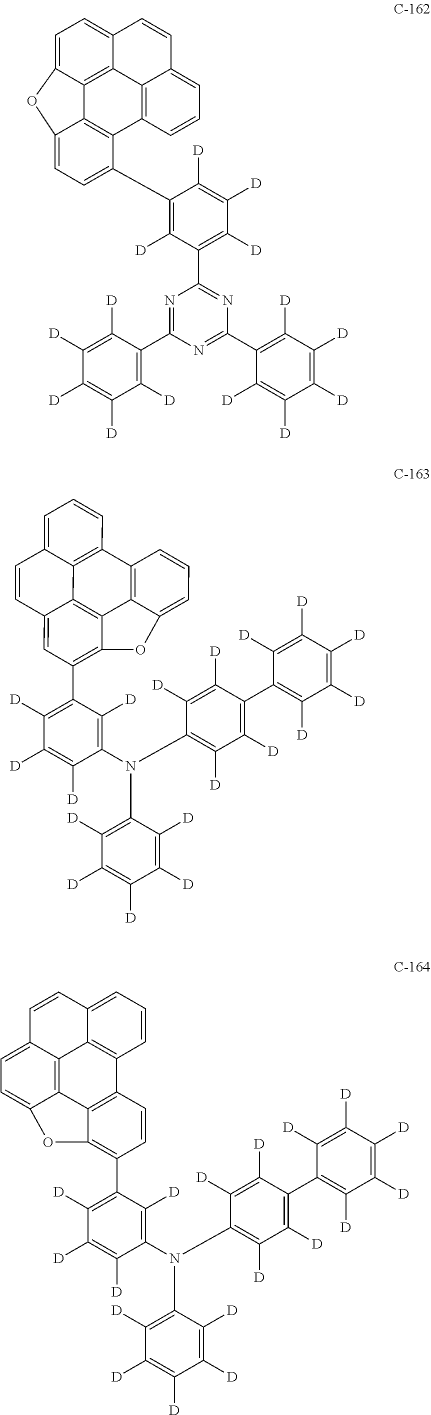

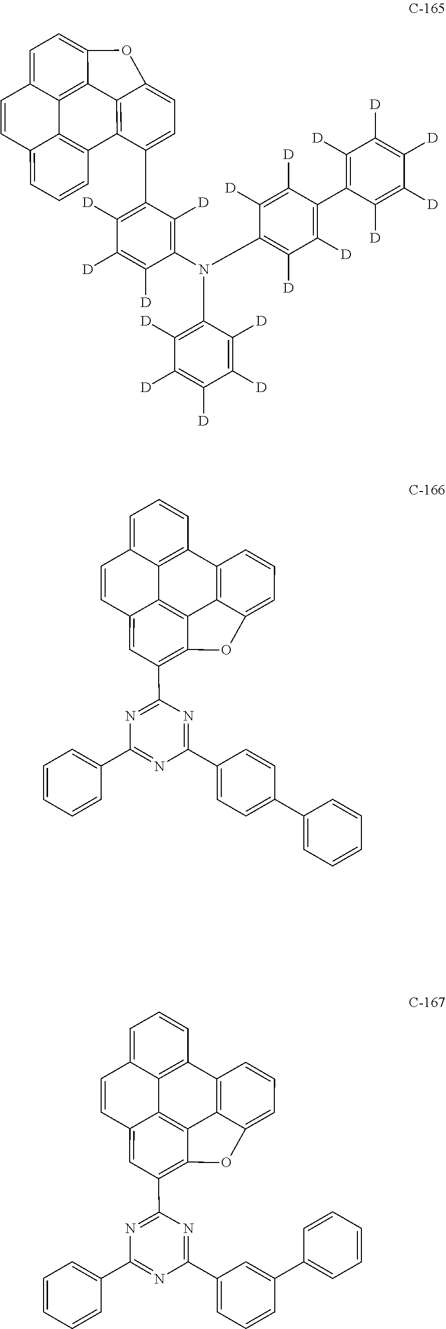 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4ccc5oc6ccc7ccc8cccc9c4c5c6c7c89)c3[2H])c([2H])c2[2H])c([2H])c1[2H] ; c1ccc(-c2ccc(-c3nc(-c4ccccc4)nc(-c4cc5ccc6cccc7c8cccc9oc4c(c98)c5c67)n3)cc2)cc1 ; c1ccc(-c2cccc(-c3nc(-c4ccccc4)nc(-c4cc5ccc6cccc7c8cccc9oc4c(c98)c5c67)n3)c2)cc1]

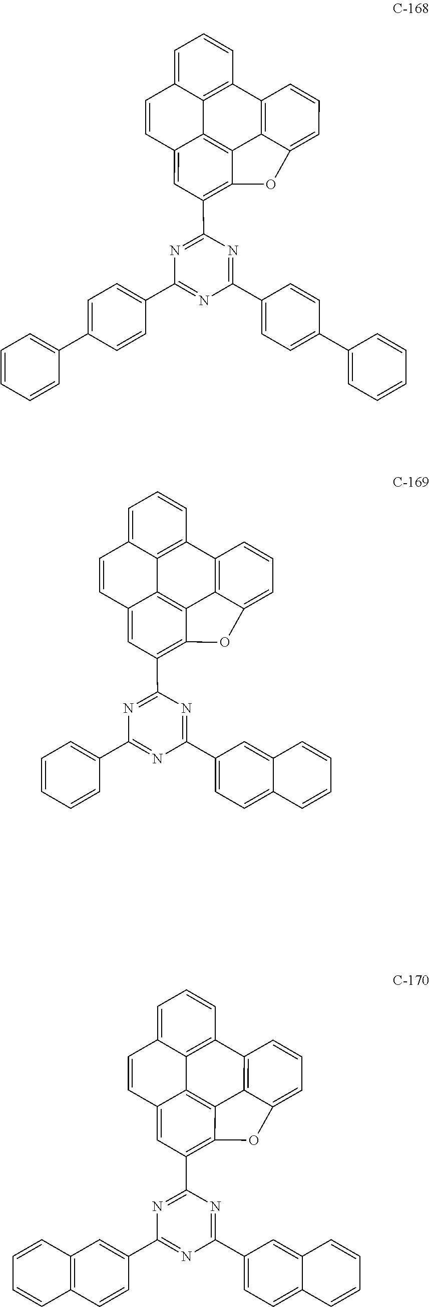 [SMILES: c1ccc(-c2ccc(-c3nc(-c4ccc(-c5ccccc5)cc4)nc(-c4cc5ccc6cccc7c8cccc9oc4c(c98)c5c67)n3)cc2)cc1 ; c1ccc(-c2nc(-c3ccc4ccccc4c3)nc(-c3cc4ccc5cccc6c7cccc8oc3c(c87)c4c56)n2)cc1 ; c1ccc2cc(-c3nc(-c4ccc5ccccc5c4)nc(-c4cc5ccc6cccc7c8cccc9oc4c(c98)c5c67)n3)ccc2c1]

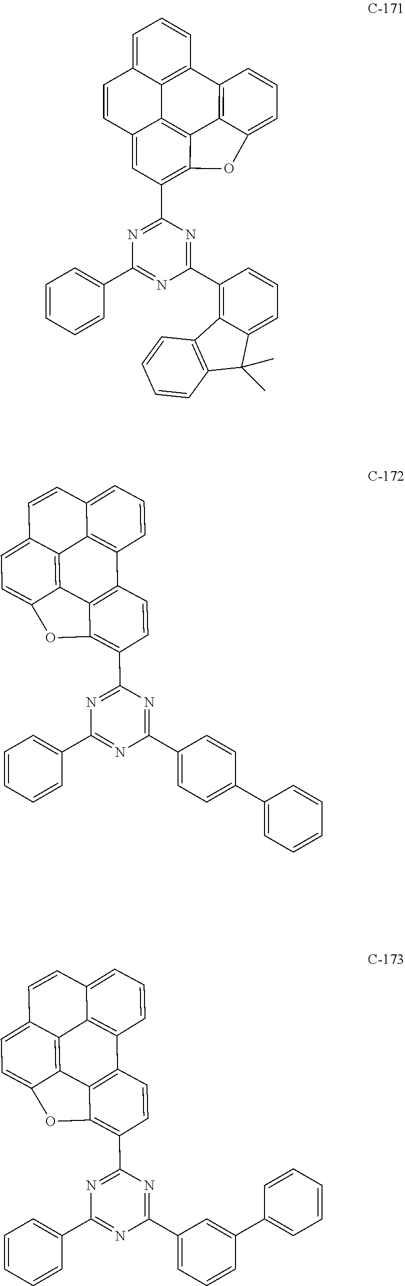 [SMILES: CC1(C)c2ccccc2-c2c(-c3nc(-c4ccccc4)nc(-c4cc5ccc6cccc7c8cccc9oc4c(c98)c5c67)n3)cccc21 ; c1ccc(-c2ccc(-c3nc(-c4ccccc4)nc(-c4ccc5c6cccc7ccc8ccc9oc4c5c9c8c76)n3)cc2)cc1 ; c1ccc(-c2cccc(-c3nc(-c4ccccc4)nc(-c4ccc5c6cccc7ccc8ccc9oc4c5c9c8c76)n3)c2)cc1]

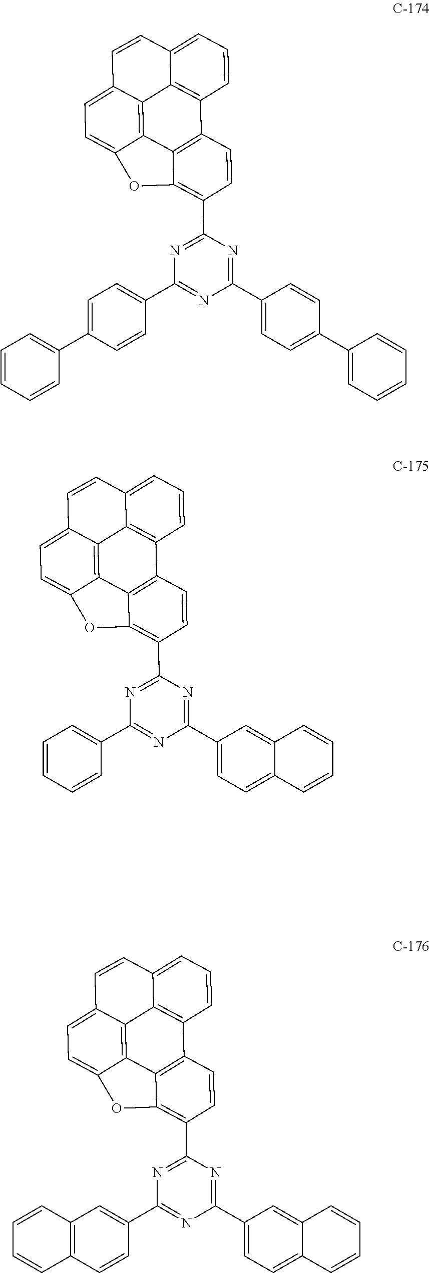 [SMILES: c1ccc(-c2ccc(-c3nc(-c4ccc(-c5ccccc5)cc4)nc(-c4ccc5c6cccc7ccc8ccc9oc4c5c9c8c76)n3)cc2)cc1 ; c1ccc(-c2nc(-c3ccc4ccccc4c3)nc(-c3ccc4c5cccc6ccc7ccc8oc3c4c8c7c65)n2)cc1 ; c1ccc2cc(-c3nc(-c4ccc5ccccc5c4)nc(-c4ccc5c6cccc7ccc8ccc9oc4c5c9c8c76)n3)ccc2c1]

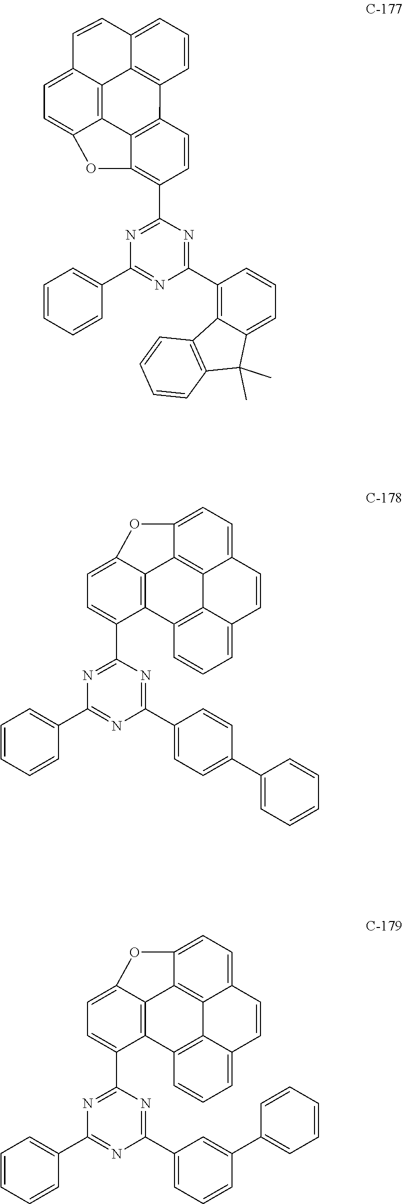 [SMILES: CC1(C)c2ccccc2-c2c(-c3nc(-c4ccccc4)nc(-c4ccc5c6cccc7ccc8ccc9oc4c5c9c8c76)n3)cccc21 ; c1ccc(-c2ccc(-c3nc(-c4ccccc4)nc(-c4ccc5oc6ccc7ccc8cccc9c4c5c6c7c89)n3)cc2)cc1 ; c1ccc(-c2cccc(-c3nc(-c4ccccc4)nc(-c4ccc5oc6ccc7ccc8cccc9c4c5c6c7c89)n3)c2)cc1]

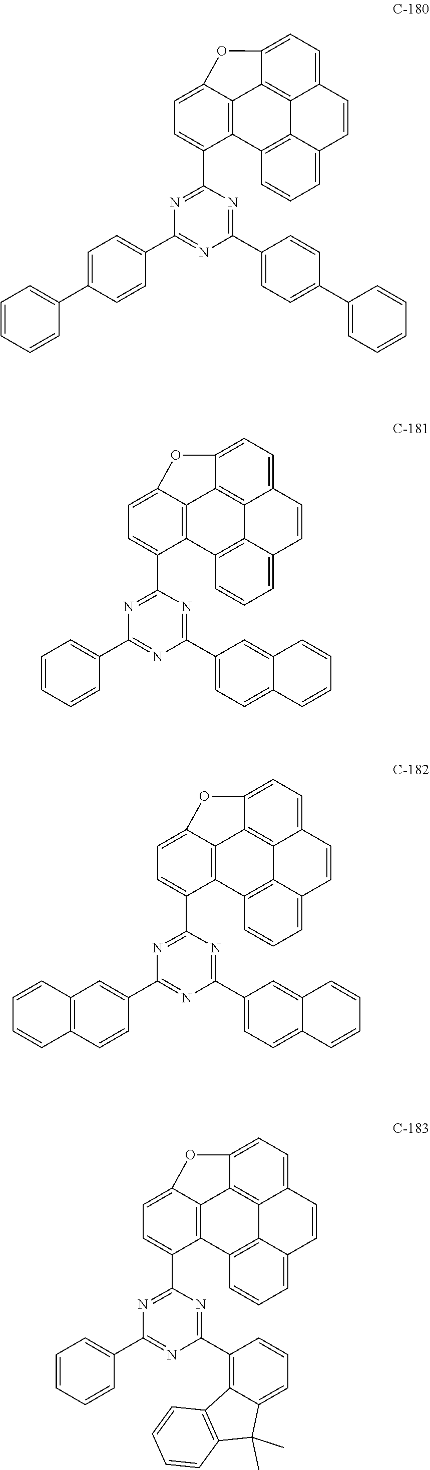 [SMILES: c1ccc(-c2ccc(-c3nc(-c4ccc(-c5ccccc5)cc4)nc(-c4ccc5oc6ccc7ccc8cccc9c4c5c6c7c89)n3)cc2)cc1 ; c1ccc(-c2nc(-c3ccc4ccccc4c3)nc(-c3ccc4oc5ccc6ccc7cccc8c3c4c5c6c78)n2)cc1 ; c1ccc2cc(-c3nc(-c4ccc5ccccc5c4)nc(-c4ccc5oc6ccc7ccc8cccc9c4c5c6c7c89)n3)ccc2c1 ; CC1(C)c2ccccc2-c2c(-c3nc(-c4ccccc4)nc(-c4ccc5oc6ccc7ccc8cccc9c4c5c6c7c89)n3)cccc21]

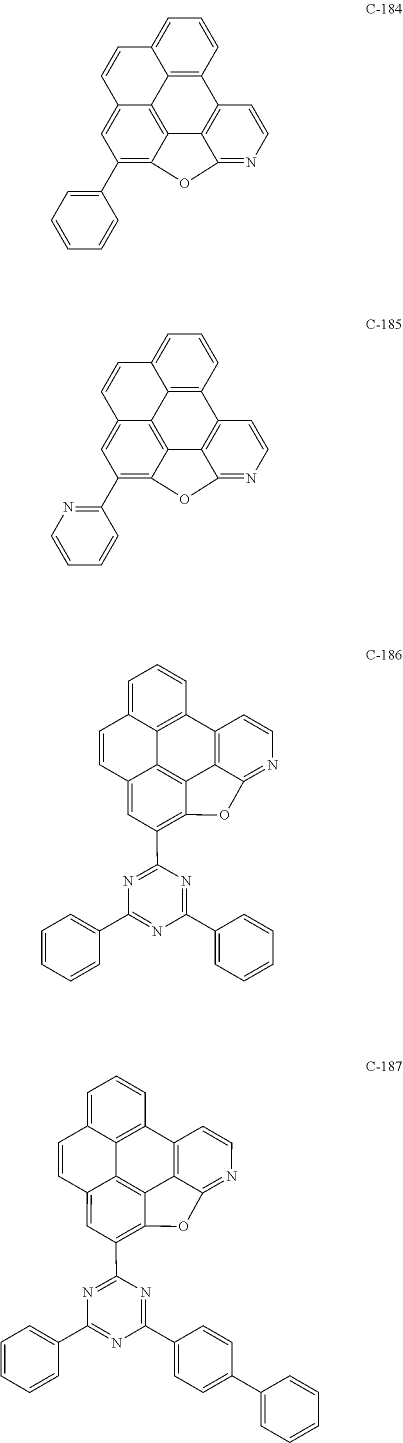 [SMILES: c1ccc(-c2cc3ccc4cccc5c6ccnc7oc2c(c76)c3c45)cc1 ; c1ccc(-c2cc3ccc4cccc5c6ccnc7oc2c(c76)c3c45)nc1 ; c1ccc(-c2nc(-c3ccccc3)nc(-c3cc4ccc5cccc6c7ccnc8oc3c(c87)c4c56)n2)cc1 ; c1ccc(-c2ccc(-c3nc(-c4ccccc4)nc(-c4cc5ccc6cccc7c8ccnc9oc4c(c98)c5c67)n3)cc2)cc1]

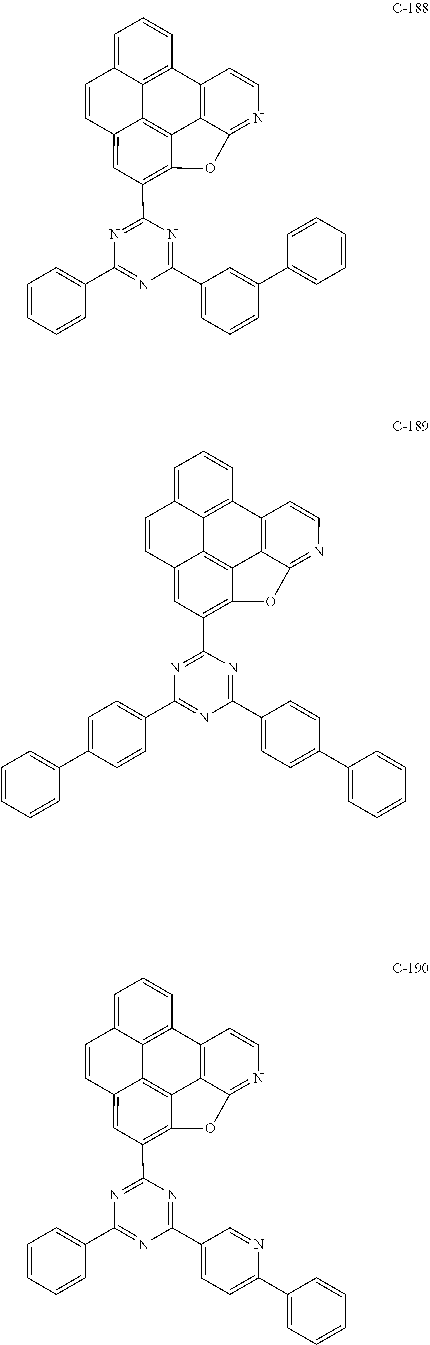 [SMILES: c1ccc(-c2cccc(-c3nc(-c4ccccc4)nc(-c4cc5ccc6cccc7c8ccnc9oc4c(c98)c5c67)n3)c2)cc1 ; c1ccc(-c2ccc(-c3nc(-c4ccc(-c5ccccc5)cc4)nc(-c4cc5ccc6cccc7c8ccnc9oc4c(c98)c5c67)n3)cc2)cc1 ; c1ccc(-c2ccc(-c3nc(-c4ccccc4)nc(-c4cc5ccc6cccc7c8ccnc9oc4c(c98)c5c67)n3)cn2)cc1]

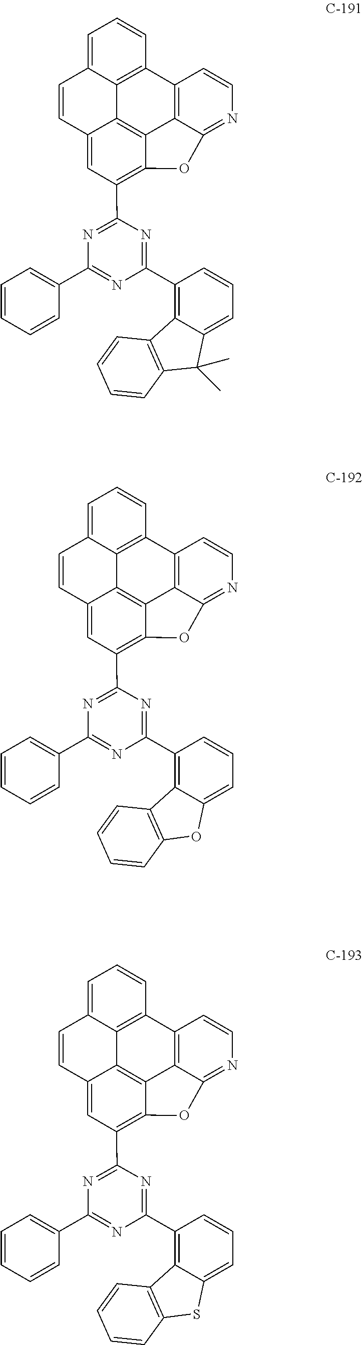 [SMILES: CC1(C)c2ccccc2-c2c(-c3nc(-c4ccccc4)nc(-c4cc5ccc6cccc7c8ccnc9oc4c(c98)c5c67)n3)cccc21 ; c1ccc(-c2nc(-c3cc4ccc5cccc6c7ccnc8oc3c(c87)c4c56)nc(-c3cccc4oc5ccccc5c34)n2)cc1 ; c1ccc(-c2nc(-c3cc4ccc5cccc6c7ccnc8oc3c(c87)c4c56)nc(-c3cccc4sc5ccccc5c34)n2)cc1]

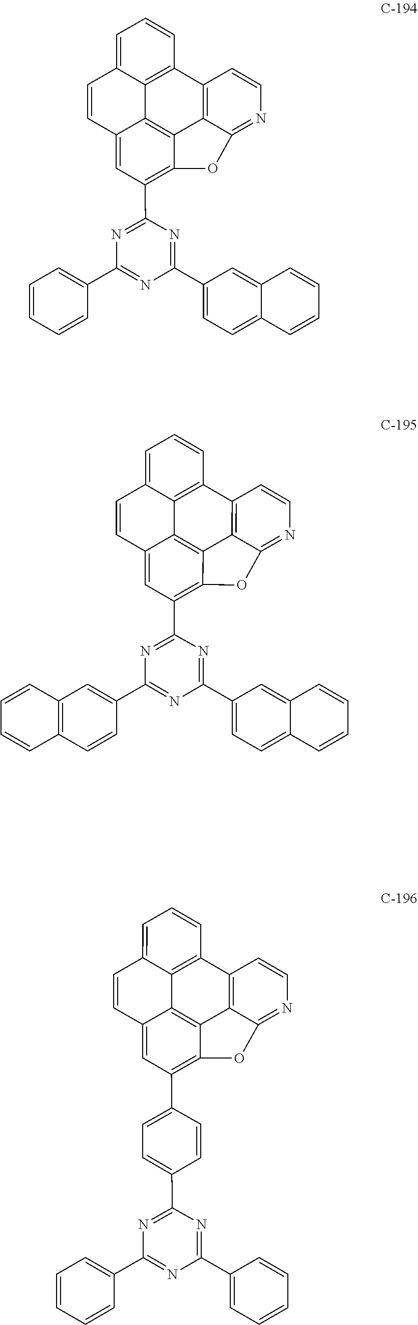 [SMILES: c1ccc(-c2nc(-c3ccc4ccccc4c3)nc(-c3cc4ccc5cccc6c7ccnc8oc3c(c87)c4c56)n2)cc1 ; c1ccc2cc(-c3nc(-c4ccc5ccccc5c4)nc(-c4cc5ccc6cccc7c8ccnc9oc4c(c98)c5c67)n3)ccc2c1 ; c1ccc(-c2nc(-c3ccccc3)nc(-c3ccc(-c4cc5ccc6cccc7c8ccnc9oc4c(c98)c5c67)cc3)n2)cc1]

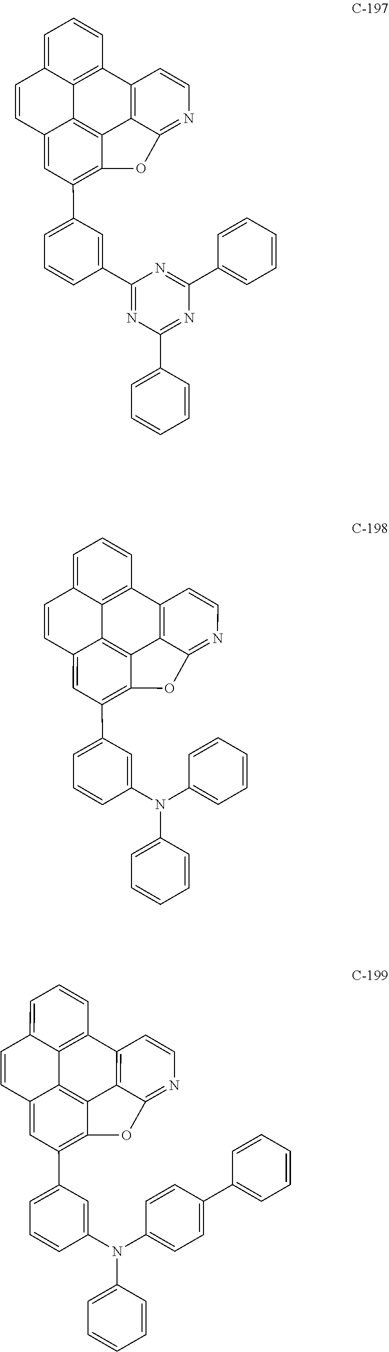 [SMILES: c1ccc(-c2nc(-c3ccccc3)nc(-c3cccc(-c4cc5ccc6cccc7c8ccnc9oc4c(c98)c5c67)c3)n2)cc1 ; c1ccc(N(c2ccccc2)c2cccc(-c3cc4ccc5cccc6c7ccnc8oc3c(c87)c4c56)c2)cc1 ; c1ccc(-c2ccc(N(c3ccccc3)c3cccc(-c4cc5ccc6cccc7c8ccnc9oc4c(c98)c5c67)c3)cc2)cc1]

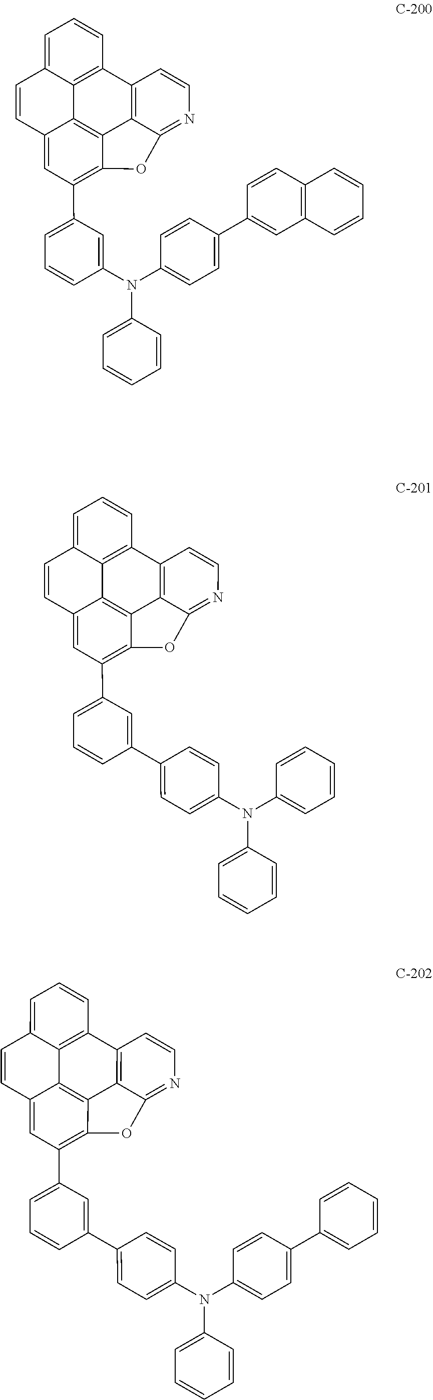 [SMILES: c1ccc(N(c2ccc(-c3ccc4ccccc4c3)cc2)c2cccc(-c3cc4ccc5cccc6c7ccnc8oc3c(c87)c4c56)c2)cc1 ; c1ccc(N(c2ccccc2)c2ccc(-c3cccc(-c4cc5ccc6cccc7c8ccnc9oc4c(c98)c5c67)c3)cc2)cc1 ; c1ccc(-c2ccc(N(c3ccccc3)c3ccc(-c4cccc(-c5cc6ccc7cccc8c9ccnc%10oc5c(c%109)c6c78)c4)cc3)cc2)cc1]

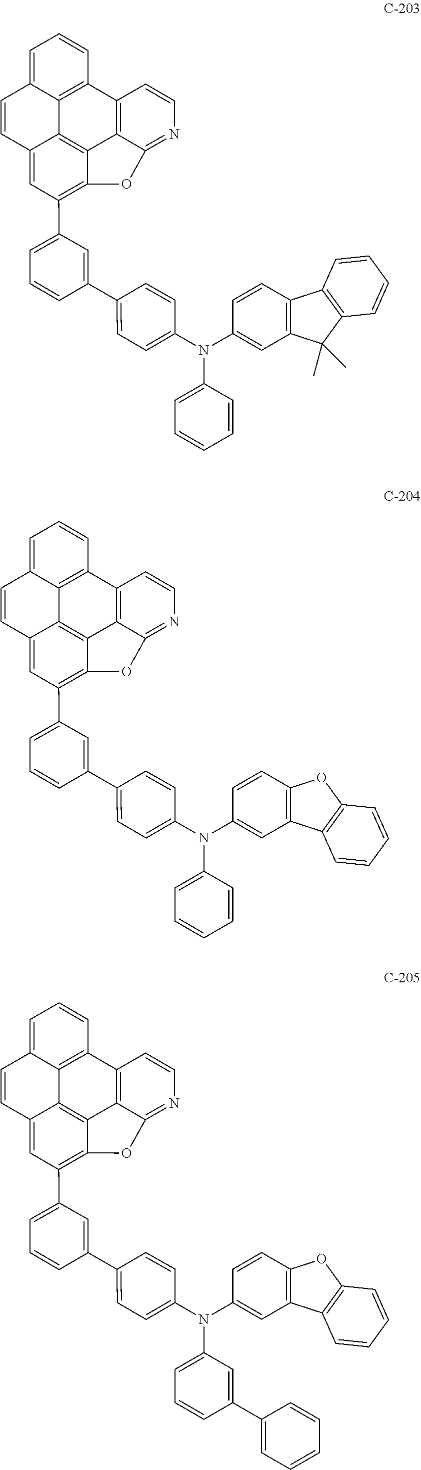 [SMILES: CC1(C)c2ccccc2-c2ccc(N(c3ccccc3)c3ccc(-c4cccc(-c5cc6ccc7cccc8c9ccnc%10oc5c(c%109)c6c78)c4)cc3)cc21 ; c1ccc(N(c2ccc(-c3cccc(-c4cc5ccc6cccc7c8ccnc9oc4c(c98)c5c67)c3)cc2)c2ccc3oc4ccccc4c3c2)cc1 ; c1ccc(-c2cccc(N(c3ccc(-c4cccc(-c5cc6ccc7cccc8c9ccnc%10oc5c(c%109)c6c78)c4)cc3)c3ccc4oc5ccccc5c4c3)c2)cc1]

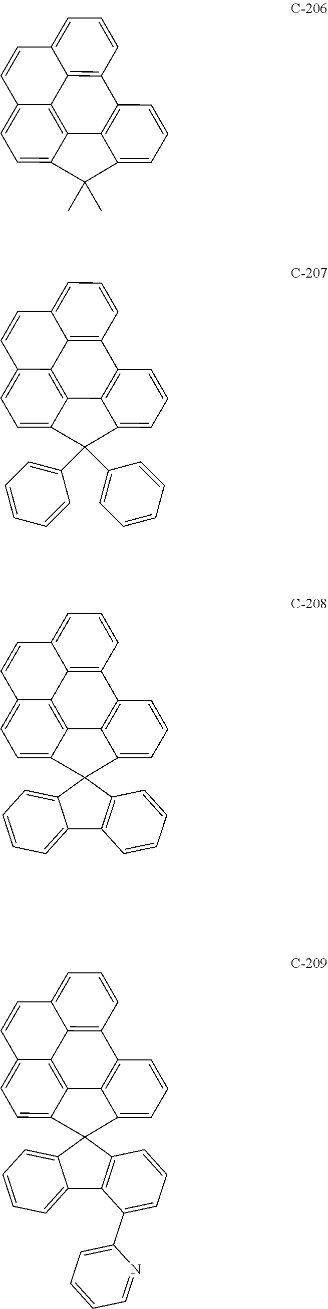

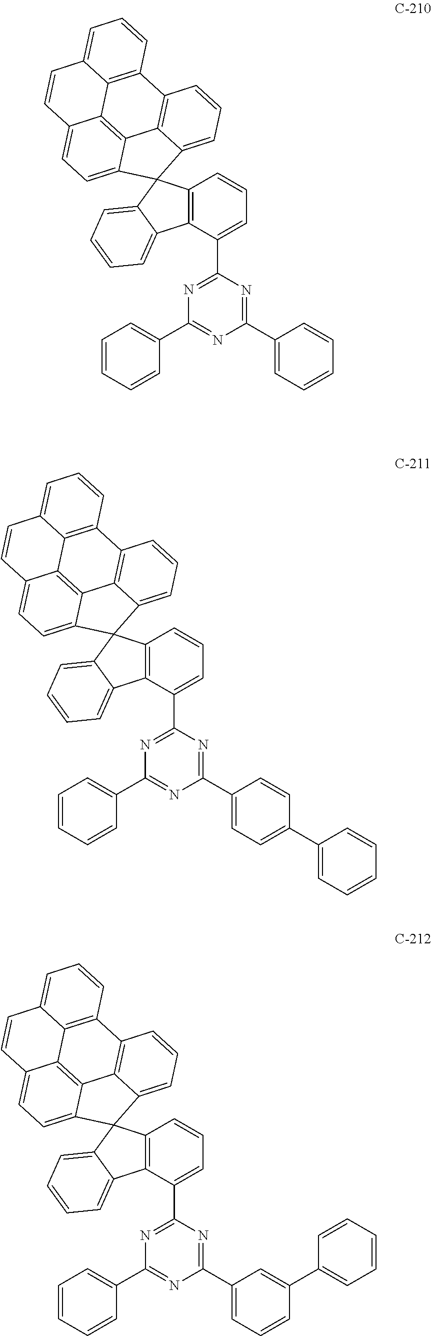 [SMILES: c1ccc(-c2nc(-c3ccccc3)nc(-c3cccc4c3-c3ccccc3C43c4cccc5c6cccc7ccc8ccc3c(c45)c8c76)n2)cc1 ; c1ccc(-c2ccc(-c3nc(-c4ccccc4)nc(-c4cccc5c4-c4ccccc4C54c5cccc6c7cccc8ccc9ccc4c(c56)c9c87)n3)cc2)cc1 ; c1ccc(-c2cccc(-c3nc(-c4ccccc4)nc(-c4cccc5c4-c4ccccc4C54c5cccc6c7cccc8ccc9ccc4c(c56)c9c87)n3)c2)cc1]

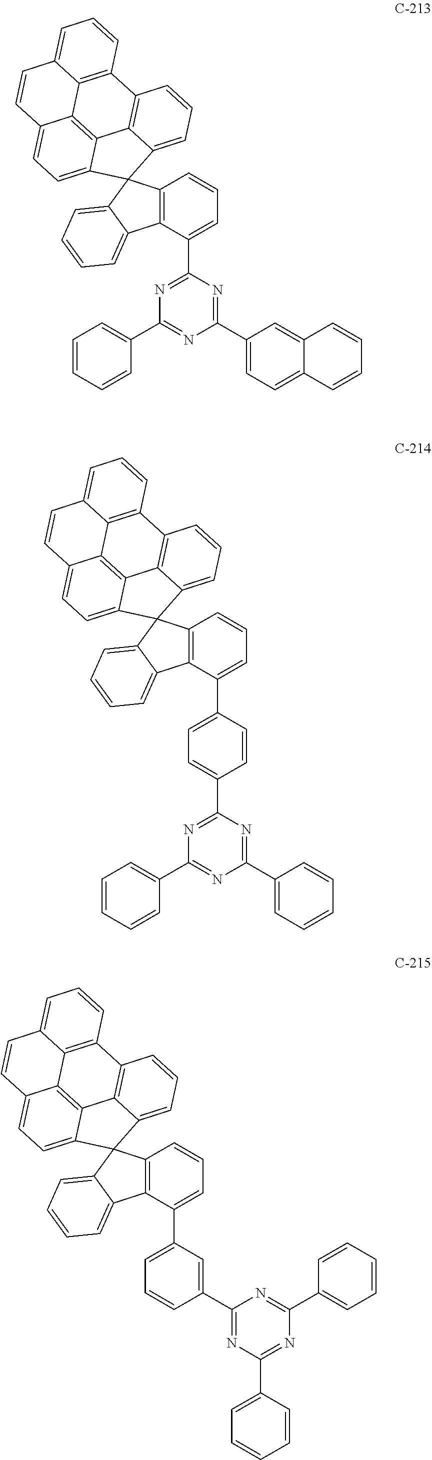 [SMILES: c1ccc(-c2nc(-c3ccc4ccccc4c3)nc(-c3cccc4c3-c3ccccc3C43c4cccc5c6cccc7ccc8ccc3c(c45)c8c76)n2)cc1 ; c1ccc(-c2nc(-c3ccccc3)nc(-c3ccc(-c4cccc5c4-c4ccccc4C54c5cccc6c7cccc8ccc9ccc4c(c56)c9c87)cc3)n2)cc1 ; c1ccc(-c2nc(-c3ccccc3)nc(-c3cccc(-c4cccc5c4-c4ccccc4C54c5cccc6c7cccc8ccc9ccc4c(c56)c9c87)c3)n2)cc1]

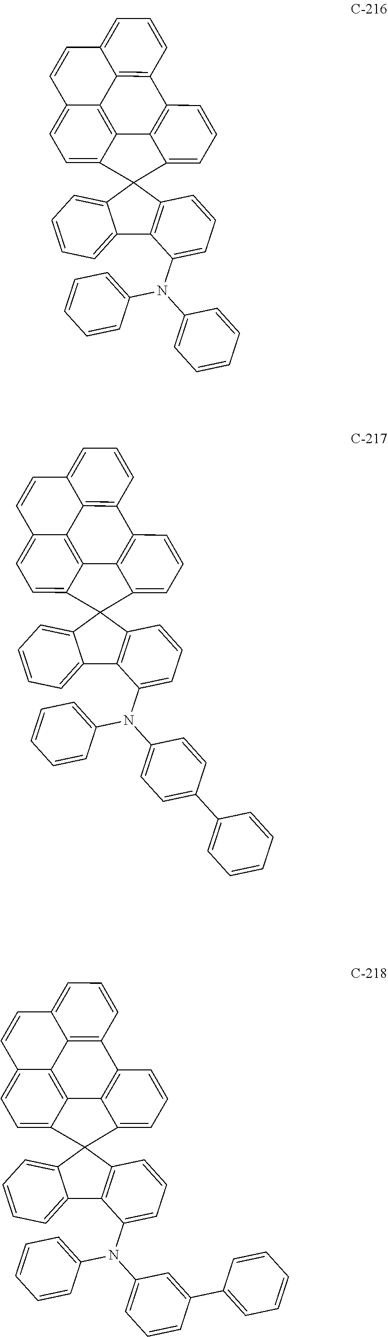 [SMILES: c1ccc(N(c2ccccc2)c2cccc3c2-c2ccccc2C32c3cccc4c5cccc6ccc7ccc2c(c34)c7c65)cc1 ; c1ccc(-c2ccc(N(c3ccccc3)c3cccc4c3-c3ccccc3C43c4cccc5c6cccc7ccc8ccc3c(c45)c8c76)cc2)cc1 ; c1ccc(-c2cccc(N(c3ccccc3)c3cccc4c3-c3ccccc3C43c4cccc5c6cccc7ccc8ccc3c(c45)c8c76)c2)cc1]

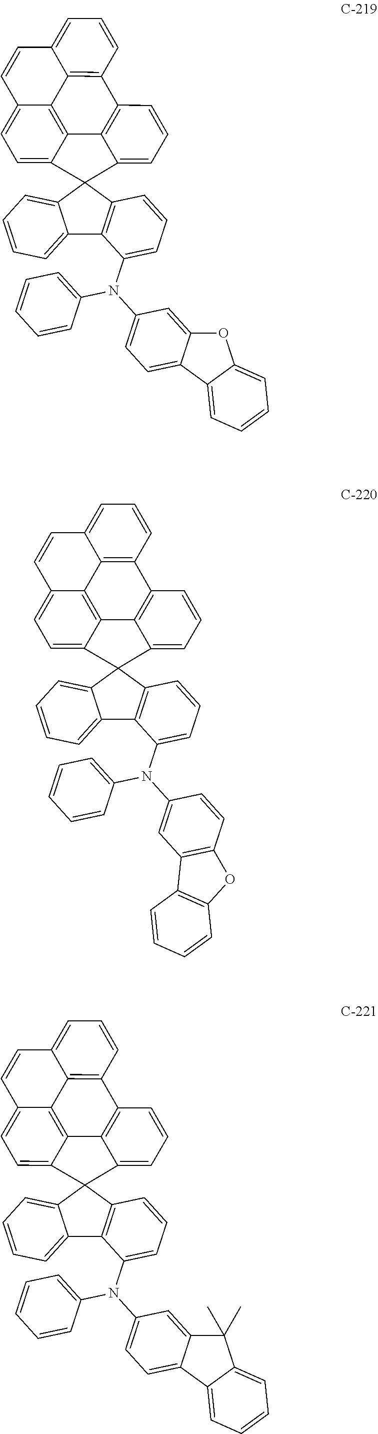 [SMILES: c1ccc(N(c2ccc3c(c2)oc2ccccc23)c2cccc3c2-c2ccccc2C32c3cccc4c5cccc6ccc7ccc2c(c34)c7c65)cc1 ; c1ccc(N(c2ccc3oc4ccccc4c3c2)c2cccc3c2-c2ccccc2C32c3cccc4c5cccc6ccc7ccc2c(c34)c7c65)cc1 ; CC1(C)c2ccccc2-c2ccc(N(c3ccccc3)c3cccc4c3-c3ccccc3C43c4cccc5c6cccc7ccc8ccc3c(c45)c8c76)cc21]

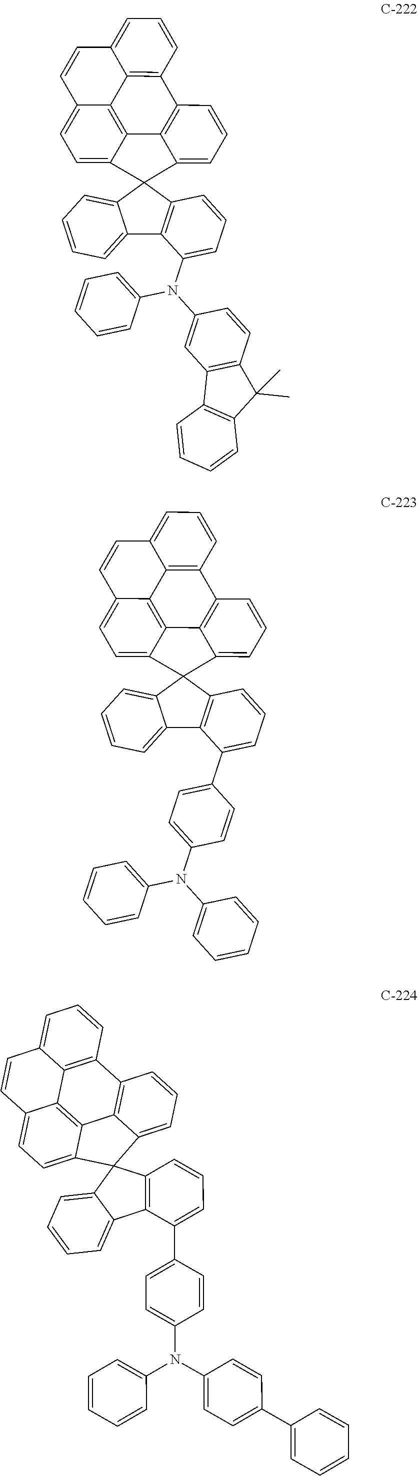

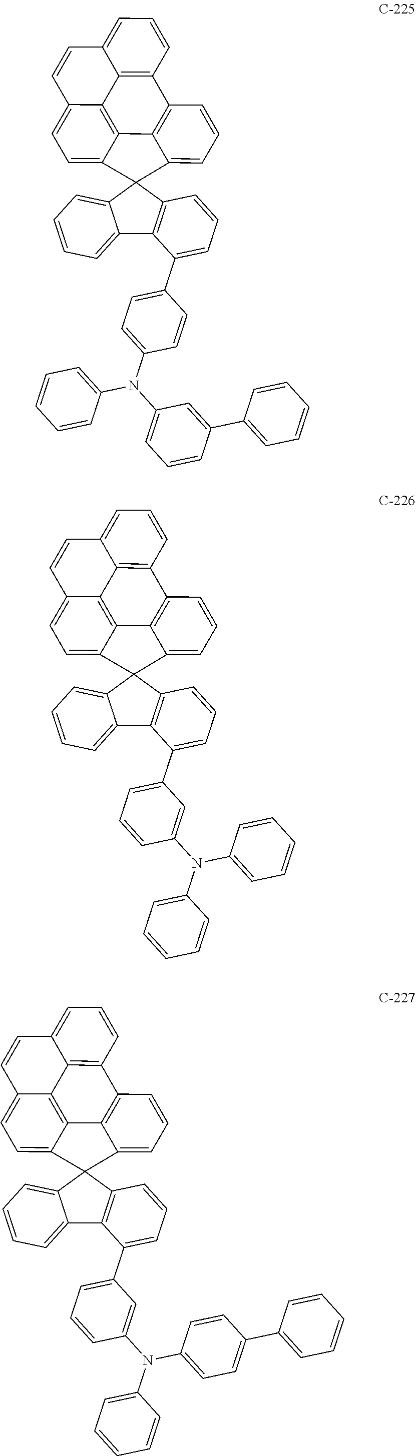 [SMILES: c1ccc(-c2cccc(N(c3ccccc3)c3ccc(-c4cccc5c4-c4ccccc4C54c5cccc6c7cccc8ccc9ccc4c(c56)c9c87)cc3)c2)cc1 ; c1ccc(N(c2ccccc2)c2cccc(-c3cccc4c3-c3ccccc3C43c4cccc5c6cccc7ccc8ccc3c(c45)c8c76)c2)cc1 ; c1ccc(-c2ccc(N(c3ccccc3)c3cccc(-c4cccc5c4-c4ccccc4C54c5cccc6c7cccc8ccc9ccc4c(c56)c9c87)c3)cc2)cc1]

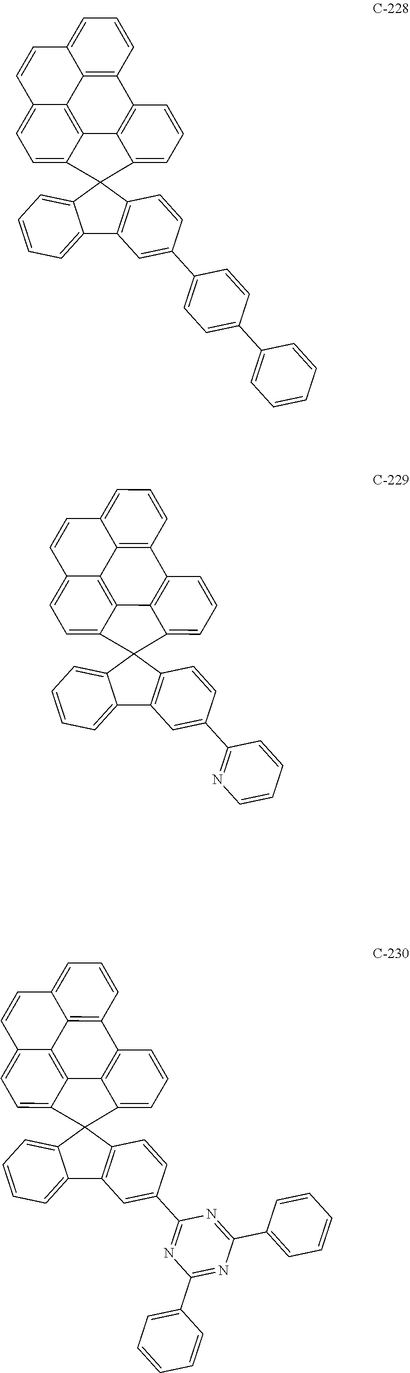

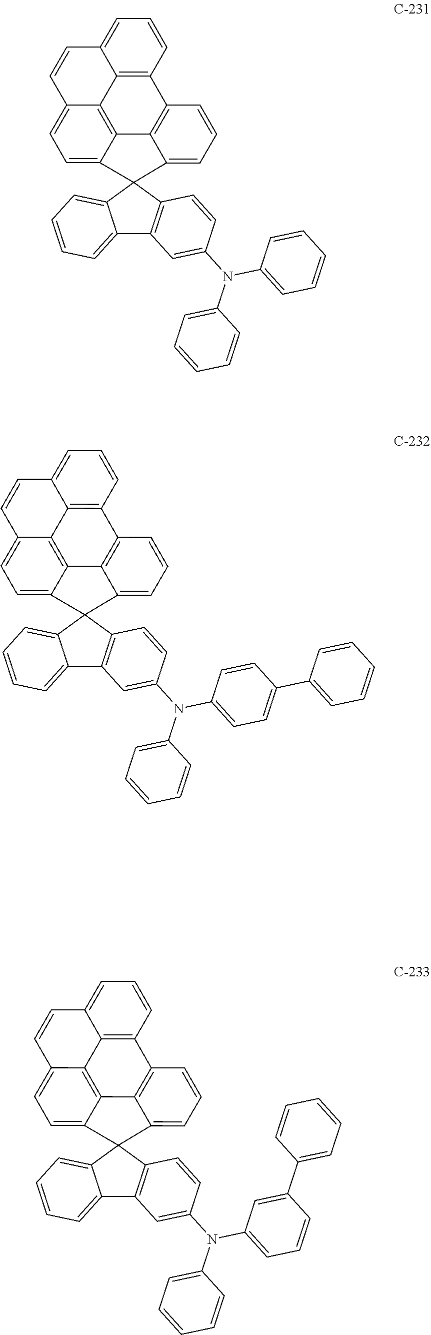 [SMILES: c1ccc(N(c2ccccc2)c2ccc3c(c2)-c2ccccc2C32c3cccc4c5cccc6ccc7ccc2c(c34)c7c65)cc1 ; c1ccc(-c2ccc(N(c3ccccc3)c3ccc4c(c3)-c3ccccc3C43c4cccc5c6cccc7ccc8ccc3c(c45)c8c76)cc2)cc1 ; c1ccc(-c2cccc(N(c3ccccc3)c3ccc4c(c3)-c3ccccc3C43c4cccc5c6cccc7ccc8ccc3c(c45)c8c76)c2)cc1]

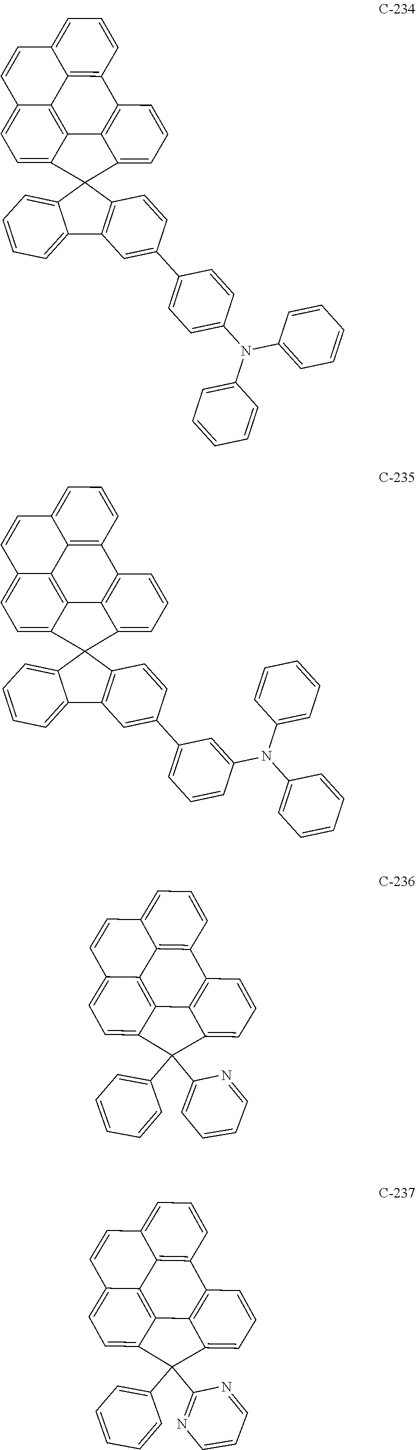 [SMILES: c1ccc(N(c2ccccc2)c2ccc(-c3ccc4c(c3)-c3ccccc3C43c4cccc5c6cccc7ccc8ccc3c(c45)c8c76)cc2)cc1 ; c1ccc(N(c2ccccc2)c2cccc(-c3ccc4c(c3)-c3ccccc3C43c4cccc5c6cccc7ccc8ccc3c(c45)c8c76)c2)cc1 ; c1ccc(C2(c3ccccn3)c3cccc4c5cccc6ccc7ccc2c(c34)c7c65)cc1 ; c1ccc(C2(c3ncccn3)c3cccc4c5cccc6ccc7ccc2c(c34)c7c65)cc1]

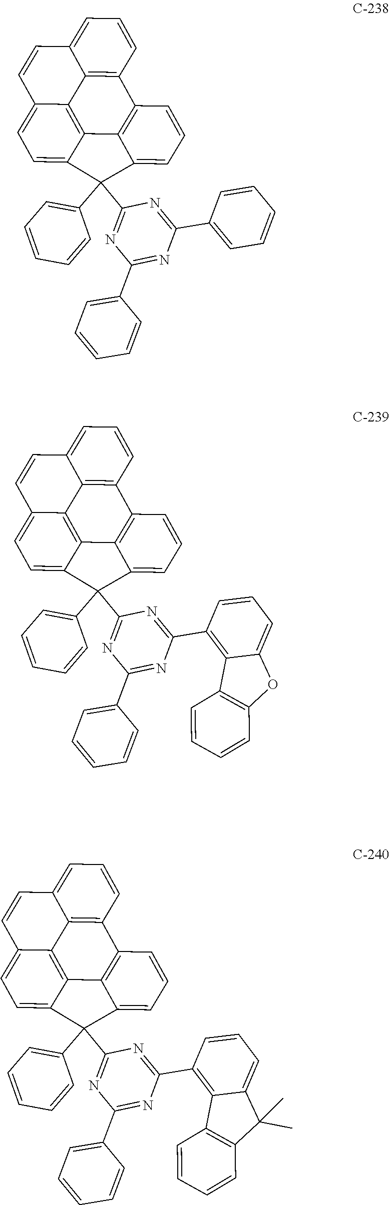 [SMILES: c1ccc(-c2nc(-c3ccccc3)nc(C3(c4ccccc4)c4cccc5c6cccc7ccc8ccc3c(c45)c8c76)n2)cc1 ; c1ccc(-c2nc(-c3cccc4oc5ccccc5c34)nc(C3(c4ccccc4)c4cccc5c6cccc7ccc8ccc3c(c45)c8c76)n2)cc1 ; CC1(C)c2ccccc2-c2c(-c3nc(-c4ccccc4)nc(C4(c5ccccc5)c5cccc6c7cccc8ccc9ccc4c(c56)c9c87)n3)cccc21]

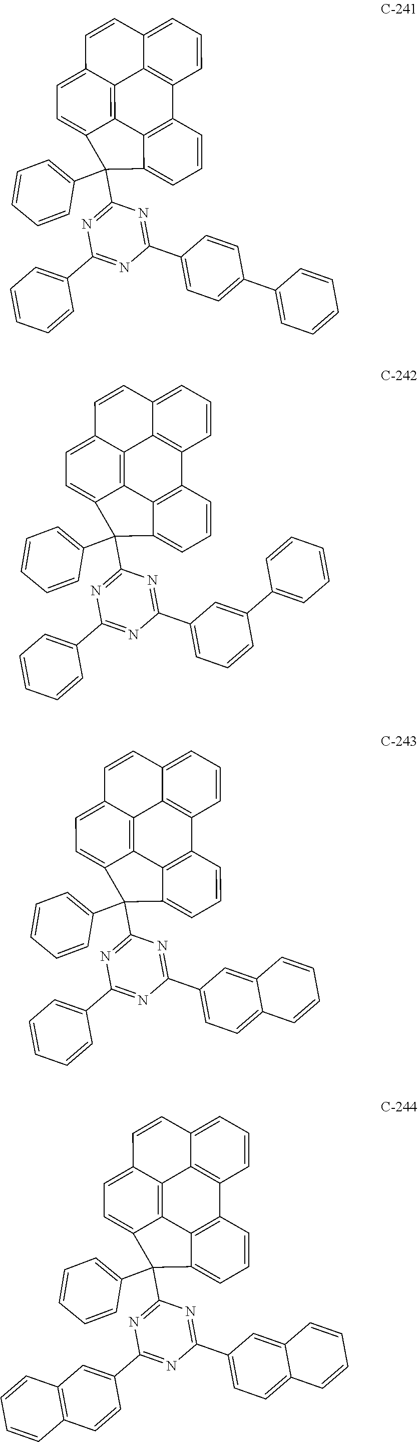 [SMILES: c1ccc(-c2ccc(-c3nc(-c4ccccc4)nc(C4(c5ccccc5)c5cccc6c7cccc8ccc9ccc4c(c56)c9c87)n3)cc2)cc1 ; c1ccc(-c2cccc(-c3nc(-c4ccccc4)nc(C4(c5ccccc5)c5cccc6c7cccc8ccc9ccc4c(c56)c9c87)n3)c2)cc1 ; c1ccc(-c2nc(-c3ccc4ccccc4c3)nc(C3(c4ccccc4)c4cccc5c6cccc7ccc8ccc3c(c45)c8c76)n2)cc1 ; c1ccc(C2(c3nc(-c4ccc5ccccc5c4)nc(-c4ccc5ccccc5c4)n3)c3cccc4c5cccc6ccc7ccc2c(c34)c7c65)cc1]

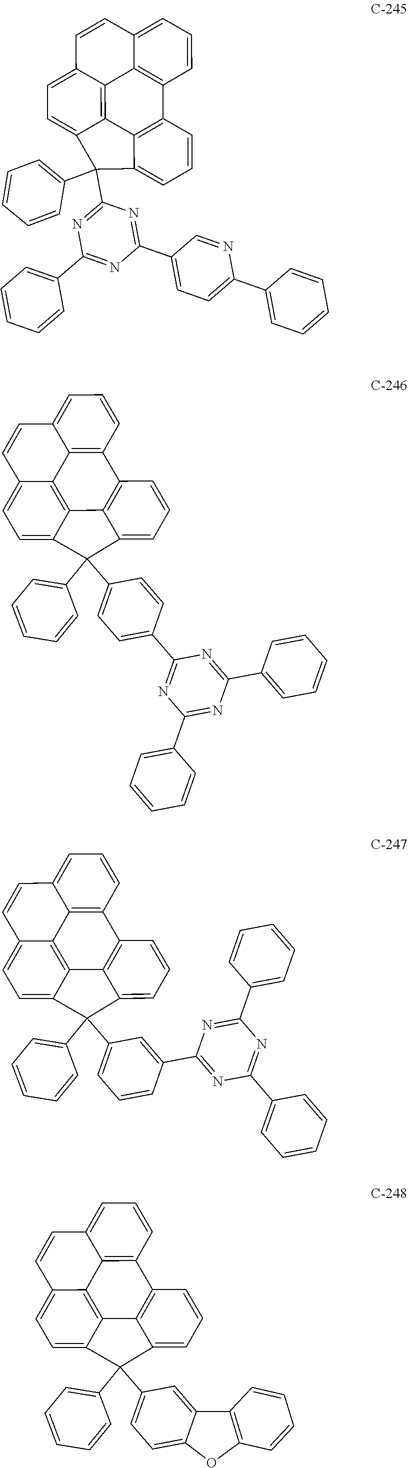 [SMILES: c1ccc(-c2ccc(-c3nc(-c4ccccc4)nc(C4(c5ccccc5)c5cccc6c7cccc8ccc9ccc4c(c56)c9c87)n3)cn2)cc1 ; c1ccc(-c2nc(-c3ccccc3)nc(-c3ccc(C4(c5ccccc5)c5cccc6c7cccc8ccc9ccc4c(c56)c9c87)cc3)n2)cc1 ; c1ccc(-c2nc(-c3ccccc3)nc(-c3cccc(C4(c5ccccc5)c5cccc6c7cccc8ccc9ccc4c(c56)c9c87)c3)n2)cc1 ; c1ccc(C2(c3ccc4oc5ccccc5c4c3)c3cccc4c5cccc6ccc7ccc2c(c34)c7c65)cc1]

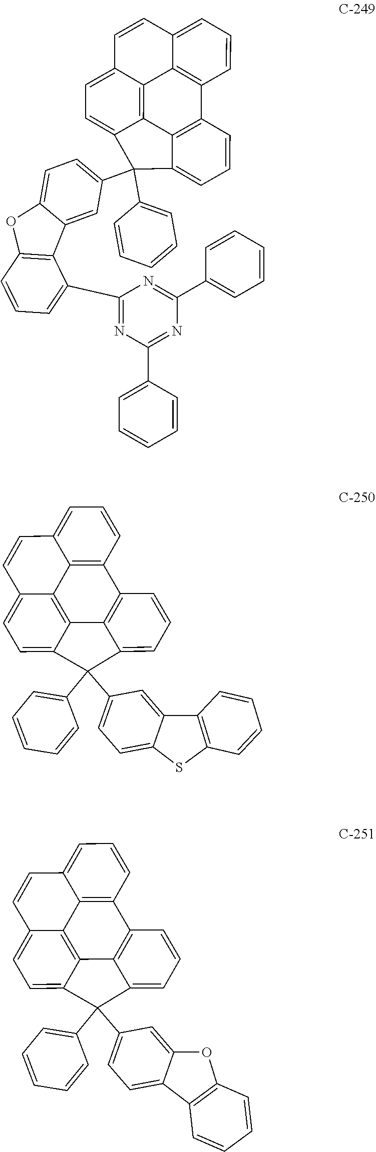 [SMILES: c1ccc(-c2nc(-c3ccccc3)nc(-c3cccc4oc5ccc(C6(c7ccccc7)c7cccc8c9cccc%10ccc%11ccc6c(c78)c%11c%109)cc5c34)n2)cc1 ; c1ccc(C2(c3ccc4sc5ccccc5c4c3)c3cccc4c5cccc6ccc7ccc2c(c34)c7c65)cc1 ; c1ccc(C2(c3ccc4c(c3)oc3ccccc34)c3cccc4c5cccc6ccc7ccc2c(c34)c7c65)cc1]

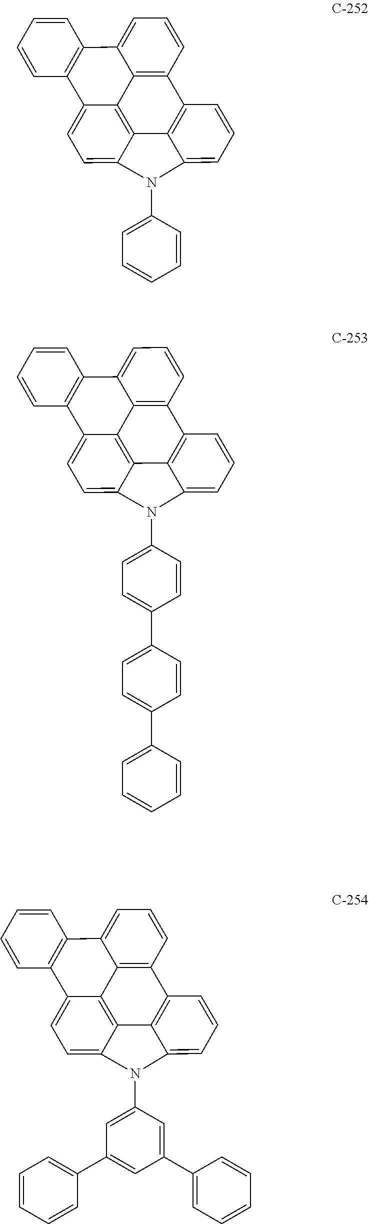 [SMILES: c1ccc(-n2c3cccc4c5cccc6c7ccccc7c7ccc2c(c7c65)c43)cc1 ; c1ccc(-c2ccc(-c3ccc(-n4c5cccc6c7cccc8c9ccccc9c9ccc4c(c9c87)c65)cc3)cc2)cc1 ; c1ccc(-c2cc(-c3ccccc3)cc(-n3c4cccc5c6cccc7c8ccccc8c8ccc3c(c8c76)c54)c2)cc1]

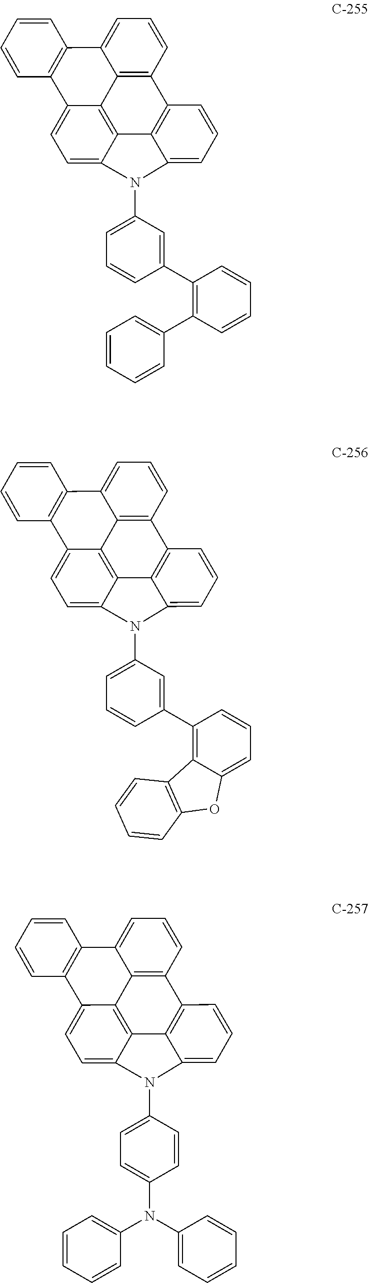 [SMILES: c1ccc(-c2ccccc2-c2cccc(-n3c4cccc5c6cccc7c8ccccc8c8ccc3c(c8c76)c54)c2)cc1 ; c1cc(-c2cccc3oc4ccccc4c23)cc(-n2c3cccc4c5cccc6c7ccccc7c7ccc2c(c7c65)c43)c1 ; c1ccc(N(c2ccccc2)c2ccc(-n3c4cccc5c6cccc7c8ccccc8c8ccc3c(c8c76)c54)cc2)cc1]

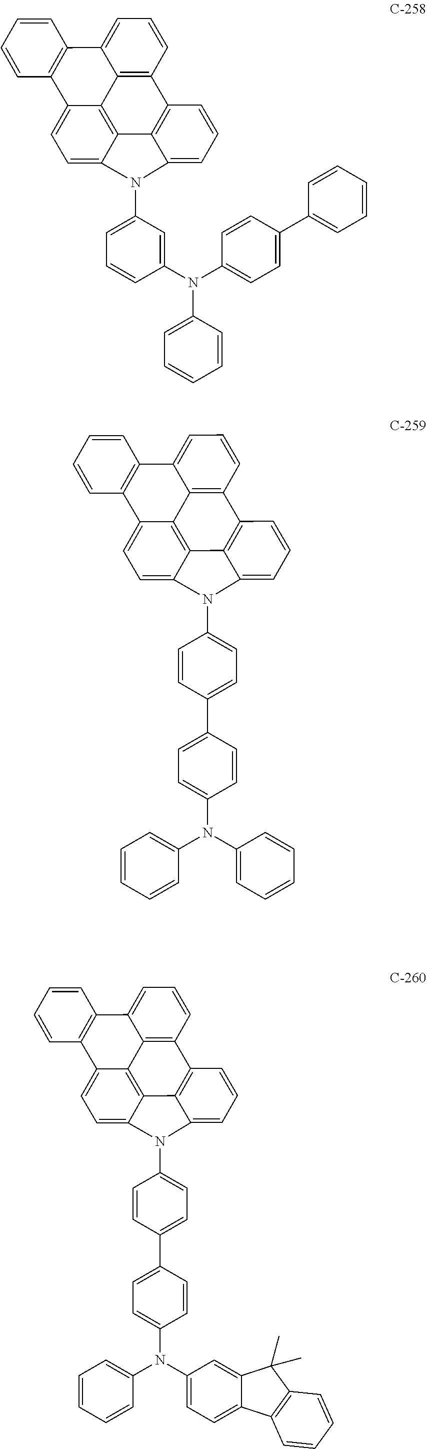 [SMILES: c1ccc(-c2ccc(N(c3ccccc3)c3cccc(-n4c5cccc6c7cccc8c9ccccc9c9ccc4c(c9c87)c65)c3)cc2)cc1 ; c1ccc(N(c2ccccc2)c2ccc(-c3ccc(-n4c5cccc6c7cccc8c9ccccc9c9ccc4c(c9c87)c65)cc3)cc2)cc1 ; CC1(C)c2ccccc2-c2ccc(N(c3ccccc3)c3ccc(-c4ccc(-n5c6cccc7c8cccc9c%10ccccc%10c%10ccc5c(c%10c98)c76)cc4)cc3)cc21]

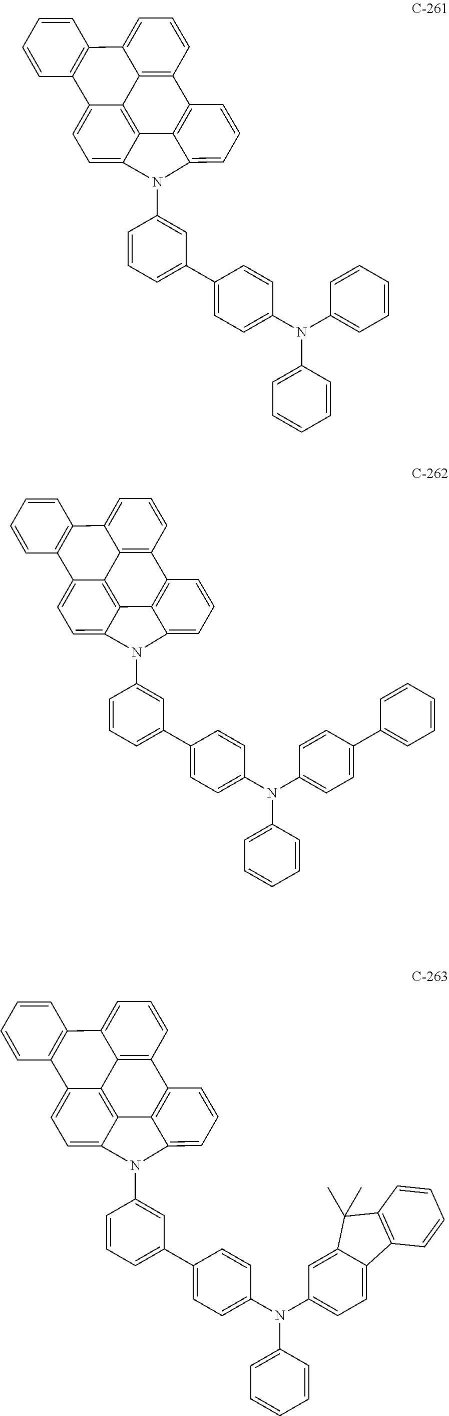 [SMILES: c1ccc(N(c2ccccc2)c2ccc(-c3cccc(-n4c5cccc6c7cccc8c9ccccc9c9ccc4c(c9c87)c65)c3)cc2)cc1 ; c1ccc(-c2ccc(N(c3ccccc3)c3ccc(-c4cccc(-n5c6cccc7c8cccc9c%10ccccc%10c%10ccc5c(c%10c98)c76)c4)cc3)cc2)cc1 ; CC1(C)c2ccccc2-c2ccc(N(c3ccccc3)c3ccc(-c4cccc(-n5c6cccc7c8cccc9c%10ccccc%10c%10ccc5c(c%10c98)c76)c4)cc3)cc21]

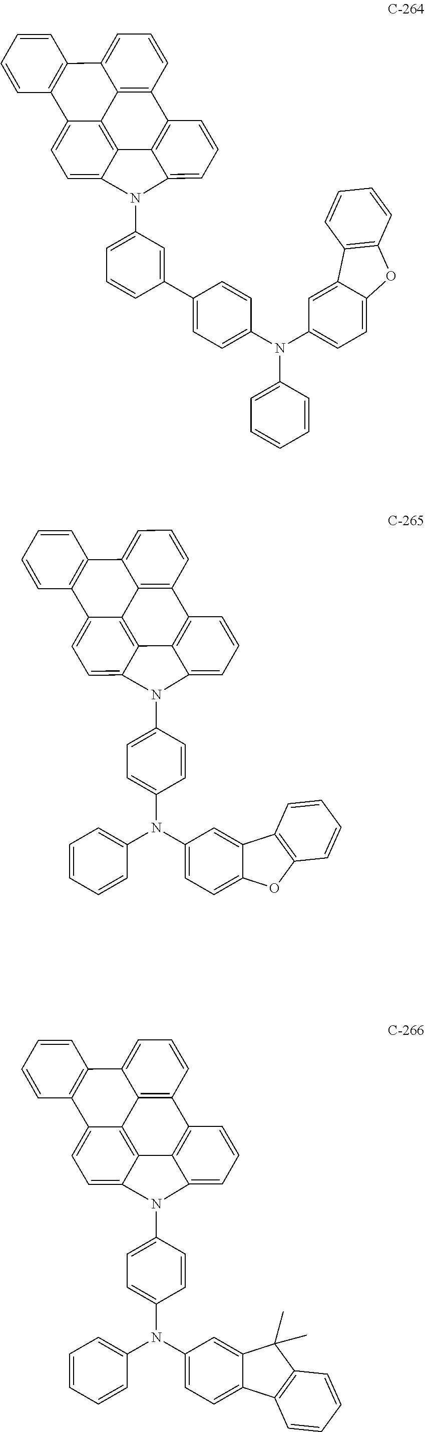 [SMILES: c1ccc(N(c2ccc(-c3cccc(-n4c5cccc6c7cccc8c9ccccc9c9ccc4c(c9c87)c65)c3)cc2)c2ccc3oc4ccccc4c3c2)cc1 ; c1ccc(N(c2ccc(-n3c4cccc5c6cccc7c8ccccc8c8ccc3c(c8c76)c54)cc2)c2ccc3oc4ccccc4c3c2)cc1 ; CC1(C)c2ccccc2-c2ccc(N(c3ccccc3)c3ccc(-n4c5cccc6c7cccc8c9ccccc9c9ccc4c(c9c87)c65)cc3)cc21]

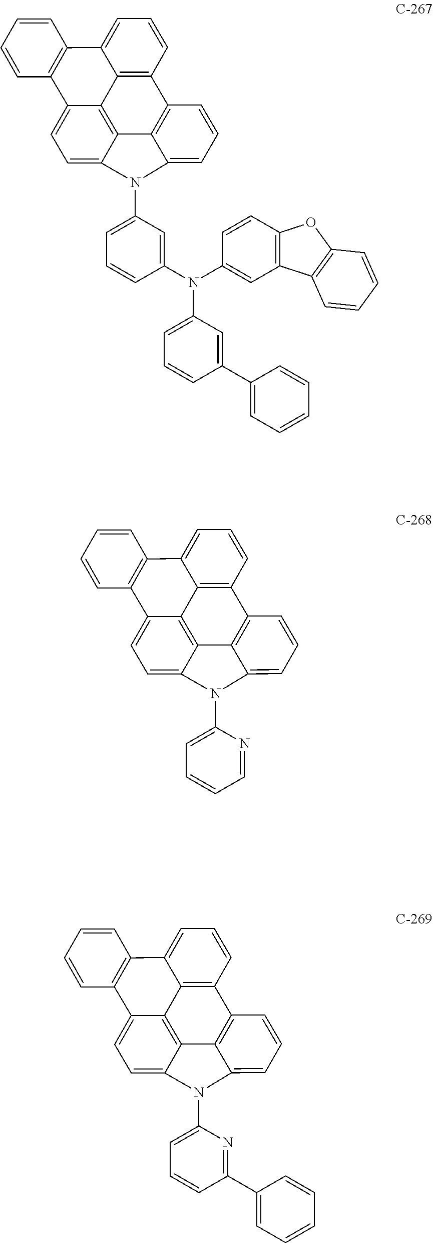 [SMILES: c1ccc(-c2cccc(N(c3cccc(-n4c5cccc6c7cccc8c9ccccc9c9ccc4c(c9c87)c65)c3)c3ccc4oc5ccccc5c4c3)c2)cc1 ; c1ccc(-n2c3cccc4c5cccc6c7ccccc7c7ccc2c(c7c65)c43)nc1 ; c1ccc(-c2cccc(-n3c4cccc5c6cccc7c8ccccc8c8ccc3c(c8c76)c54)n2)cc1]

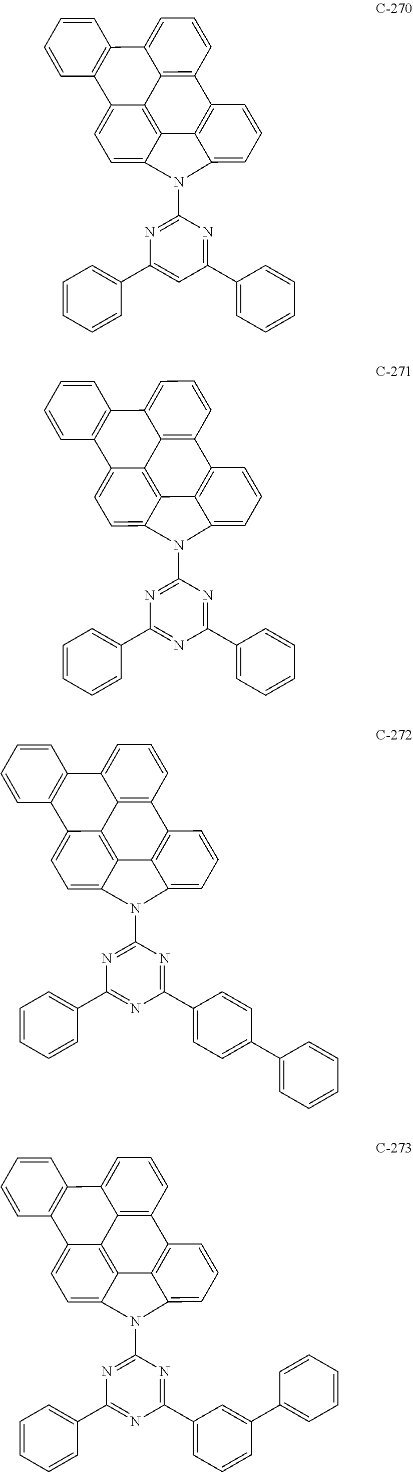 [SMILES: c1ccc(-c2cc(-c3ccccc3)nc(-n3c4cccc5c6cccc7c8ccccc8c8ccc3c(c8c76)c54)n2)cc1 ; c1ccc(-c2nc(-c3ccccc3)nc(-n3c4cccc5c6cccc7c8ccccc8c8ccc3c(c8c76)c54)n2)cc1 ; c1ccc(-c2ccc(-c3nc(-c4ccccc4)nc(-n4c5cccc6c7cccc8c9ccccc9c9ccc4c(c9c87)c65)n3)cc2)cc1 ; c1ccc(-c2cccc(-c3nc(-c4ccccc4)nc(-n4c5cccc6c7cccc8c9ccccc9c9ccc4c(c9c87)c65)n3)c2)cc1]

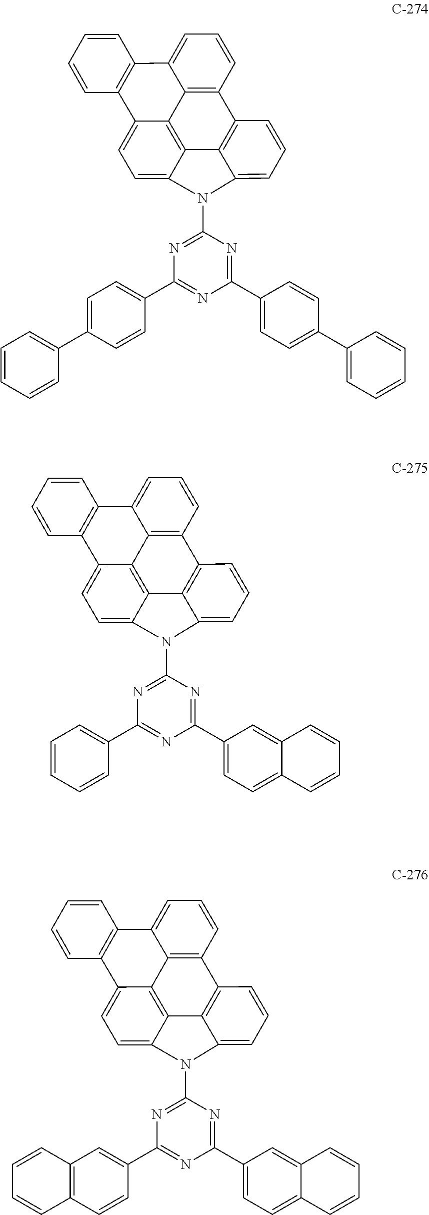 [SMILES: c1ccc(-c2ccc(-c3nc(-c4ccc(-c5ccccc5)cc4)nc(-n4c5cccc6c7cccc8c9ccccc9c9ccc4c(c9c87)c65)n3)cc2)cc1 ; c1ccc(-c2nc(-c3ccc4ccccc4c3)nc(-n3c4cccc5c6cccc7c8ccccc8c8ccc3c(c8c76)c54)n2)cc1 ; c1ccc2cc(-c3nc(-c4ccc5ccccc5c4)nc(-n4c5cccc6c7cccc8c9ccccc9c9ccc4c(c9c87)c65)n3)ccc2c1]

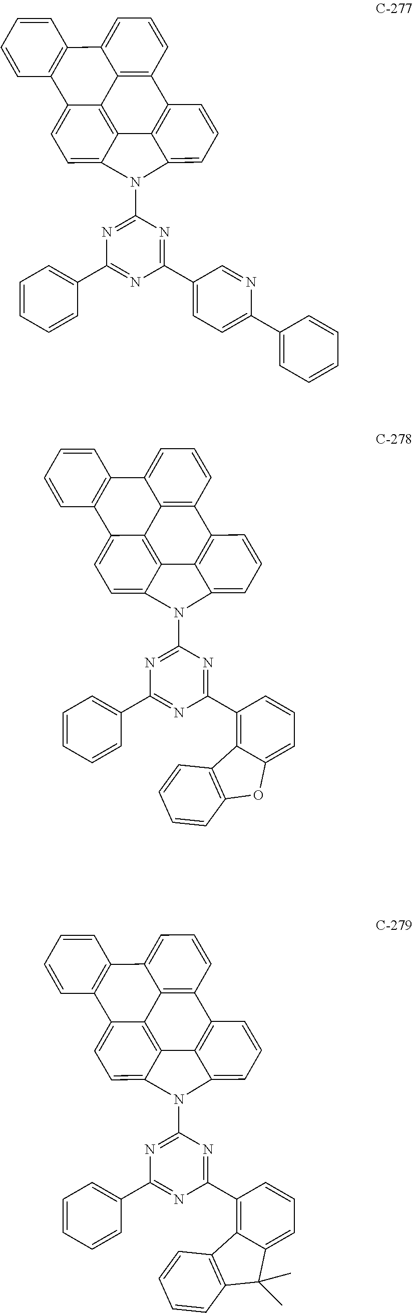 [SMILES: c1ccc(-c2ccc(-c3nc(-c4ccccc4)nc(-n4c5cccc6c7cccc8c9ccccc9c9ccc4c(c9c87)c65)n3)cn2)cc1 ; c1ccc(-c2nc(-c3cccc4oc5ccccc5c34)nc(-n3c4cccc5c6cccc7c8ccccc8c8ccc3c(c8c76)c54)n2)cc1 ; CC1(C)c2ccccc2-c2c(-c3nc(-c4ccccc4)nc(-n4c5cccc6c7cccc8c9ccccc9c9ccc4c(c9c87)c65)n3)cccc21]

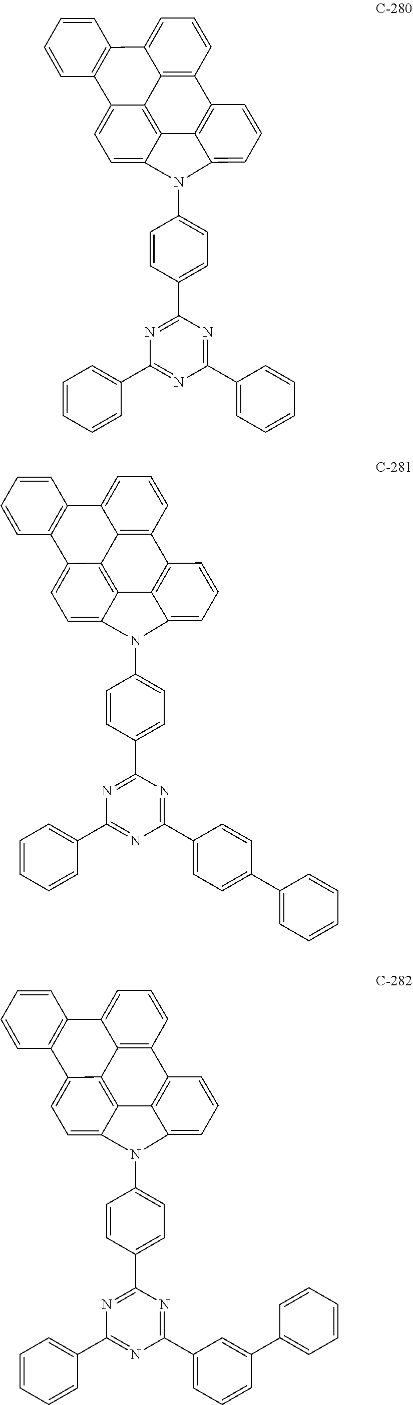 [SMILES: c1ccc(-c2nc(-c3ccccc3)nc(-c3ccc(-n4c5cccc6c7cccc8c9ccccc9c9ccc4c(c9c87)c65)cc3)n2)cc1 ; c1ccc(-c2ccc(-c3nc(-c4ccccc4)nc(-c4ccc(-n5c6cccc7c8cccc9c%10ccccc%10c%10ccc5c(c%10c98)c76)cc4)n3)cc2)cc1 ; c1ccc(-c2cccc(-c3nc(-c4ccccc4)nc(-c4ccc(-n5c6cccc7c8cccc9c%10ccccc%10c%10ccc5c(c%10c98)c76)cc4)n3)c2)cc1]

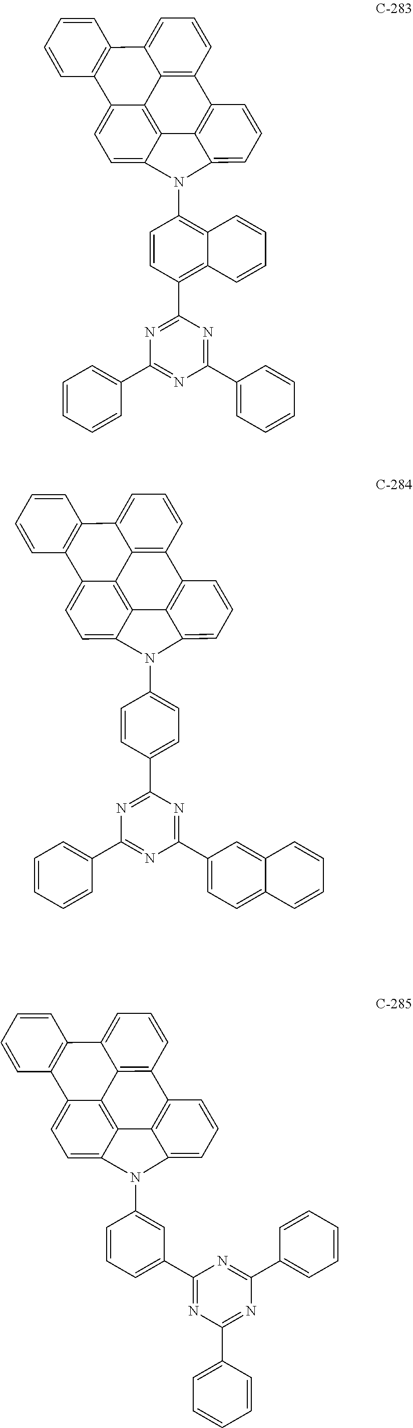 [SMILES: c1ccc(-c2nc(-c3ccccc3)nc(-c3ccc(-n4c5cccc6c7cccc8c9ccccc9c9ccc4c(c9c87)c65)c4ccccc34)n2)cc1 ; c1ccc(-c2nc(-c3ccc(-n4c5cccc6c7cccc8c9ccccc9c9ccc4c(c9c87)c65)cc3)nc(-c3ccc4ccccc4c3)n2)cc1 ; c1ccc(-c2nc(-c3ccccc3)nc(-c3cccc(-n4c5cccc6c7cccc8c9ccccc9c9ccc4c(c9c87)c65)c3)n2)cc1]

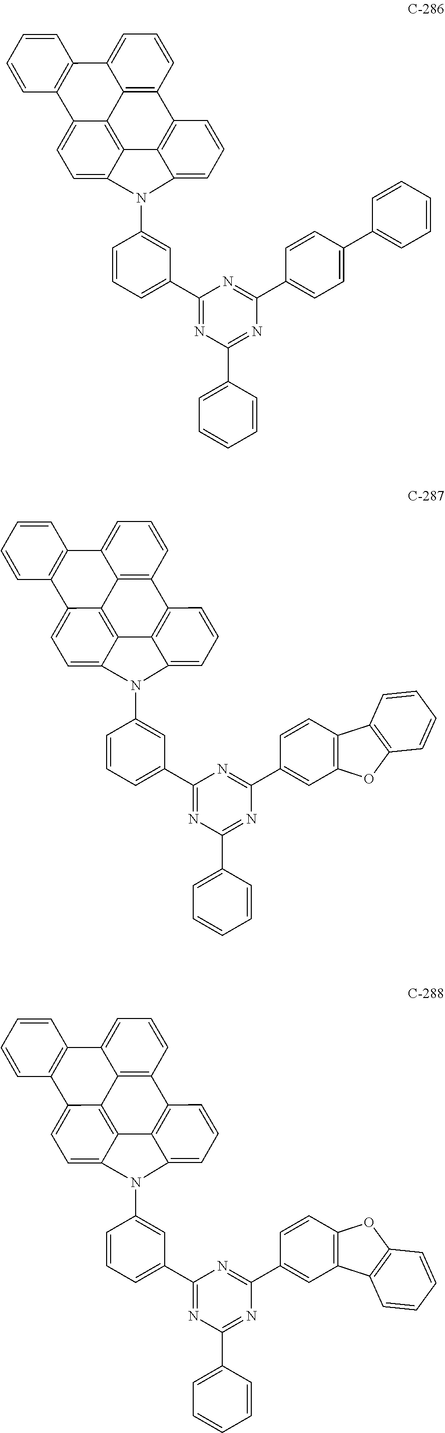 [SMILES: c1ccc(-c2ccc(-c3nc(-c4ccccc4)nc(-c4cccc(-n5c6cccc7c8cccc9c%10ccccc%10c%10ccc5c(c%10c98)c76)c4)n3)cc2)cc1 ; c1ccc(-c2nc(-c3cccc(-n4c5cccc6c7cccc8c9ccccc9c9ccc4c(c9c87)c65)c3)nc(-c3ccc4c(c3)oc3ccccc34)n2)cc1 ; c1ccc(-c2nc(-c3cccc(-n4c5cccc6c7cccc8c9ccccc9c9ccc4c(c9c87)c65)c3)nc(-c3ccc4oc5ccccc5c4c3)n2)cc1]

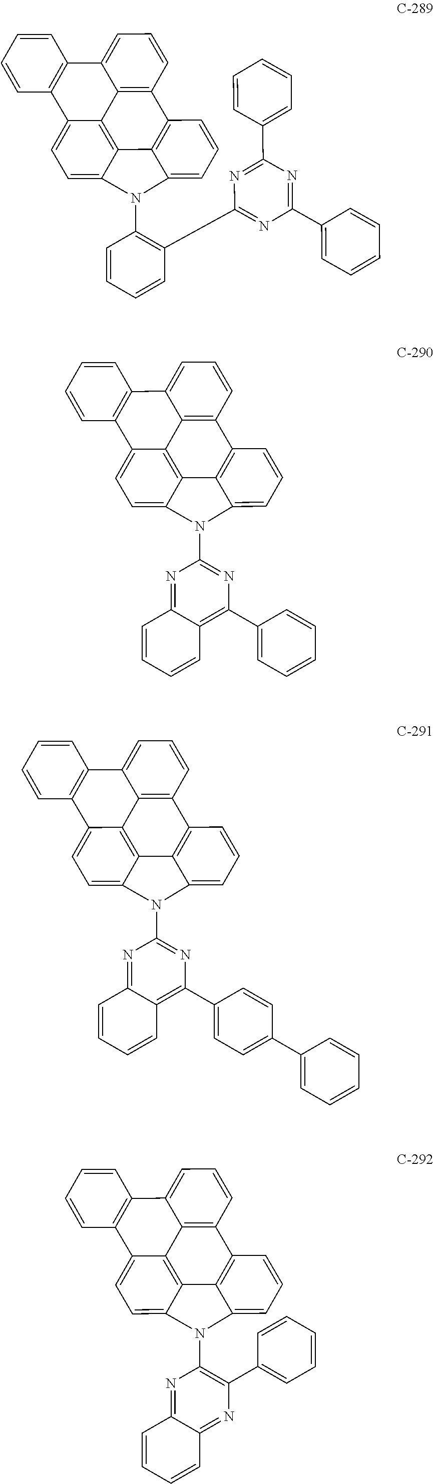 [SMILES: c1ccc(-c2nc(-c3ccccc3)nc(-c3ccccc3-n3c4cccc5c6cccc7c8ccccc8c8ccc3c(c8c76)c54)n2)cc1 ; c1ccc(-c2nc(-n3c4cccc5c6cccc7c8ccccc8c8ccc3c(c8c76)c54)nc3ccccc23)cc1 ; c1ccc(-c2ccc(-c3nc(-n4c5cccc6c7cccc8c9ccccc9c9ccc4c(c9c87)c65)nc4ccccc34)cc2)cc1 ; c1ccc(-c2nc3ccccc3nc2-n2c3cccc4c5cccc6c7ccccc7c7ccc2c(c7c65)c43)cc1]

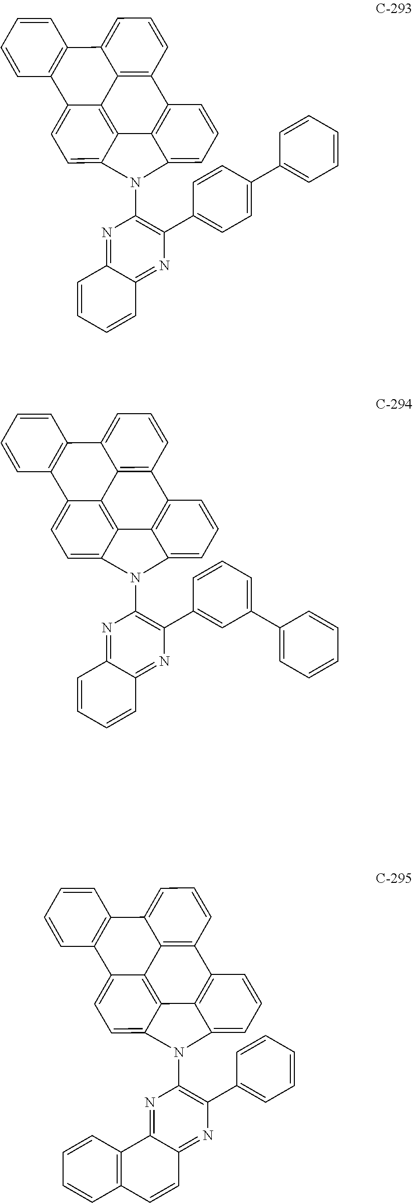 [SMILES: c1ccc(-c2ccc(-c3nc4ccccc4nc3-n3c4cccc5c6cccc7c8ccccc8c8ccc3c(c8c76)c54)cc2)cc1 ; c1ccc(-c2cccc(-c3nc4ccccc4nc3-n3c4cccc5c6cccc7c8ccccc8c8ccc3c(c8c76)c54)c2)cc1 ; c1ccc(-c2nc3ccc4ccccc4c3nc2-n2c3cccc4c5cccc6c7ccccc7c7ccc2c(c7c65)c43)cc1]

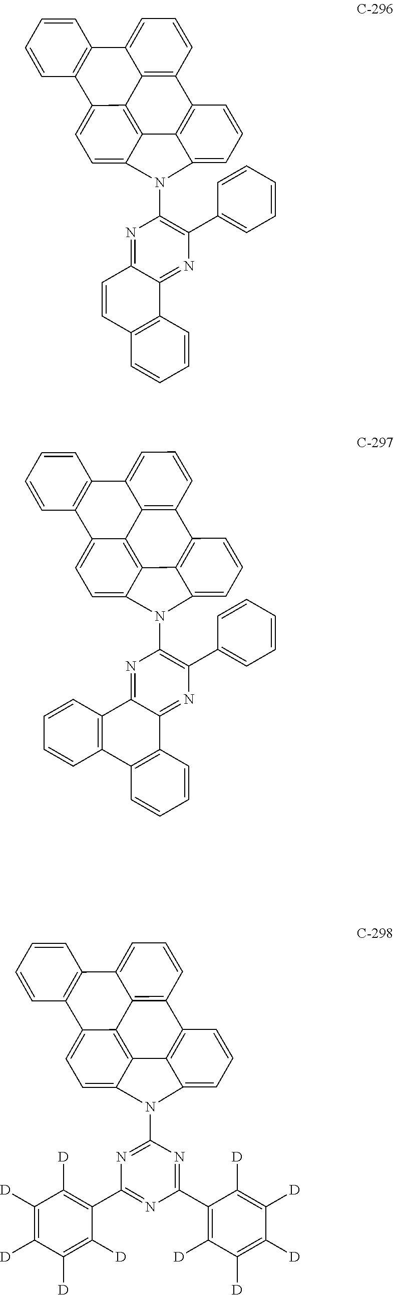 [SMILES: c1ccc(-c2nc3c(ccc4ccccc43)nc2-n2c3cccc4c5cccc6c7ccccc7c7ccc2c(c7c65)c43)cc1 ; c1ccc(-c2nc3c4ccccc4c4ccccc4c3nc2-n2c3cccc4c5cccc6c7ccccc7c7ccc2c(c7c65)c43)cc1 ; [2H]c1c([2H])c([2H])c(-c2nc(-c3c([2H])c([2H])c([2H])c([2H])c3[2H])nc(-n3c4cccc5c6cccc7c8ccccc8c8ccc3c(c8c76)c54)n2)c([2H])c1[2H]]

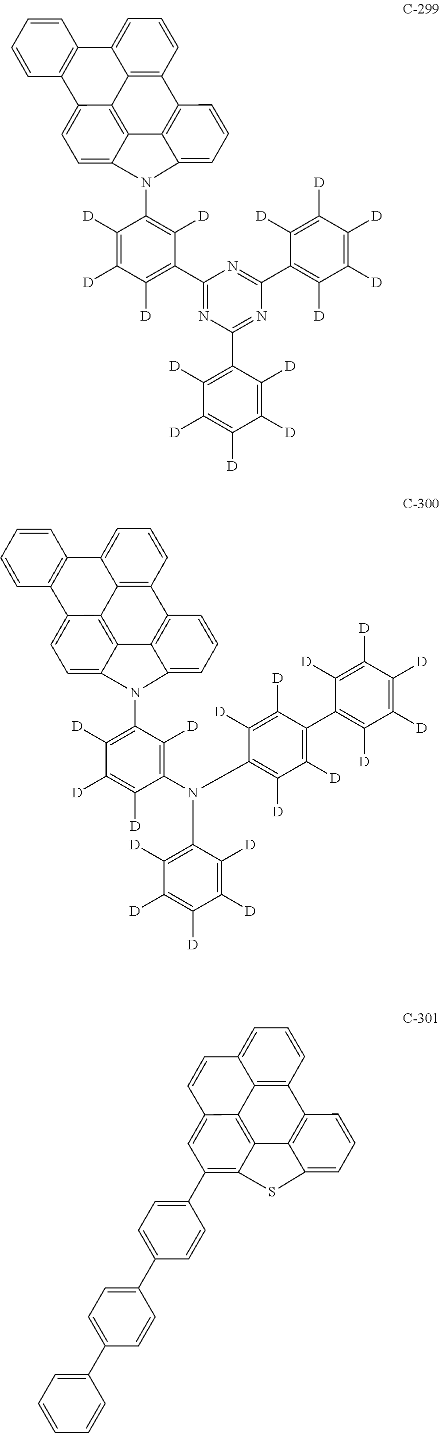 [SMILES: [2H]c1c([2H])c([2H])c(-c2nc(-c3c([2H])c([2H])c([2H])c([2H])c3[2H])nc(-c3c([2H])c([2H])c([2H])c(-n4c5cccc6c7cccc8c9ccccc9c9ccc4c(c9c87)c65)c3[2H])n2)c([2H])c1[2H] ; [2H]c1c([2H])c([2H])c(-c2c([2H])c([2H])c(N(c3c([2H])c([2H])c([2H])c([2H])c3[2H])c3c([2H])c([2H])c([2H])c(-n4c5cccc6c7cccc8c9ccccc9c9ccc4c(c9c87)c65)c3[2H])c([2H])c2[2H])c([2H])c1[2H] ; c1ccc(-c2ccc(-c3ccc(-c4cc5ccc6cccc7c8cccc9sc4c(c98)c5c67)cc3)cc2)cc1]

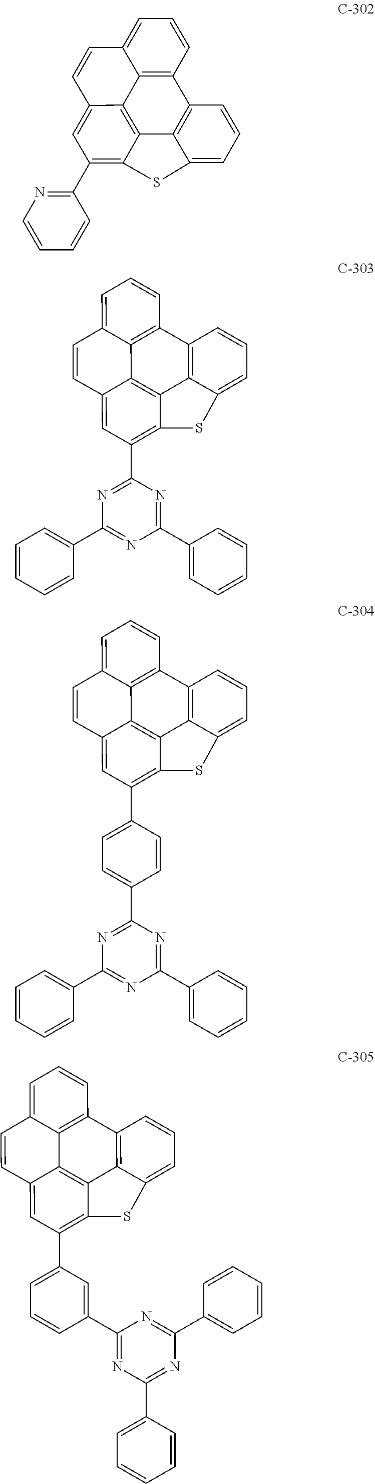

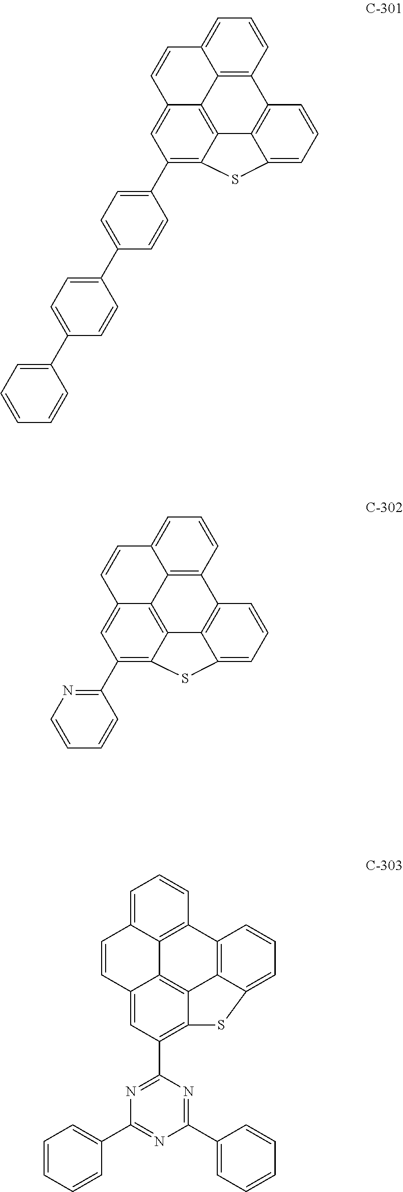 [SMILES: c1ccc(-c2ccc(-c3ccc(-c4cc5ccc6cccc7c8cccc9sc4c(c98)c5c67)cc3)cc2)cc1 ; c1ccc(-c2cc3ccc4cccc5c6cccc7sc2c(c76)c3c45)nc1 ; c1ccc(-c2nc(-c3ccccc3)nc(-c3cc4ccc5cccc6c7cccc8sc3c(c87)c4c56)n2)cc1]

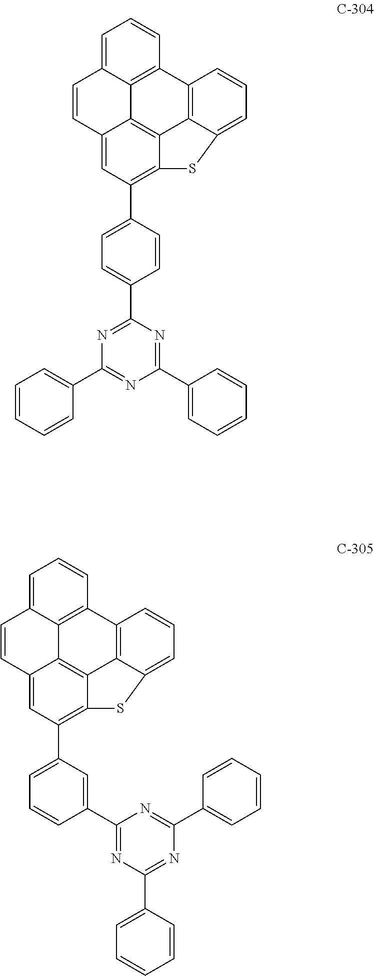 [SMILES: c1ccc(-c2nc(-c3ccccc3)nc(-c3ccc(-c4cc5ccc6cccc7c8cccc9sc4c(c98)c5c67)cc3)n2)cc1 ; c1ccc(-c2nc(-c3ccccc3)nc(-c3cccc(-c4cc5ccc6cccc7c8cccc9sc4c(c98)c5c67)c3)n2)cc1]

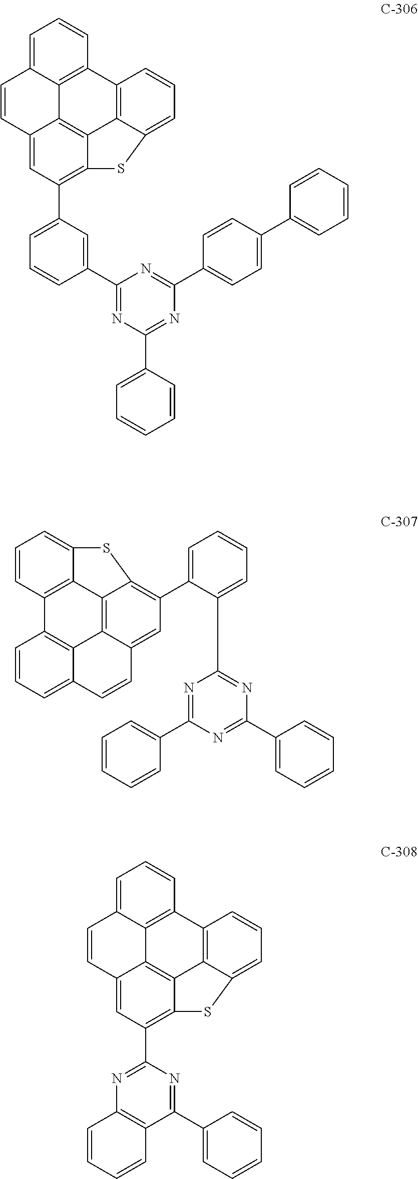 [SMILES: c1ccc(-c2ccc(-c3nc(-c4ccccc4)nc(-c4cccc(-c5cc6ccc7cccc8c9cccc%10sc5c(c%109)c6c78)c4)n3)cc2)cc1 ; c1ccc(-c2nc(-c3ccccc3)nc(-c3ccccc3-c3cc4ccc5cccc6c7cccc8sc3c(c87)c4c56)n2)cc1 ; c1ccc(-c2nc(-c3cc4ccc5cccc6c7cccc8sc3c(c87)c4c56)nc3ccccc23)cc1]

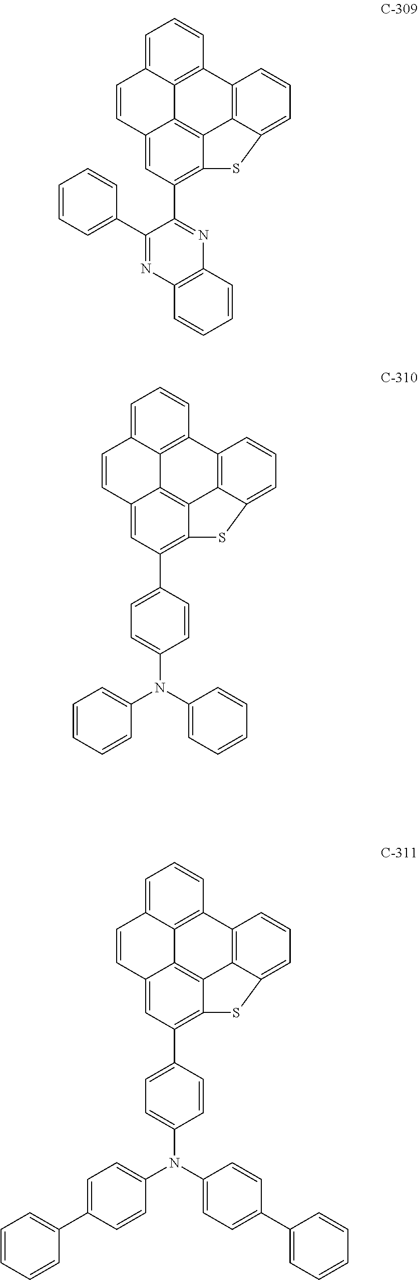 [SMILES: c1ccc(-c2nc3ccccc3nc2-c2cc3ccc4cccc5c6cccc7sc2c(c76)c3c45)cc1 ; c1ccc(N(c2ccccc2)c2ccc(-c3cc4ccc5cccc6c7cccc8sc3c(c87)c4c56)cc2)cc1 ; c1ccc(-c2ccc(N(c3ccc(-c4ccccc4)cc3)c3ccc(-c4cc5ccc6cccc7c8cccc9sc4c(c98)c5c67)cc3)cc2)cc1]

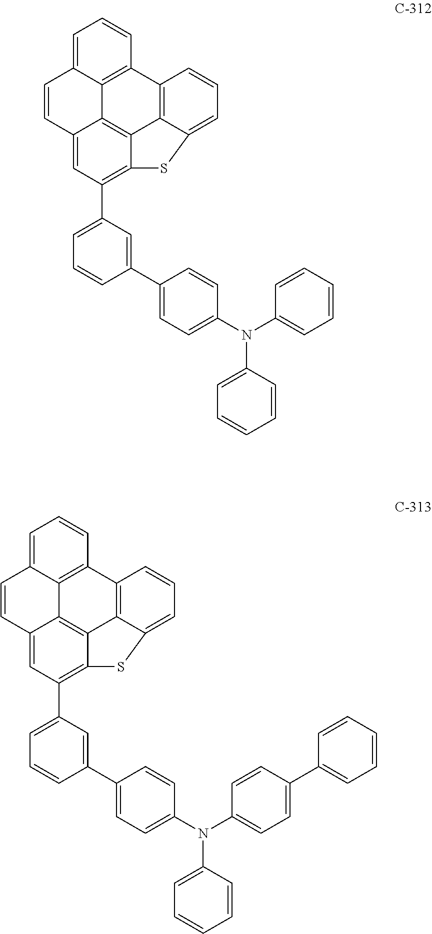 [SMILES: c1ccc(N(c2ccccc2)c2ccc(-c3cccc(-c4cc5ccc6cccc7c8cccc9sc4c(c98)c5c67)c3)cc2)cc1 ; c1ccc(-c2ccc(N(c3ccccc3)c3ccc(-c4cccc(-c5cc6ccc7cccc8c9cccc%10sc5c(c%109)c6c78)c4)cc3)cc2)cc1]

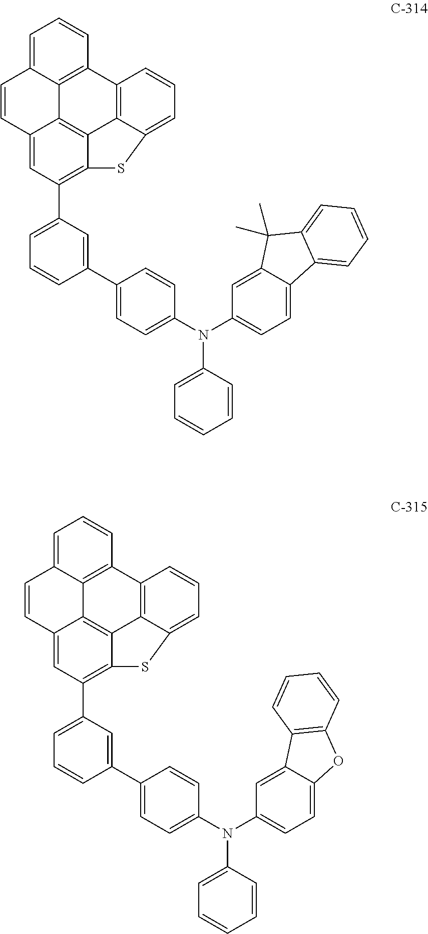 [SMILES: CC1(C)c2ccccc2-c2ccc(N(c3ccccc3)c3ccc(-c4cccc(-c5cc6ccc7cccc8c9cccc%10sc5c(c%109)c6c78)c4)cc3)cc21 ; c1ccc(N(c2ccc(-c3cccc(-c4cc5ccc6cccc7c8cccc9sc4c(c98)c5c67)c3)cc2)c2ccc3oc4ccccc4c3c2)cc1]

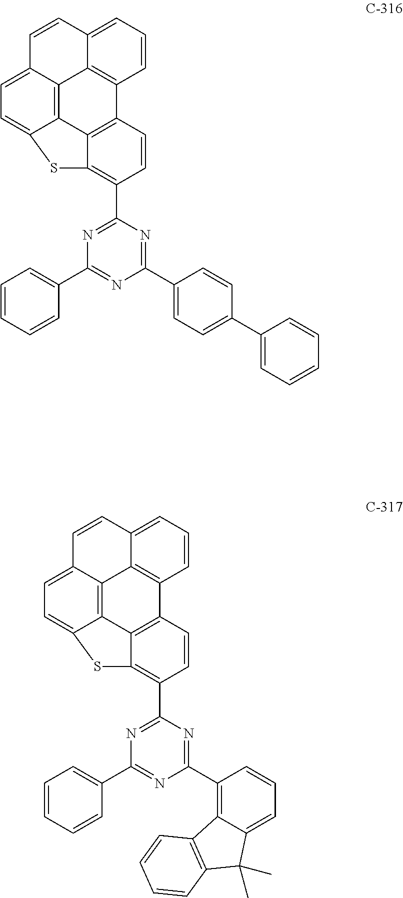 [SMILES: c1ccc(-c2ccc(-c3nc(-c4ccccc4)nc(-c4ccc5c6cccc7ccc8ccc9sc4c5c9c8c76)n3)cc2)cc1 ; CC1(C)c2ccccc2-c2c(-c3nc(-c4ccccc4)nc(-c4ccc5c6cccc7ccc8ccc9sc4c5c9c8c76)n3)cccc21]

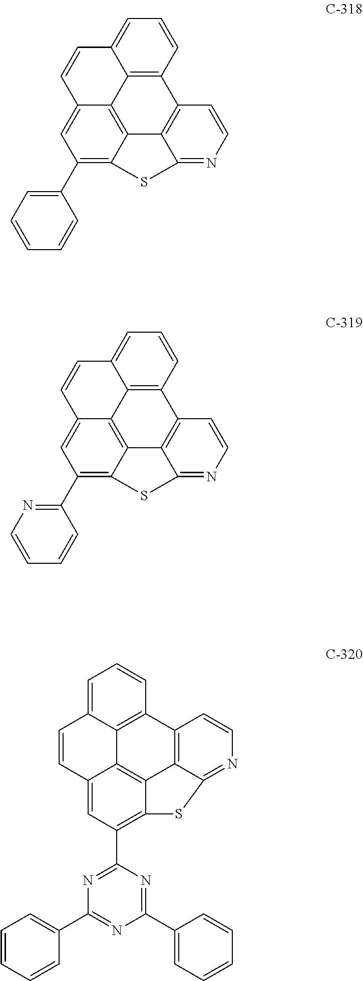 [SMILES: c1ccc(-c2cc3ccc4cccc5c6ccnc7sc2c(c76)c3c45)cc1 ; c1ccc(-c2cc3ccc4cccc5c6ccnc7sc2c(c76)c3c45)nc1 ; c1ccc(-c2nc(-c3ccccc3)nc(-c3cc4ccc5cccc6c7ccnc8sc3c(c87)c4c56)n2)cc1]

The compounds represented by formula 1 according to the present disclosure may be prepared as represented by the following reaction schemes 1 to 3, but is not limited thereto; they may further be produced by a synthetic method known to a person skilled in the art.

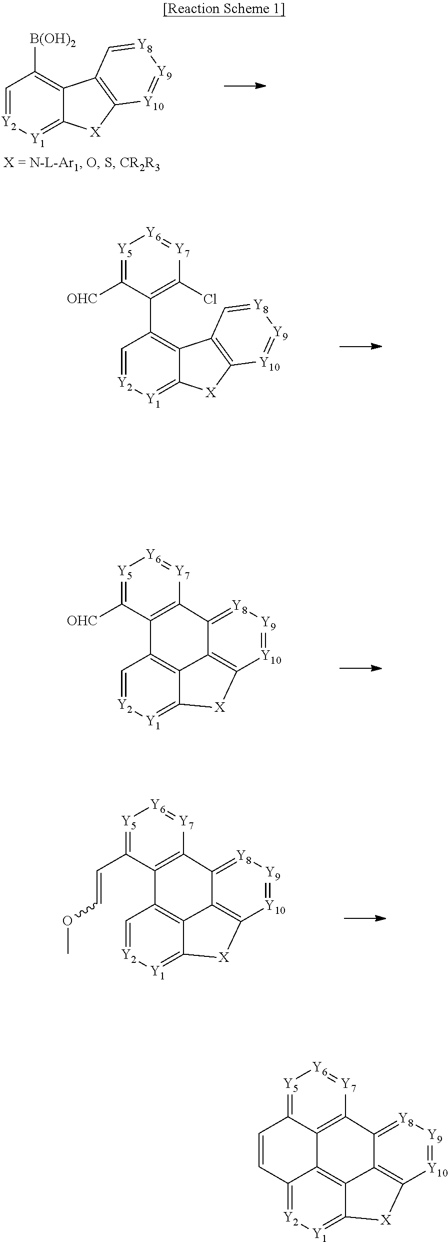

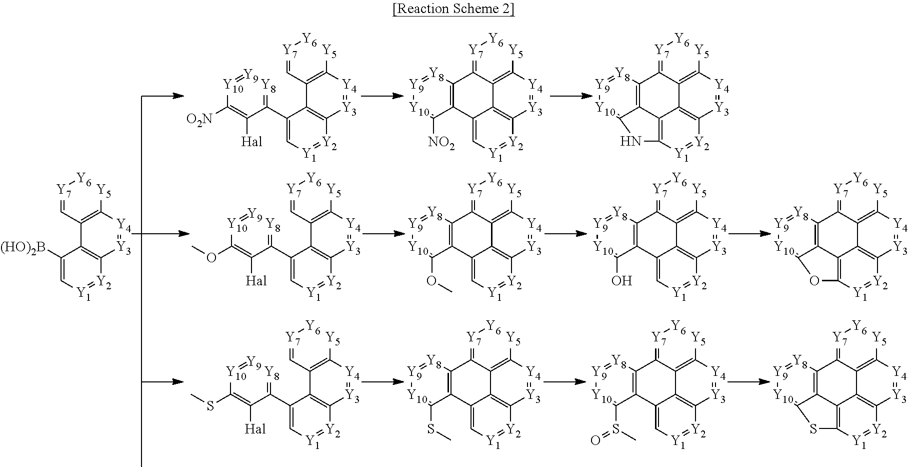

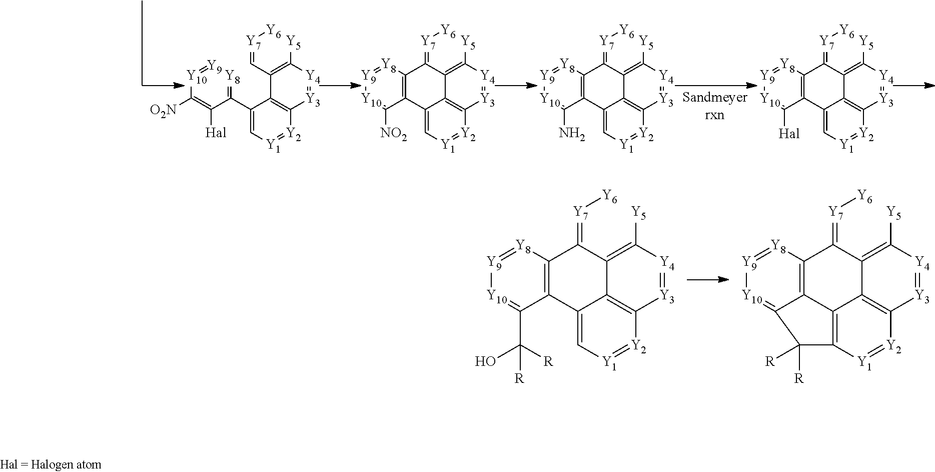

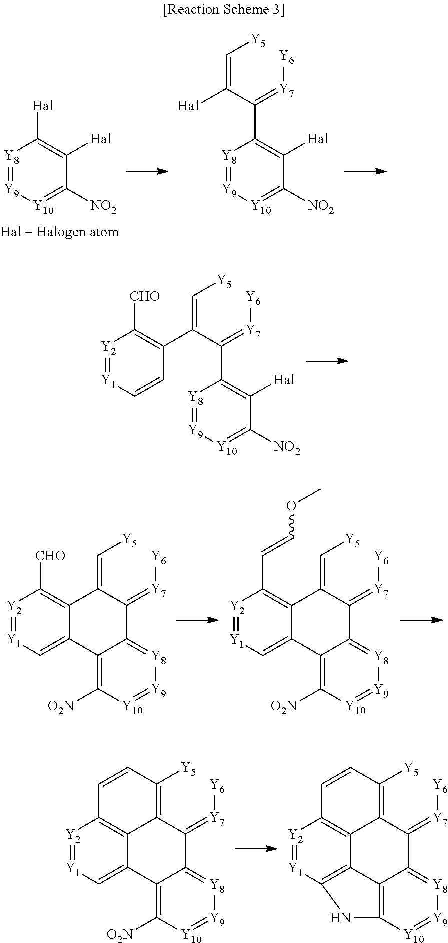

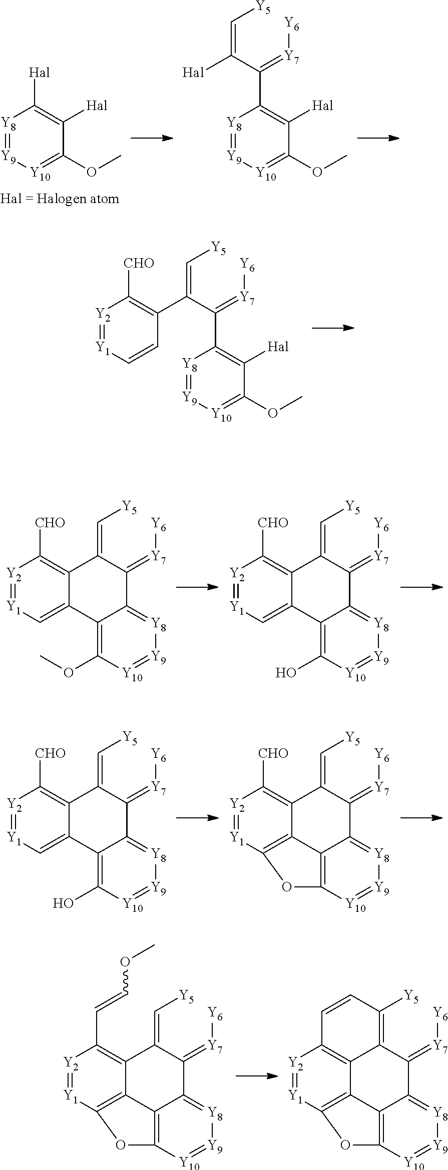

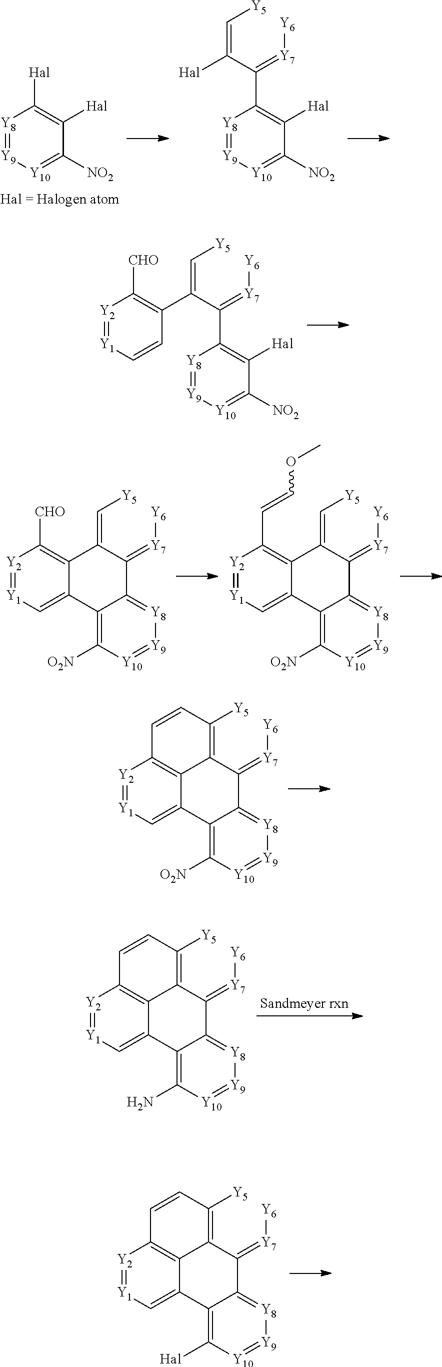

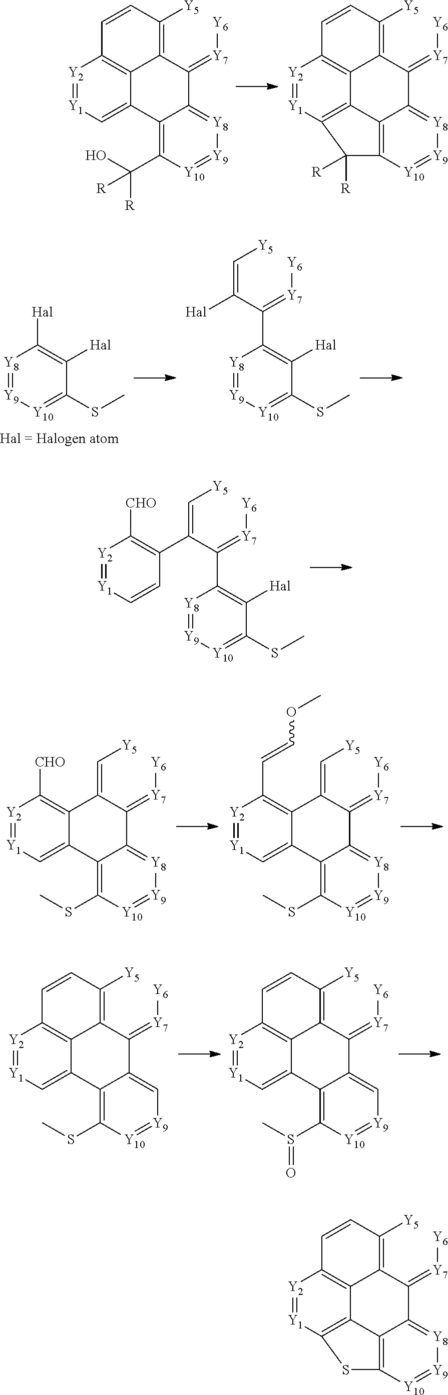

In reaction schemes 1 to 3 above, the definition of each of the substituents is as defined in formula 1, and R may be defined as -L₃-Ar₃ and/or -L₄-Ar₄ in formula 1-4.

As described above, exemplary synthesis examples of the compounds represented by formula 1 according to the present disclosure are described, but they are based on Sandmeyer reaction, Buchwald-Hartwig cross coupling reaction, N-arylation reaction, H-mont-mediated etherification reaction. Miyaura borylation reaction, Suzuki cross-coupling reaction, Intramolecular acid-induced cyclization reaction, Pd(II)-catalyzed oxidative cyclization reaction, Grignard reaction. Heck reaction, Cyclic Dehydration reaction, SN₁ substitution reaction. SN₂ substitution reaction, and Phosphine-mediated reductive cyclization reaction, etc. It will be understood by one skilled in the art that the above reaction proceeds even if other substituents defined in the formula 1 other than the substituents described in the specific synthesis examples are bonded.

According to another embodiment, the present disclosure provides a plurality of host materials comprising a first host material and a second host material.

The plurality of host materials according to one embodiment comprises a first host material including the compound represented by formula 1 and a second host material including a compound represented by the following formula 2 and may be included in a light-emitting layer of an organic electroluminescent device according to one embodiment.

The second host material according to one embodiment may comprise a compound represented by the following formula 2.

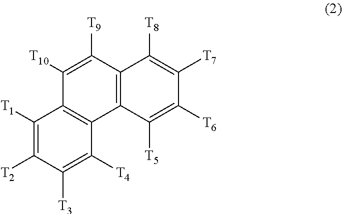

In formula 2,

T₅ and T₆ are linked to each other to form a ring of the following formula 3; or T₇ and T₈ are linked to each other to form a ring of the following formula 3; or T₅ and T₆ are linked to each other to form a ring of the following formula 3 and T₇ and T₈ are linked to each other to form a ring of the following formula 3;

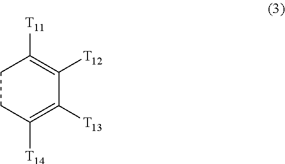

In formulas 2 and 3,

T₁ to T₄, T₉ to T₁₄, and T₅ to T₈ that do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂, provided that at least one of T₁ to T₁₄ is -L₂-Ar₂;

L₂ represents a single bond, a substituted or unsubstituted (C6-C30)arylene, or a substituted or unsubstituted (3- to 30-membered)heteroarylene:

Ar₂ represents a substituted or unsubstituted (C6-C30)aryl, or a substituted or unsubstituted (3- to 30-membered)heteroaryl;

[symbol] represents a site fused to the formula 2; and

the heteroaryl comprises at least one hetero atom selected from B, N, O, S, Si and P.

In one embodiment, T₅ and T₆ may be linked to each other to form a ring of the formula 3 and/or, T₇ and T₈ may be linked to each other to form a ring of the formula 3.

In one embodiment, T₁ to T₁₄ each independently may be hydrogen, a substituted or unsubstituted (C6-C30)aryl, or -L₂-Ar₂, preferably hydrogen, a substituted or unsubstituted (C6-C25)aryl, or -L₂-Ar₂, more preferably hydrogen, a substituted or unsubstituted (C6-C18)aryl, or -L₂-Ar₂. For example, T₁ to T₁₄ each independently may be hydrogen, phenyl, naphthyl, biphenyl, or -L₂-Ar₂.

In one embodiment, at least one of T₁ to T₄, T₁₁ to T₁₄, and T₅ to T₆ that do not form a ring, may be -L₂-Ar₂.

In one embodiment, L₂ may be a single bond, a substituted or unsubstituted (C6-C30)arylene, or a substituted or unsubstituted (5- to 30-membered)heteroarylene, preferably a single bond or a substituted or unsubstituted (C6-C25)arylene, more preferably a single bond or (C6-C18)arylene unsubstituted or substituted with (C6-C18)aryl. For example, L₂ may be a single bond, phenylene unsubstituted or substituted with phenyl, naphthylene, or biphenylene, for example, 1,2-phenylene, 1,3-phenylene, 1,4-phenylene, 1,2-biphenylene, 1,3-biphenylene, 1,4-biphenylene, 1,2-naphthylene, 1,3-naphthylene, 1,4-naphthylene, 1,5-naphthylene, 1,6-naphthylene, 1,7-naphthylene, 1,8-naphthylene, 2,3-naphthylene, 2,6-naphthylene, or 2,7-naphthylene.

In one embodiment, Ar₂ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably (5- to 25-membered)heteroaryl unsubstituted or substituted with (C6-C30)aryl or (5- to 30-membered)heteroaryl, even more preferably nitrogen-containing (5- to 25-membered)heteroaryl unsubstituted or substituted with (C6-C30)aryl or (5- to 30-membered)heteroaryl. For example, Ar₂ may be a substituted or unsubstituted pyridyl, a substituted or unsubstituted pyrimidinyl, a substituted or unsubstituted triazinyl, a substituted or unsubstituted quinazolinyl, a substituted or unsubstituted benzoquinazolinyl, a substituted or unsubstituted dibenzoquinazolinyl, a substituted or unsubstituted quinoxalinyl, a substituted or unsubstituted benzoquinoxalinyl, a substituted or unsubstituted dibenzoquinoxalinyl, a substituted or unsubstituted quinolyl, a substituted or unsubstituted benzoquinolyl, a substituted or unsubstituted isoquinolyl, a substituted or unsubstituted naphthiridinyl, a substituted or unsubstituted benzoisoquinolyl, a substituted or unsubstituted imidazolyl, a substituted or unsubstituted benzimidazolyl, a substituted or unsubstituted phenanthroimidazolyl, a substituted or unsubstituted thiazolyl, a substituted or unsubstituted benzothiazolyl, a substituted or unsubstituted phenanthrothiazolyl, a substituted or unsubstituted oxazolyl, a substituted or unsubstituted benzoxazolyl, a substituted or unsubstituted phenanthrooxazolyl, a substituted or unsubstituted naphthooxazolyl, a substituted or unsubstituted naphthothiazolyl, a substituted or unsubstituted benzothienopyrimidinyl, a substituted or unsubstituted benzothienopyrazinyl, a substituted or unsubstituted benzofuropyrimidinyl, a substituted or unsubstituted benzofuropyrazinyl, a substituted or unsubstituted benzothienoquinolyl, a substituted or unsubstituted benzofuroquinolyl, a substituted or unsubstituted acenaphthopyrimidinyl, a substituted or unsubstituted carbazolyl, a substituted or unsubstituted benzocarbazolyl, a substituted or unsubstituted dibenzocarbazolyl, a substituted or unsubstituted phenoxazinyl, a substituted or unsubstituted benzopyrimidinyl, a substituted or unsubstituted 17-membered heteroaryl containing at least two nitrogen atoms, or a substituted or unsubstituted 25-membered heteroaryl at least one nitrogen atom. For example, the substituent of the substituted groups may be at least one of methyl; phenyl; phenyl substituted with fluorine; phenyl substituted with tert-butyl; phenyl substituted with trimethylsilyl; phenyl substituted with triphenylsilyl; phenyl substituted with carbazolyl; phenyl substituted with cyclohexyl; phenyl substituted with cyano; naphthyl; biphenyl; terphenyl; naphthylphenyl; phenylnaphthyl; phenanthrenyl; anthracenyl; chrysenyl; triphenylenyl; dimethylfluorenyl; diphenylfluorenyl; spirobifluorenyl; pyridyl substituted with phenyl; dibenzothiophenyl; dibenzofuranyl; dibenzofuranyl substituted with phenyl; dibenzofuranyl substituted with biphenyl; carbazolyl unsubstituted or substituted with phenyl; phenoxazinyl; benzothiophenyl; and naphthooxazolinyl substituted with phenyl.

According to one embodiment, the compound represented by formula 2 may be represented by the following formula 2-1 or 2-2.

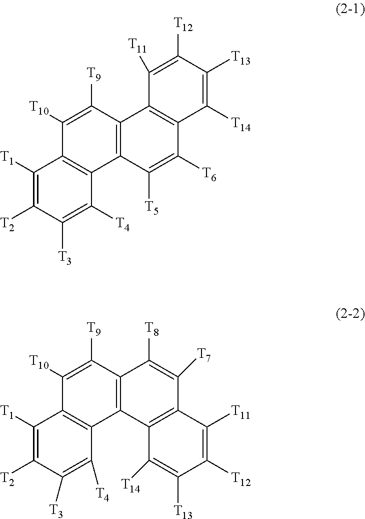

In formulas 2-1 and 2-2,

T₁ to T₁₄ are as defined in formula 2.

According to one embodiment, the compound represented by formula 2 may be represented by any one of the following formulas.

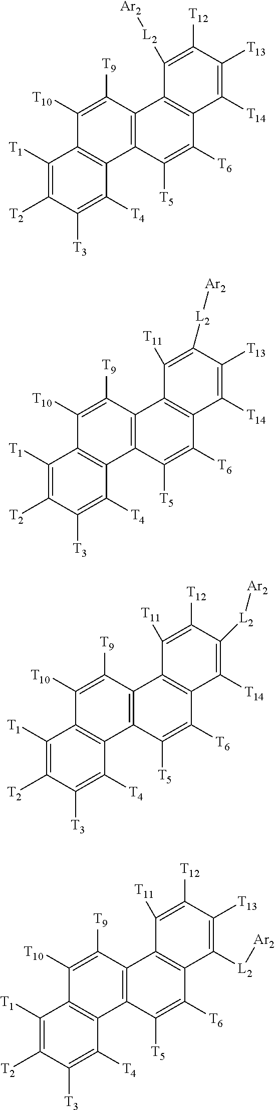

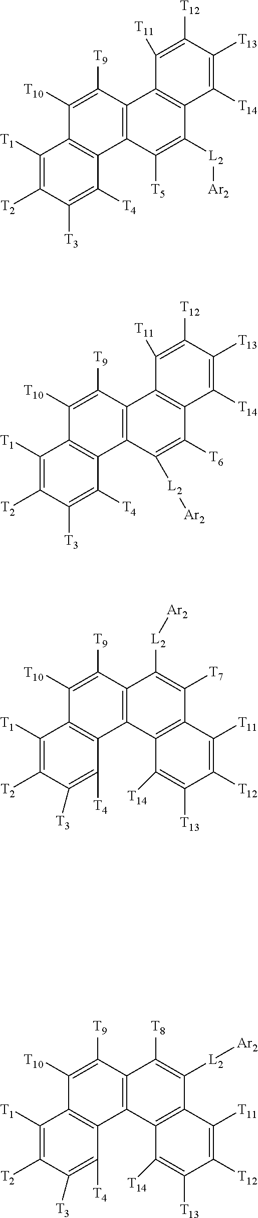

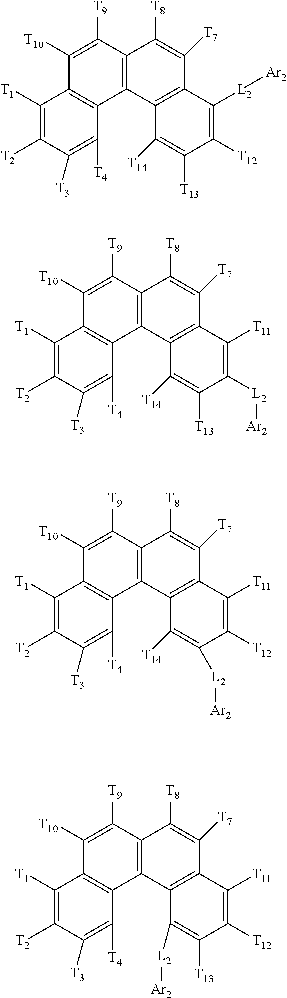

In the formulas,

T₁ to T₁₄ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and

L₂ and Ar₂ are as defined in formula 2.

In one embodiment, the compound represented by formula 2 may be more specifically illustrated by the following compounds, but is not limited thereto.

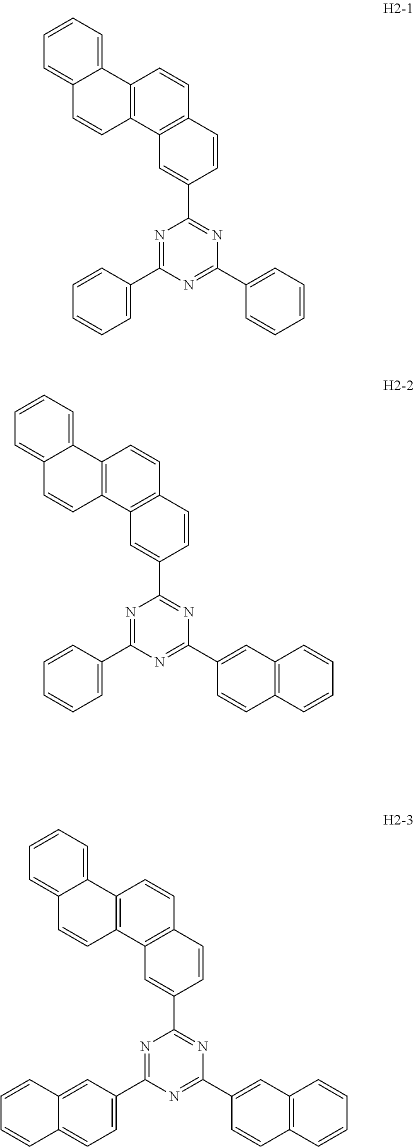 [SMILES: c1ccc(-c2nc(-c3ccccc3)nc(-c3ccc4ccc5c6ccccc6ccc5c4c3)n2)cc1 ; c1ccc(-c2nc(-c3ccc4ccccc4c3)nc(-c3ccc4ccc5c6ccccc6ccc5c4c3)n2)cc1 ; c1ccc2cc(-c3nc(-c4ccc5ccccc5c4)nc(-c4ccc5ccc6c7ccccc7ccc6c5c4)n3)ccc2c1]

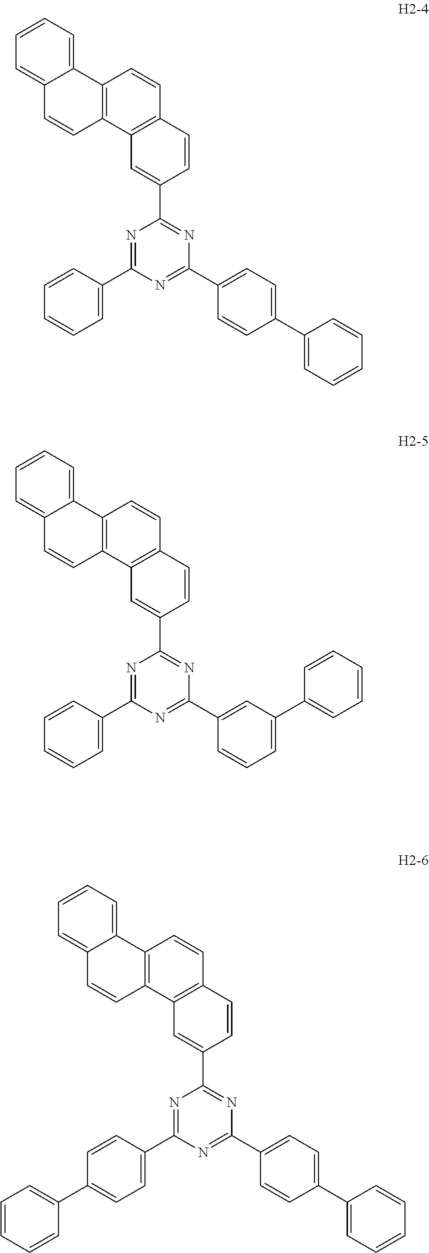 [SMILES: c1ccc(-c2ccc(-c3nc(-c4ccccc4)nc(-c4ccc5ccc6c7ccccc7ccc6c5c4)n3)cc2)cc1 ; c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1]

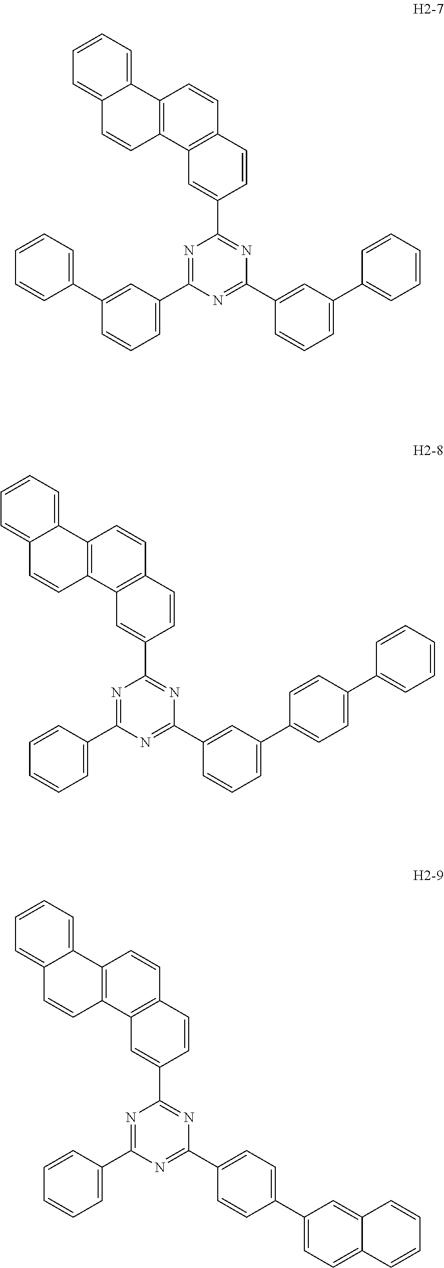 [SMILES: c1ccc(-c2cccc(-c3nc(-c4cccc(-c5ccccc5)c4)nc(-c4ccc5ccc6c7ccccc7ccc6c5c4)n3)c2)cc1 ; c1ccc(-c2ccc(-c3cccc(-c4nc(-c5ccccc5)nc(-c5ccc6ccc7c8ccccc8ccc7c6c5)n4)c3)cc2)cc1 ; c1ccc(-c2nc(-c3ccc(-c4ccc5ccccc5c4)cc3)nc(-c3ccc4ccc5c6ccccc6ccc5c4c3)n2)cc1]

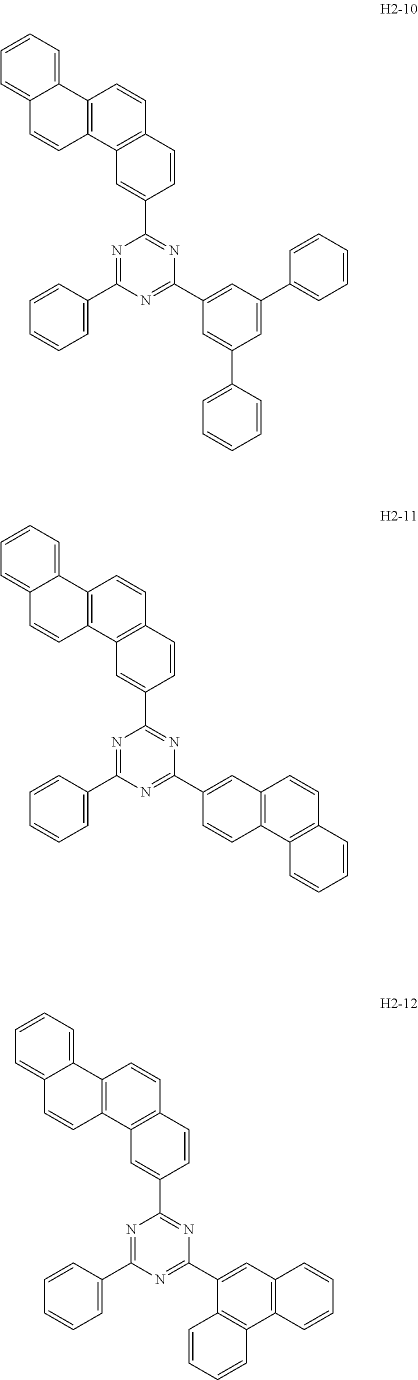 [SMILES: c1ccc(-c2cc(-c3ccccc3)cc(-c3nc(-c4ccccc4)nc(-c4ccc5ccc6c7ccccc7ccc6c5c4)n3)c2)cc1 ; c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1]

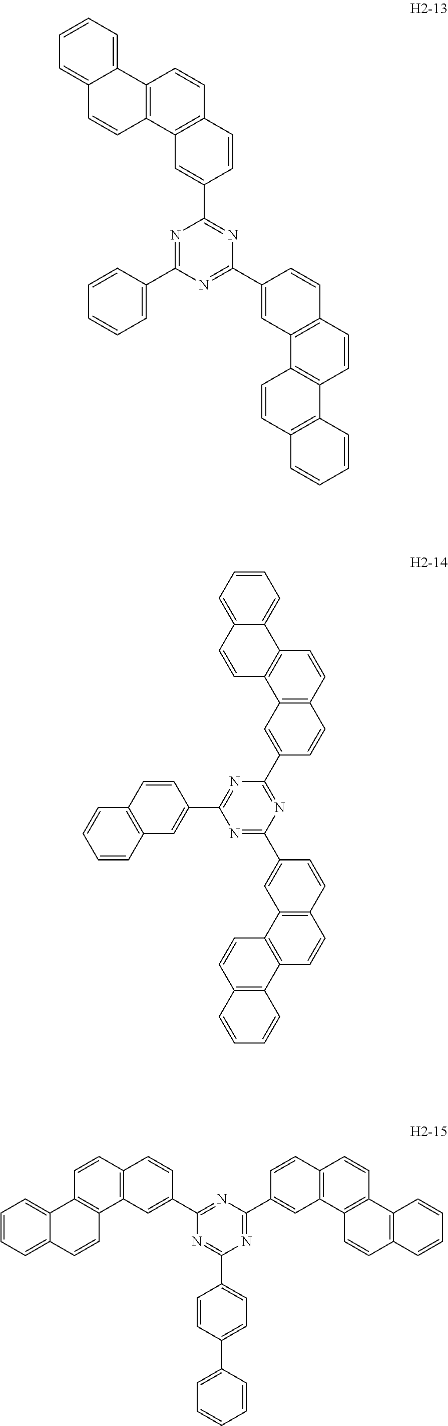 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc2cc(-c3nc(-c4ccc5ccc6c7ccccc7ccc6c5c4)nc(-c4ccc5ccc6c7ccccc7ccc6c5c4)n3)ccc2c1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1]

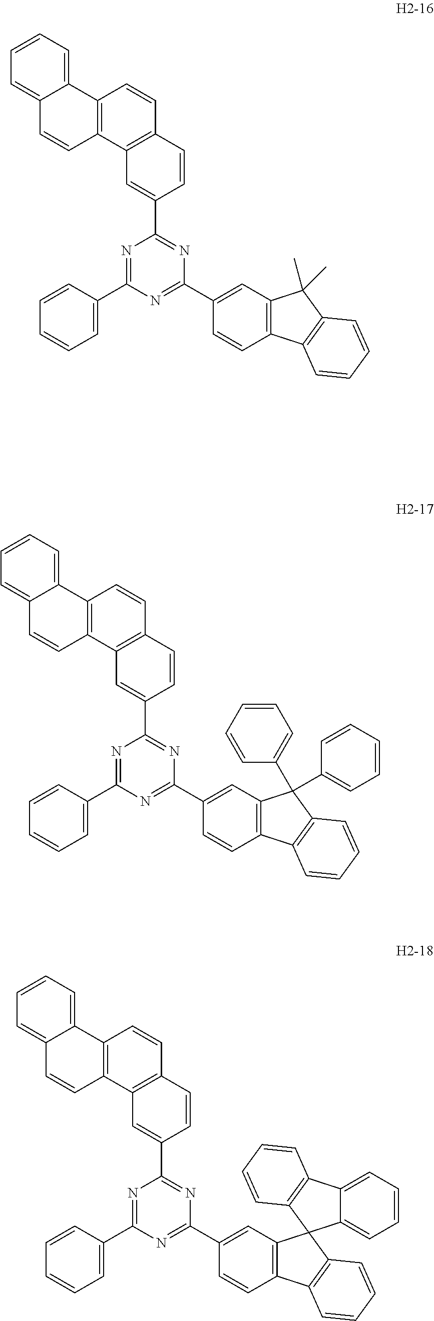 [SMILES: CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

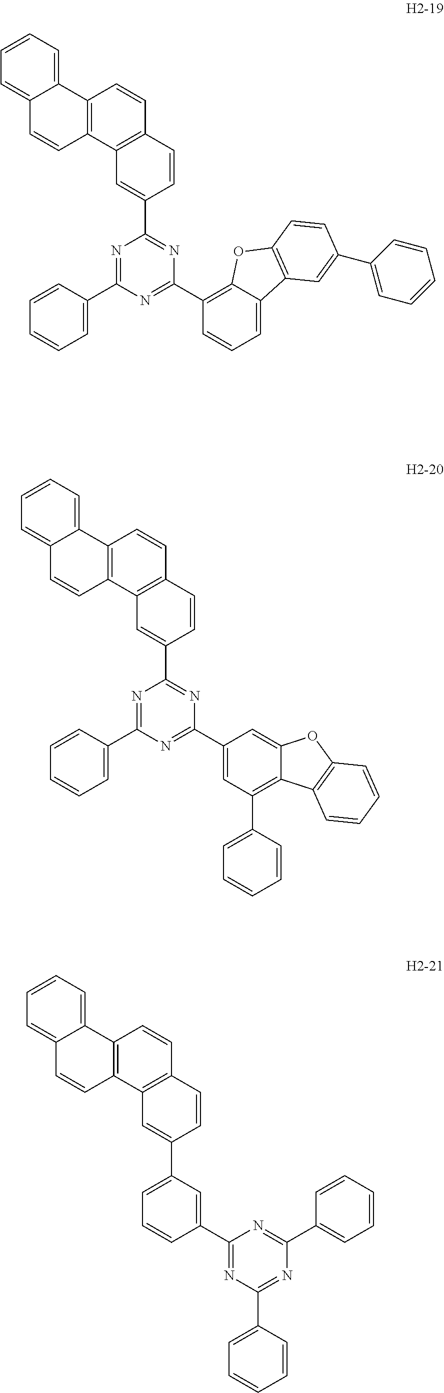 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1]

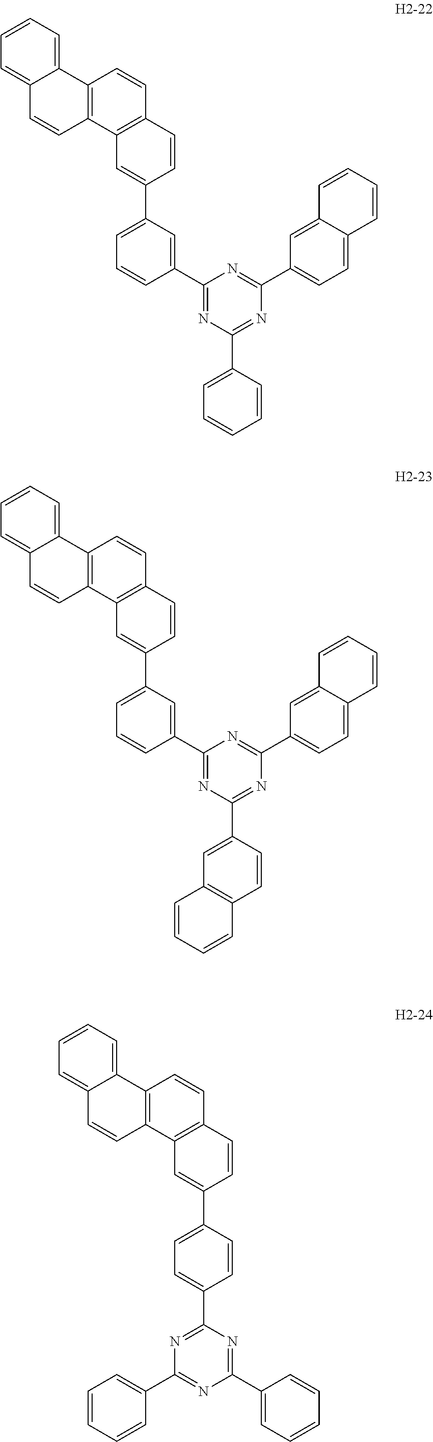 [SMILES: c1ccc(-c2nc(-c3cccc(-c4ccc5ccc6c7ccccc7ccc6c5c4)c3)nc(-c3ccc4ccccc4c3)n2)cc1 ; c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1]

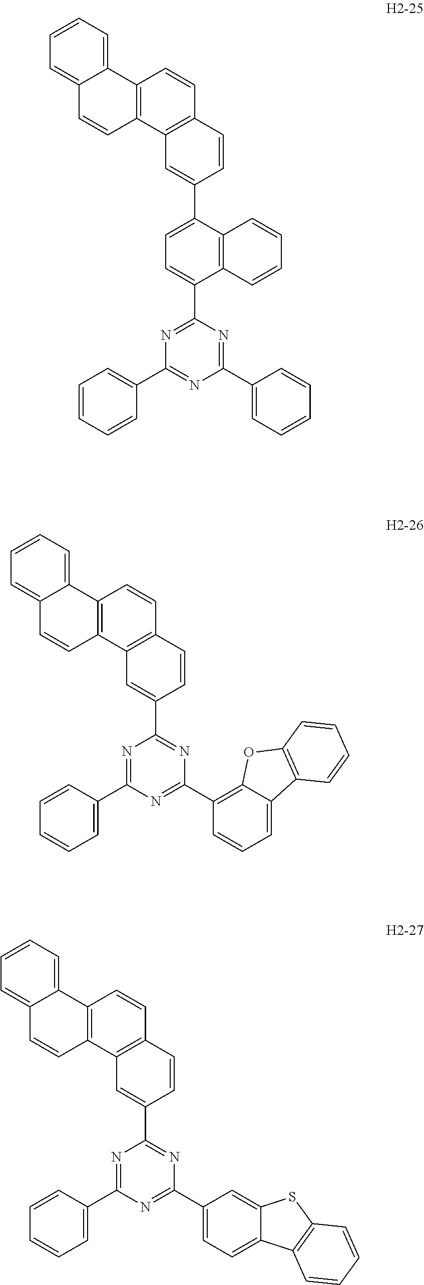 [SMILES: c1ccc(-c2nc(-c3ccccc3)nc(-c3ccc(-c4ccc5ccc6c7ccccc7ccc6c5c4)c4ccccc34)n2)cc1 ; c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1]

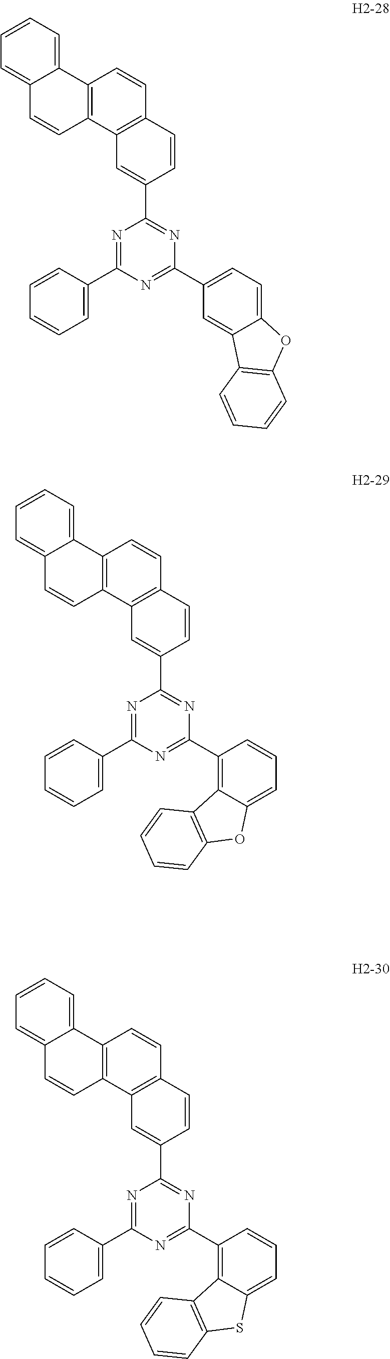 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

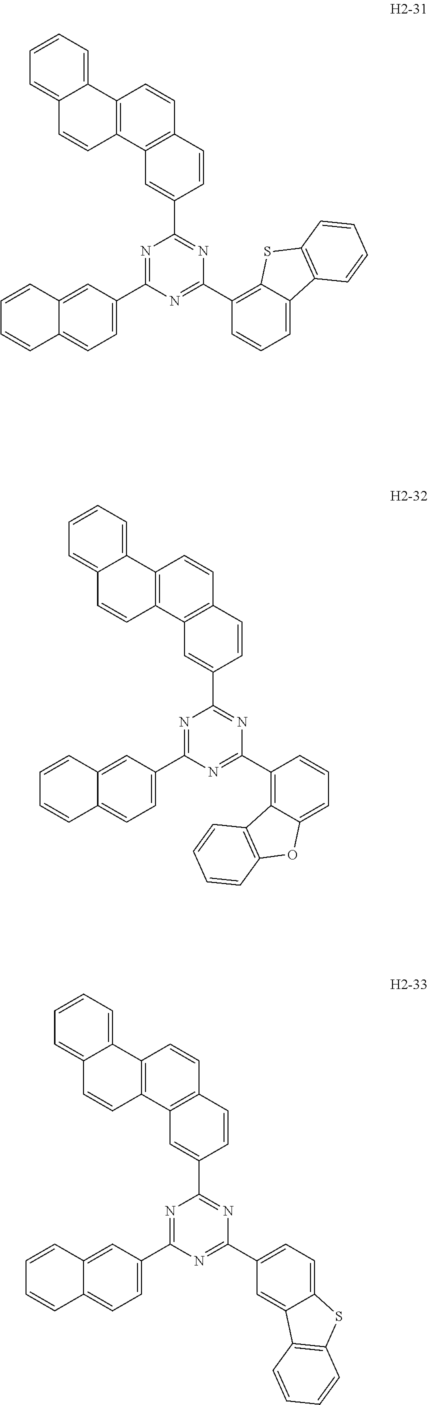 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

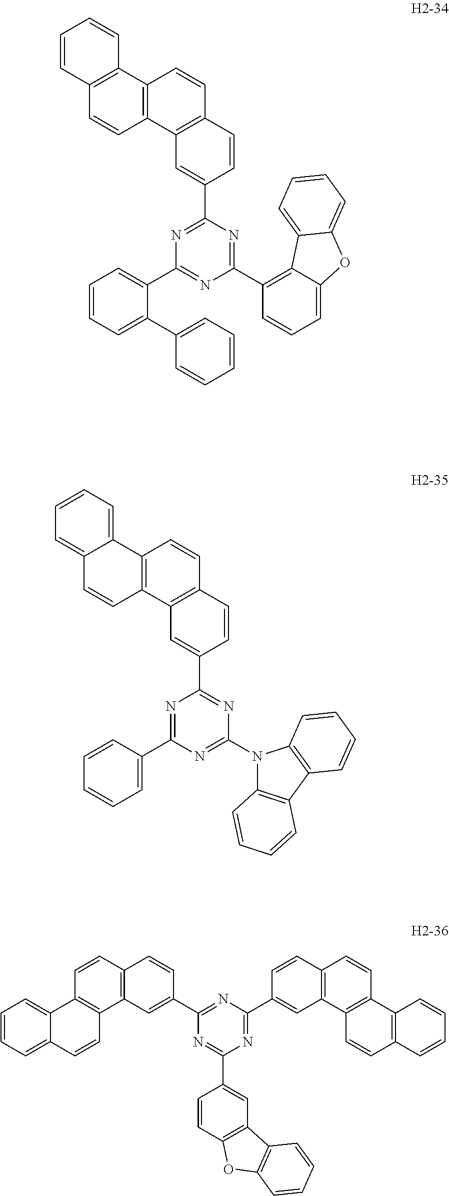 [SMILES: c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-n3c4ccccc4c4ccccc43)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21]

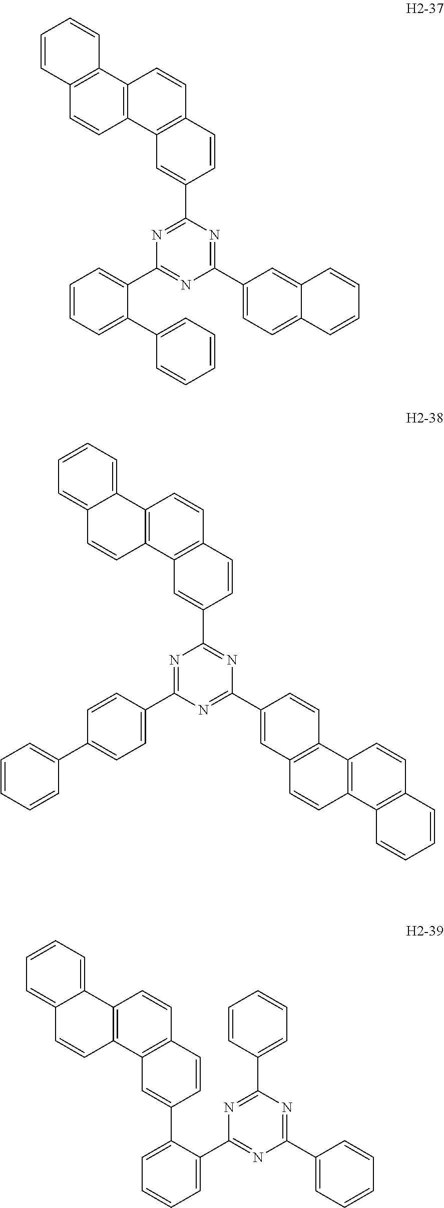 [SMILES: c1ccc(-c2ccccc2-c2nc(-c3ccc4ccccc4c3)nc(-c3ccc4ccc5c6ccccc6ccc5c4c3)n2)cc1 ; c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1]

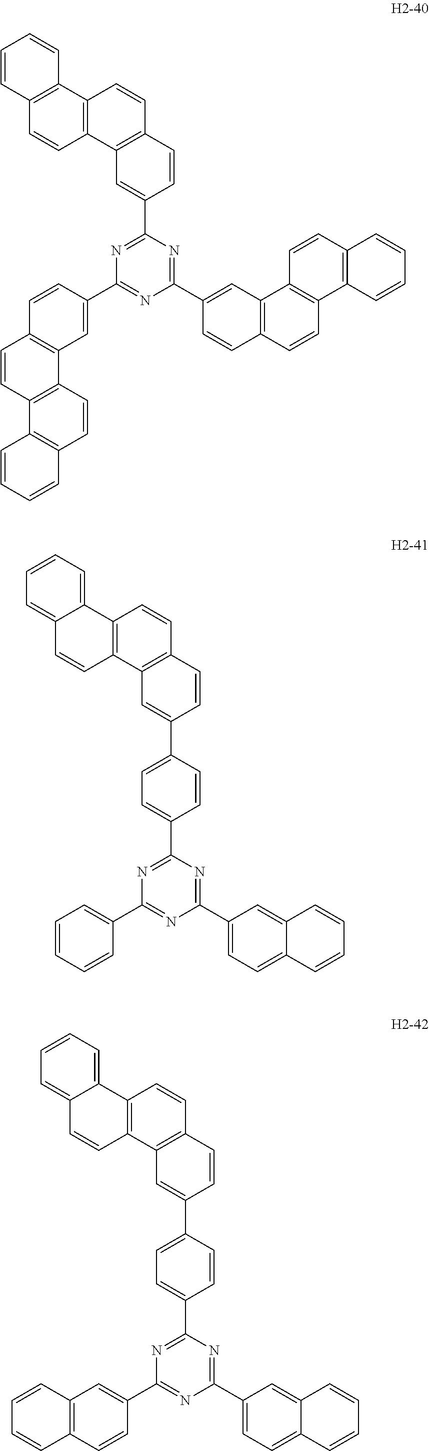 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21 ; c1ccc(-c2nc(-c3ccc(-c4ccc5ccc6c7ccccc7ccc6c5c4)cc3)nc(-c3ccc4ccccc4c3)n2)cc1 ; c1ccc2cc(-c3nc(-c4ccc(-c5ccc6ccc7c8ccccc8ccc7c6c5)cc4)nc(-c4ccc5ccccc5c4)n3)ccc2c1]

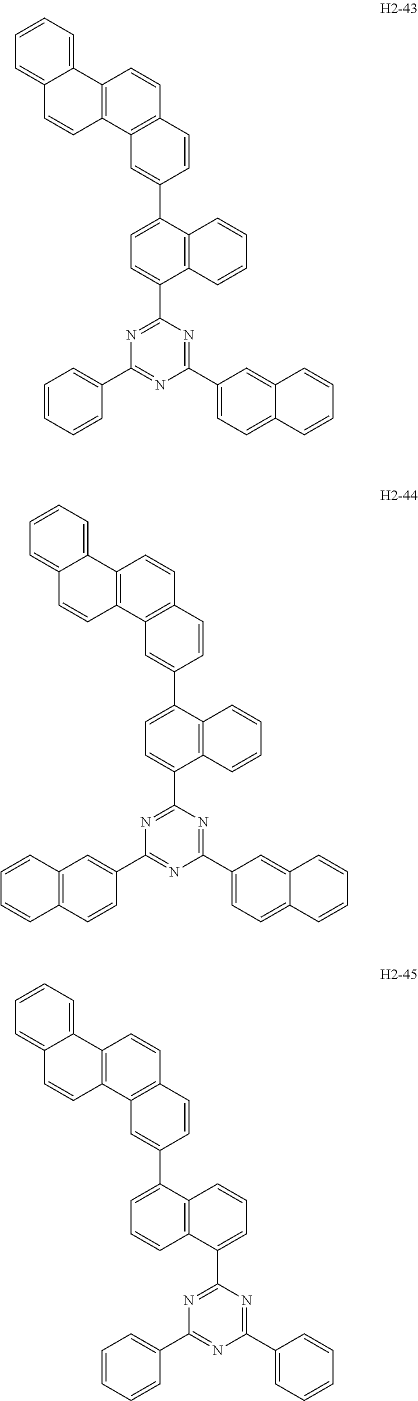 [SMILES: c1ccc(-c2nc(-c3ccc4ccccc4c3)nc(-c3ccc(-c4ccc5ccc6c7ccccc7ccc6c5c4)c4ccccc34)n2)cc1 ; c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1]

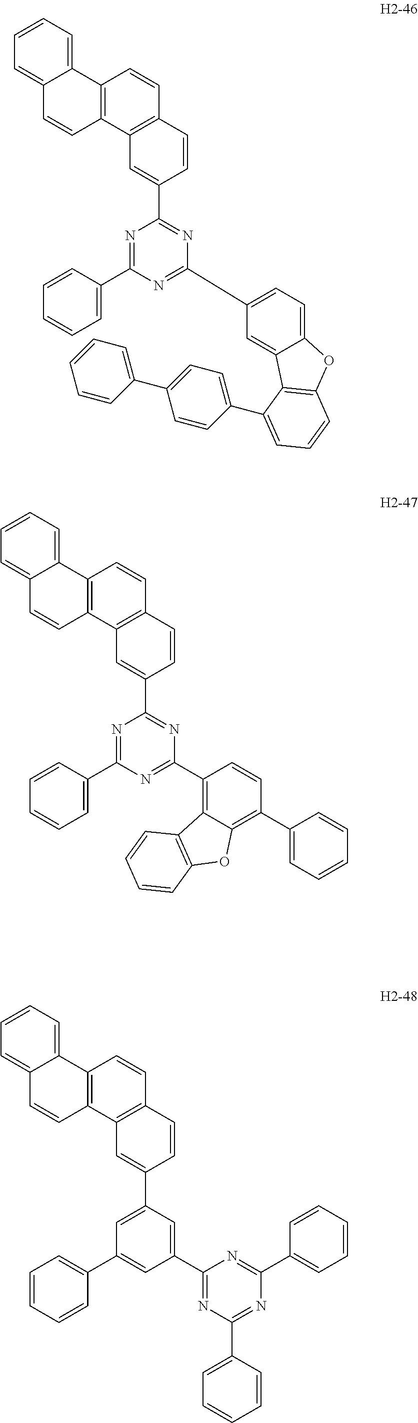 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

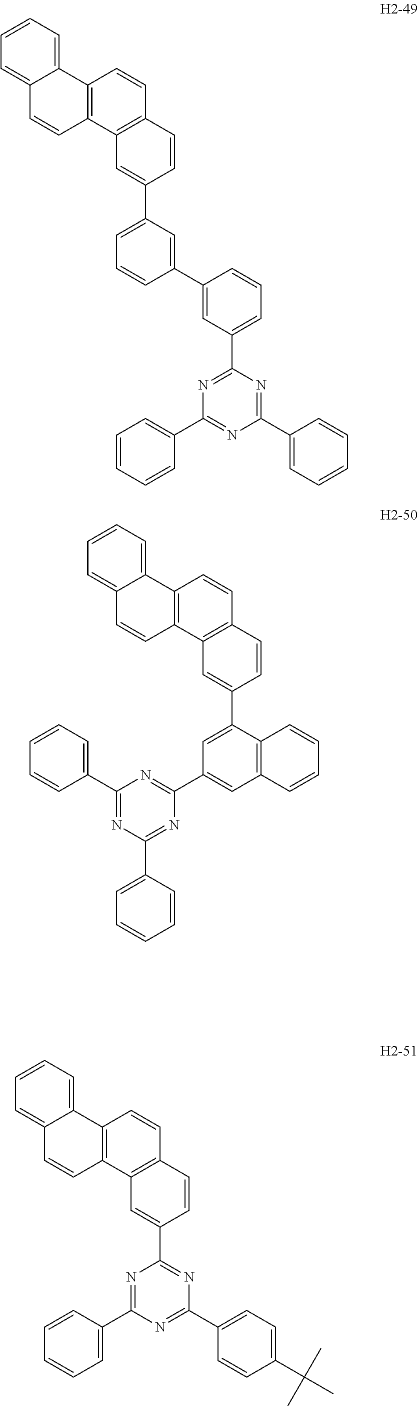 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1]

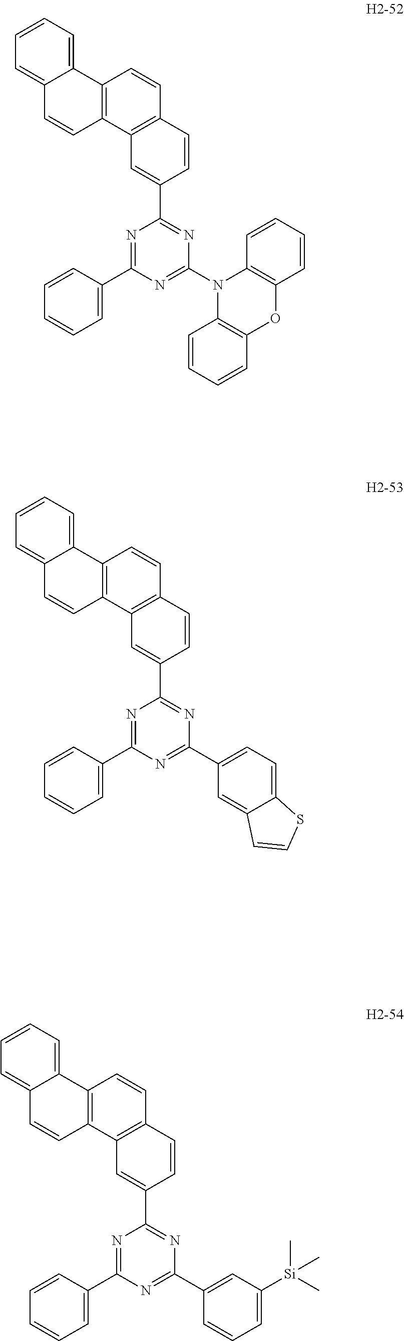 [SMILES: c1ccc(-c2nc(-c3ccc4ccc5c6ccccc6ccc5c4c3)nc(N3c4ccccc4Oc4ccccc43)n2)cc1 ; c1ccc(-c2nc(-c3ccc4sccc4c3)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1]

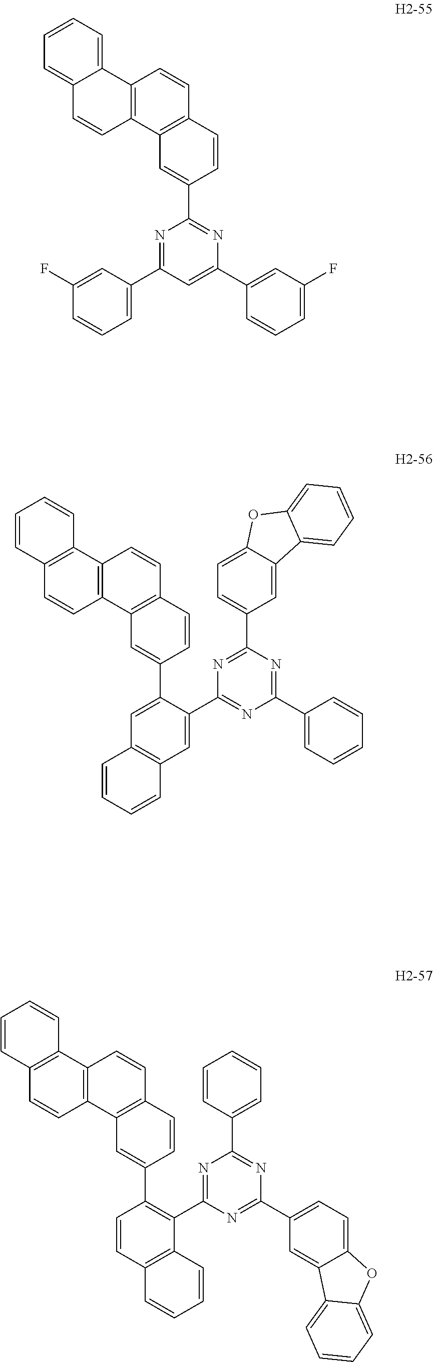 [SMILES: Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4oc5ccccc5c4c3)nc(-c3c(-c4ccc5ccc6c7ccccc7ccc6c5c4)ccc4ccccc34)n2)cc1]

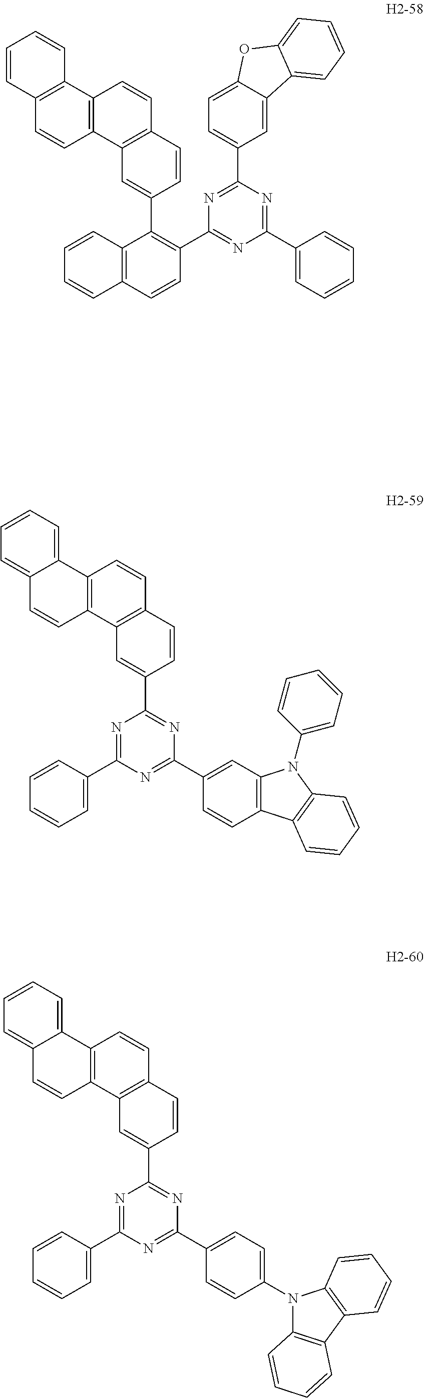 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc(-c2nc(-c3ccc4ccc5c6ccccc6ccc5c4c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3ccc4ccc5c6ccccc6ccc5c4c3)n2)cc1]

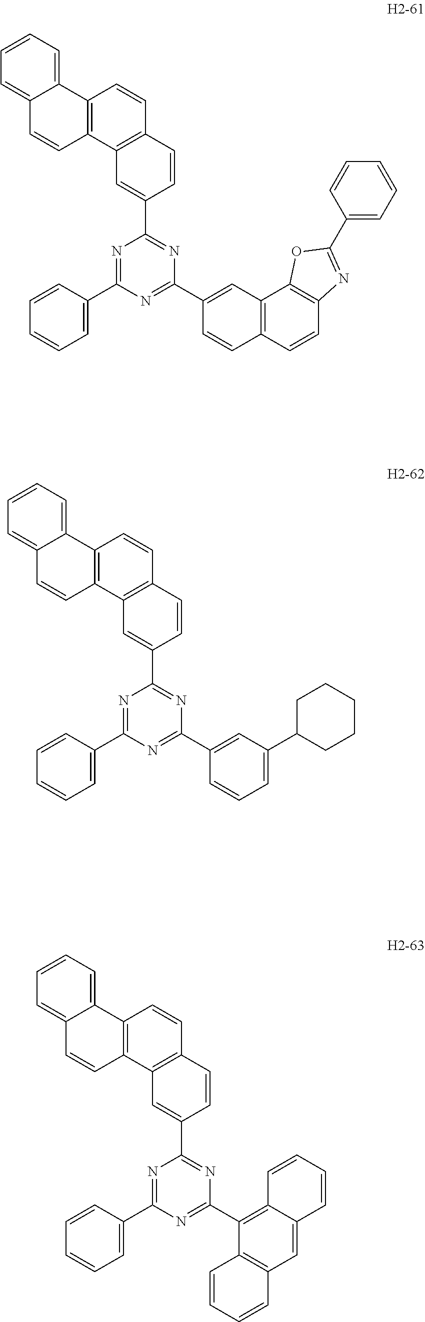 [SMILES: c1ccc(-c2nc(-c3ccc4ccc5c6ccccc6ccc5c4c3)nc(-c3ccc4ccc5nc(-c6ccccc6)oc5c4c3)n2)cc1 ; c1ccc(-c2nc(-c3cccc(C4CCCCC4)c3)nc(-c3ccc4ccc5c6ccccc6ccc5c4c3)n2)cc1 ; c1ccc(-c2nc(-c3ccc4ccc5c6ccccc6ccc5c4c3)nc(-c3c4ccccc4cc4ccccc34)n2)cc1]

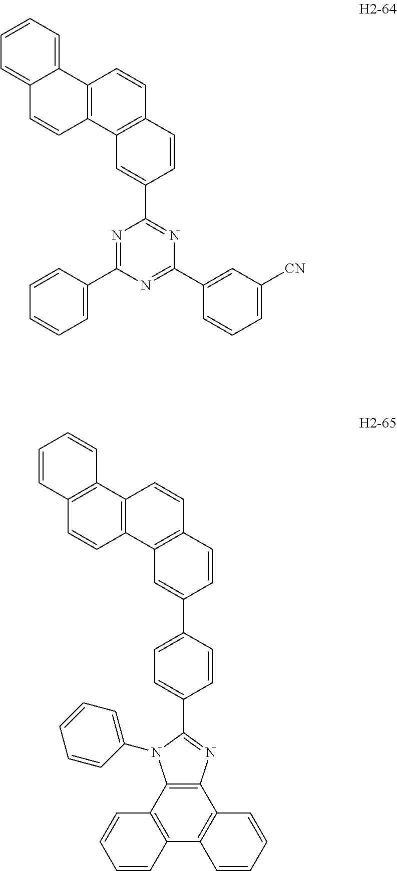 [SMILES: N#Cc1cccc(-c2nc(-c3ccccc3)nc(-c3ccc4ccc5c6ccccc6ccc5c4c3)n2)c1 ; c1ccc(-n2c(-c3ccc(-c4ccc5ccc6c7ccccc7ccc6c5c4)cc3)nc3c4ccccc4c4ccccc4c32)cc1]

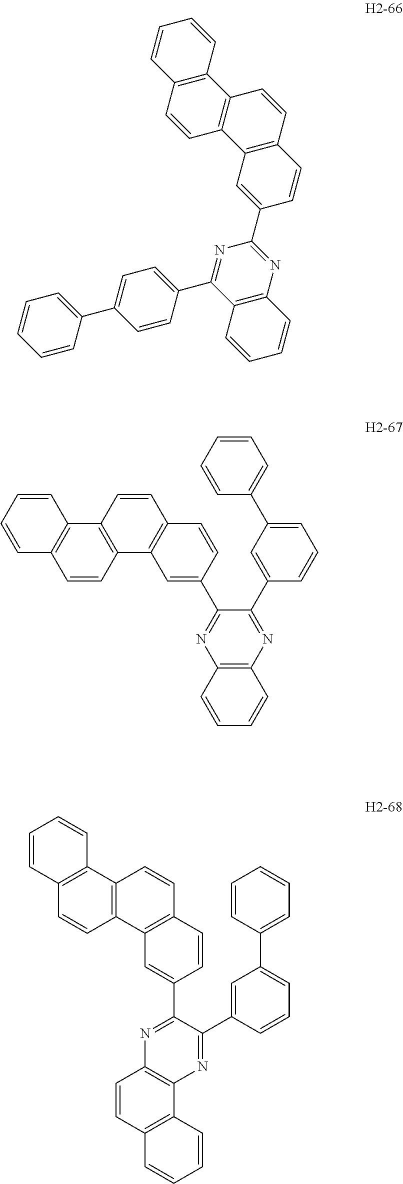 [SMILES: c1ccc(-c2ccc(-c3nc(-c4ccc5ccc6c7ccccc7ccc6c5c4)nc4ccccc34)cc2)cc1 ; c1ccc(-c2cccc(-c3nc4ccccc4nc3-c3ccc4ccc5c6ccccc6ccc5c4c3)c2)cc1 ; c1ccc(-c2cccc(-c3nc4c(ccc5ccccc54)nc3-c3ccc4ccc5c6ccccc6ccc5c4c3)c2)cc1]

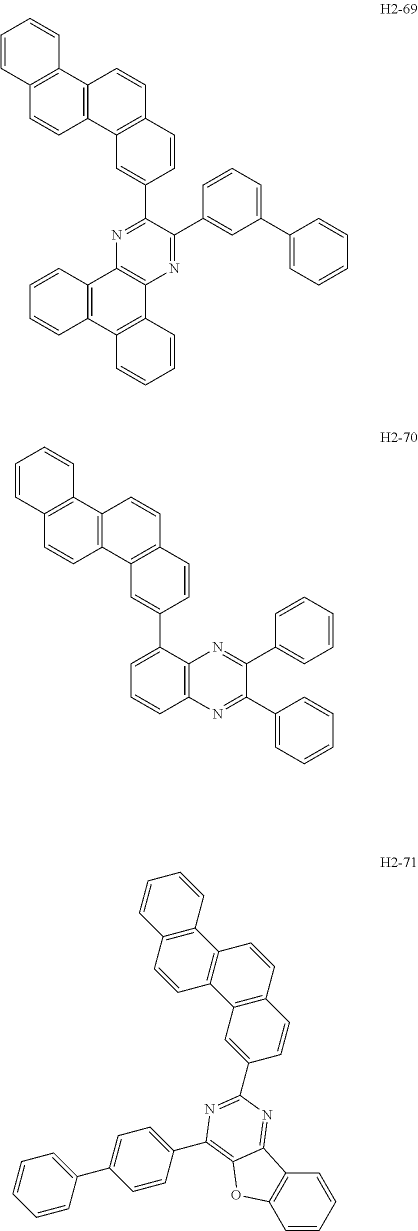 [SMILES: c1ccc(-c2cccc(-c3nc4c5ccccc5c5ccccc5c4nc3-c3ccc4ccc5c6ccccc6ccc5c4c3)c2)cc1 ; c1ccc(-c2nc3cccc(-c4ccc5ccc6c7ccccc7ccc6c5c4)c3nc2-c2ccccc2)cc1 ; c1ccc(-c2ccc(-c3nc(-c4ccc5ccc6c7ccccc7ccc6c5c4)nc4c3oc3ccccc34)cc2)cc1]

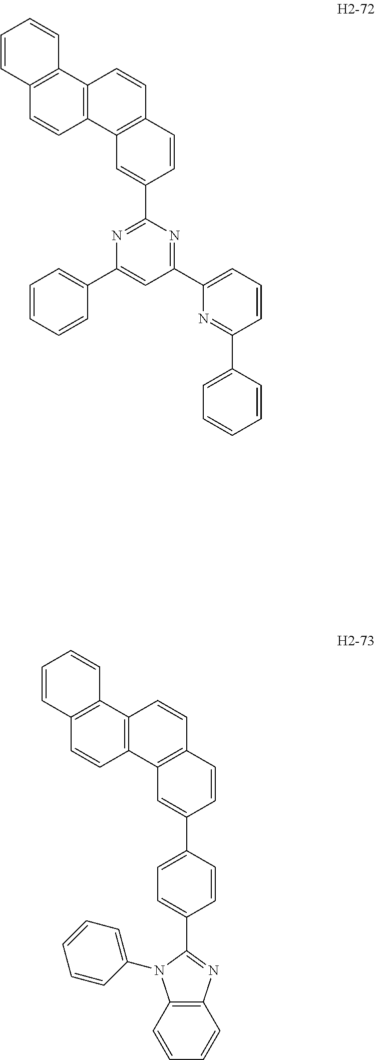 [SMILES: c1ccc(-c2cccc(-c3cc(-c4ccccc4)nc(-c4ccc5ccc6c7ccccc7ccc6c5c4)n3)n2)cc1 ; c1ccc(-n2c(-c3ccc(-c4ccc5ccc6c7ccccc7ccc6c5c4)cc3)nc3ccccc32)cc1]

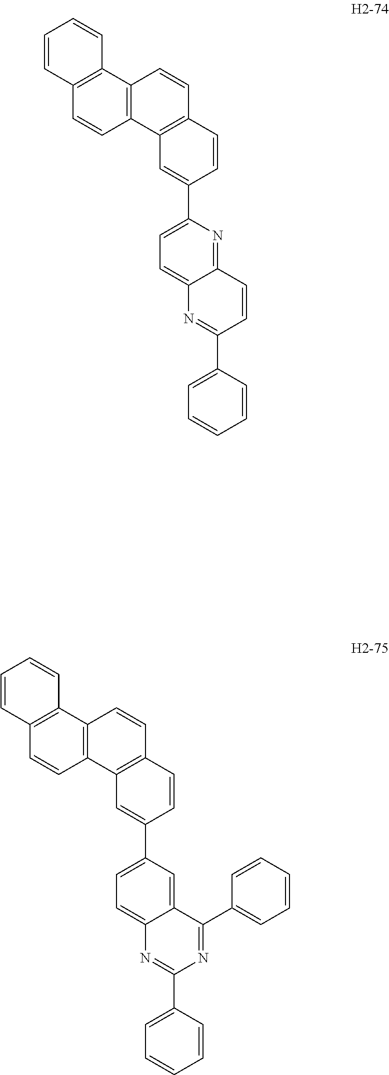 [SMILES: c1ccc(-c2ccc3nc(-c4ccc5ccc6c7ccccc7ccc6c5c4)ccc3n2)cc1 ; c1ccc(-c2nc(-c3ccccc3)c3cc(-c4ccc5ccc6c7ccccc7ccc6c5c4)ccc3n2)cc1]

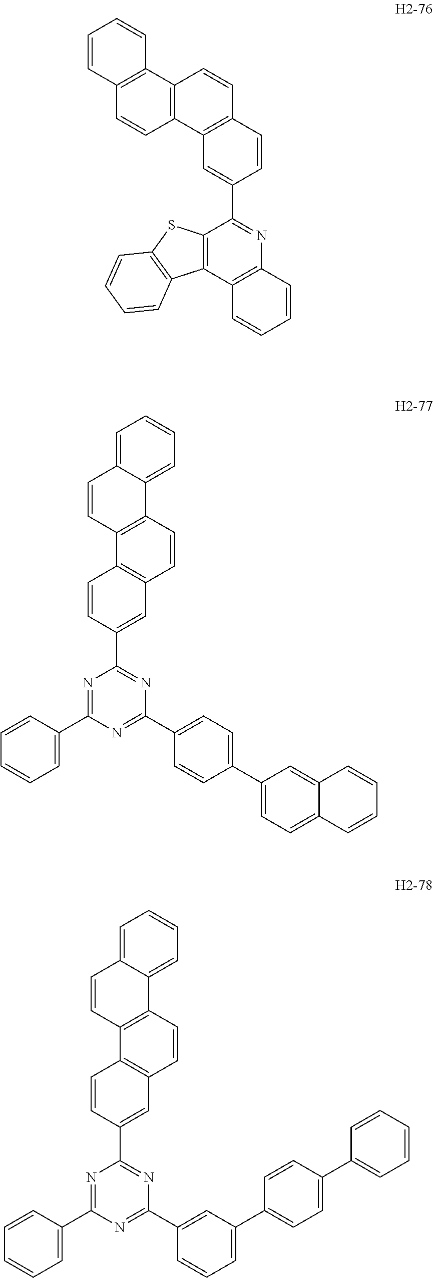 [SMILES: c1ccc2c(c1)ccc1c3cc(-c4nc5ccccc5c5c4sc4ccccc45)ccc3ccc21 ; c1ccc(-c2nc(-c3ccc(-c4ccc5ccccc5c4)cc3)nc(-c3ccc4c(ccc5c6ccccc6ccc45)c3)n2)cc1 ; c1ccc(-c2ccc(-c3cccc(-c4nc(-c5ccccc5)nc(-c5ccc6c(ccc7c8ccccc8ccc67)c5)n4)c3)cc2)cc1]

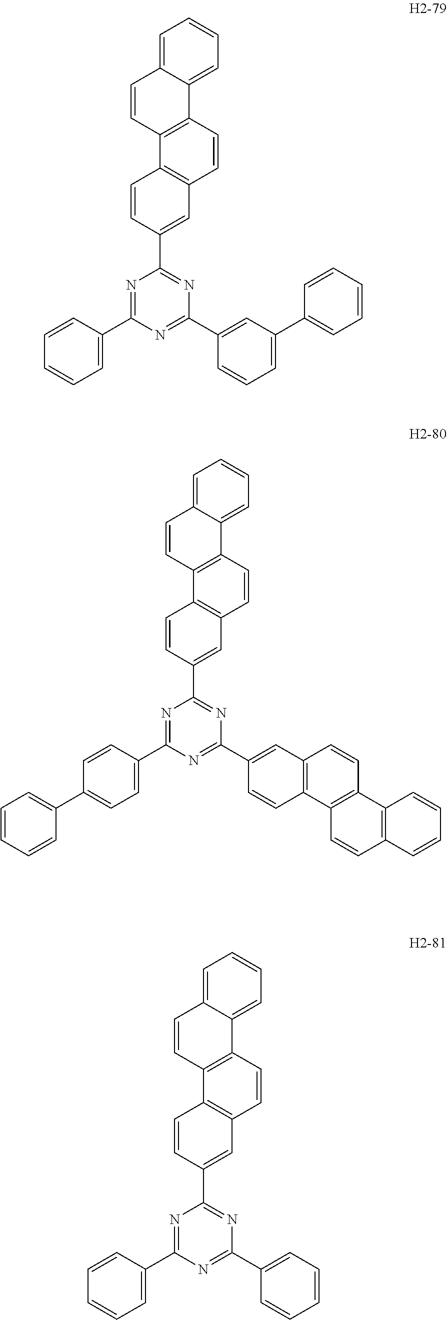 [SMILES: c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1 ; c1ccc(-c2nc(-c3ccccc3)nc(-c3ccc4c(ccc5c6ccccc6ccc45)c3)n2)cc1]

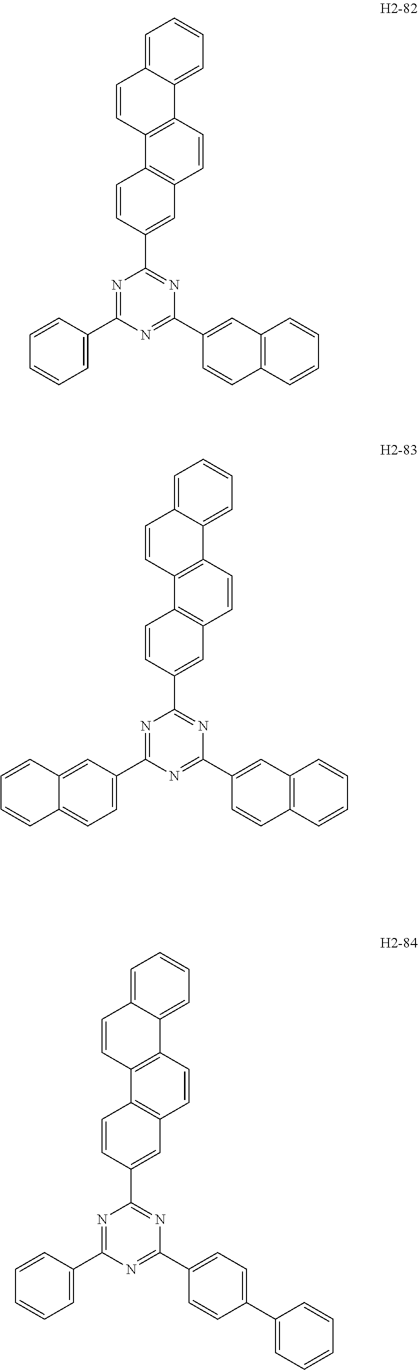 [SMILES: c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1 ; c1ccc(-c2ccc(-c3nc(-c4ccccc4)nc(-c4ccc5c(ccc6c7ccccc7ccc56)c4)n3)cc2)cc1]

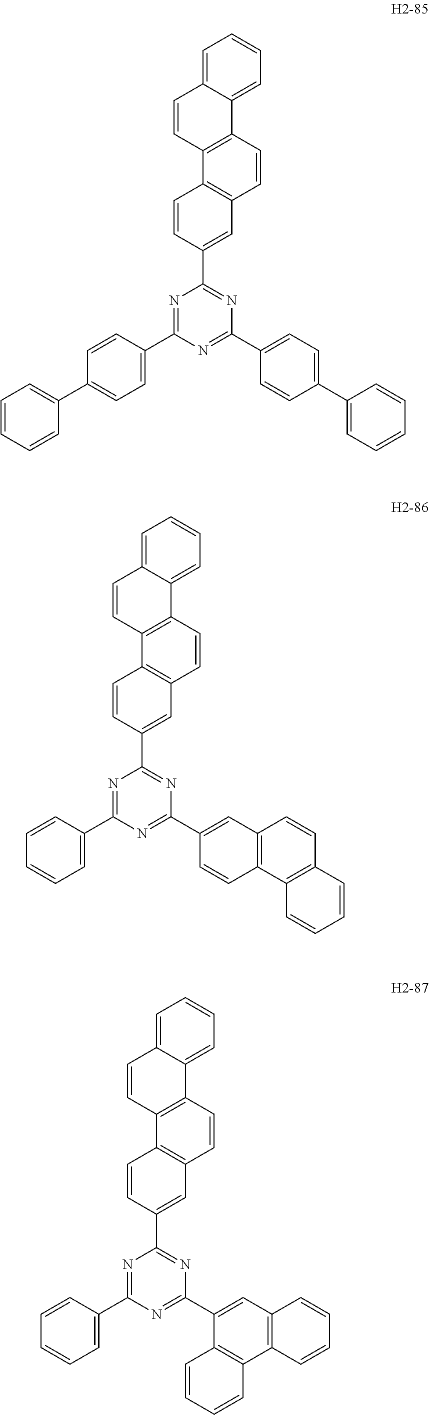 [SMILES: c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1 ; c1ccc(-c2nc(-c3ccc4c(ccc5c6ccccc6ccc45)c3)nc(-c3cc4ccccc4c4ccccc34)n2)cc1]

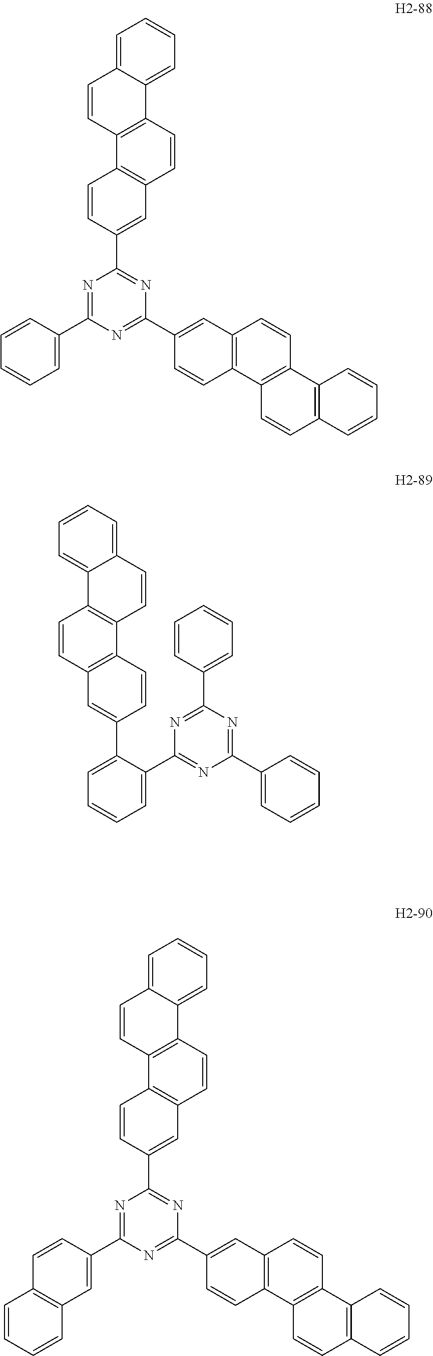 [SMILES: c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1 ; c1ccc2cc(-c3nc(-c4ccc5c(ccc6c7ccccc7ccc56)c4)nc(-c4ccc5c(ccc6c7ccccc7ccc56)c4)n3)ccc2c1]

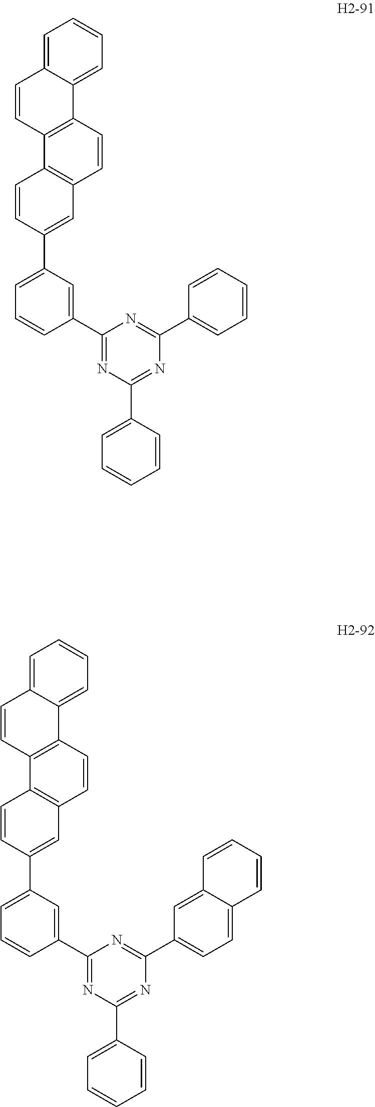 [SMILES: c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

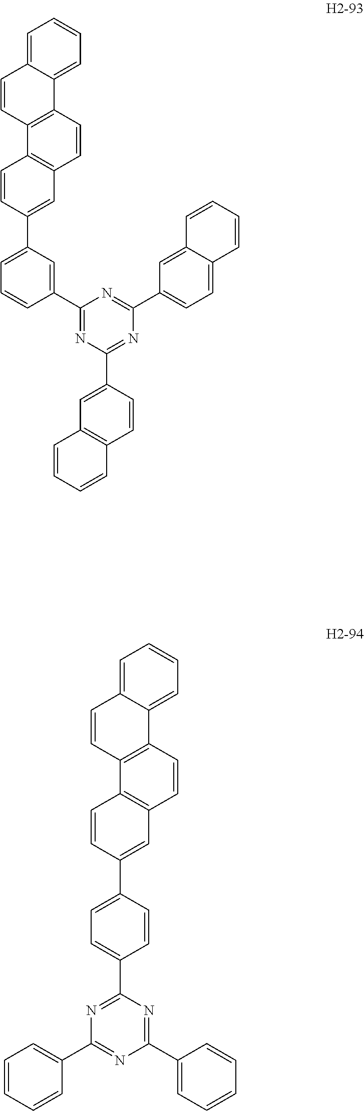 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

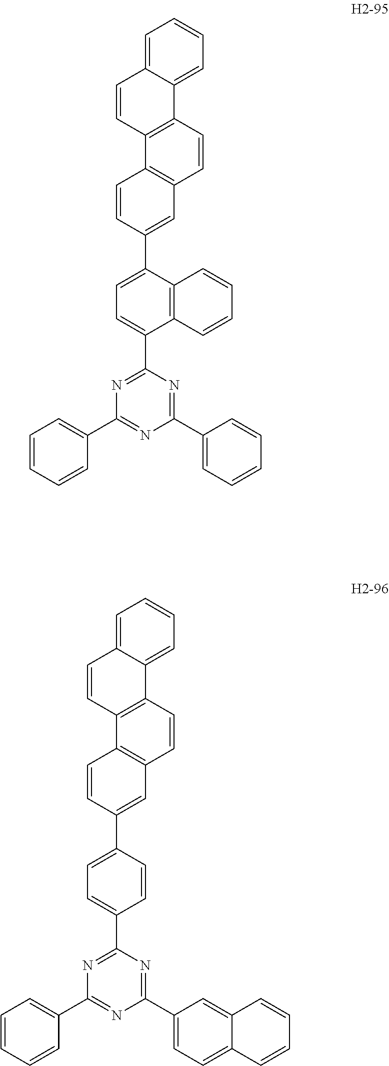 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

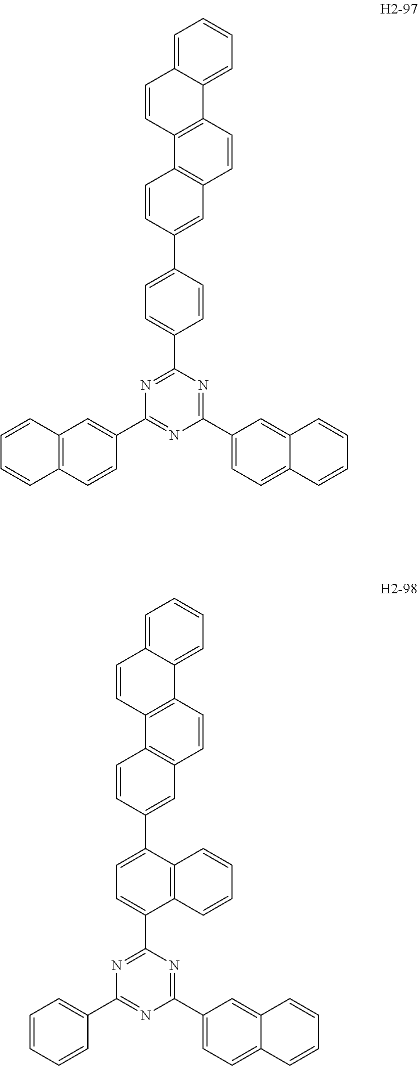 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

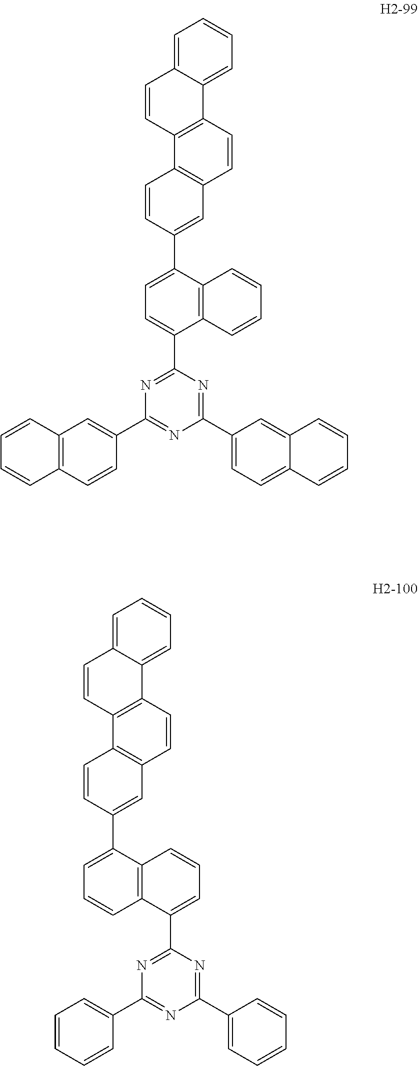 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3cccc4c(-c5ccc6c(ccc7c8ccccc8ccc67)c5)cccc34)n2)cc1]

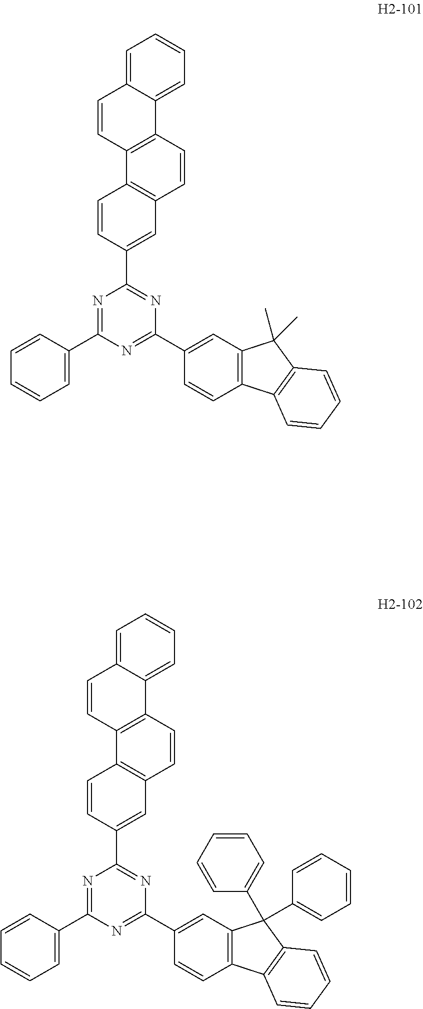 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

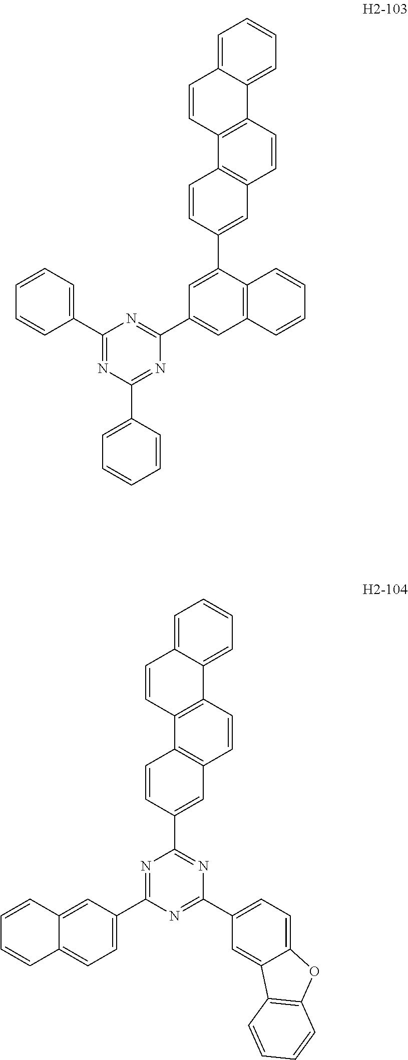 [SMILES: c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1]

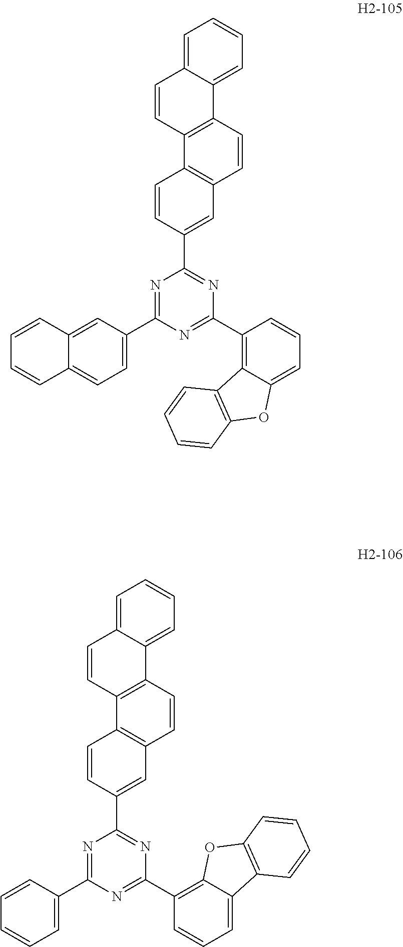 [SMILES: c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1]

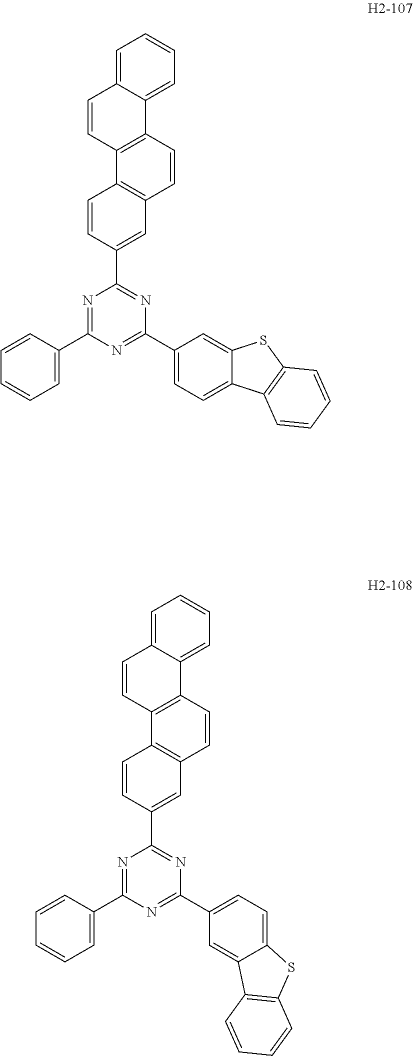 [SMILES: c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

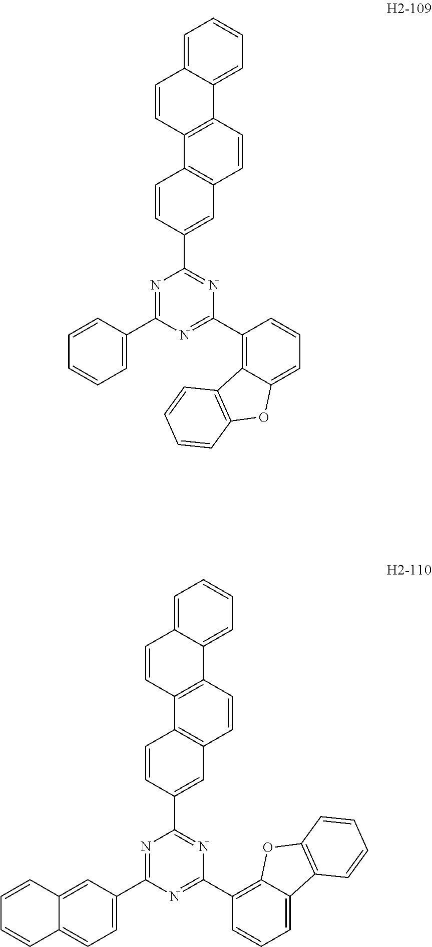 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

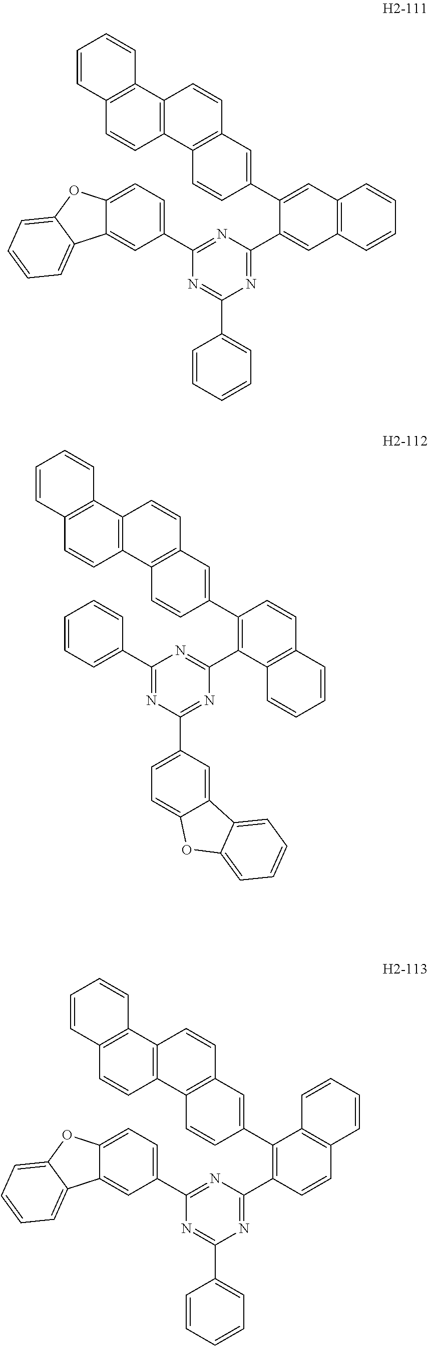 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1]

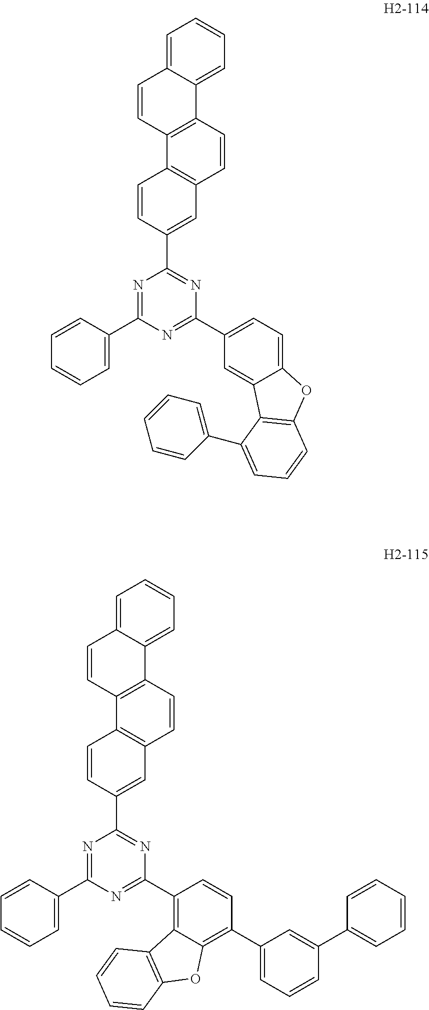 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1]

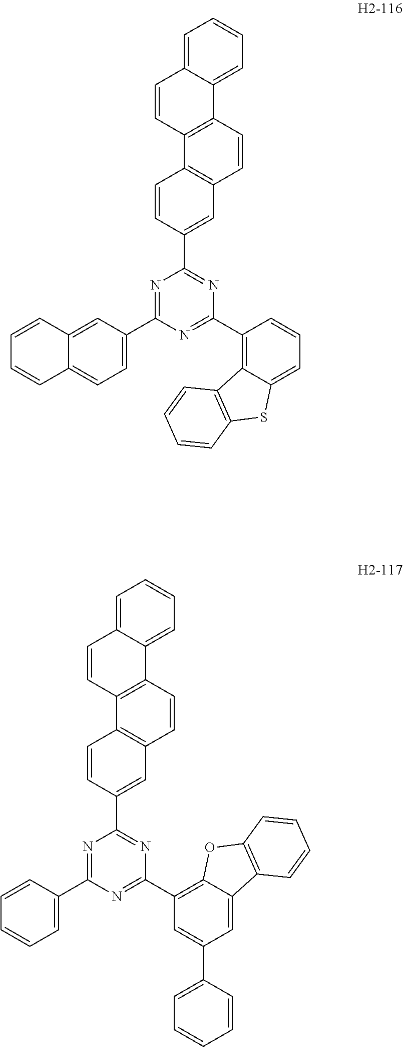 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

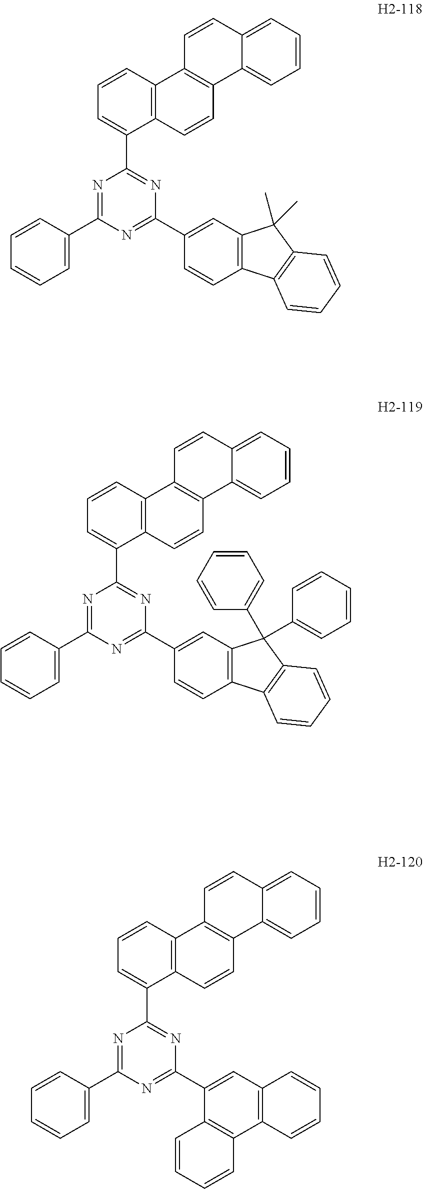 [SMILES: CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1]

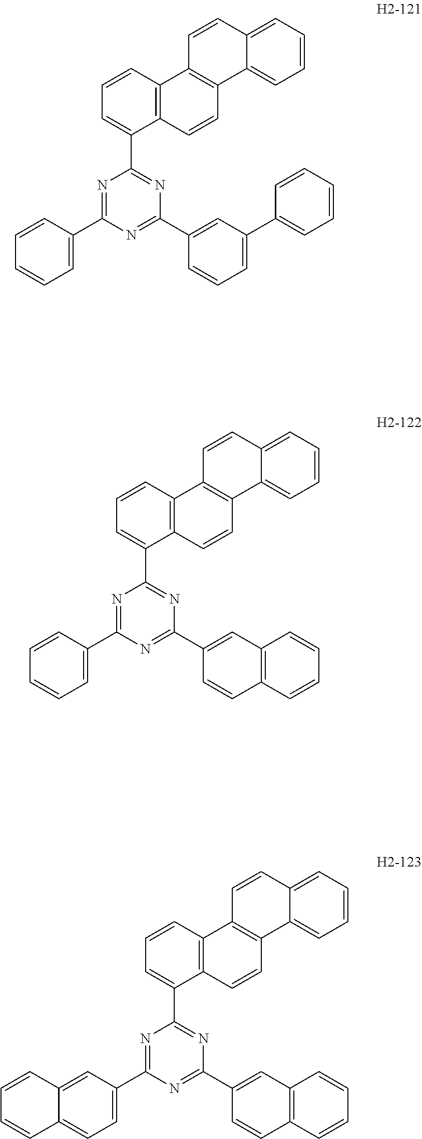 [SMILES: c1ccc(-c2cccc(-c3nc(-c4ccccc4)nc(-c4cccc5c4ccc4c6ccccc6ccc54)n3)c2)cc1 ; c1ccc(-c2nc(-c3ccc4ccccc4c3)nc(-c3cccc4c3ccc3c5ccccc5ccc43)n2)cc1 ; c1ccc2cc(-c3nc(-c4ccc5ccccc5c4)nc(-c4cccc5c4ccc4c6ccccc6ccc54)n3)ccc2c1]

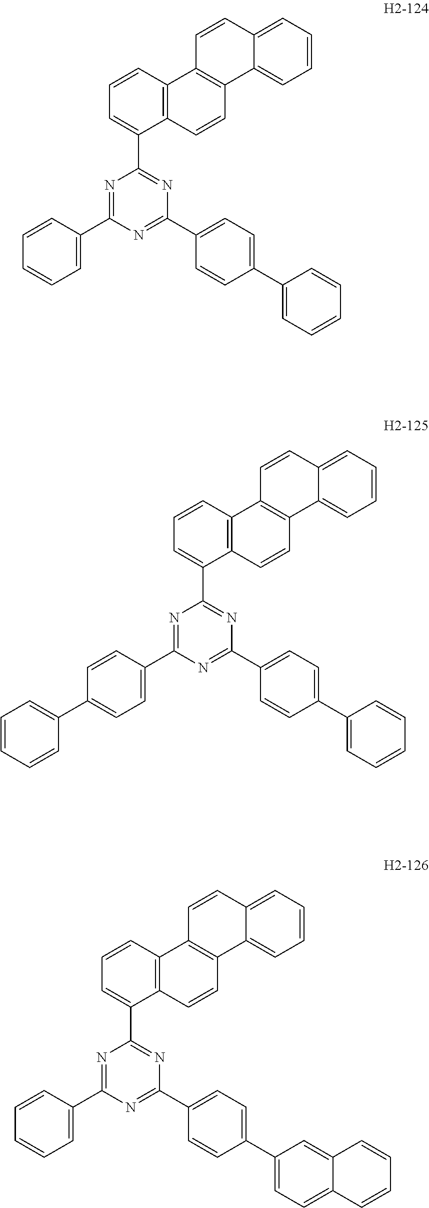 [SMILES: c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

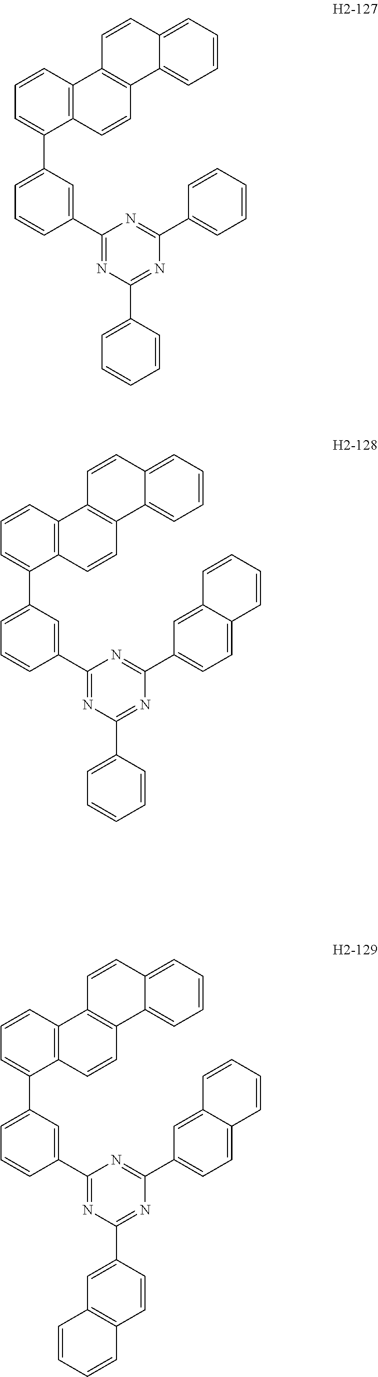 [SMILES: c1ccc(-c2nc(-c3ccccc3)nc(-c3cccc(-c4cccc5c4ccc4c6ccccc6ccc54)c3)n2)cc1 ; c1ccc(-c2nc(-c3cccc(-c4cccc5c4ccc4c6ccccc6ccc54)c3)nc(-c3ccc4ccccc4c3)n2)cc1 ; c1cc(-c2nc(-c3ccc4ccccc4c3)nc(-c3ccc4ccccc4c3)n2)cc(-c2cccc3c2ccc2c4ccccc4ccc32)c1]

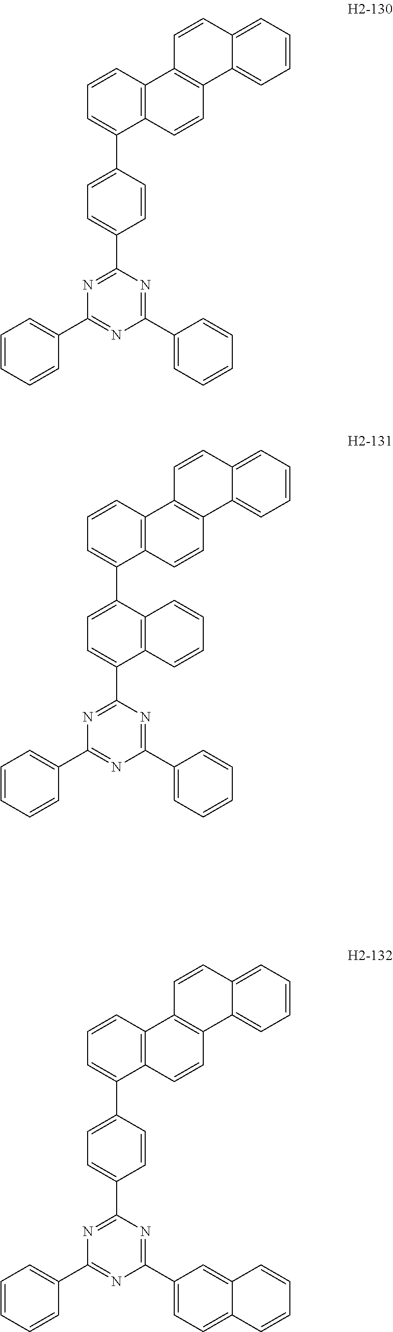 [SMILES: c1ccc(-c2nc(-c3ccccc3)nc(-c3ccc(-c4cccc5c4ccc4c6ccccc6ccc54)cc3)n2)cc1 ; c1ccc(-c2nc(-c3ccccc3)nc(-c3ccc(-c4cccc5c4ccc4c6ccccc6ccc54)c4ccccc34)n2)cc1 ; c1ccc(-c2nc(-c3ccc(-c4cccc5c4ccc4c6ccccc6ccc54)cc3)nc(-c3ccc4ccccc4c3)n2)cc1]

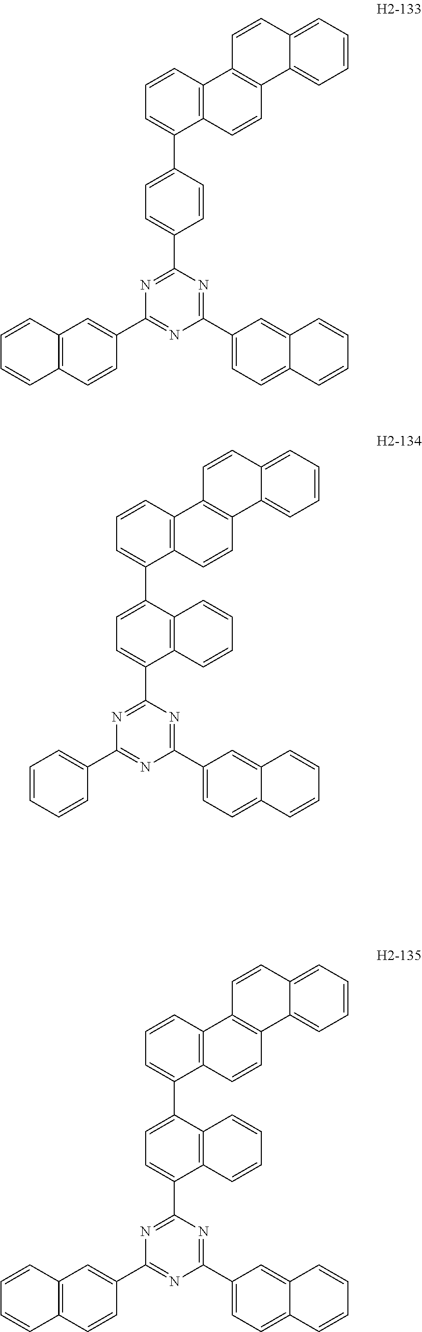 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

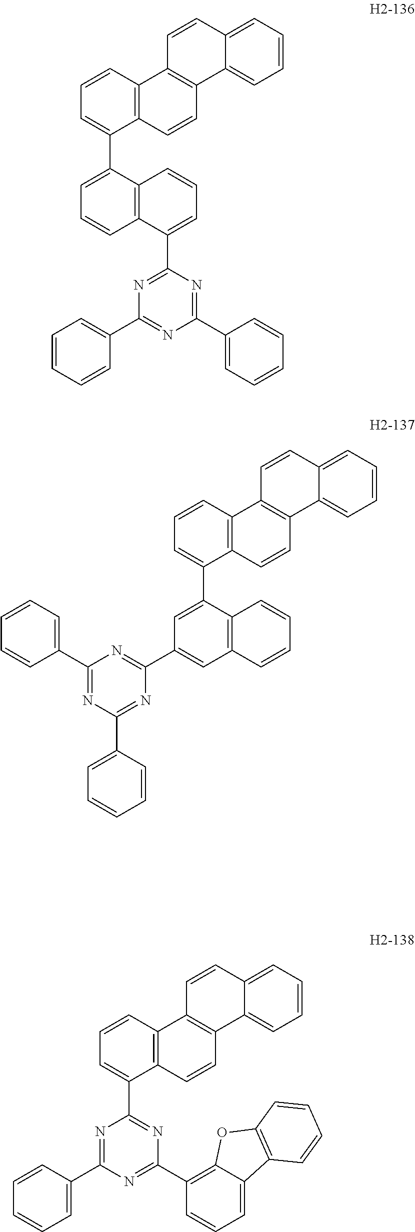 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1]

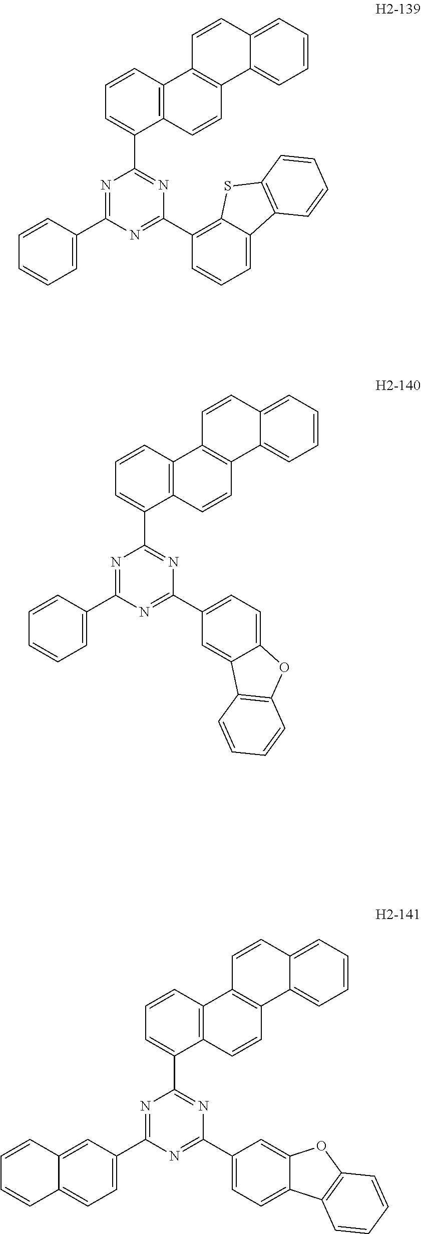 [SMILES: c1ccc(-c2nc(-c3cccc4c3ccc3c5ccccc5ccc43)nc(-c3cccc4c3sc3ccccc34)n2)cc1 ; c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1]

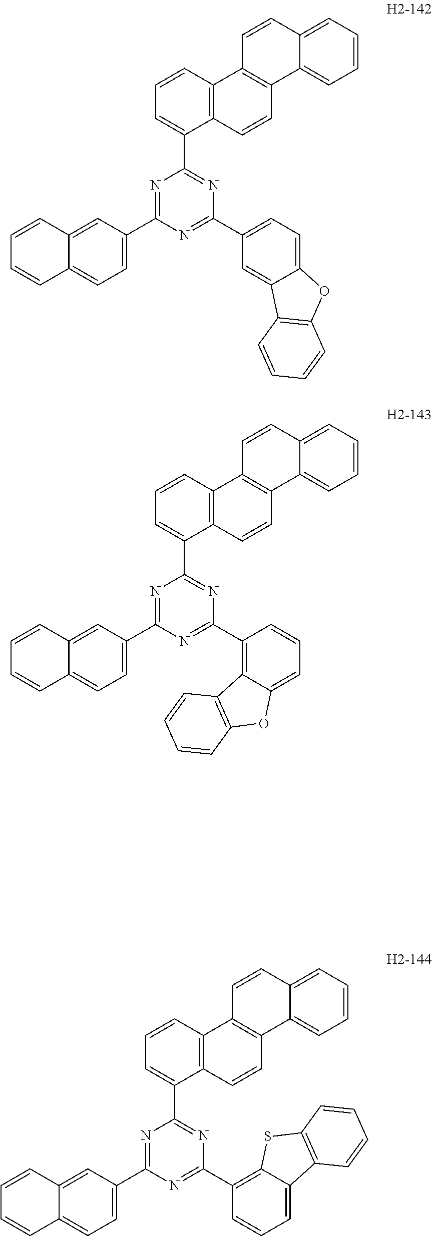 [SMILES: c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1]

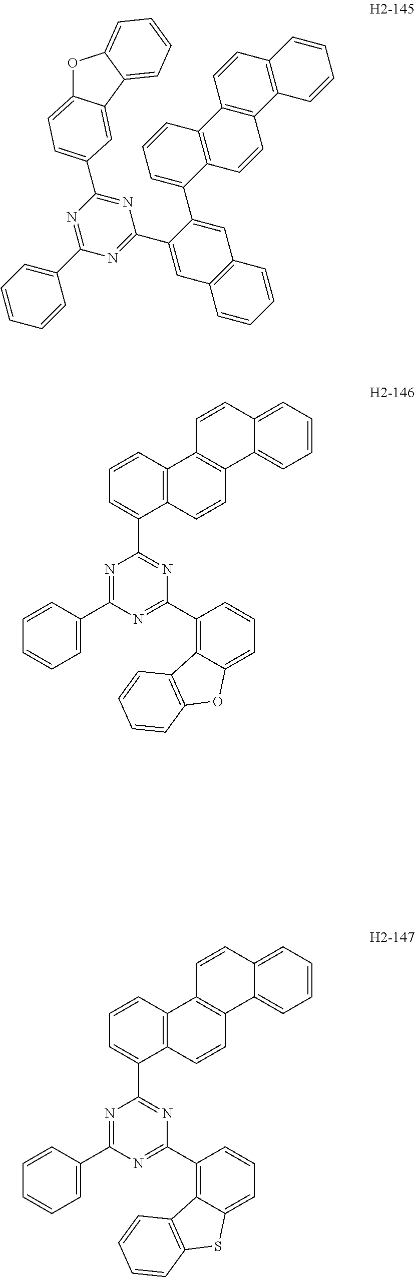 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1]

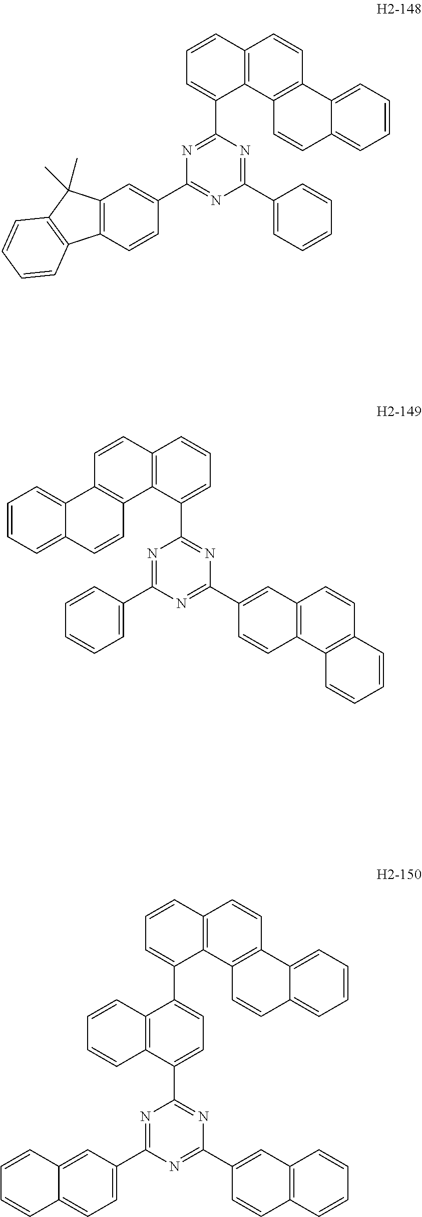 [SMILES: CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1]

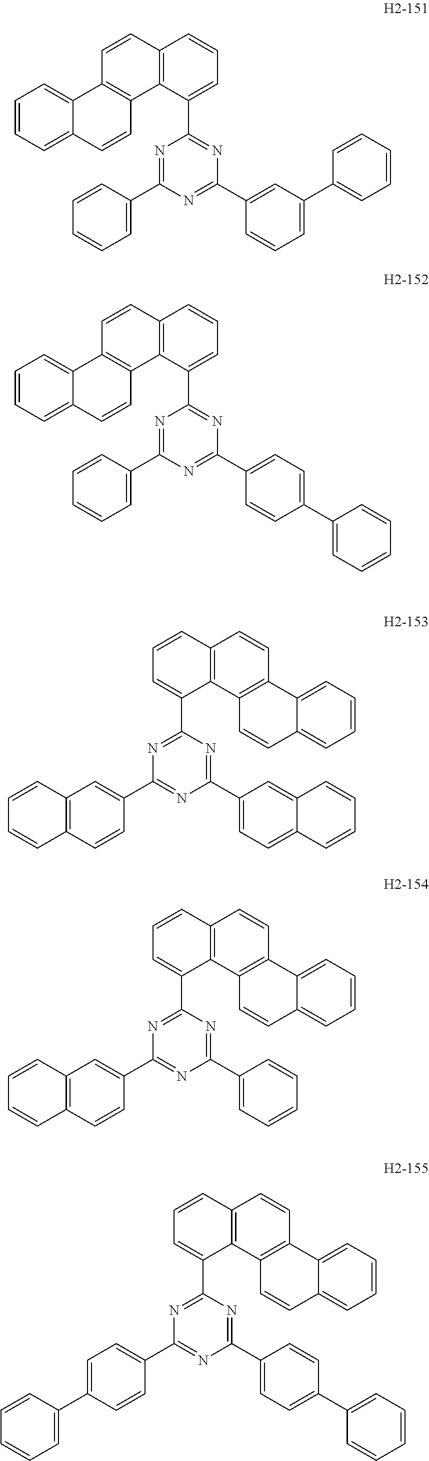 [SMILES: c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1 ; c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1]

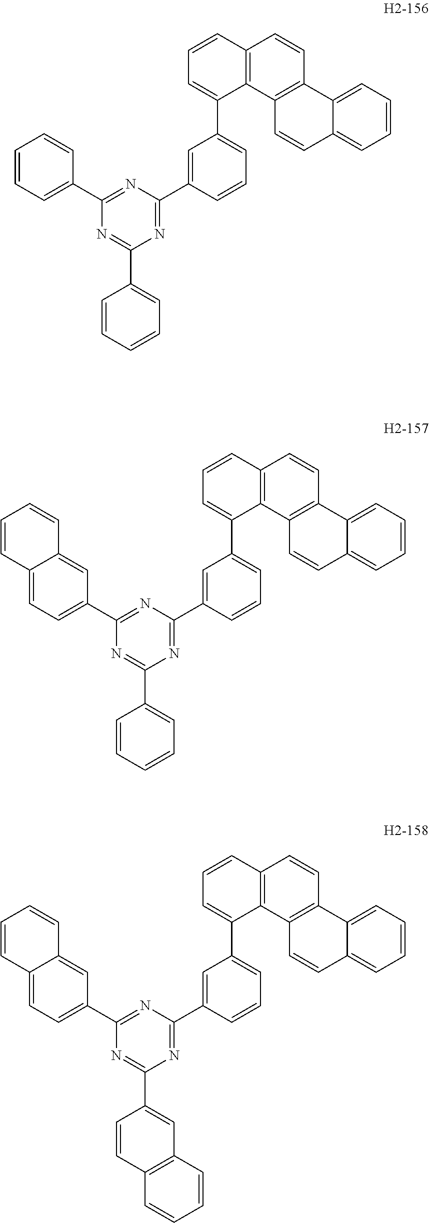 [SMILES: c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1 ; c1cc(-c2nc(-c3ccc4ccccc4c3)nc(-c3ccc4ccccc4c3)n2)cc(-c2cccc3ccc4c5ccccc5ccc4c23)c1]

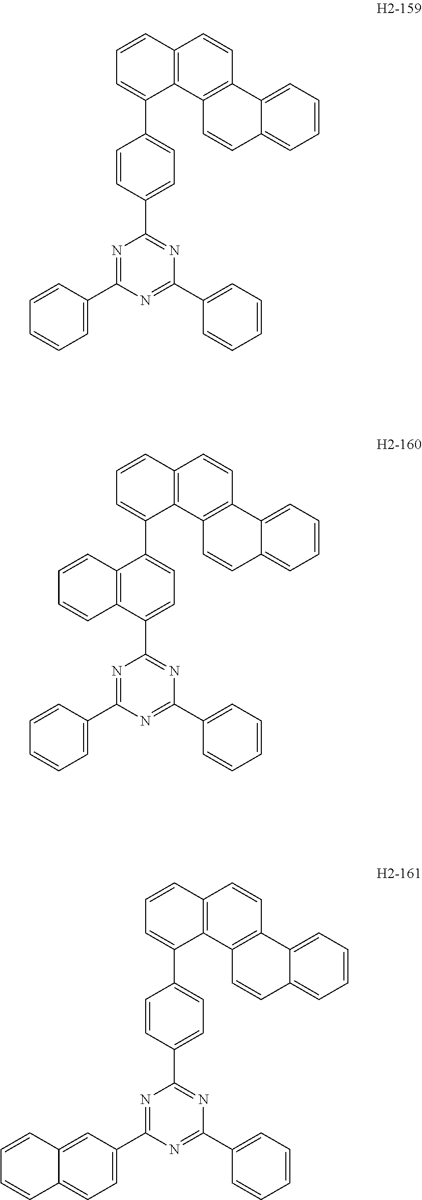 [SMILES: c1ccc(-c2nc(-c3ccccc3)nc(-c3ccc(-c4cccc5ccc6c7ccccc7ccc6c45)cc3)n2)cc1 ; c1ccc(-c2nc(-c3ccccc3)nc(-c3ccc(-c4cccc5ccc6c7ccccc7ccc6c45)c4ccccc34)n2)cc1 ; c1ccc(-c2nc(-c3ccc(-c4cccc5ccc6c7ccccc7ccc6c45)cc3)nc(-c3ccc4ccccc4c3)n2)cc1]

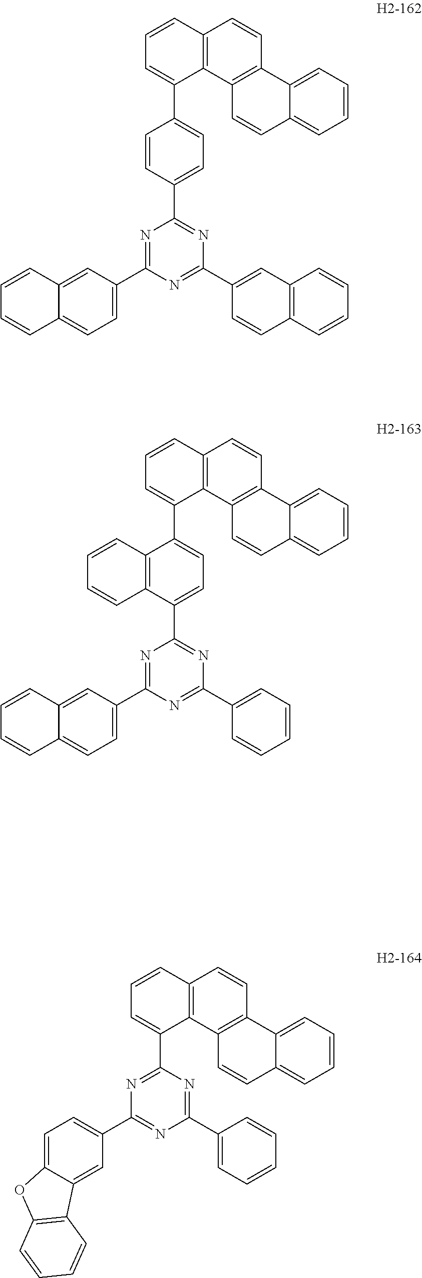 [SMILES: c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1 ; c1ccc(-c2nc(-c3ccc4oc5ccccc5c4c3)nc(-c3cccc4ccc5c6ccccc6ccc5c34)n2)cc1]

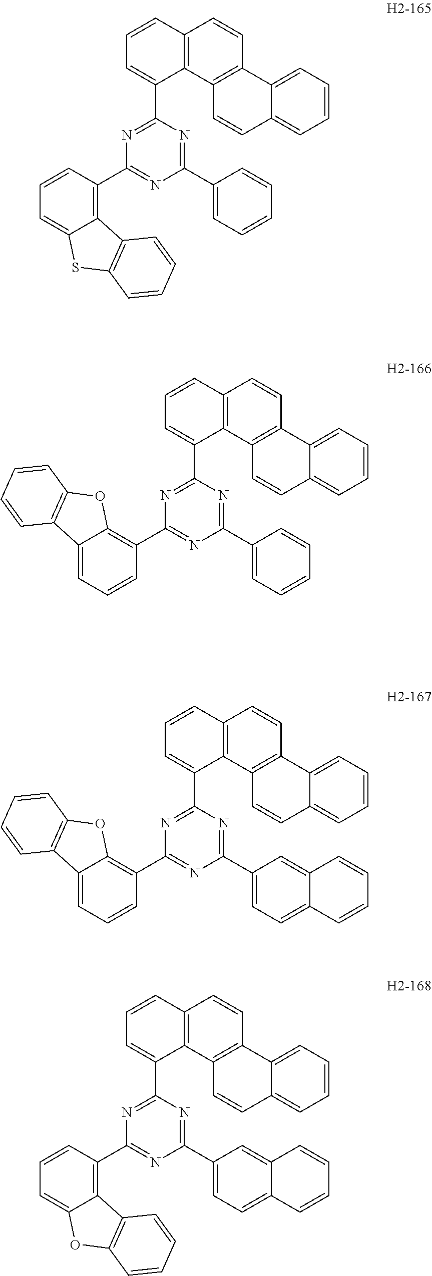 [SMILES: c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1]

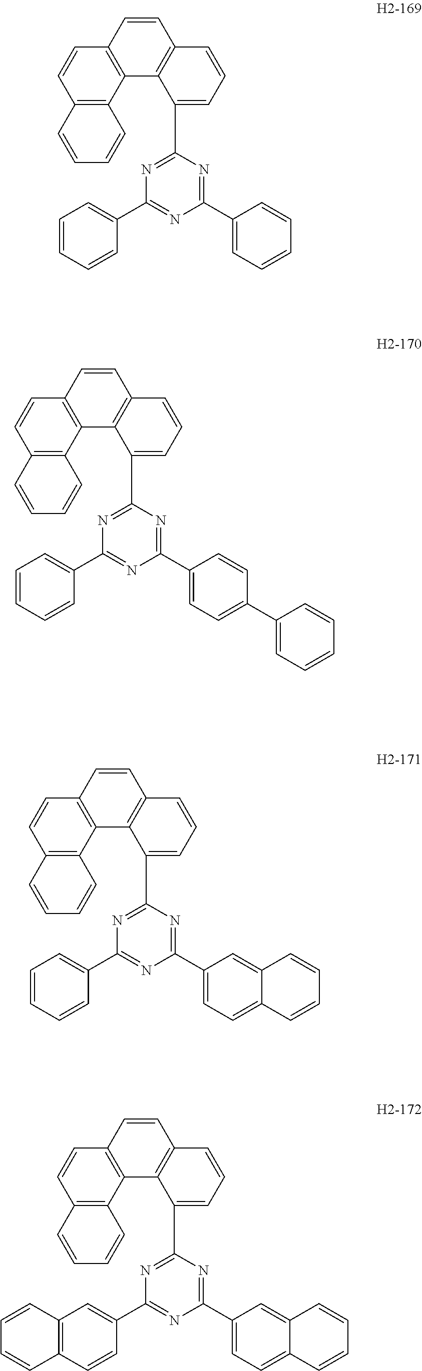 [SMILES: c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1 ; c1ccc2cc(-c3nc(-c4ccc5ccccc5c4)nc(-c4cccc5ccc6ccc7ccccc7c6c45)n3)ccc2c1]

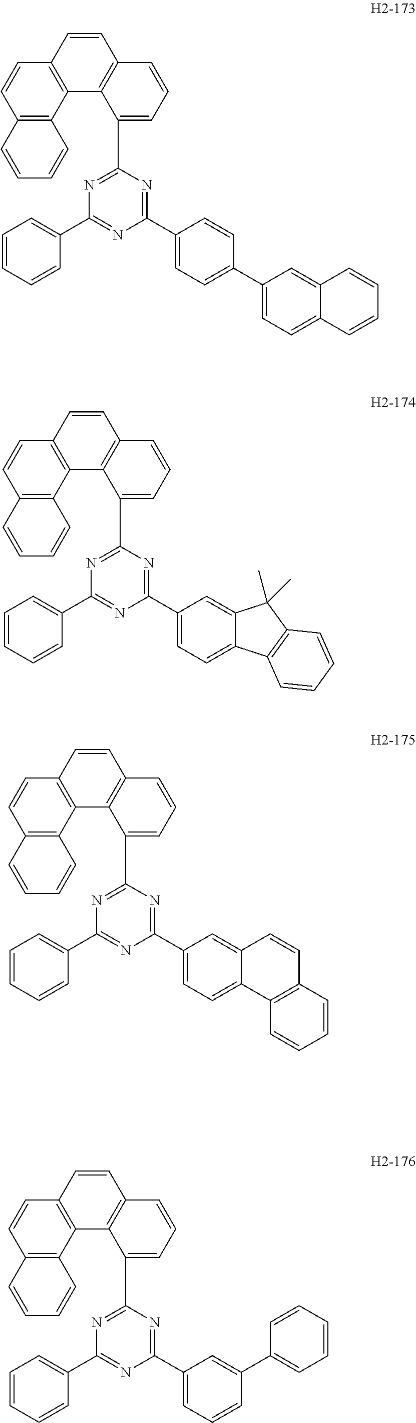 [SMILES: c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1 ; c1ccc(-c2cccc(-c3nc(-c4ccccc4)nc(-c4cccc5ccc6ccc7ccccc7c6c45)n3)c2)cc1]

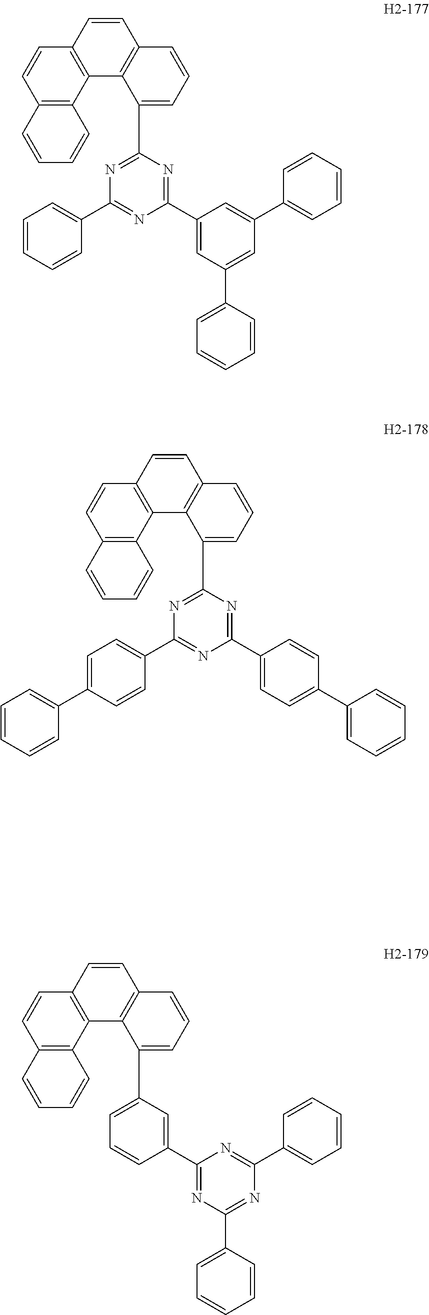 [SMILES: c1ccc(-c2cc(-c3ccccc3)cc(-c3nc(-c4ccccc4)nc(-c4cccc5ccc6ccc7ccccc7c6c45)n3)c2)cc1 ; c1ccc(-c2ccc(-c3nc(-c4ccc(-c5ccccc5)cc4)nc(-c4cccc5ccc6ccc7ccccc7c6c45)n3)cc2)cc1 ; c1ccc(-c2nc(-c3ccccc3)nc(-c3cccc(-c4cccc5ccc6ccc7ccccc7c6c45)c3)n2)cc1]

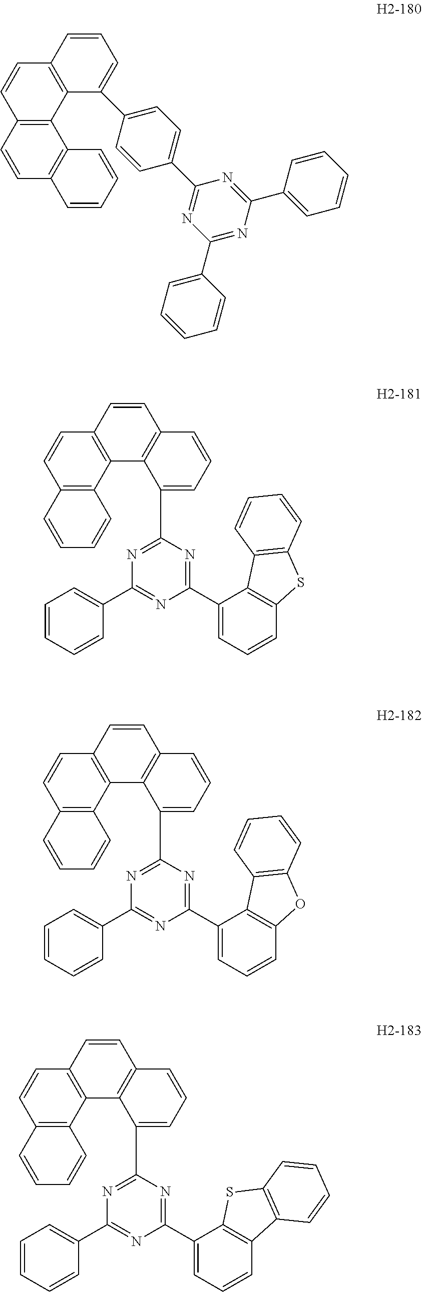 [SMILES: c1ccc(-c2nc(-c3ccccc3)nc(-c3ccc(-c4cccc5ccc6ccc7ccccc7c6c45)cc3)n2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

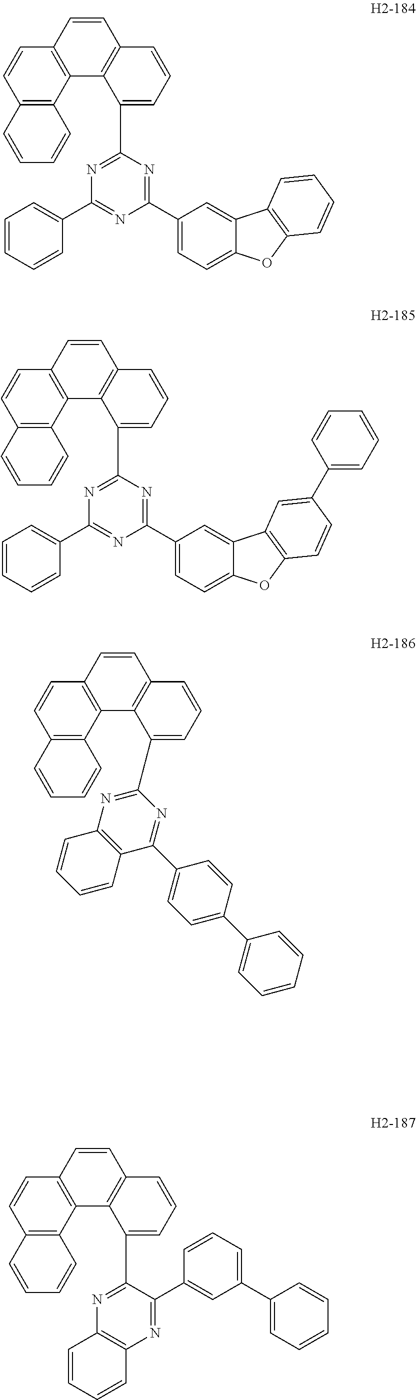 [SMILES: c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1 ; c1ccc(-c2ccc(-c3nc(-c4cccc5ccc6ccc7ccccc7c6c45)nc4ccccc34)cc2)cc1 ; c1ccc(-c2cccc(-c3nc4ccccc4nc3-c3cccc4ccc5ccc6ccccc6c5c34)c2)cc1]

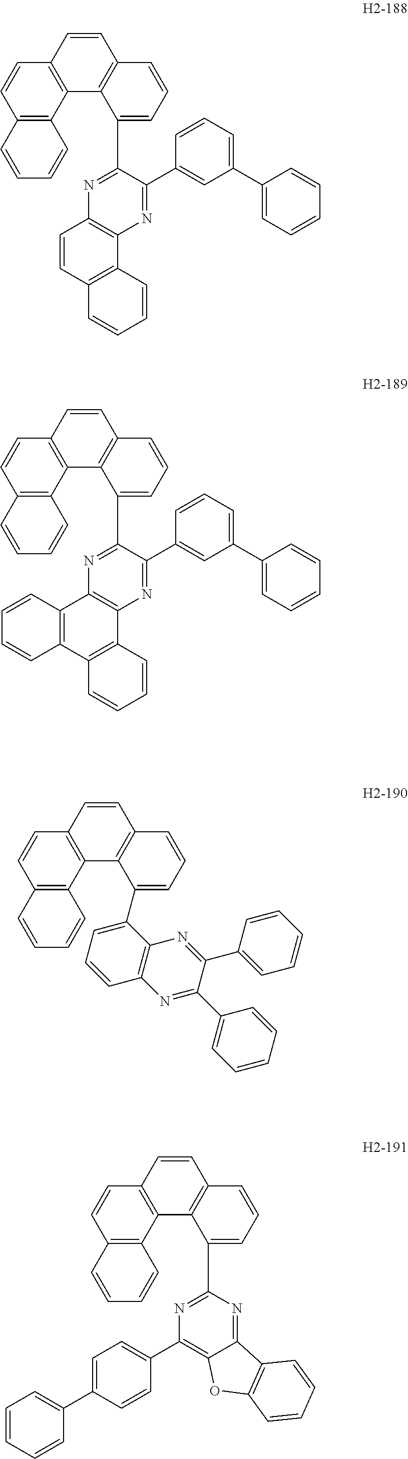 [SMILES: c1ccc(-c2cccc(-c3nc4c(ccc5ccccc54)nc3-c3cccc4ccc5ccc6ccccc6c5c34)c2)cc1 ; c1ccc(-c2cccc(-c3nc4c5ccccc5c5ccccc5c4nc3-c3cccc4ccc5ccc6ccccc6c5c34)c2)cc1 ; c1ccc(-c2nc3cccc(-c4cccc5ccc6ccc7ccccc7c6c45)c3nc2-c2ccccc2)cc1 ; c1ccc(-c2ccc(-c3nc(-c4cccc5ccc6ccc7ccccc7c6c45)nc4c3oc3ccccc34)cc2)cc1]

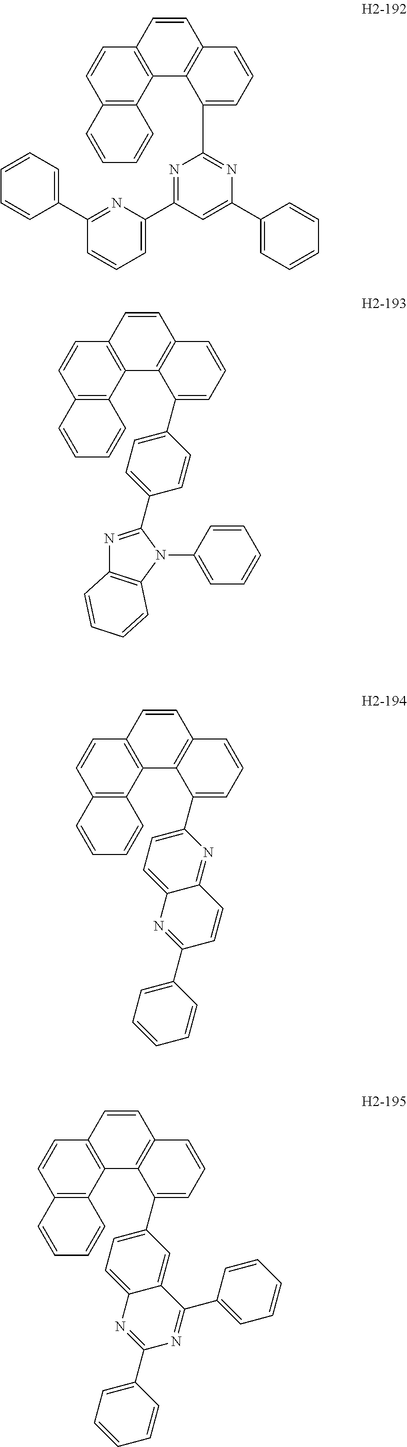

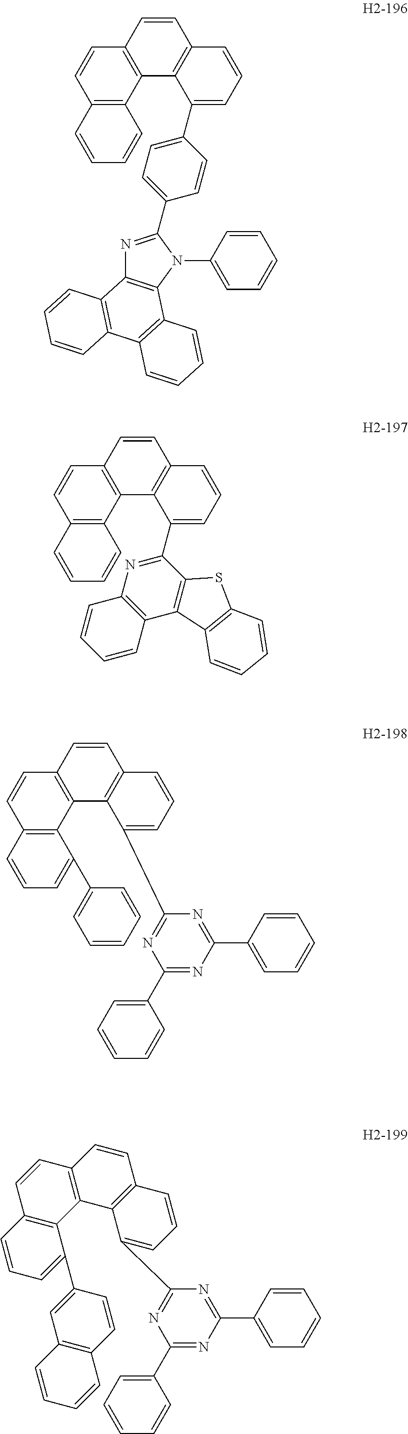 [SMILES: c1ccc(-n2c(-c3ccc(-c4cccc5ccc6ccc7ccccc7c6c45)cc3)nc3c4ccccc4c4ccccc4c32)cc1 ; c1ccc2c(c1)ccc1ccc3cccc(-c4nc5ccccc5c5c4sc4ccccc45)c3c12 ; c1ccc(-c2nc(-c3ccccc3)nc(-c3cccc4ccc5ccc6cccc(-c7ccccc7)c6c5c34)n2)cc1 ; c1ccc(-c2nc(-c3ccccc3)nc(-c3cccc4ccc5ccc6cccc(-c7ccc8ccccc8c7)c6c5c34)n2)cc1]

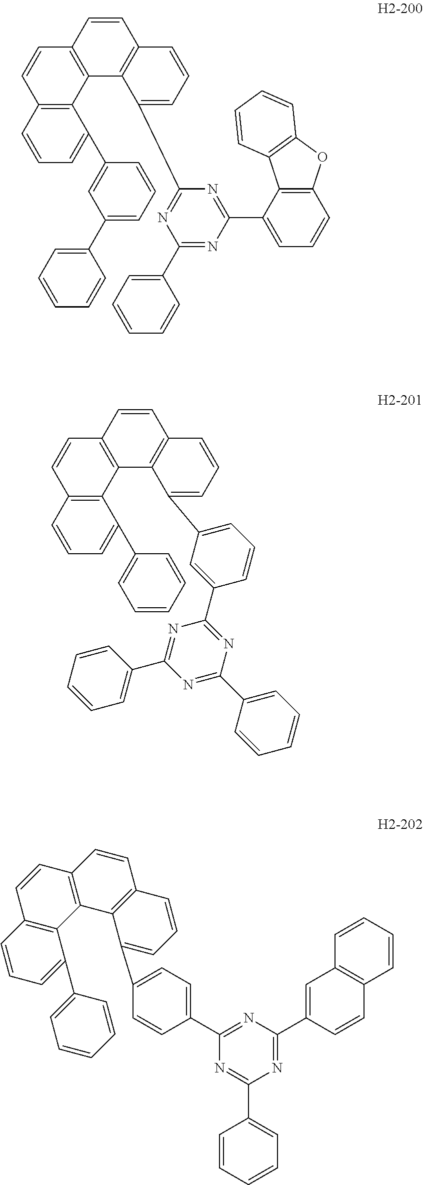 [SMILES: c1ccc(-c2cccc(-c3cccc4ccc5ccc6cccc(-c7nc(-c8ccccc8)nc(-c8cccc9oc%10ccccc%10c89)n7)c6c5c34)c2)cc1 ; c1ccc(-c2nc(-c3ccccc3)nc(-c3cccc(-c4cccc5ccc6ccc7cccc(-c8ccccc8)c7c6c45)c3)n2)cc1 ; c1ccc(-c2nc(-c3ccc(-c4cccc5ccc6ccc7cccc(-c8ccccc8)c7c6c45)cc3)nc(-c3ccc4ccccc4c3)n2)cc1]

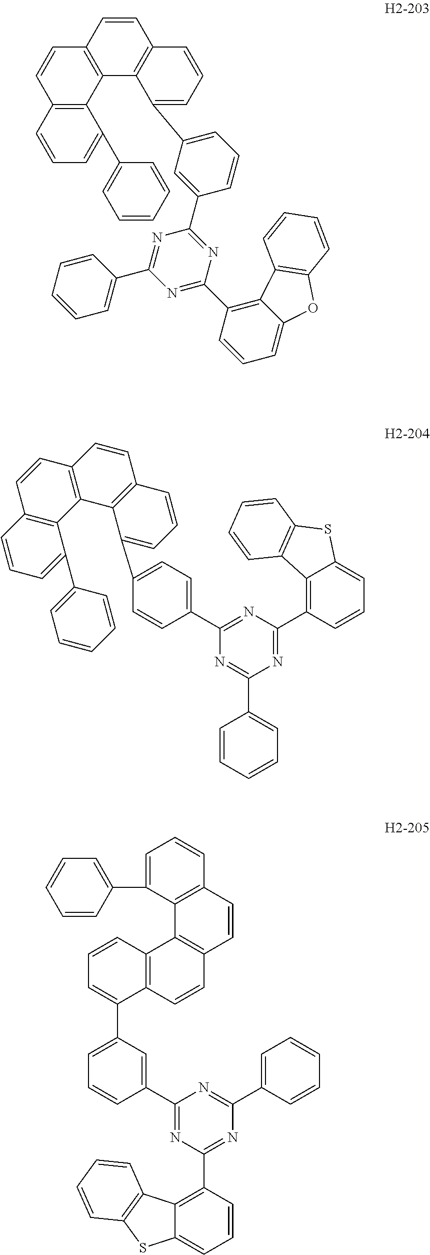 [SMILES: c1ccc(-c2nc(-c3cccc(-c4cccc5ccc6ccc7cccc(-c8ccccc8)c7c6c45)c3)nc(-c3cccc4oc5ccccc5c34)n2)cc1 ; c1ccc(-c2nc(-c3ccc(-c4cccc5ccc6ccc7cccc(-c8ccccc8)c7c6c45)cc3)nc(-c3cccc4sc5ccccc5c34)n2)cc1 ; c1ccc(-c2nc(-c3cccc(-c4cccc5c4ccc4ccc6cccc(-c7ccccc7)c6c45)c3)nc(-c3cccc4sc5ccccc5c34)n2)cc1]

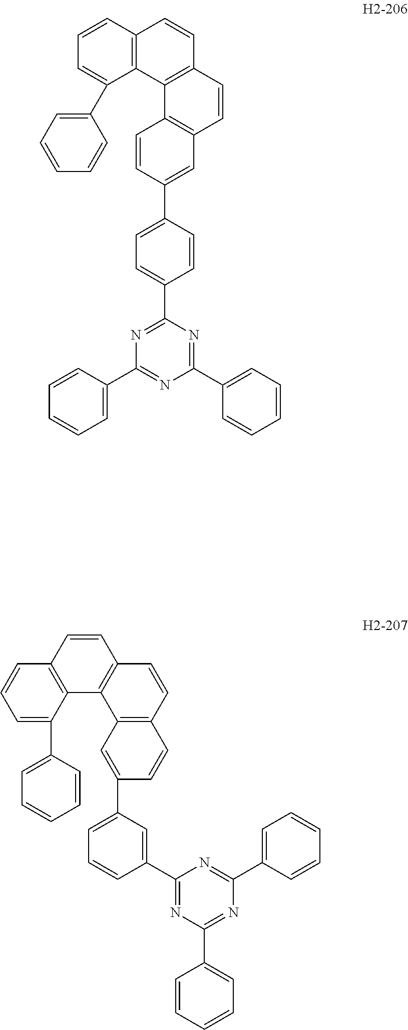 [SMILES: c1ccc(-c2nc(-c3ccccc3)nc(-c3ccc(-c4ccc5c(ccc6ccc7cccc(-c8ccccc8)c7c65)c4)cc3)n2)cc1 ; c1ccc(-c2nc(-c3ccccc3)nc(-c3cccc(-c4ccc5ccc6ccc7cccc(-c8ccccc8)c7c6c5c4)c3)n2)cc1]

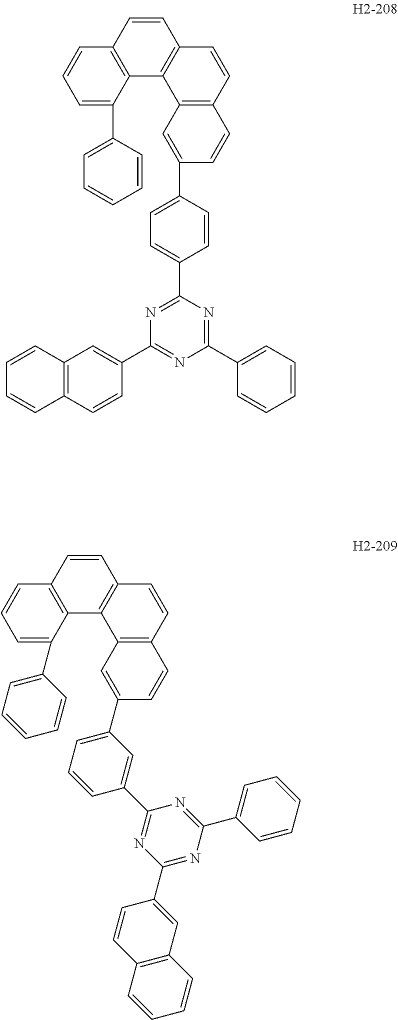 [SMILES: c1ccc(-c2nc(-c3ccc(-c4ccc5ccc6ccc7cccc(-c8ccccc8)c7c6c5c4)cc3)nc(-c3ccc4ccccc4c3)n2)cc1 ; c1ccc(-c2nc(-c3cccc(-c4ccc5ccc6ccc7cccc(-c8ccccc8)c7c6c5c4)c3)nc(-c3ccc4ccccc4c3)n2)cc1]

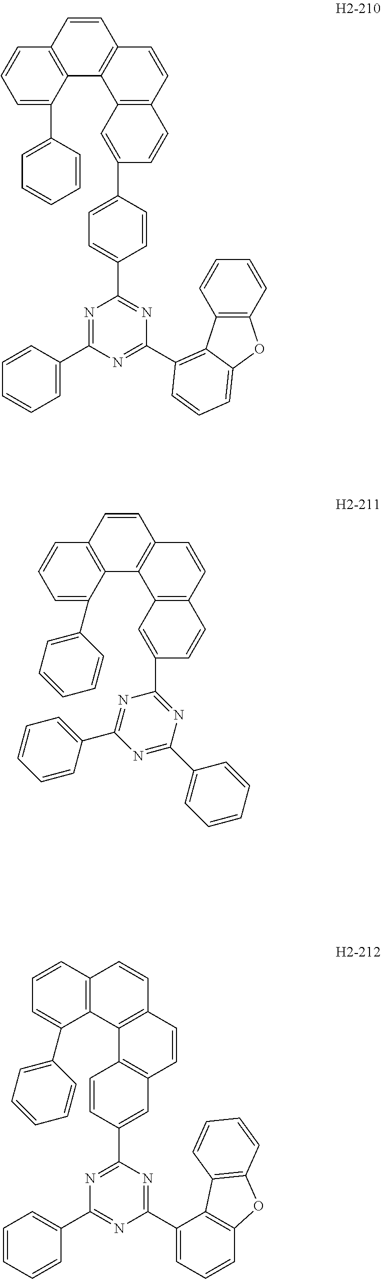 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1 ; c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1]

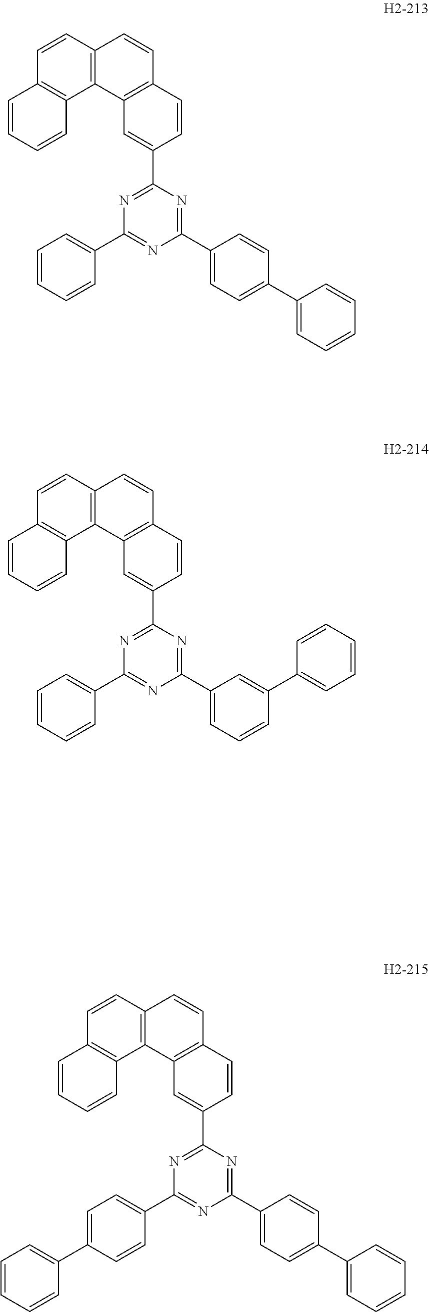 [SMILES: c1ccc(-c2ccc(-c3nc(-c4ccccc4)nc(-c4ccc5ccc6ccc7ccccc7c6c5c4)n3)cc2)cc1 ; c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1]

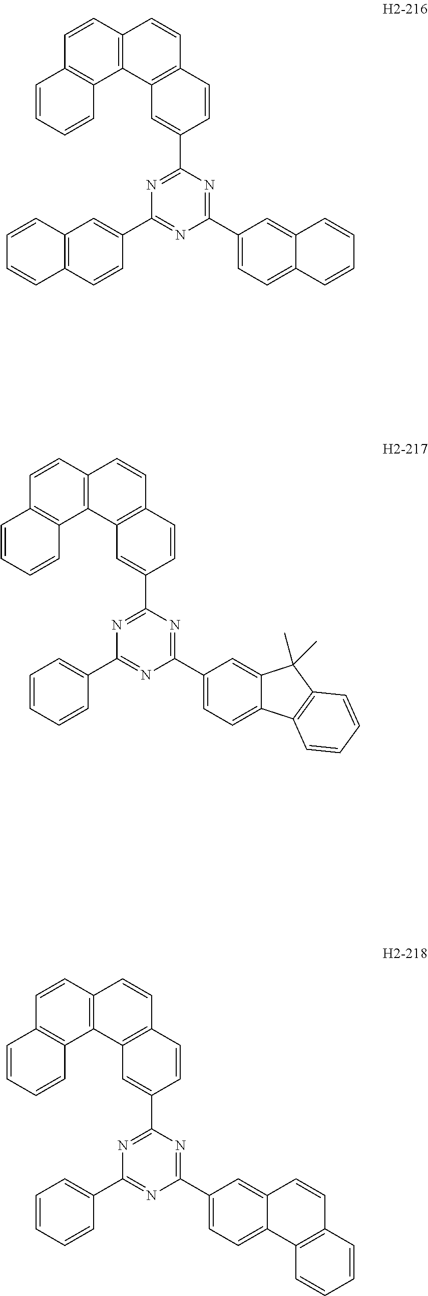 [SMILES: c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21 ; c1ccc(-c2nc(-c3ccc4c(ccc5ccccc54)c3)nc(-c3ccc4ccc5ccc6ccccc6c5c4c3)n2)cc1]

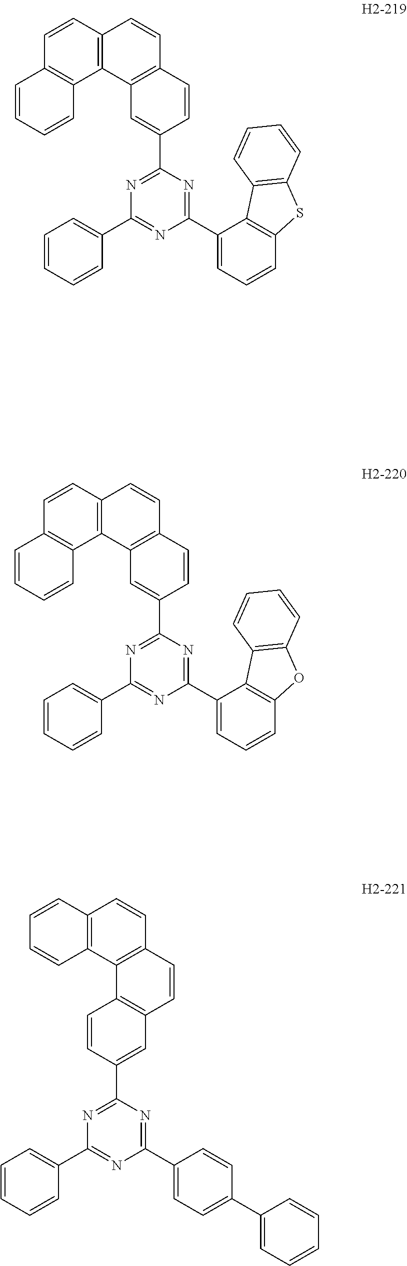 [SMILES: c1ccc(-c2nc(-c3ccc4ccc5ccc6ccccc6c5c4c3)nc(-c3cccc4sc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc(-c2ccc(-c3nc(-c4ccccc4)nc(-c4ccc5c(ccc6ccc7ccccc7c65)c4)n3)cc2)cc1]

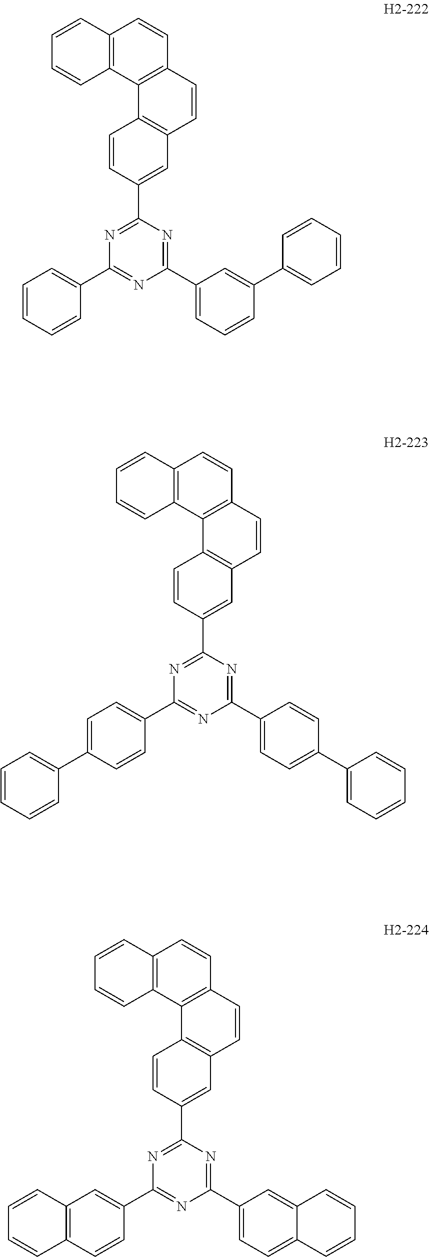 [SMILES: c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1 ; c1ccc2cc(-c3nc(-c4ccc5ccccc5c4)nc(-c4ccc5c(ccc6ccc7ccccc7c65)c4)n3)ccc2c1]

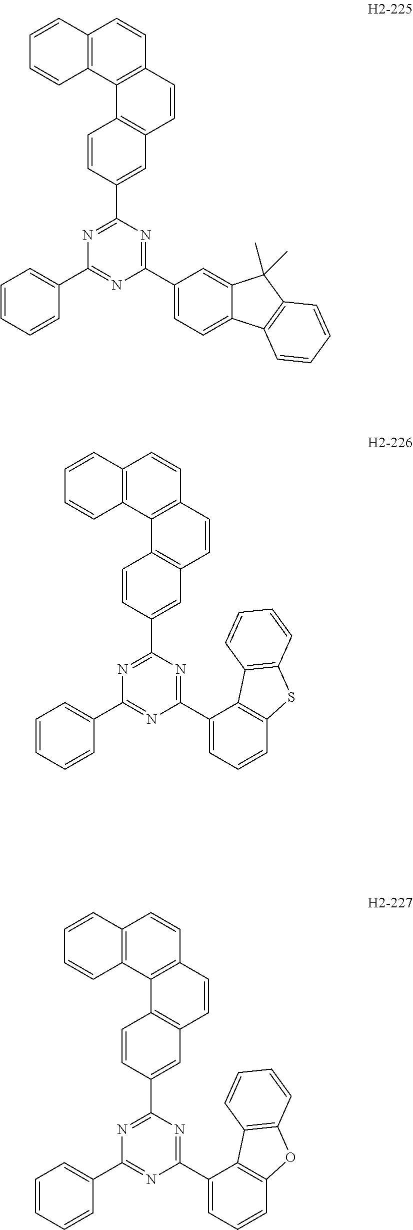 [SMILES: CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1 ; c1ccc(-c2nc(-c3ccc4c(ccc5ccc6ccccc6c54)c3)nc(-c3cccc4oc5ccccc5c34)n2)cc1]

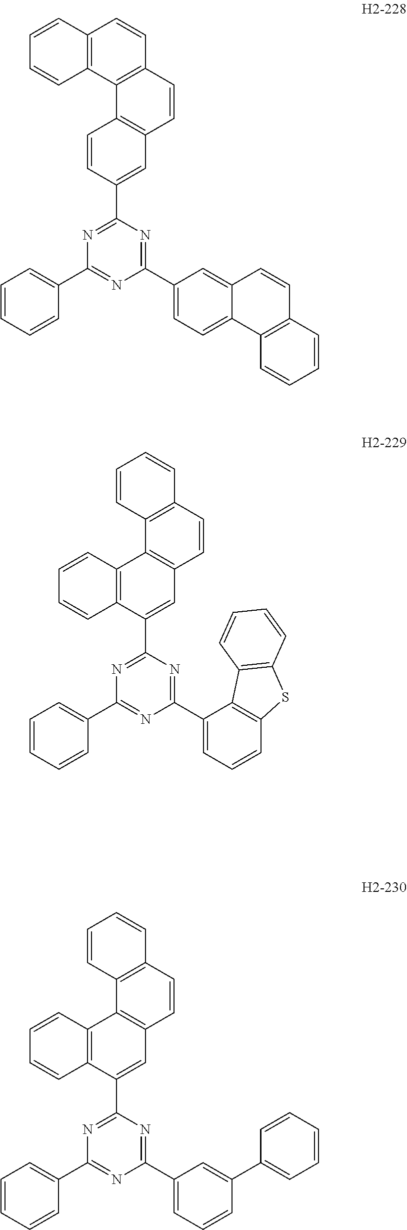 [SMILES: c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1 ; c1ccc(-c2cccc(-c3nc(-c4ccccc4)nc(-c4cc5ccc6ccccc6c5c5ccccc45)n3)c2)cc1]

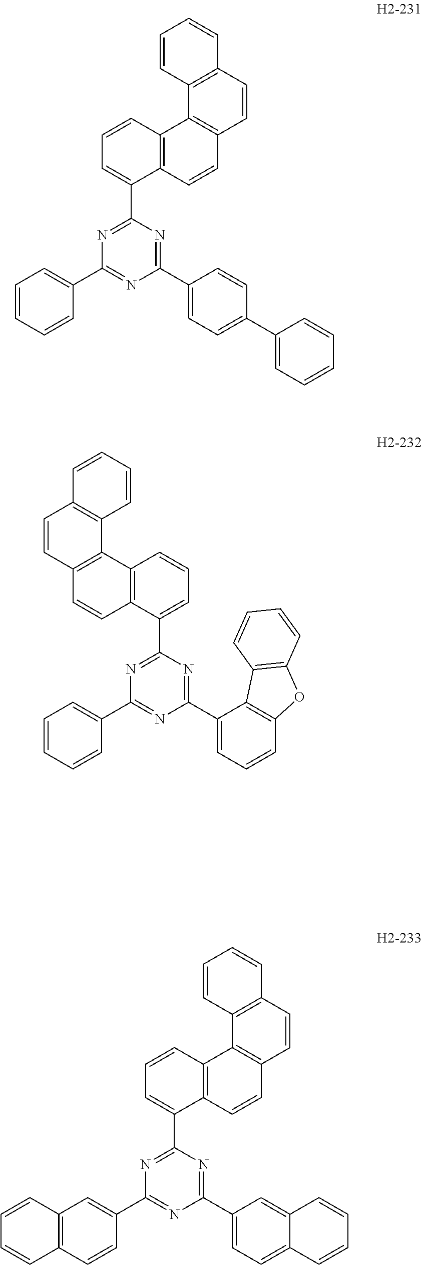 [SMILES: c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1 ; c1ccc2cc(-c3nc(-c4ccc5ccccc5c4)nc(-c4cccc5c4ccc4ccc6ccccc6c45)n3)ccc2c1]

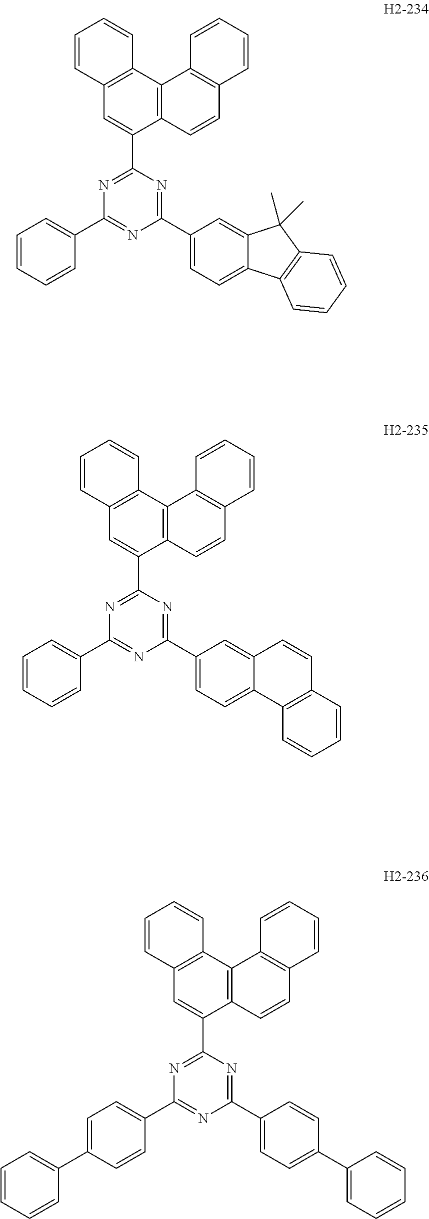 [SMILES: CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc(-c2ccc(-c3nc(-c4ccc(-c5ccccc5)cc4)nc(-c4cc5ccccc5c5c4ccc4ccccc45)n3)cc2)cc1]

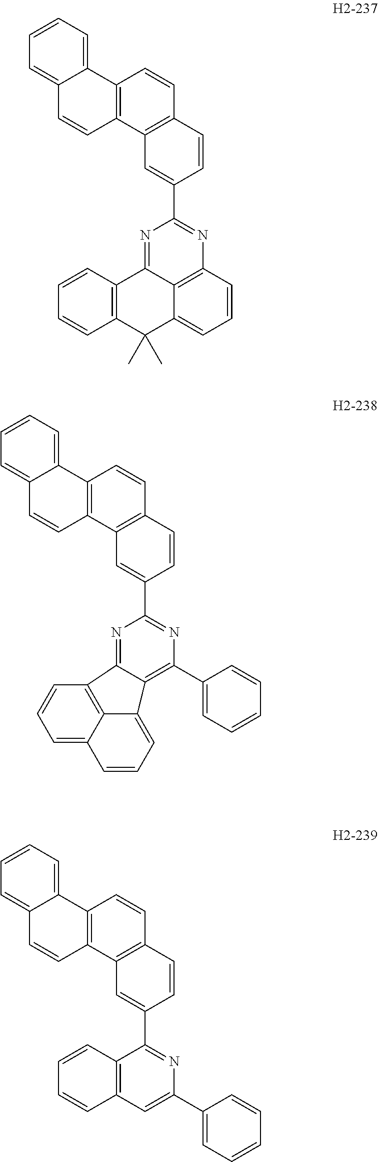 [SMILES: CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1 ; c1ccc(-c2cc3ccccc3c(-c3ccc4ccc5c6ccccc6ccc5c4c3)n2)cc1]

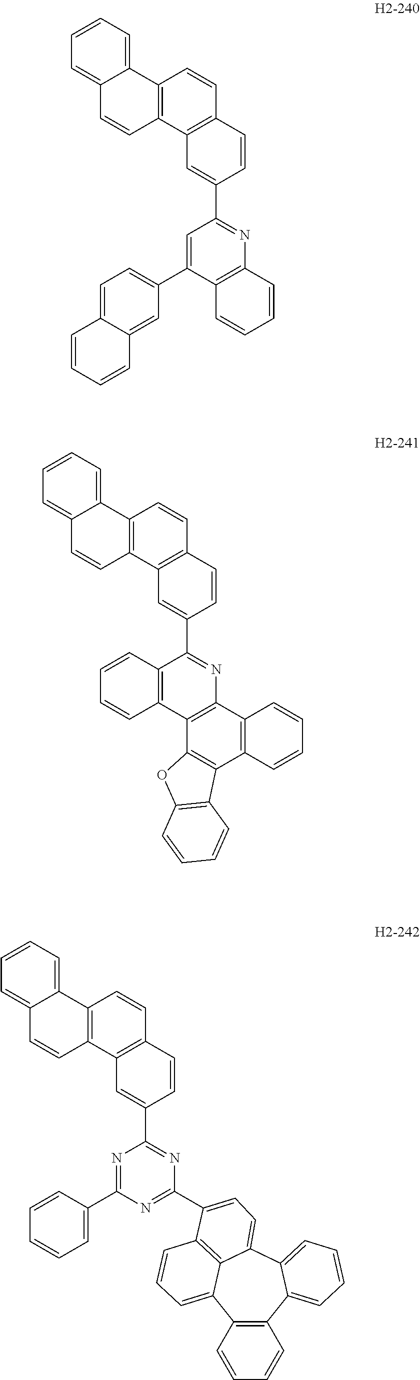 [SMILES: c1ccc2cc(-c3cc(-c4ccc5ccc6c7ccccc7ccc6c5c4)nc4ccccc34)ccc2c1 ; c1ccc2c(c1)ccc1c3cc(-c4nc5c6ccccc6c6c7ccccc7oc6c5c5ccccc45)ccc3ccc21 ; c1ccc(-c2nc(-c3ccc4ccc5c6ccccc6ccc5c4c3)nc(-c3ccc4c5c(cccc35)-c3ccccc3-c3ccccc3-4)n2)cc1]

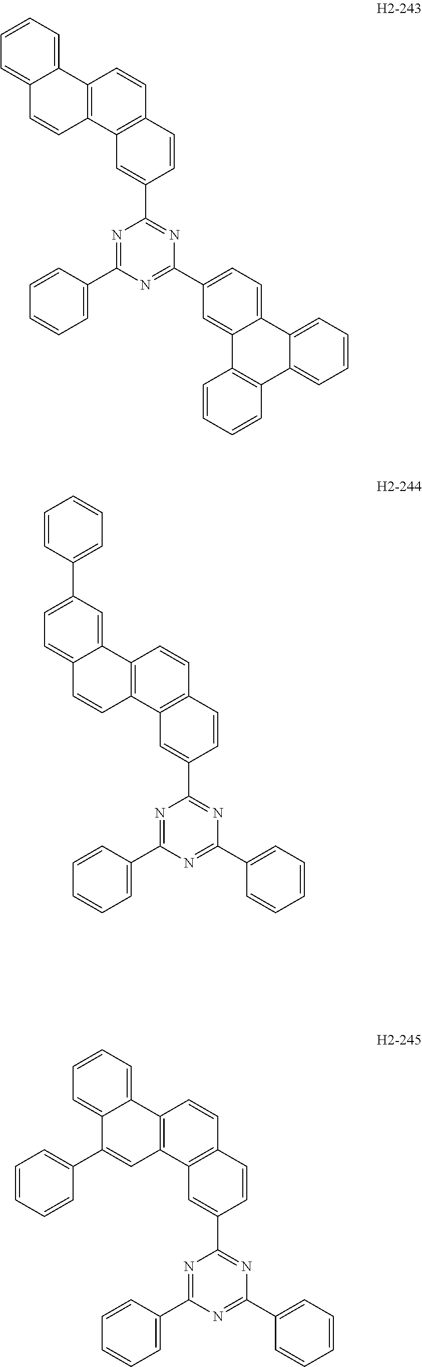 [SMILES: c1ccc(-c2nc(-c3ccc4ccc5c6ccccc6ccc5c4c3)nc(-c3ccc4c5ccccc5c5ccccc5c4c3)n2)cc1 ; c1ccc(-c2ccc3ccc4c5cc(-c6nc(-c7ccccc7)nc(-c7ccccc7)n6)ccc5ccc4c3c2)cc1 ; c1ccc(-c2nc(-c3ccccc3)nc(-c3ccc4ccc5c6ccccc6c(-c6ccccc6)cc5c4c3)n2)cc1]

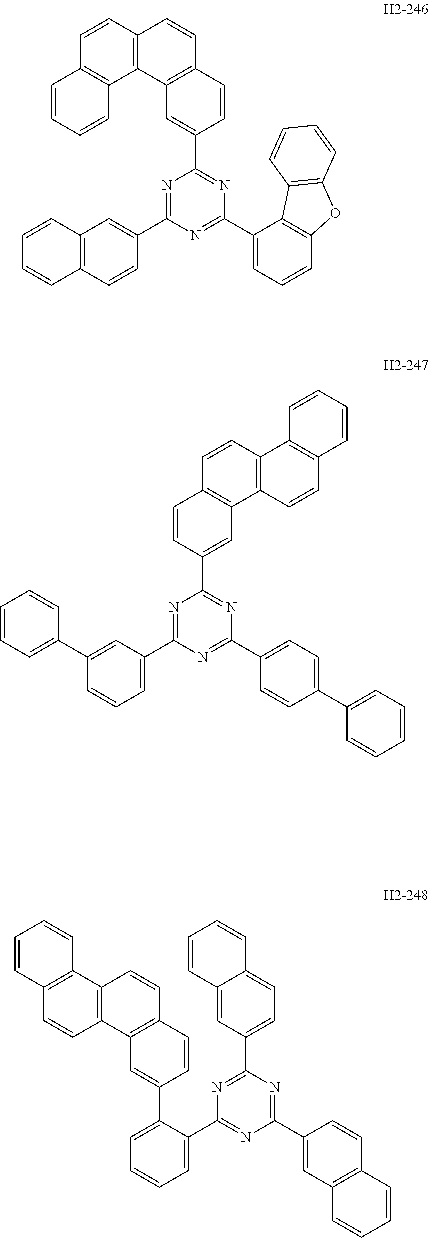 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1]

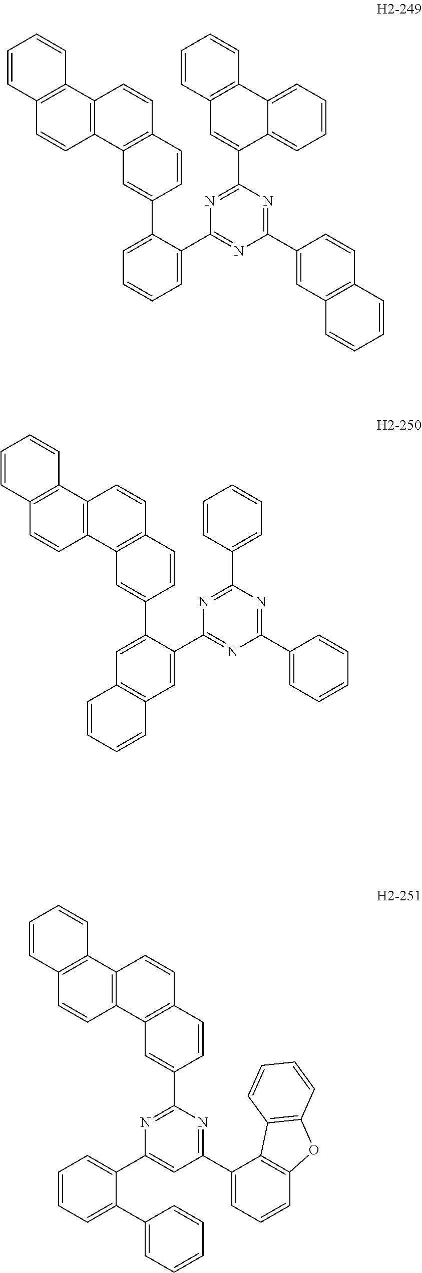 [SMILES: c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1]

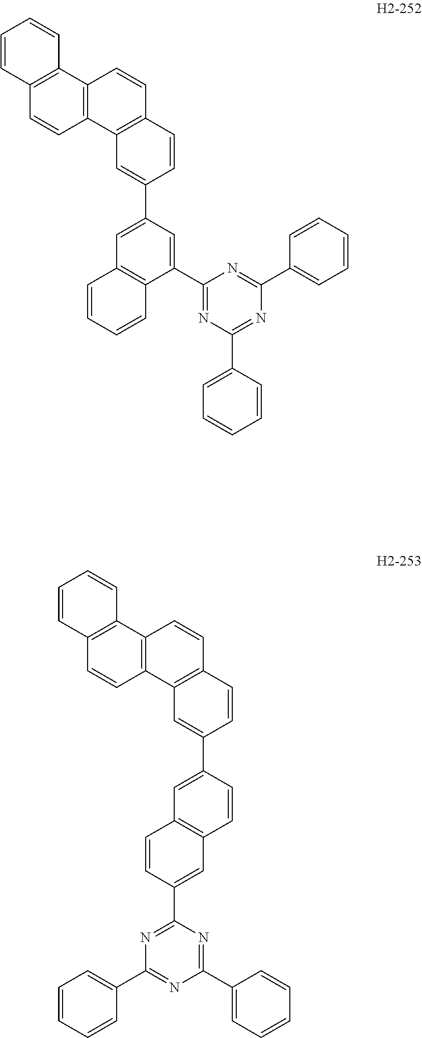 [SMILES: c1ccc(-c2nc(-c3ccccc3)nc(-c3cc(-c4ccc5ccc6c7ccccc7ccc6c5c4)cc4ccccc34)n2)cc1 ; c1ccc(-c2nc(-c3ccccc3)nc(-c3ccc4cc(-c5ccc6ccc7c8ccccc8ccc7c6c5)ccc4c3)n2)cc1]

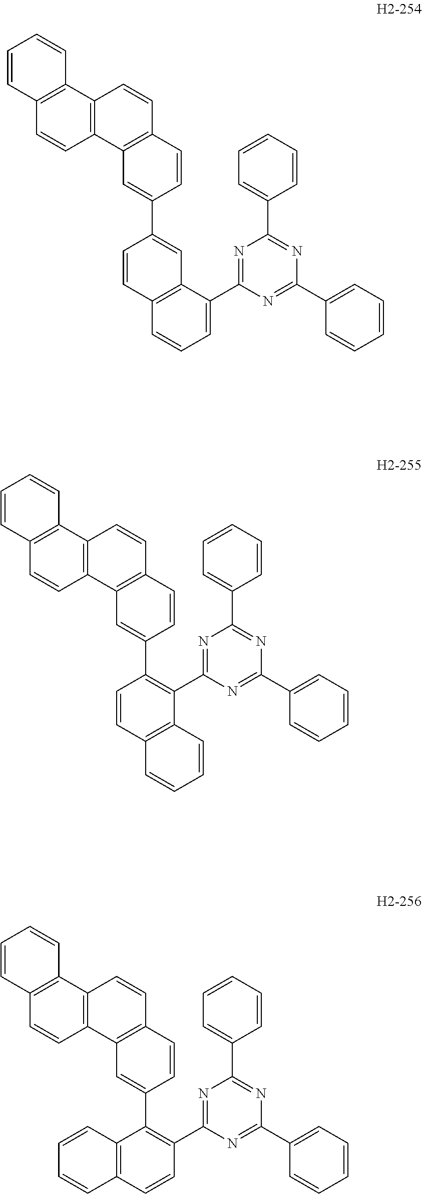 [SMILES: c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1 ; c1ccc(-c2nc(-c3ccccc3)nc(-c3ccc4ccccc4c3-c3ccc4ccc5c6ccccc6ccc5c4c3)n2)cc1]

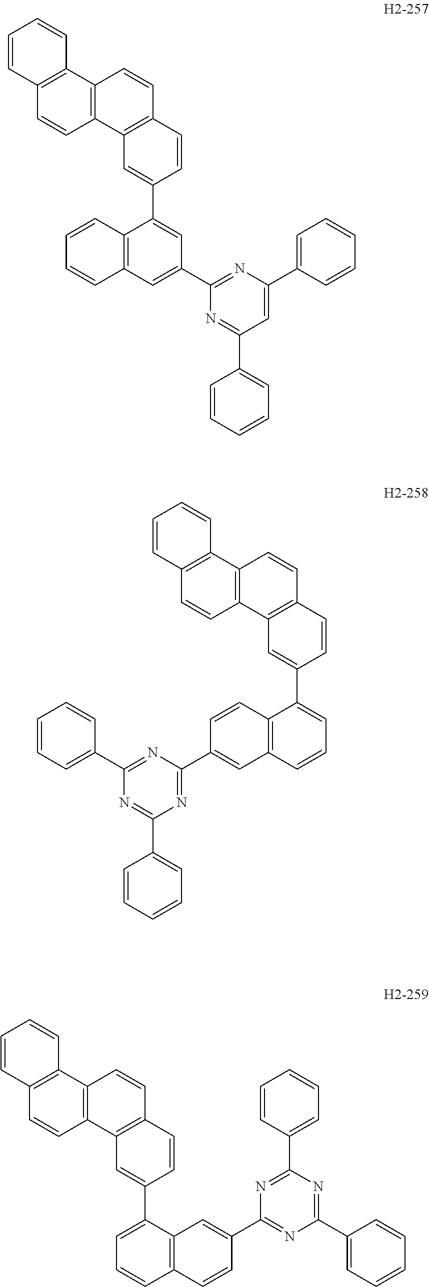 [SMILES: c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1 ; c1ccc(-c2nc(-c3ccccc3)nc(-c3ccc4cccc(-c5ccc6ccc7c8ccccc8ccc7c6c5)c4c3)n2)cc1]

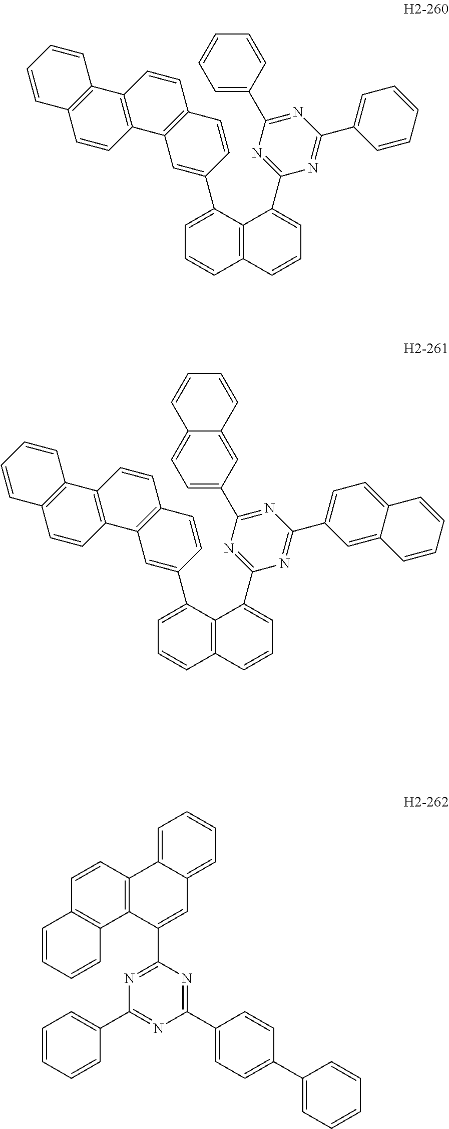 [SMILES: c1ccc(-c2nc(-c3ccccc3)nc(-c3cccc4cccc(-c5ccc6ccc7c8ccccc8ccc7c6c5)c34)n2)cc1 ; c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2ccc(-c3nc(-c4ccccc4)nc(-c4cc5ccccc5c5ccc6ccccc6c45)n3)cc2)cc1]

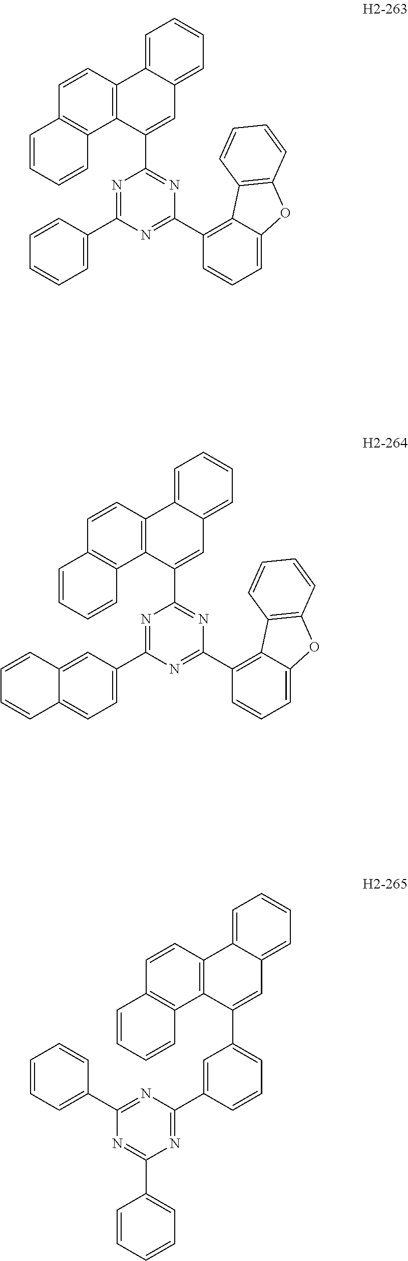 [SMILES: c1ccc(-c2nc(-c3cccc4oc5ccccc5c34)nc(-c3cc4ccccc4c4ccc5ccccc5c34)n2)cc1 ; c1ccc2cc(-c3nc(-c4cccc5oc6ccccc6c45)nc(-c4cc5ccccc5c5ccc6ccccc6c45)n3)ccc2c1 ; c1ccc(-c2nc(-c3ccccc3)nc(-c3cccc(-c4cc5ccccc5c5ccc6ccccc6c45)c3)n2)cc1]

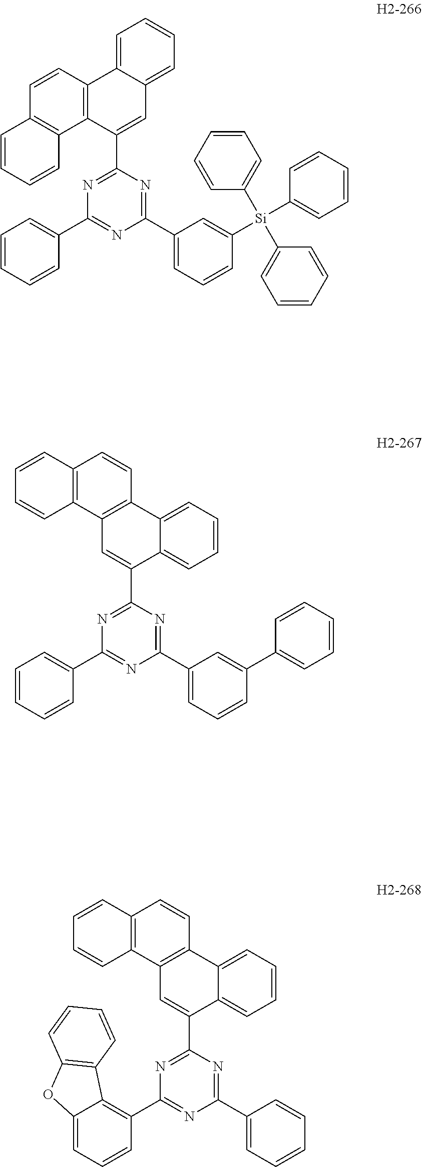 [SMILES: c1ccc(-c2nc(-c3cccc([Si](c4ccccc4)(c4ccccc4)c4ccccc4)c3)nc(-c3cc4ccccc4c4ccc5ccccc5c34)n2)cc1 ; c1ccc(-c2cccc(-c3nc(-c4ccccc4)nc(-c4cc5c6ccccc6ccc5c5ccccc45)n3)c2)cc1 ; c1ccc(-c2nc(-c3cc4c5ccccc5ccc4c4ccccc34)nc(-c3cccc4oc5ccccc5c34)n2)cc1]

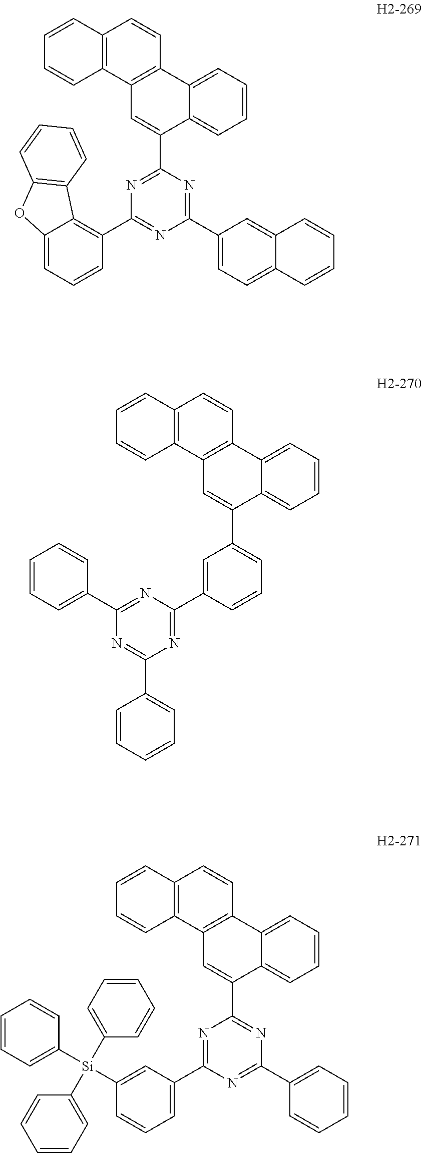 [SMILES: c1ccc2cc(-c3nc(-c4cc5c6ccccc6ccc5c5ccccc45)nc(-c4cccc5oc6ccccc6c45)n3)ccc2c1 ; c1ccc(-c2nc(-c3ccccc3)nc(-c3cccc(-c4cc5c6ccccc6ccc5c5ccccc45)c3)n2)cc1 ; c1ccc(-c2nc(-c3cccc([Si](c4ccccc4)(c4ccccc4)c4ccccc4)c3)nc(-c3cc4c5ccccc5ccc4c4ccccc34)n2)cc1]

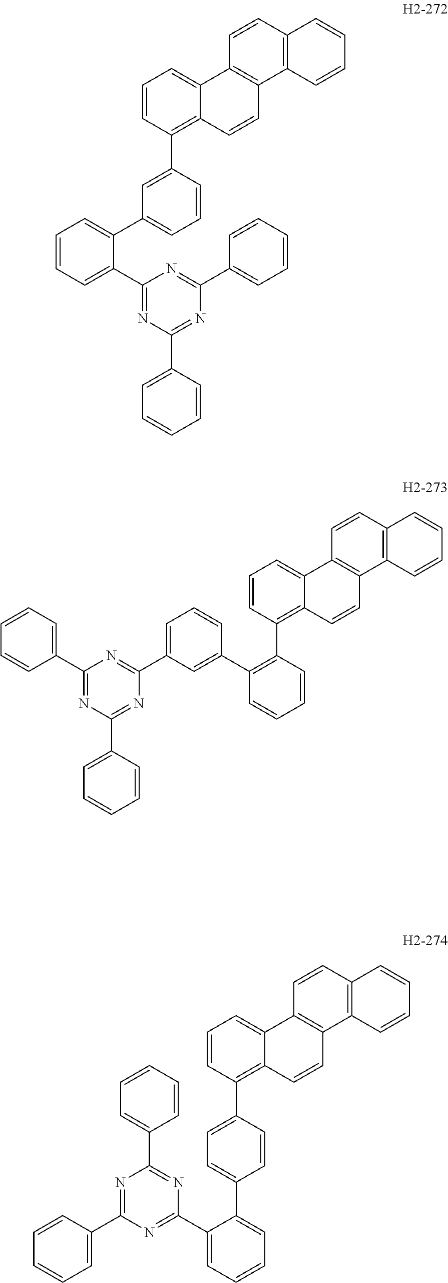 [SMILES: c1ccc(-c2nc(-c3ccccc3)nc(-c3ccccc3-c3cccc(-c4cccc5c4ccc4c6ccccc6ccc54)c3)n2)cc1 ; c1ccc(-c2nc(-c3ccccc3)nc(-c3cccc(-c4ccccc4-c4cccc5c4ccc4c6ccccc6ccc54)c3)n2)cc1 ; c1ccc(-c2nc(-c3ccccc3)nc(-c3ccccc3-c3ccc(-c4cccc5c4ccc4c6ccccc6ccc54)cc3)n2)cc1]

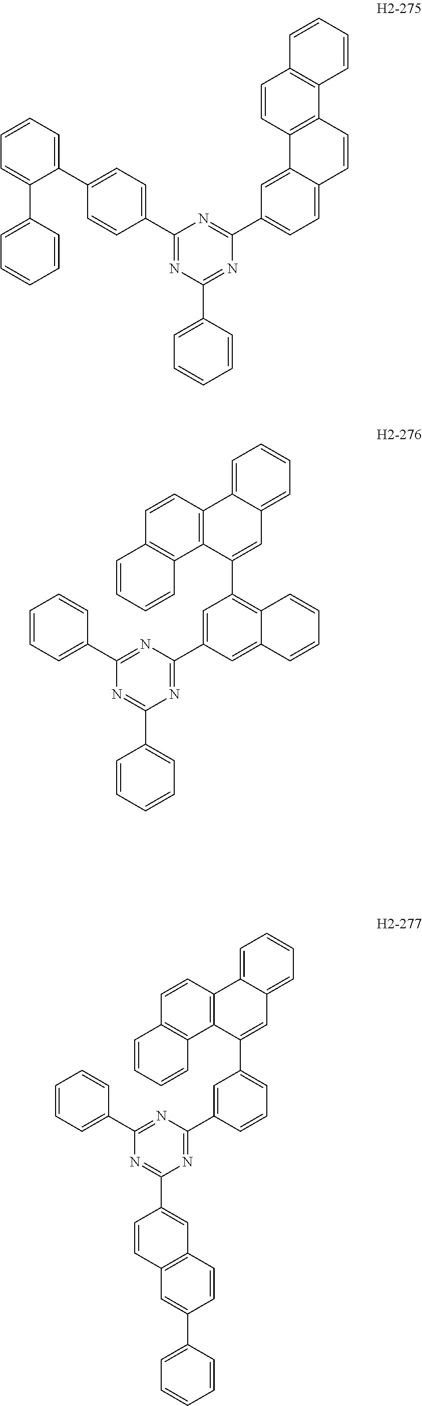 [SMILES: c1ccc(-c2nc(-c3ccc(-c4ccccc4-c4ccccc4)cc3)nc(-c3ccc4ccc5c6ccccc6ccc5c4c3)n2)cc1 ; c1ccc(-c2nc(-c3ccccc3)nc(-c3cc(-c4cc5ccccc5c5ccc6ccccc6c45)c4ccccc4c3)n2)cc1 ; c1ccc(-c2ccc3cc(-c4nc(-c5ccccc5)nc(-c5cccc(-c6cc7ccccc7c7ccc8ccccc8c67)c5)n4)ccc3c2)cc1]

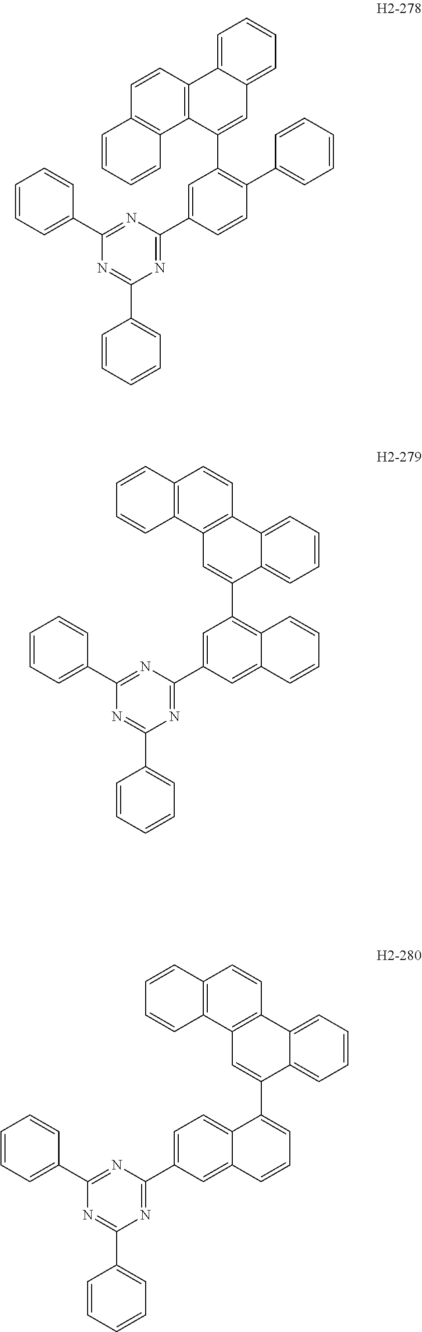 [SMILES: c1ccc(-c2nc(-c3ccccc3)nc(-c3ccc(-c4ccccc4)c(-c4cc5ccccc5c5ccc6ccccc6c45)c3)n2)cc1 ; c1ccc(-c2nc(-c3ccccc3)nc(-c3cc(-c4cc5c6ccccc6ccc5c5ccccc45)c4ccccc4c3)n2)cc1 ; c1ccc(-c2nc(-c3ccccc3)nc(-c3ccc4c(-c5cc6c7ccccc7ccc6c6ccccc56)cccc4c3)n2)cc1]

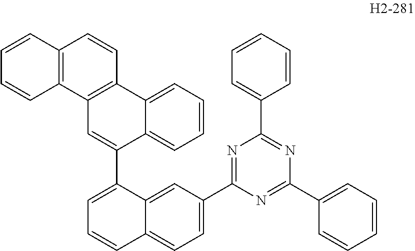 [SMILES: c1ccc(-c2nc(-c3ccccc3)nc(-c3ccc4cccc(-c5cc6c7ccccc7ccc6c6ccccc56)c4c3)n2)cc1]

The compounds represented by formula 2 according to the present disclosure may be produced by a synthetic method known to a person skilled in the art. for example they may be prepared as represented by the following reaction schemes 4 and 5.

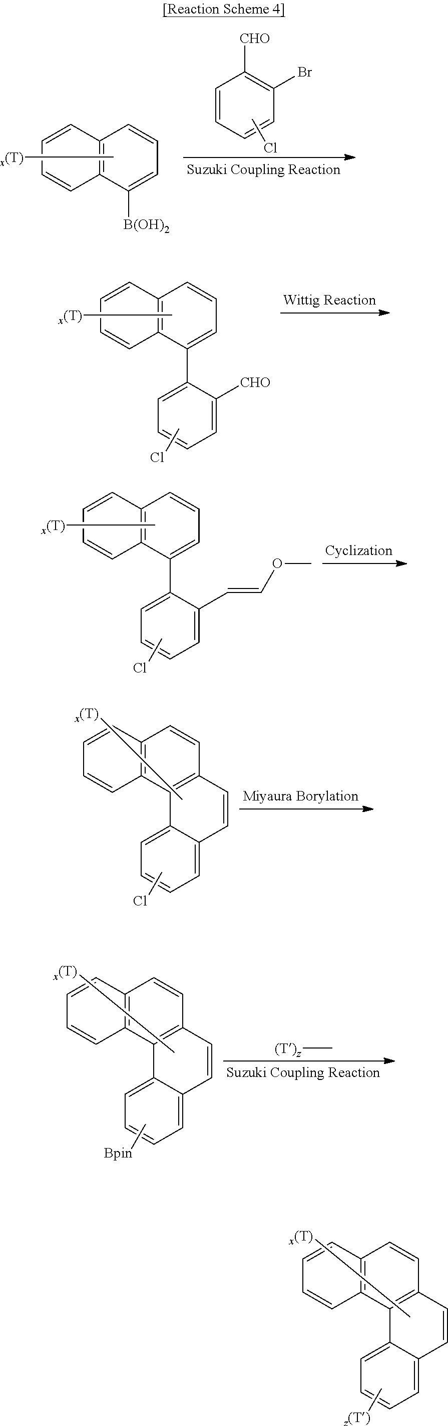

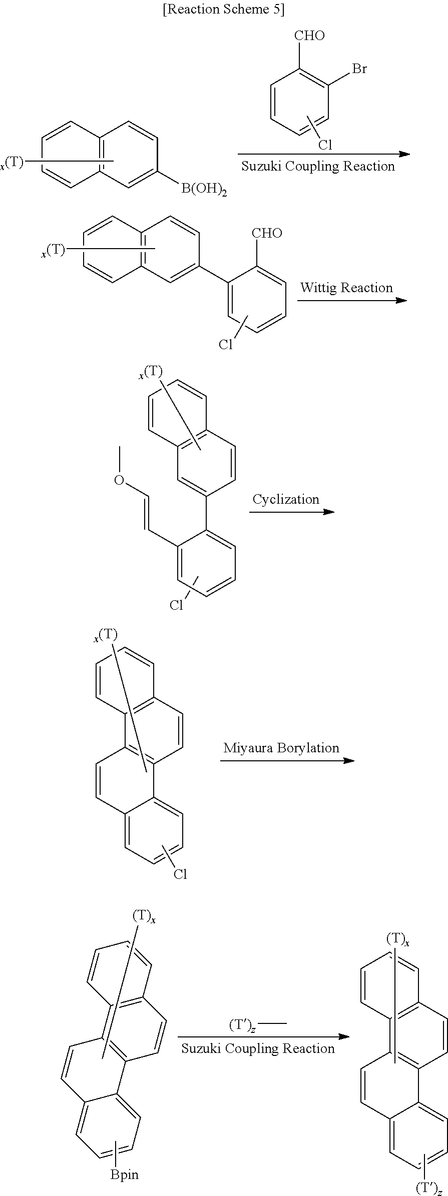

In reaction schemes 4 and 5 above, T and T′ each independently are as defined as T₁ to T₁₄ in formula 2, x represents an integer of 1 to 7, z represents an integer of 1 to 4, and when x and z are an integer of 2 or more, each of T and each of T′ may be the same or different.

As described above, exemplary synthesis examples of the compounds represented by formula 2 according to the present disclosure are described, but they are based on Buchwald-Hartwig cross coupling reaction, N-arylation reaction. H-mont-mediated etherification reaction, Miyaura borylation reaction, Suzuki cross-coupling reaction, Intramolecular acid-induced cyclization reaction, Pd(II)-catalyzed oxidative cyclization reaction, Grignard reaction, Heck reaction, Cyclic Dehydration reaction, SN1 substitution reaction, SN2 substitution reaction, and Phosphine-mediated reductive cyclization reaction, etc. It will be understood by one skilled in the art that the above reaction proceeds even if other substituents defined in the formula 2 other than the substituents described in the specific synthesis examples are bonded.

According to one embodiment, the present disclosure may provide an organic electroluminescent material comprising an organic electroluminescent compound of formula 1, and an organic electroluminescent device comprising the organic electroluminescent material.

According to another embodiment, the present disclosure may provide a plurality of host materials comprising a first host material including a compound of formula 1 and a second host material including a compound of formula 2, and an organic electroluminescent device comprising the plurality of host materials.

The organic electroluminescent material may be comprised solely of the organic electroluminescent compound of the present disclosure, or may further comprise conventional materials included in the organic electroluminescent material. When two or more species of materials are included in one layer, the at least two host materials may be mixture-evaporated to form a layer or may be individually and simultaneously co-evaporated to form a layer. The organic electroluminescent material according to one embodiment may comprise at least one compound represented by formula 1 above. The organic electroluminescent compound of formula 1 of the present disclosure may preferably be included in the hole transport layer, the light-emitting layer, the buffer layer, and/or the electron transport layer of the organic electroluminescent device, and more preferably, the light-emitting layer or the electron buffer layer. When included in the light-emitting layer, the compound of formula 1 may be included as a host, and more specifically, may be included as a phosphorescent red host. The organic electroluminescent material according to another embodiment may comprise a first host material including the compound represented by formula 1 and a second host material including the compound represented by formula 2, specifically at least one compound(s) of compounds C-1 to C-320 as the first host material including the compound represented by formula 1 and at least one compound(s) of compounds H2-1 to H2-281 as the second host material including the compound represented by formula 2, and the plurality of host materials may be included in the same organic layer, for example, a light-emitting layer, or may be included in different light-emitting layers, respectively.

The organic electroluminescent material according to the present disclosure may comprise at least one dopant in addition to the organic electroluminescent compound represented by formula 1 and the plurality of host materials comprising the same.

The dopant comprised in the organic electroluminescent material of the present disclosure may use at least one phosphorescent or fluorescent dopant, preferably a phosphorescent dopant. The applied phosphorescent dopant material in the present disclosure is not particularly limited, but may be preferably a metallated complex compound(s) of a metal atom(s) selected from iridium (Ir), osmium (Os), copper (Cu), and platinum (Pt), as necessary; more preferably an ortho-metallated complex compound(s) of a metal atom(s) selected from iridium (Ir), osmium (Os), copper (Cu), and platinum (Pt), as necessary; and even more preferably ortho-metallated iridium complex compound(s), as necessary.

The dopant according to the present disclosure may use the compound represented by the following formula 101, but is not limited thereto.

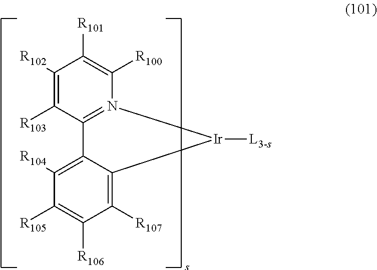

In formula 101,

L is selected from any one of the following structures 1 to 3;

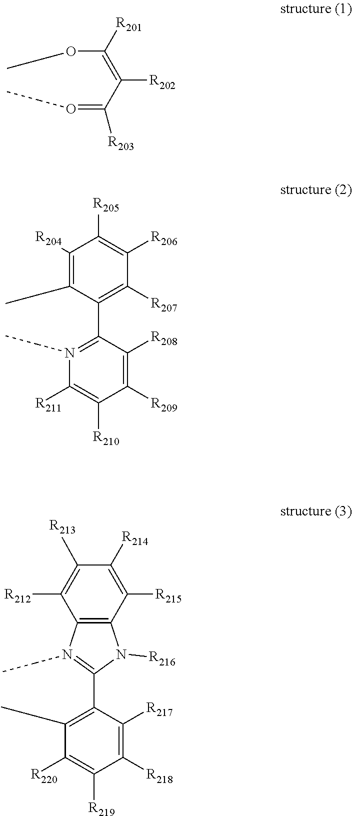

R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to the adjacent substituent to form a ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to the adjacent substituent to form a substituted or unsubstituted ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀ each independently represent, hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to the adjacent substituent to form a substituted or unsubstituted ring(s); and

s represents an integer of 1 to 3.

Specifically, the specific examples of the dopant compound include the following, but are not limited thereto.

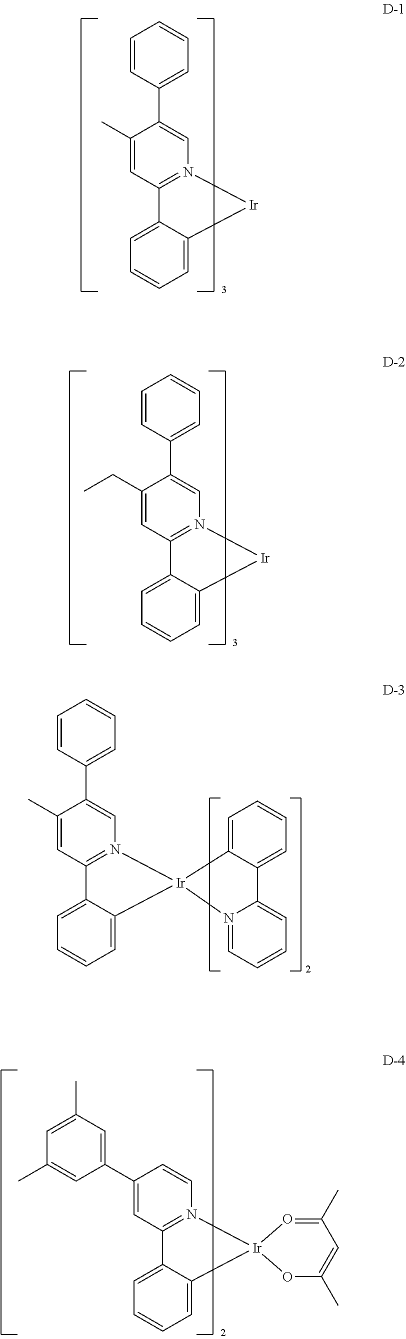

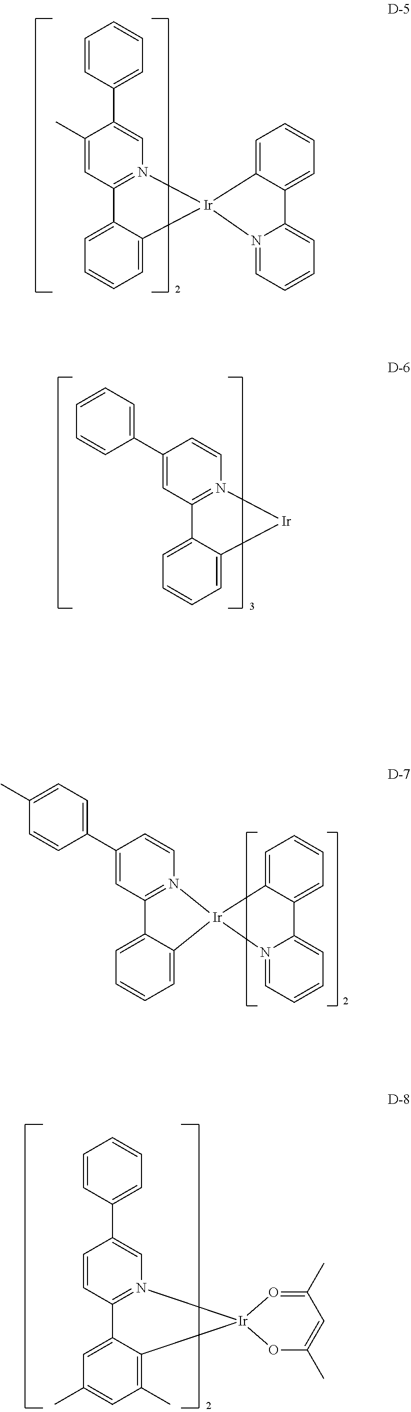

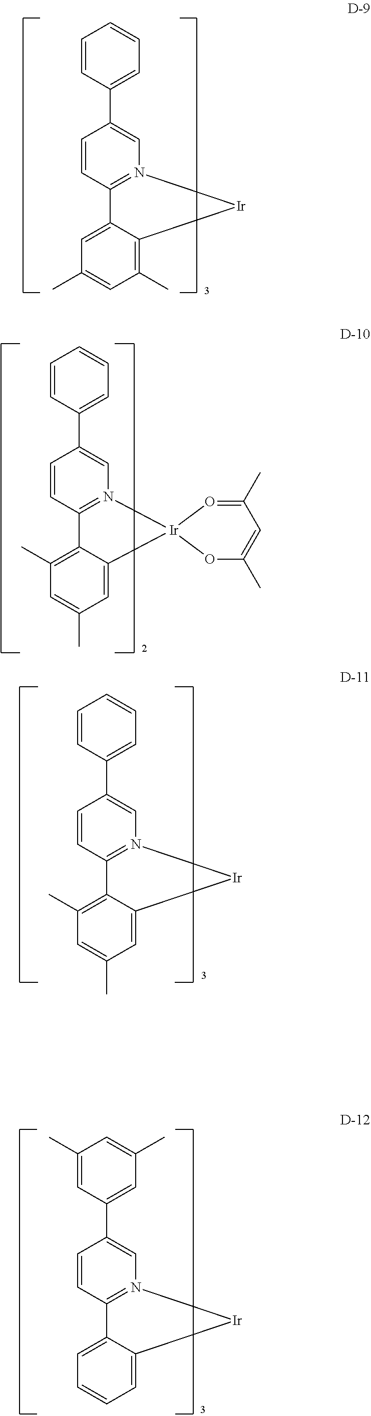

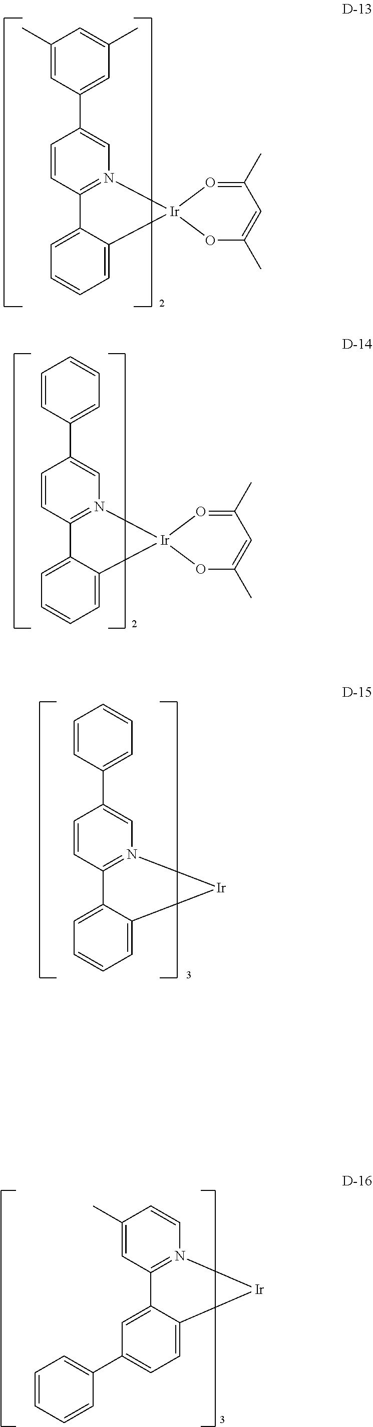

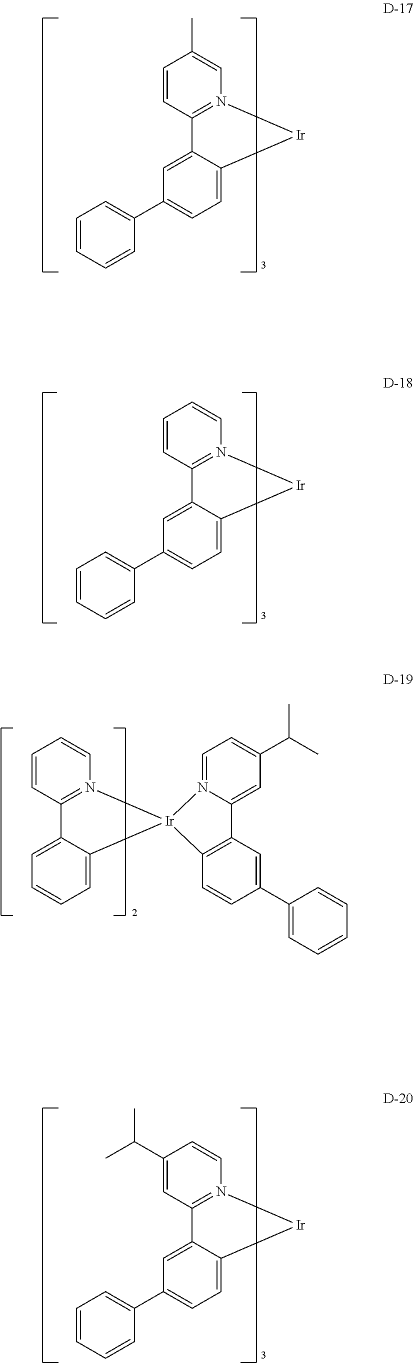

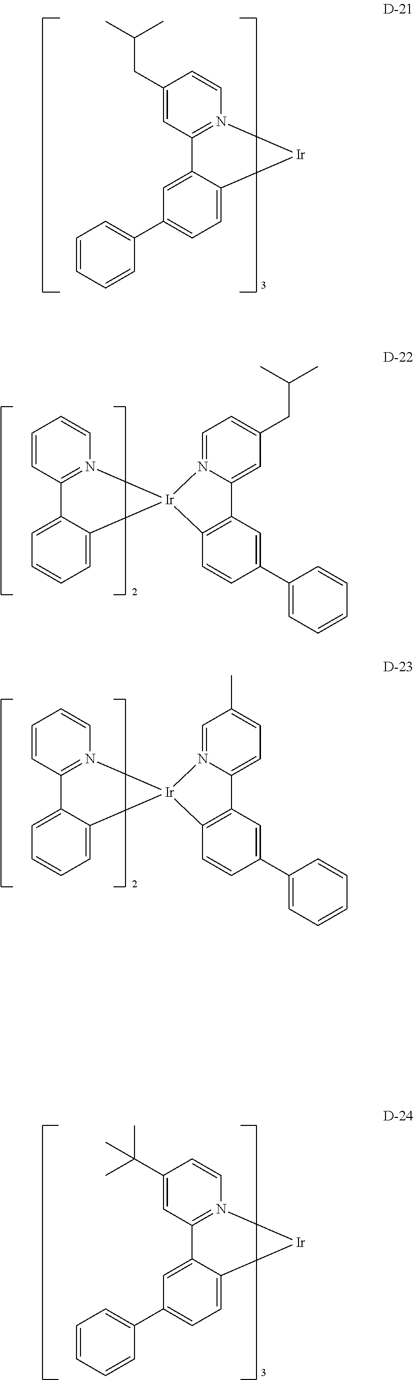

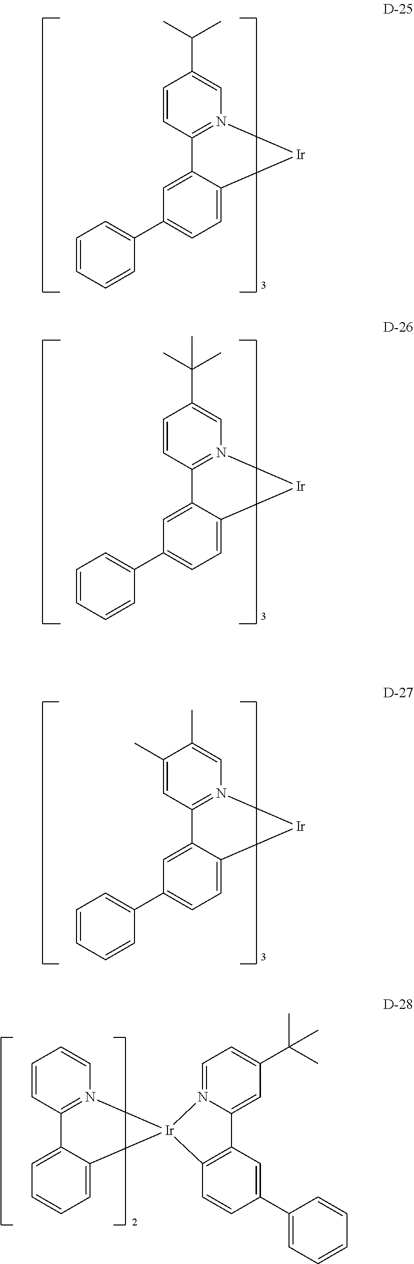

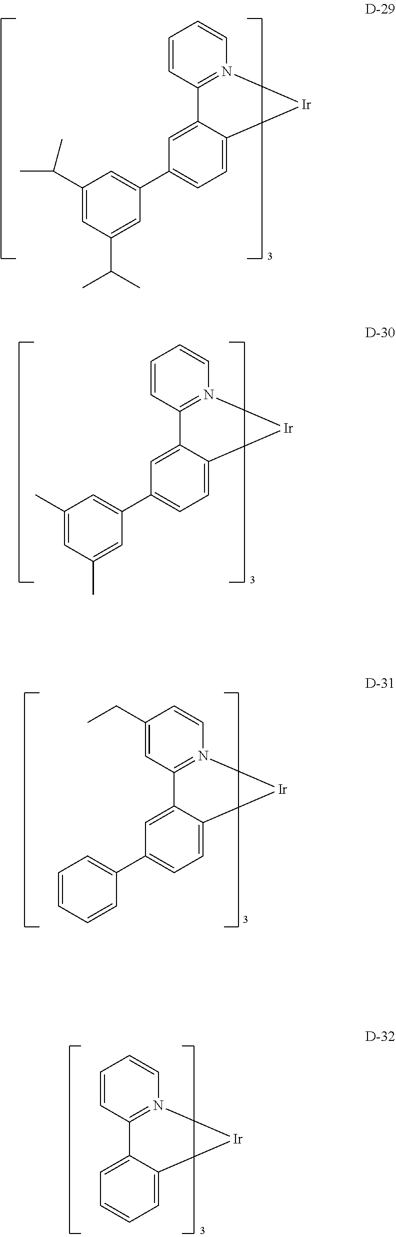

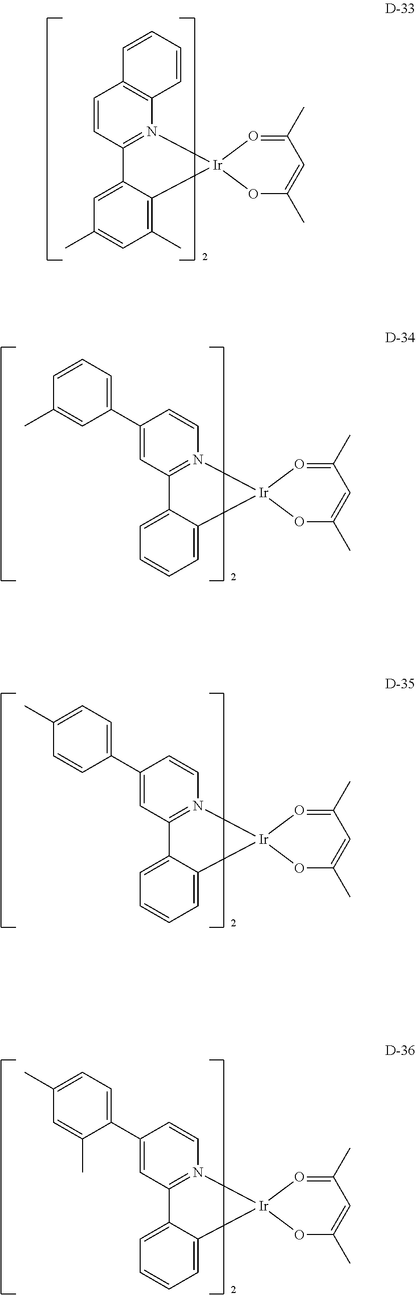

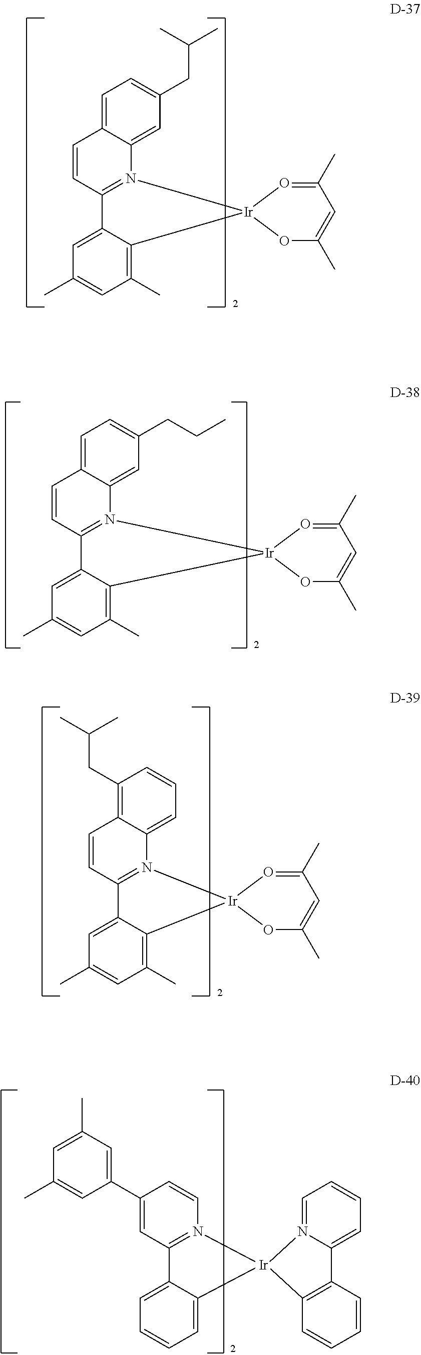

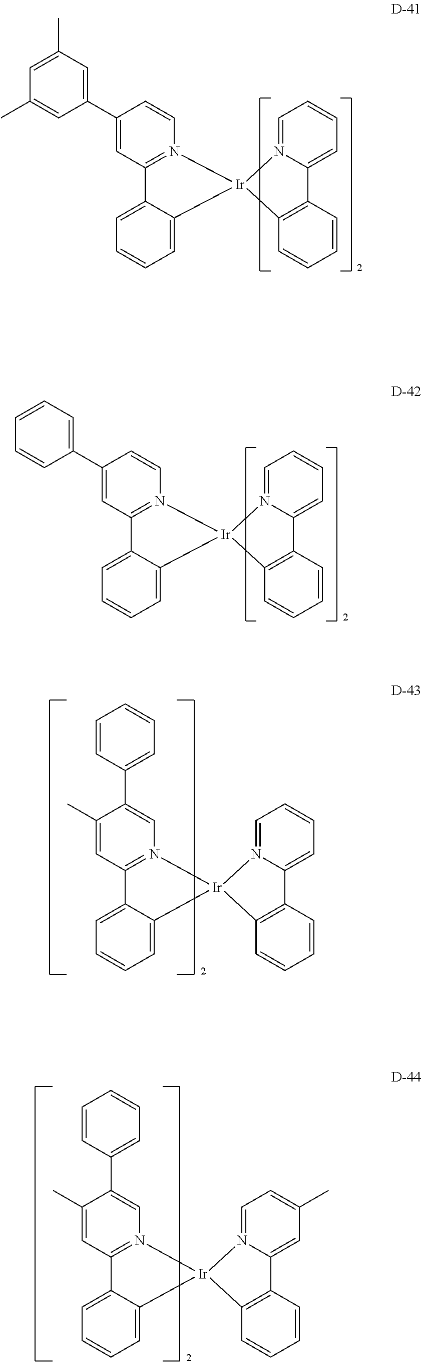

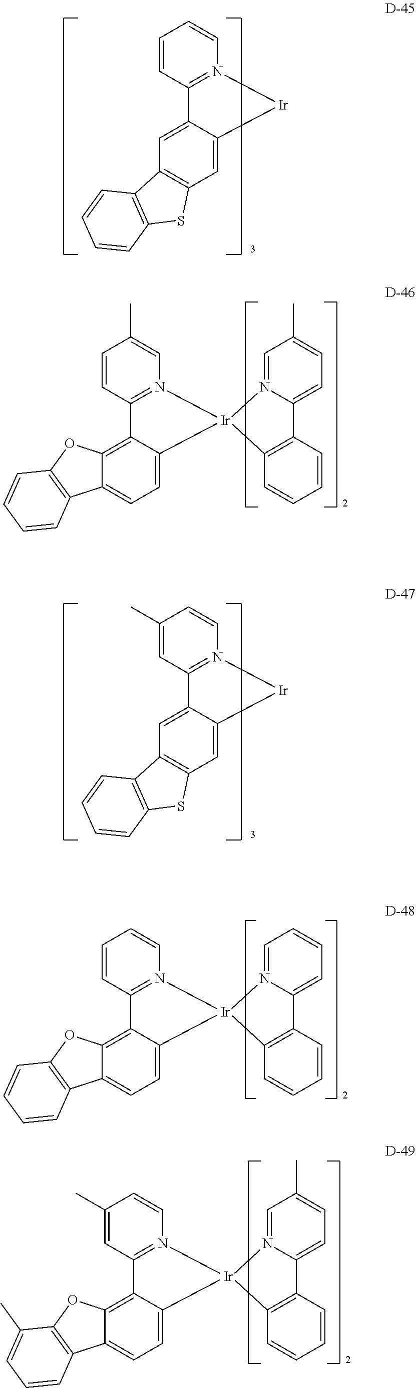

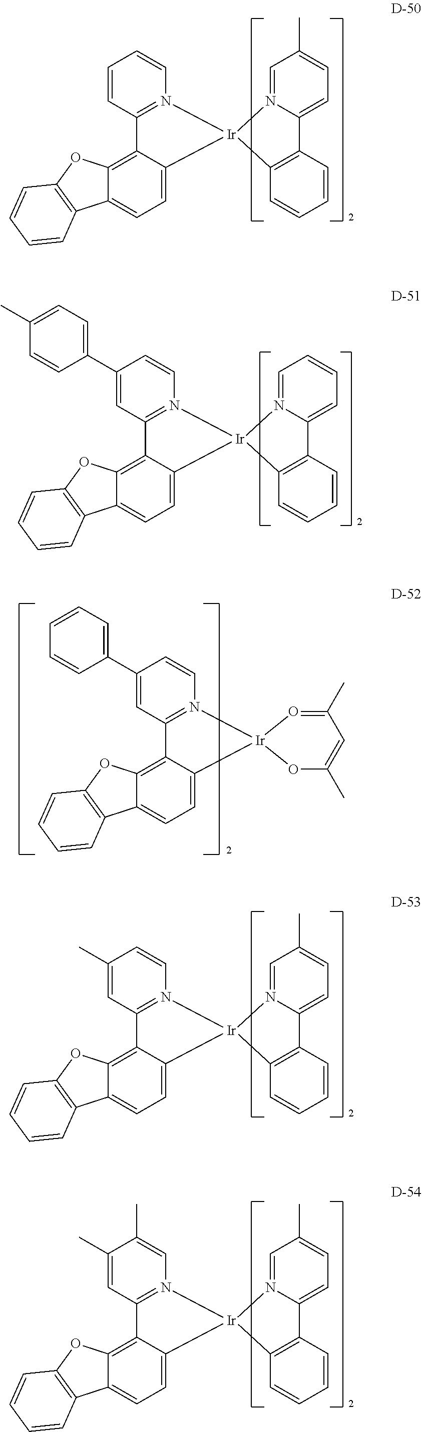

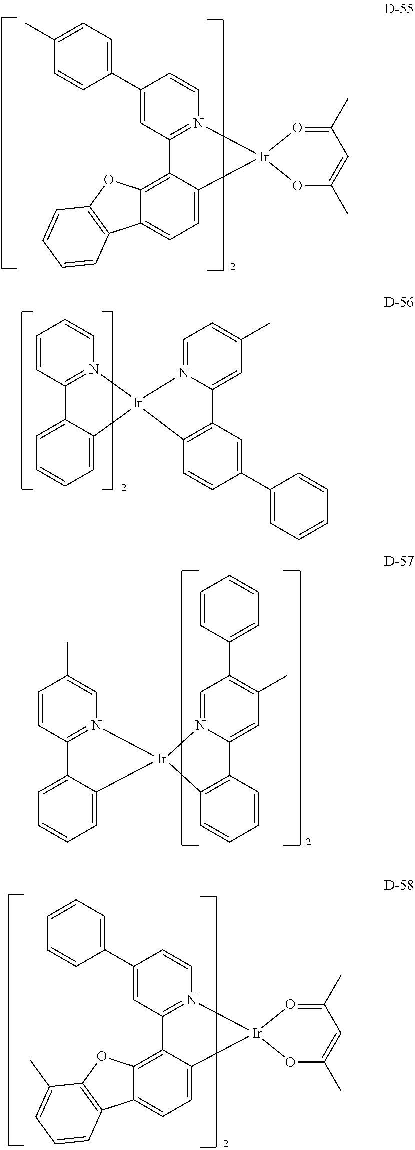

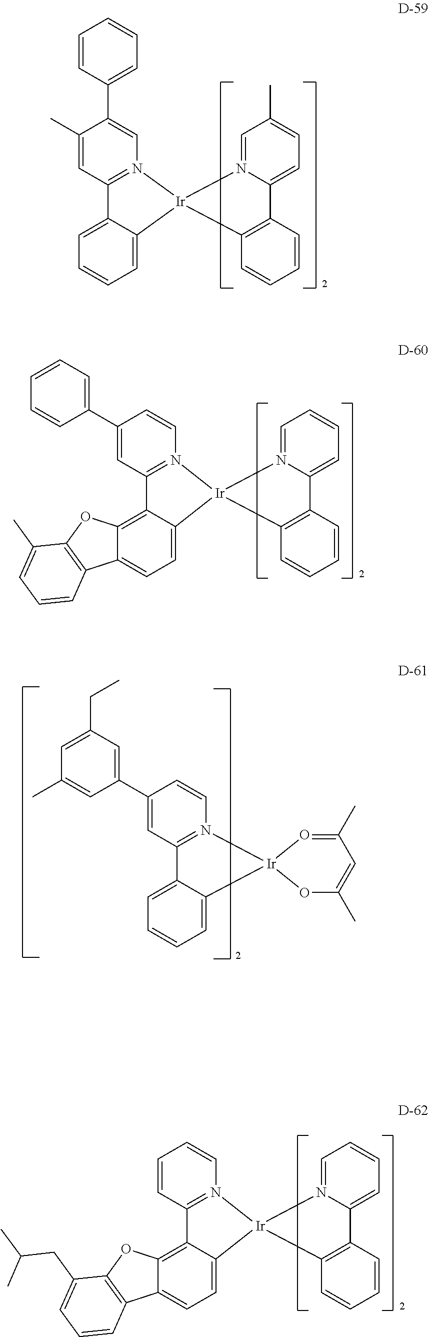

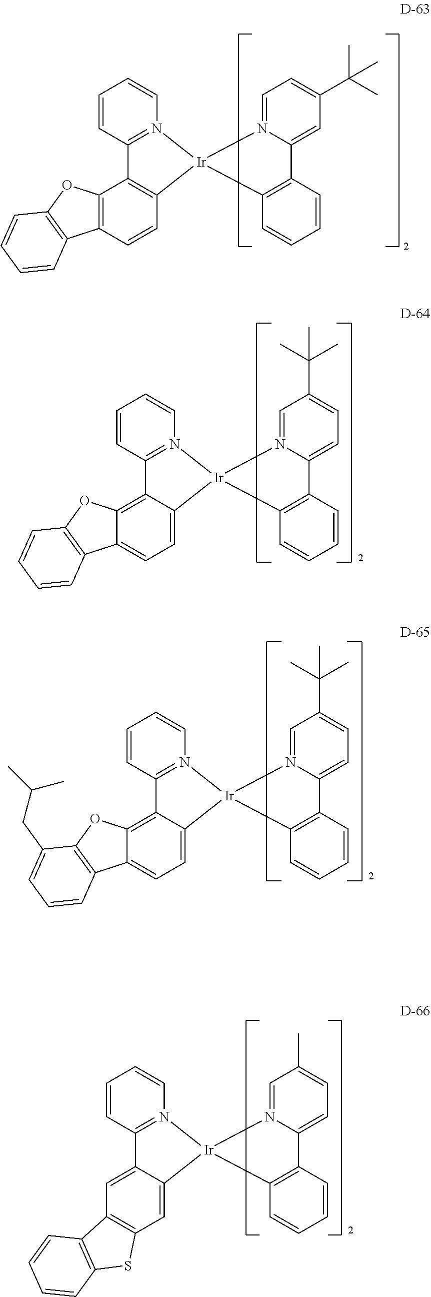

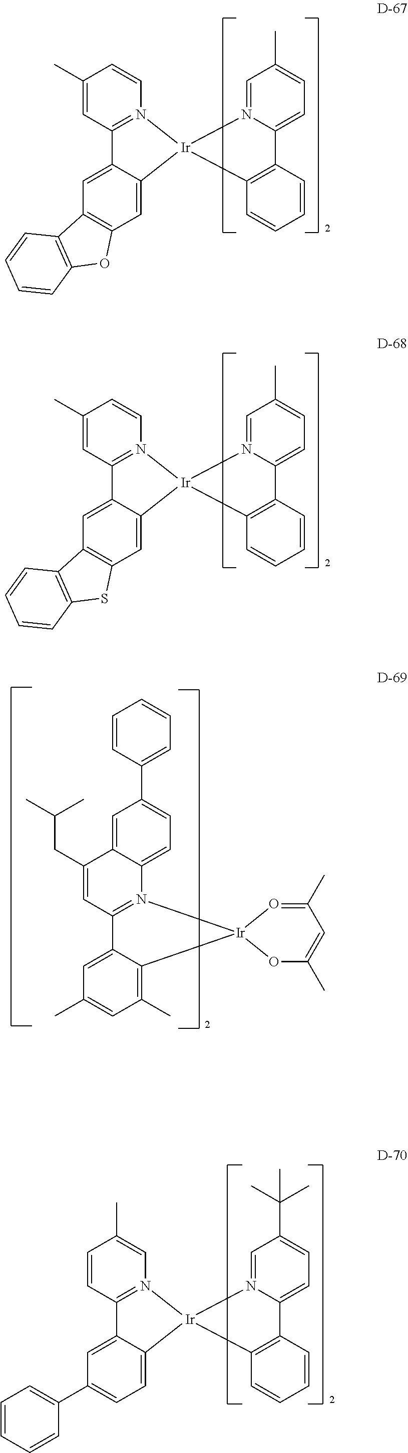

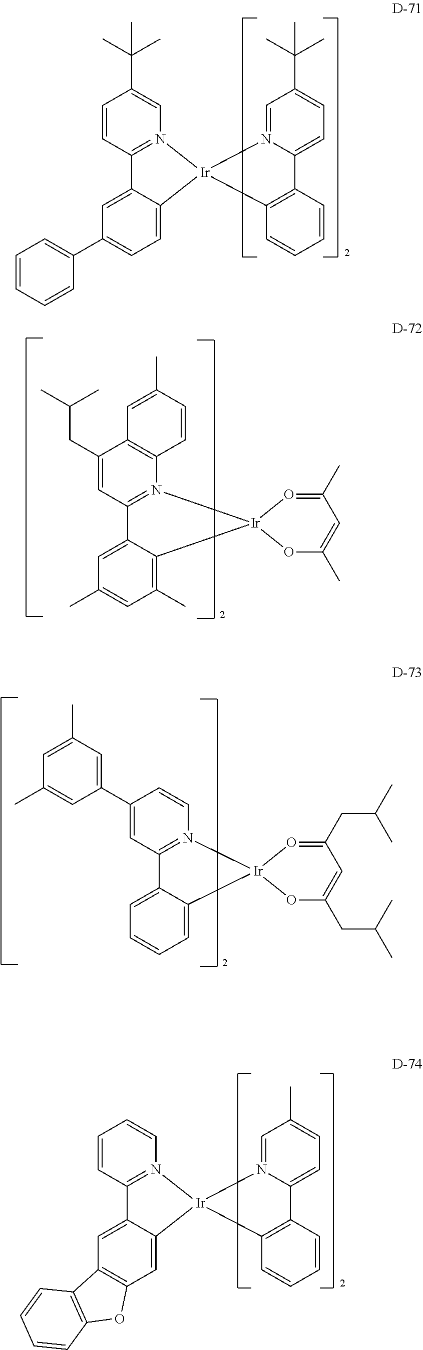

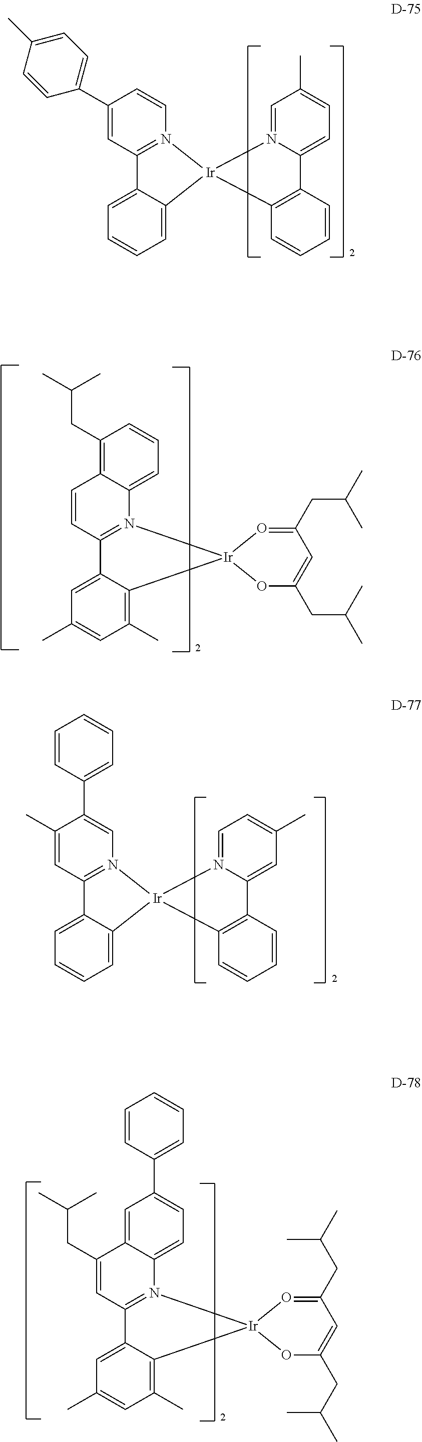

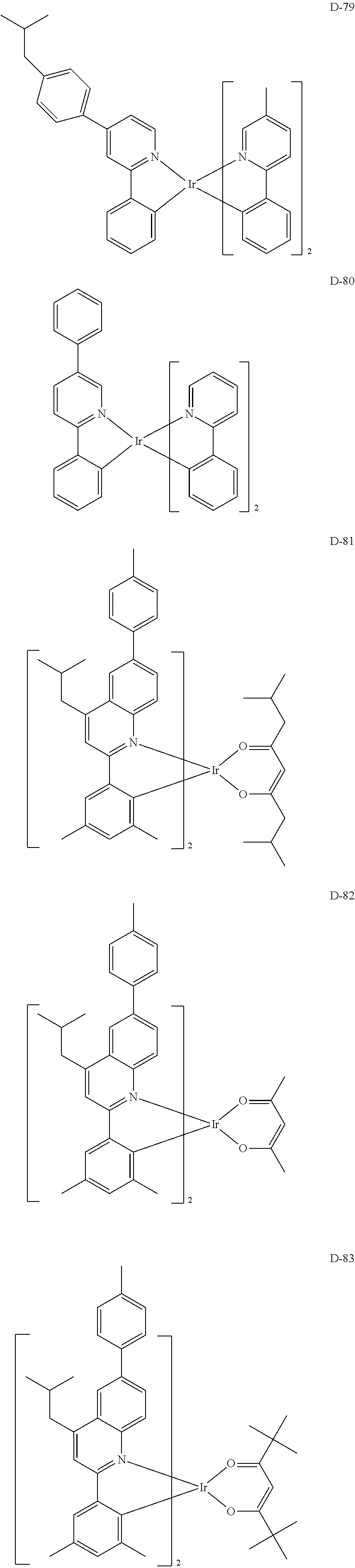

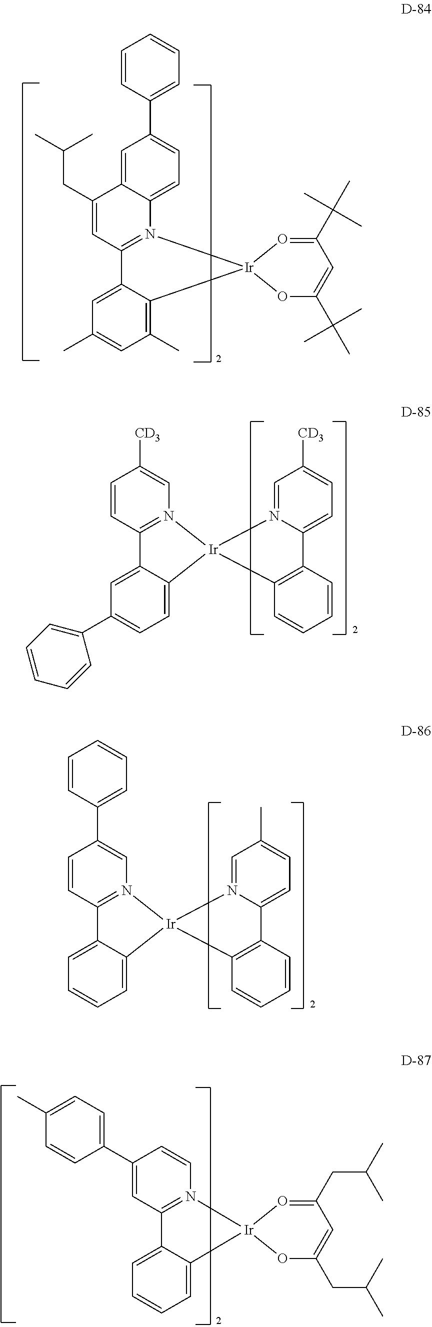

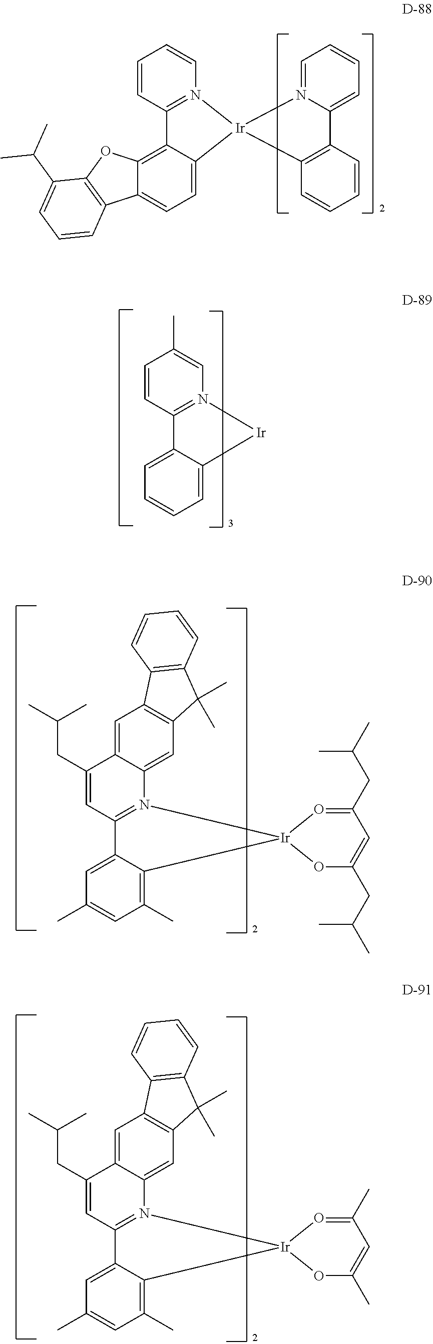

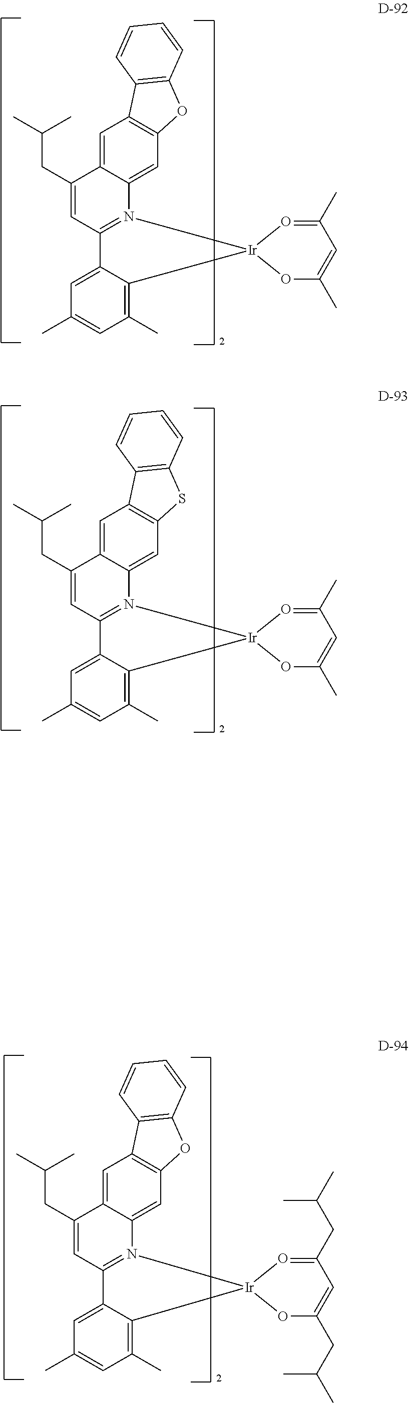

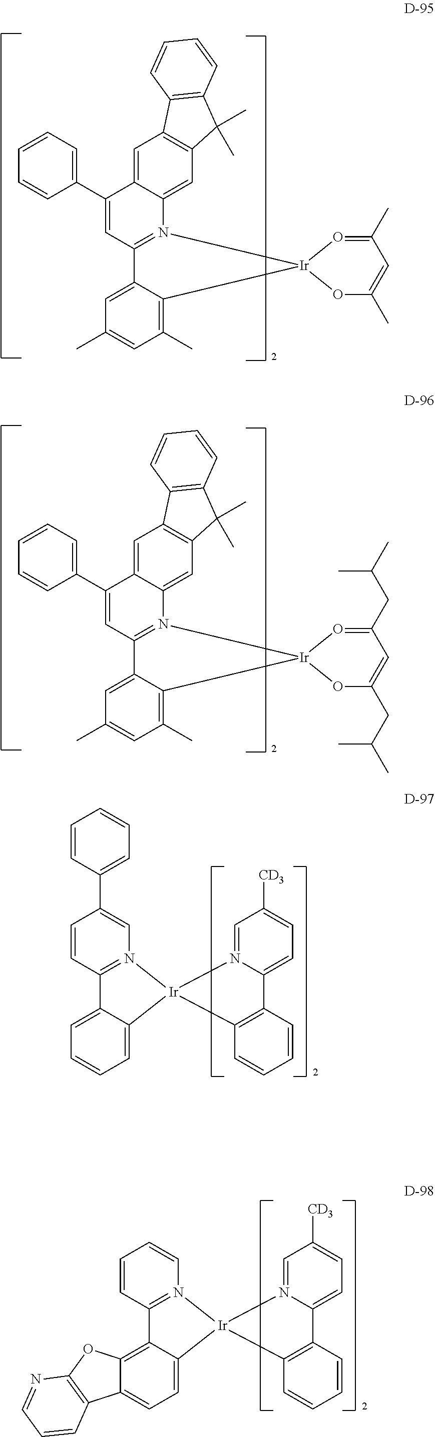

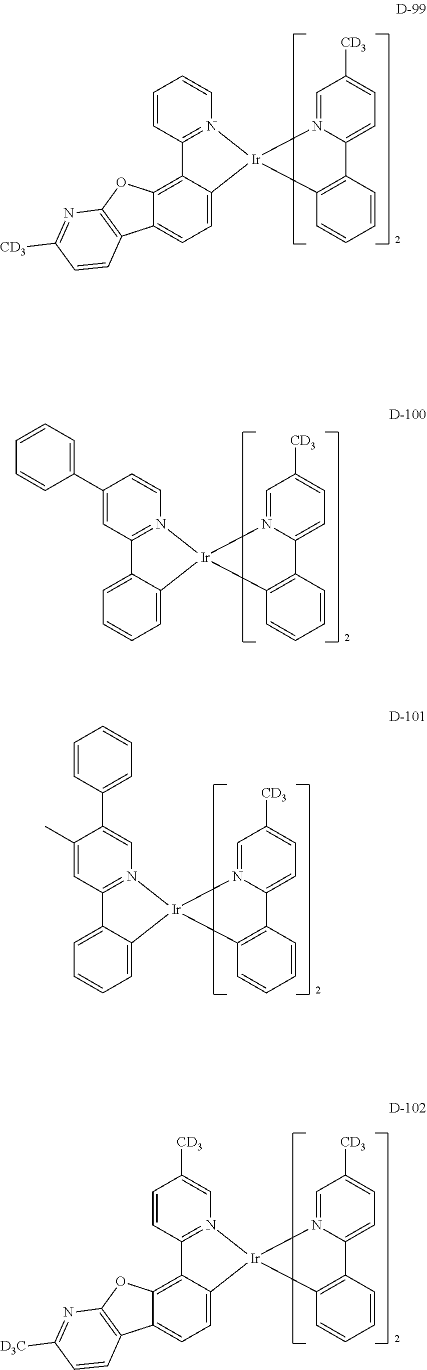

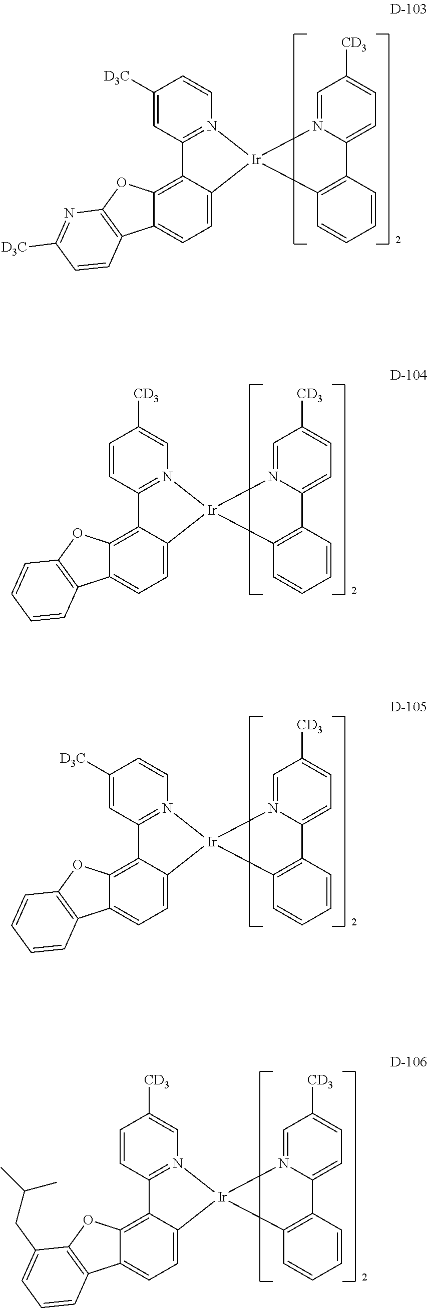

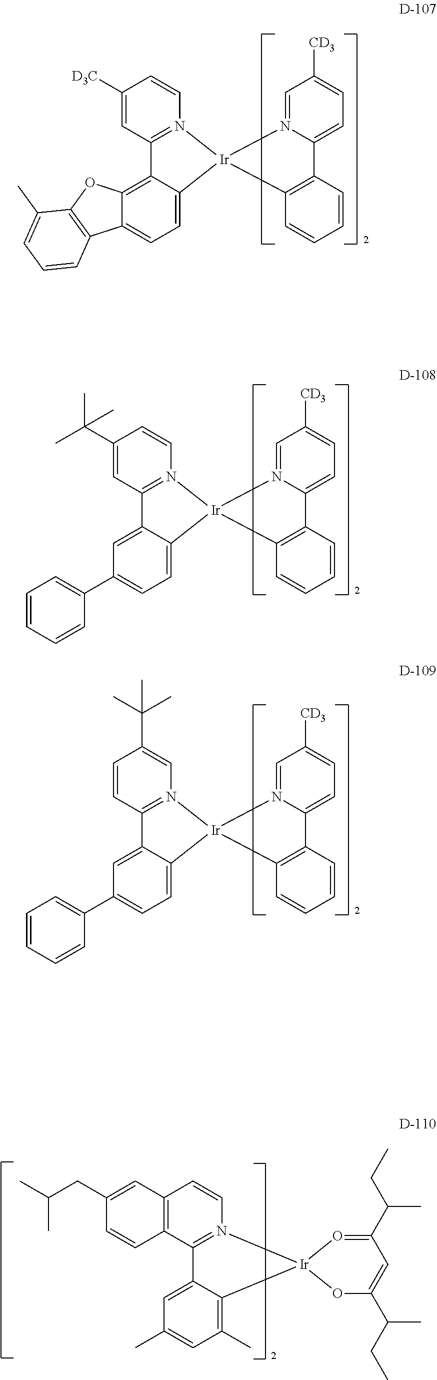

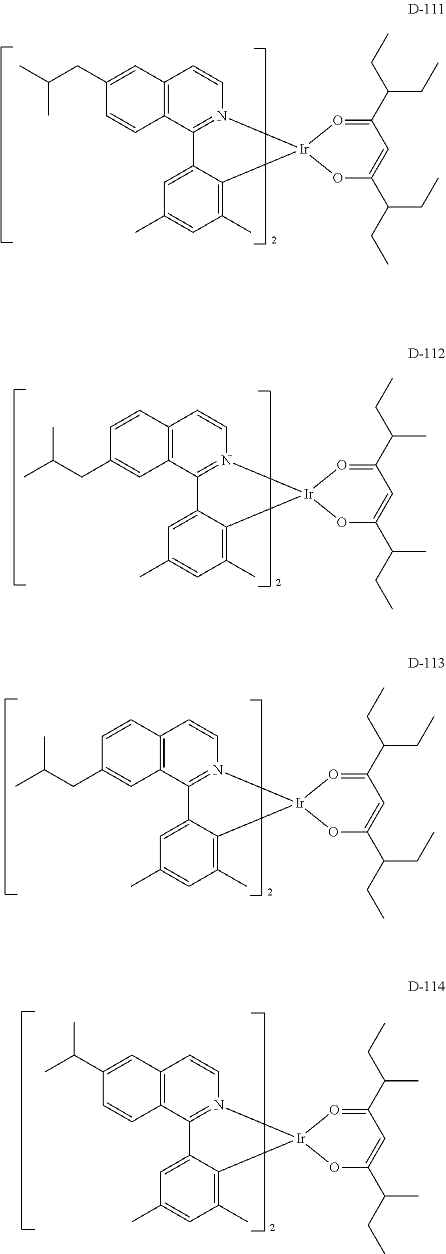

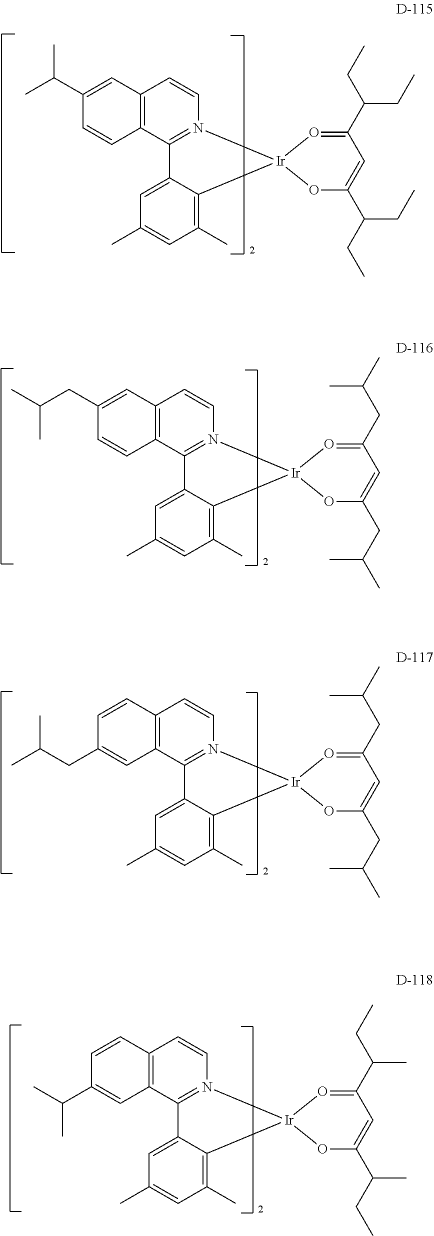

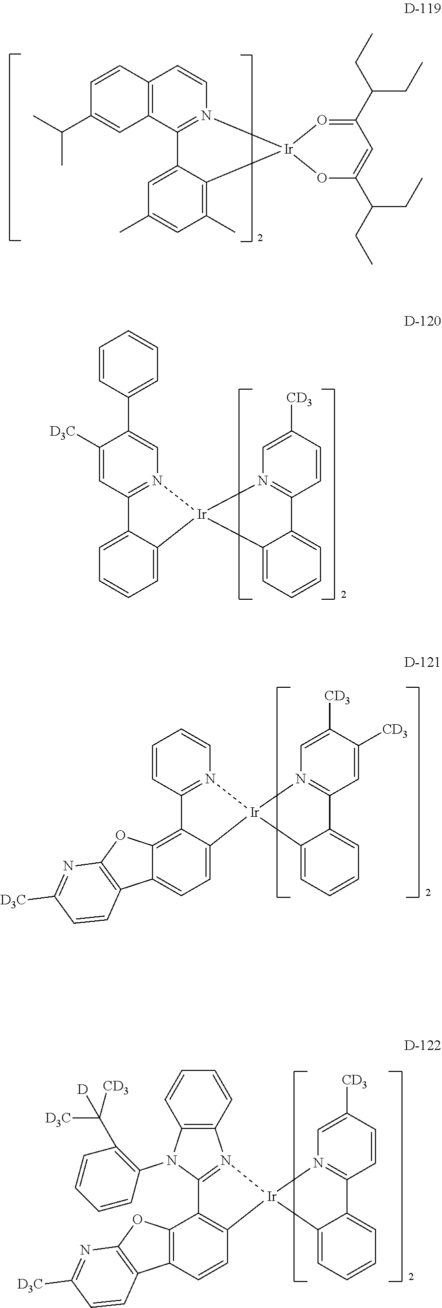

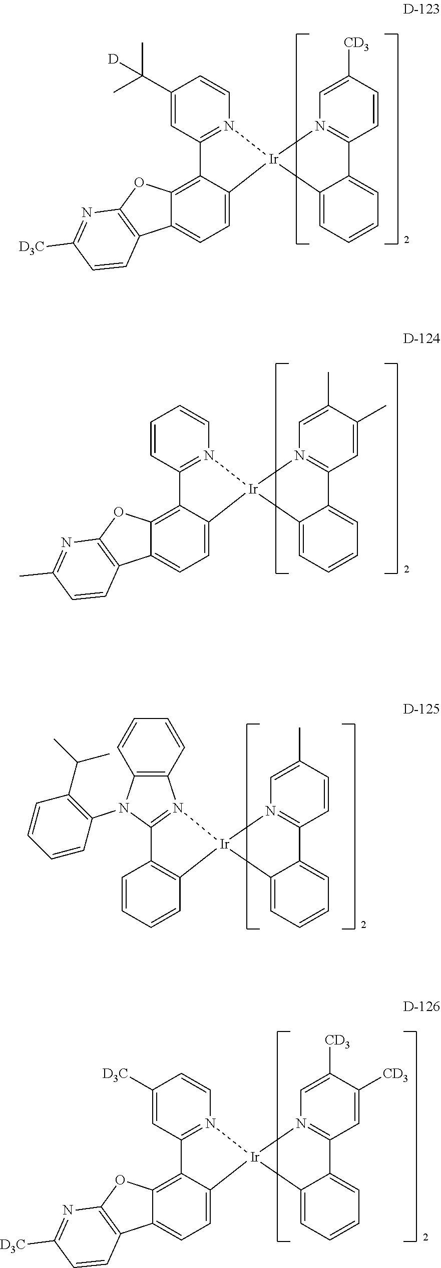

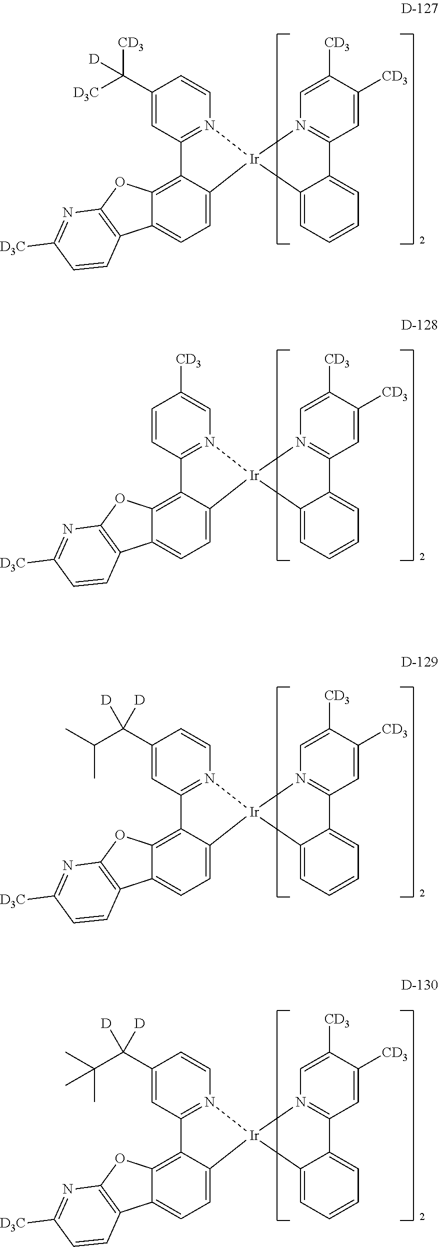

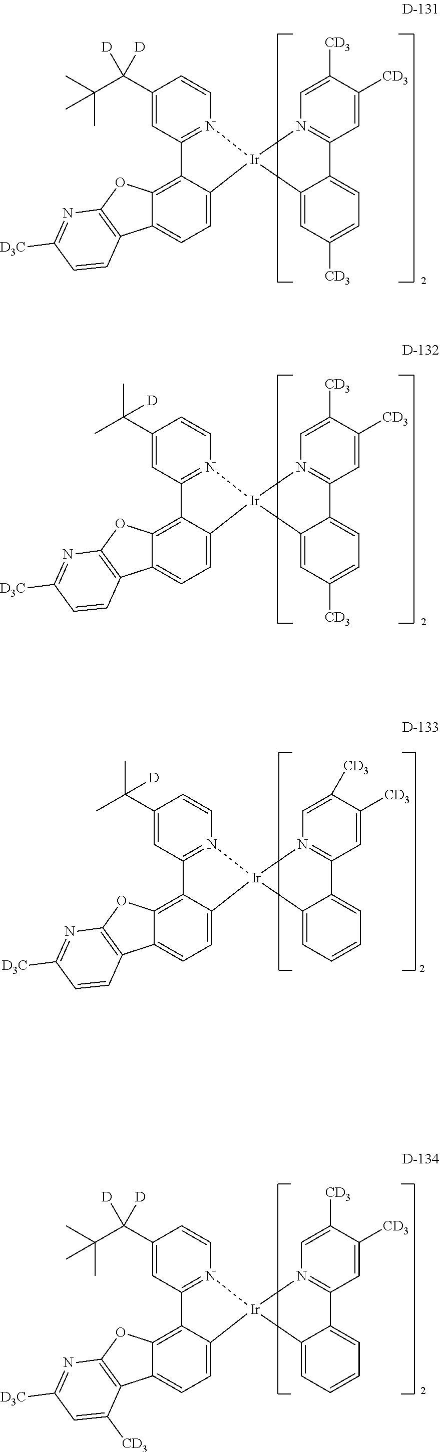

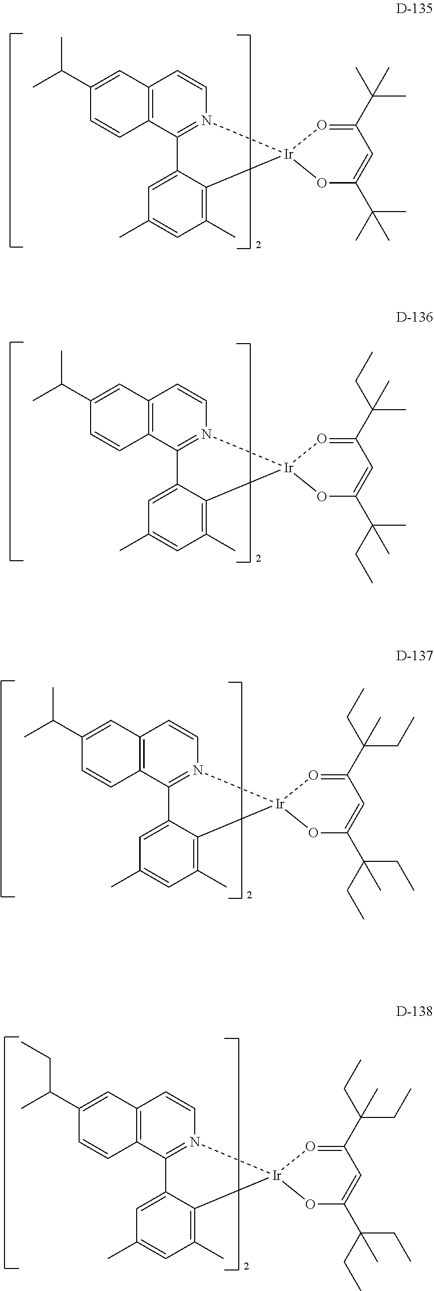

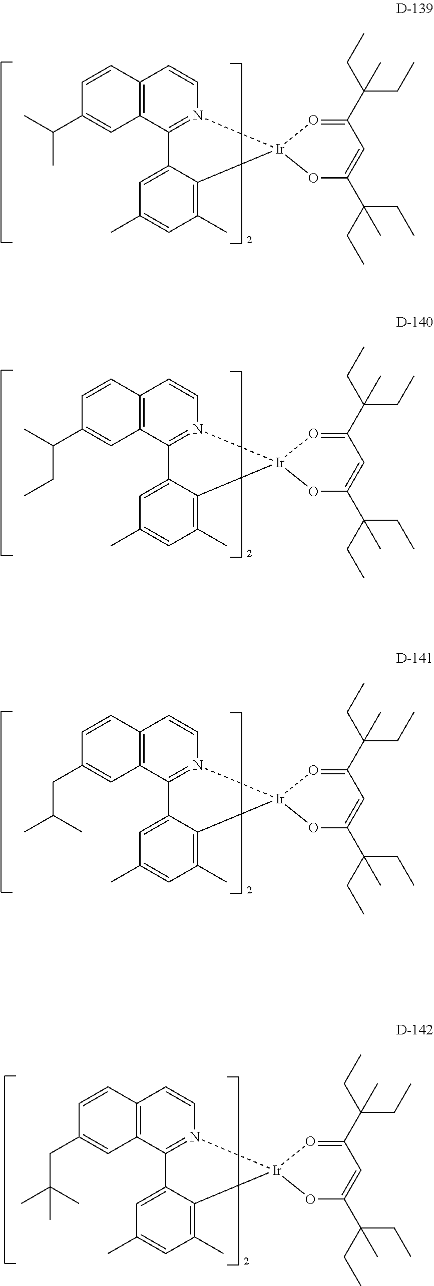

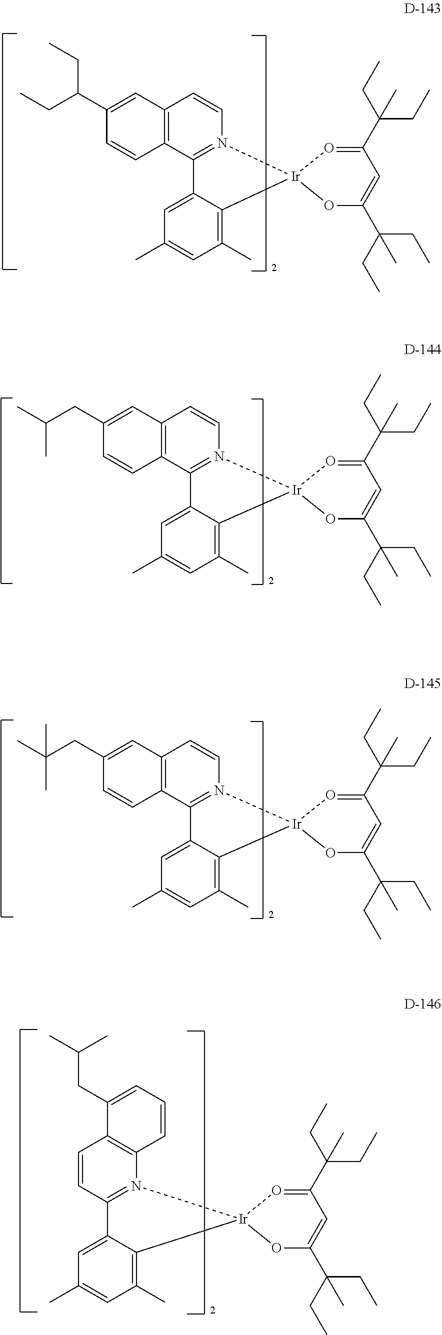

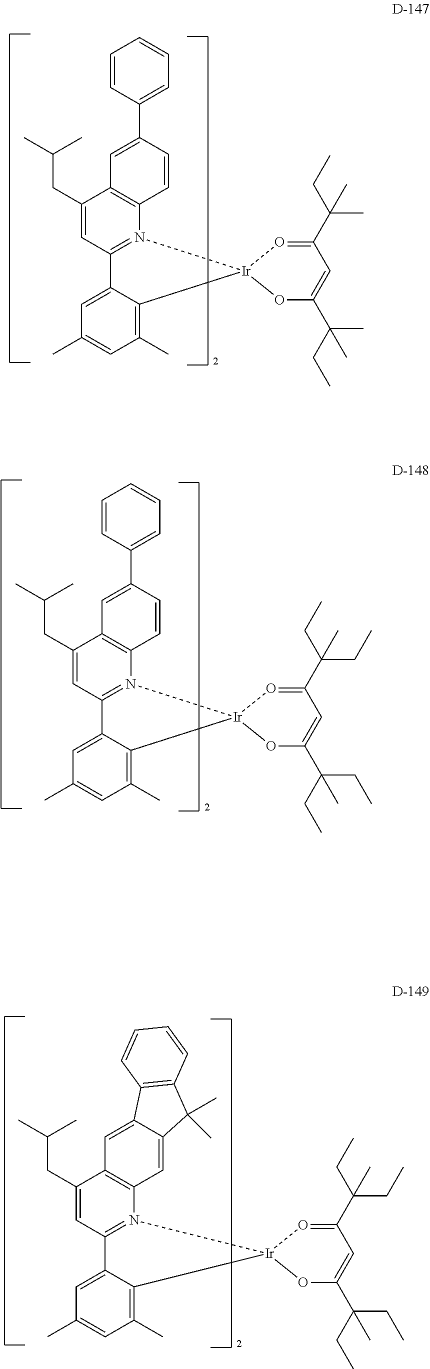

Hereinafter, an organic electroluminescent device to which the aforementioned organic electroluminescent compound or organic electroluminescent material is applied will be described.

The organic electroluminescent device according to one embodiment includes a first electrode; a second electrode; and at least one organic layer(s) interposed between the first electrode and the second electrode. In one embodiment, the organic layer includes a hole transport layer, a light-emitting layer, a buffer layer and/or an electron transport layer, preferably, a light-emitting layer or an electron buffer layer, comprising the organic electroluminescent compound according to the present disclosure. For example, the light-emitting layer may comprise the organic electroluminescent compound of the present disclosure alone, or a mixture of at least two of the organic electroluminescent compounds, and may further include conventional materials included in the organic electroluminescent material. In addition, the organic layer may further comprise at least one layer selected from a hole injection layer, a hole auxiliary layer, a light-emitting auxiliary layer, an electron injection layer, an interlayer, a hole blocking layer, and an electron blocking layer, in addition to the hole transport layer, the light-emitting layer, the buffer layer and the electron transport layer, and each layer may be further comprised of a plurality of layers.

In addition, the organic layer may further include at least one selected from an arylamine-based compound and a styrylarylamine-based compound. In addition, the organic layer further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The organic electroluminescent material according to one embodiment may be used as light-emitting materials for a white organic light-emitting device. The white organic electroluminescent device has suggested various structures such as a parallel side-by-side arrangement method, a stacking arrangement method, or color conversion material (CCM) method, etc., according to the arrangement of R (Red), G (Green), YG (yellowish green), or B (Blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X} (15≤X≤2), AlO_{X} (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by a host compound and a dopant compound according to one embodiment, the layer can be formed by co-deposition or mixed deposition, but is not limited thereto. The co-deposition is a mixed deposition method in which two or more materials are put into respective individual crucible sources and a current is applied to both cells simultaneously to evaporate the materials; and the mixed deposition is a method in which two or more materials are mixed in one crucible source before deposition, and then a current is applied to one cell to evaporate the materials.

According to one embodiment, the present disclosure can provide display devices comprising an organic electroluminescent compound represented by the formula 1, and/or a plurality of host materials including the same. In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of compounds according to the present disclosure will be explained with reference to the synthesis method of a representative compound or intermediate compound in order to understand the present disclosure in detail.

### [Example 1] Preparation of Compound C-32

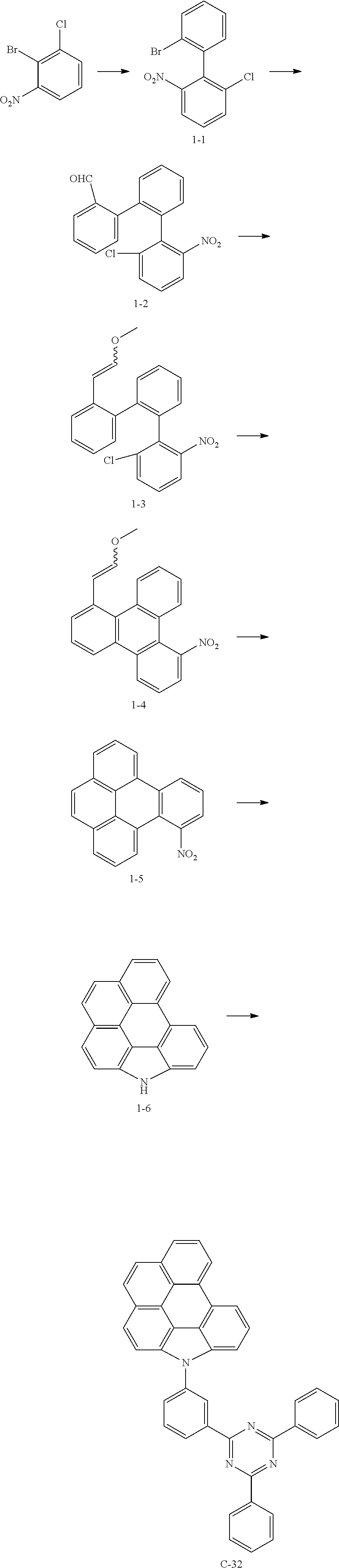

1) Synthesis of Compound 1-1

2-bromo-1-chloro-3-nitrobenzene (60 g, 253.75 mmol), 2-bromophenylboronic acid (51 g, 253.75 mmol), tetrakis(trphenylphosphine)palladium(0) (Pd(PPh₃)₄) (20.5 g, 17.76 mmol), sodium hydroxide (25.4 g, 634.40 mmol), 1,200 mL of tetrahydrofuran, and 300 mL of H₂O were added to the reaction vessel and then refluxed for 12 hours. After completion of the reaction, the mixture was cooled to room temperature and then the organic layers were extracted with ethyl acetate. The extracted organic layer was dried over magnesium sulfate, and then the solvent was removed using a rotary evaporator. Next, it was purified by column chromatography to obtain compound 1-1 (44 g, yield: 56%).

2) Synthesis of Compound 1-2

Compound 1-1 (44 g, 141.51 mmol), 2-formylphenylboronic acid (53 g, 353.78 mmol), Pd(PPh₃)₄ (11.4 g, 9.91 mmol), potassium hydroxide (17.4 g, 311.32 mmol), 440 mL of o-xylene, 88 mL of acetonitrile, and 176 mL of H₂O were added to the reaction vessel and then refluxed for 12 hours. After completion of the reaction, the mixture was cooled to room temperature and then the organic layers were extracted with ethyl acetate. The extracted organic layer was dried over magnesium sulfate, and then the solvent was removed using a rotary evaporator. Next, it was purified by column chromatography to obtain Compound 1-2 (28.3 g. yield: 59%).

3) Synthesis of Compound 1-3

Compound 1-2 (28.3 g, 83.79 mmol), (methoxymethyl)triphenylphosphonium chloride (43 g, 125.68 mmol,) and 380 mL of tetrahydrofuran were added to the reaction vessel and stirred for 5 minutes. Next, potassium t-butoxide (1M in THF, 126 mL) was slowly added dropwise to the reaction mixture under the condition of 0° C. The temperature was slowly raised to room temperature and stirred for 4 hours. Distilled water was added to the reaction solution to end the reaction, and then the organic layers were extracted with ethyl acetate. The extracted organic layer was dried over magnesium sulfate, and then the solvent was removed using a rotary evaporator. Next, it was purified by column chromatography to obtain compound 1-3 (23 g, yield: 75%).

4) Synthesis of Compound 1-4

Compound 1-3 (23 g, 62.87 mmol), palladium(II) acetate (710 mg, 3.14 mmol), tricyclohexylphosphine tetrafluoroborate (2.3 g, 6.29 mmol), cesium carbonate (61.4 g, 188.61 mmol), and 320 mL of o-xylene were added to the reaction vessel and then refluxed for 12 hours. After completion of the reaction, the mixture was cooled to room temperature and then the organic layers were extracted with ethyl acetate. The extracted organic layer was dried over magnesium sulfate, and then the solvent was removed using a rotary evaporator. Next, it was purified by column chromatography to obtain compound 1-4 (13.6 g, yield: 66%).

5) Synthesis of Compound 1-5

Compound 1-4 (13.6 g, 41.29 mmol) and 200 mL of methylene chloride were added to the reaction vessel and stirred at 0° C. for 5 minutes. Next, boron trifluoride diethyl ether (15.6 mL, 123.88 mmol) was slowly added dropwise to the reaction mixture under the condition of 0° C. The temperature was slowly raised to room temperature and stirred for 3 hours. Distilled water was added to the reaction solution to end the reaction. After neutralization with aqueous sodium bicarbonate solution, the organic layers were extracted with methylene chloride. The extracted organic layer was dried over magnesium sulfate, and then the solvent was removed using a rotary evaporator. Next, it was purified by column chromatography to obtain compound 1-5 (10.2 g, yield: 83%).

6) Synthesis of Compound 1-6

Compound 1-5 (10.2 g, 34.48 mmol), triphenylphosphine (27.1 g, 103.43 mmol), and 170 mL of o-dichlorobenzene were added to the reaction vessel and then refluxed for 12 hours. After completion of the reaction, the mixture was cooled to room temperature. Next, it was purified by column chromatography to obtain compound 1-6 (6.7 g, yield: 74%).

¹H NMR (400 MHz, CDCl₃) 8.86-8.84 (d, 1H), 8.40 (s, 1H), 8.33-8.31 (d, 1H), 8.21-8.17 (t, 2H), 8.11-8.09 (d, 1H), 8.05-8.01 (t, 1H), 7.95-7.94 (d, 1H), 7.93-7.92 (d, 1H), 7.87-7.83 (t, 1H), 7.67-7.65 (d, 1H)

7) Synthesis of Compound C-32

Compound 1-6 (3.8 g, 14.32 mmol), 2-(3-bromophenyl)-4,6-diphenyl-1,3,5-triazine (6.1 g, 15.76 mmol), palladium(II)acetate (160 mg, 0.72 mmol), 2-dicyclohexylphosphino-2′,6′-dimethoxybiphenyl (590 mg, 1.43 mmol), sodium t-butoxide (3.4 g, 35.80 mmol), and 72 mL of o-xylene were added to the reaction vessel and then refluxed for 4 hours. After completion of the reaction, the mixture was cooled to room temperature, the reaction mixture was poured into methanol to precipitate a solid, and the precipitated solid was filtered and dried. Next, it was purified by column chromatography to obtain compound C-32 (4.9 g, yield: 60%).

| | MW | M.P |
|---|---|---|
| C-32 | 572.67 | 344° C. |

### [Example 2] Preparation of Compound C-53

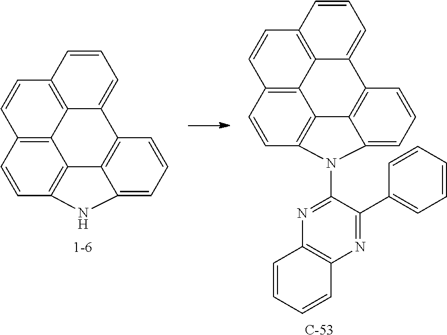

Compound 1-6 (3 g, 11.61 mmol), 2-chloro-3-phenylquinoxaline (4.54 g, 18.86 mmol), cesium carbonate (7.5 g, 23.01 mmol), 4-dimethylaminopyridine (715 mg, 5.85 mmol), and 60 mL of dimethylsulfoxide were added to the reaction vessel and then refluxed for 4 hours. After completion of the reaction, the mixture was cooled to room temperature, the reaction mixture was poured into distilled water to precipitate a solid, and the precipitated solid was filtered and dried. Next, it was purified by column chromatography to obtain compound C-53 (2.8 g, yield: 52%).

| | MW | M.P |
|---|---|---|
| C-53 | 469.55 | 275° C. |

### [Example 3] Preparation of Compound C-4

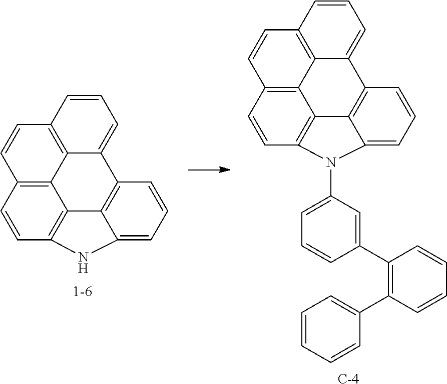

Compound 1-6 (3.35 g, 12.63 mmol), 3-bromo-1,1′:2′,1″-terphenyl (4.3 g, 13.89 mmol), palladium(II)acetate (141 mg, 0.63 mmol), 2-dicyclohexylphosphino-2′,6′-dimethoxybiphenyl (520 mg, 1.26 mmol), sodium t-butoxide (3 g, 31.58 mmol), and 80 mL of o-xylene were added to the reaction vessel and then refluxed for 12 hours. After completion of the reaction, the mixture was cooled to room temperature and then purified by column chromatography to obtain compound C-4 (2.58 g, yield: 41%).

| | MW | M.P |
|---|---|---|
| C-4 | 493.61 | 157° C. |

### [Example 4] Preparation of Compound C-7

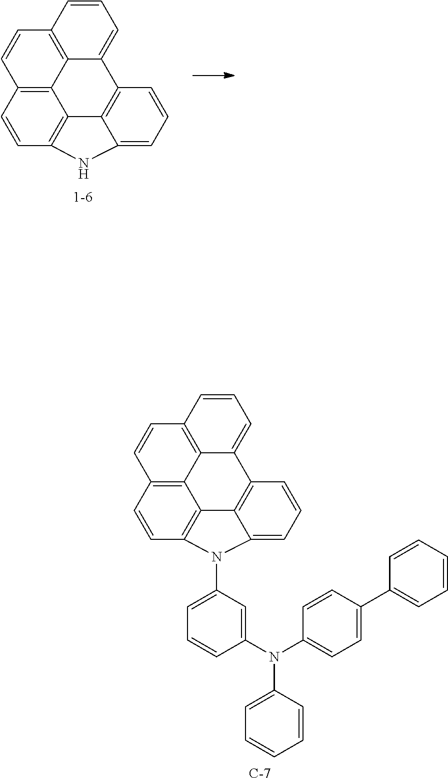

Compound 1-6 (3.35 g, 12.63 mmol), N-(3-bromophenyl)-N-phenyl-[1,1′-biphenyl]-4-amine (5.56 g, 13.89 mmol), tris(dibenzylideneacetone)dipalladium(0) (580 mg, 0.63 mmol), tri t-butylphosphine (50 wt % in toluene, 0.62 mL), sodium t-butoxide (3 g, 31.58 mmol), and 80 mL of toluene were added to the reaction vessel and then refluxed for 12 hours. After completion of the reaction, the mixture was cooled to room temperature and then purified by column chromatography to obtain compound C-7 (4.36 g, yield: 59%).

| | MW | M.P |
|---|---|---|
| C-7 | 584.72 | 199° C. |

### [Example 5] Preparation of Compound H2-29

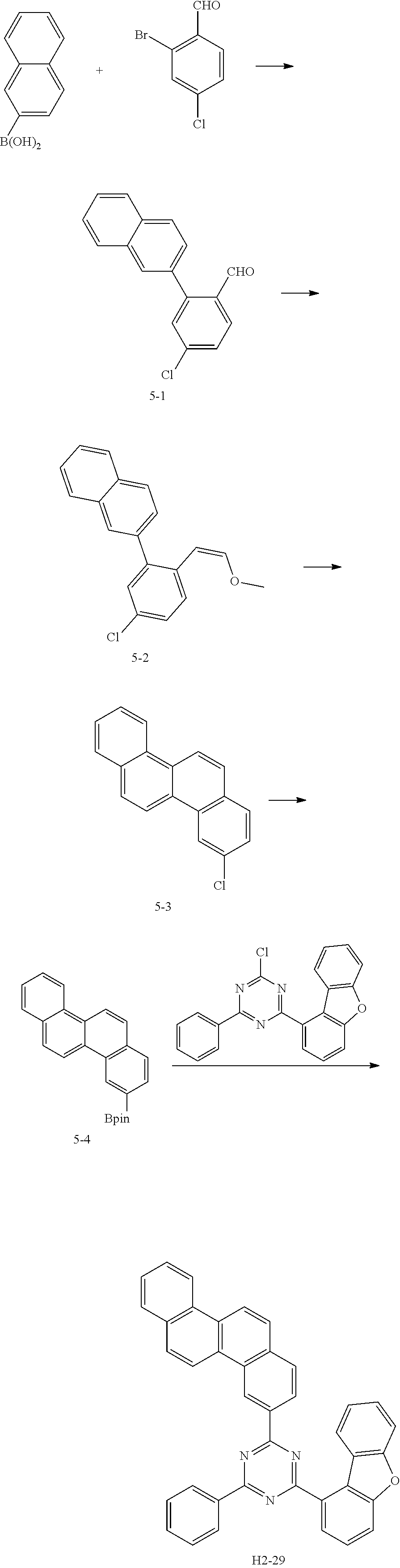

1) Synthesis of Compound 5-1

Naphthalen-2-ylboronic acid (50 g, 291 mmol), 2-bromo-4-chlorobenzaldehyde (63 g, 291 mmol), tetrakis(trphenylphosphine)palladium (16.8 g, 14.5 mmol), sodium carbonate (77 g, 727 mmol), toluene (1,080 mL), ethanol (240 mL), and distilled water (360 mL) were added to the reaction vessel and stirred at 140° C. for 5 hours. After completion of the reaction, the precipitated solid was washed with distilled water and methanol. Next, it was purified by column chromatography to obtain compound 5-1 (71 g, yield: 92%).

2) Synthesis of Compound 5-2

Compound 5-1 (71 g, 268 mmol), (methoxymethyl)tiphenylphosphonium chloride (110 g, 321 mmol) and tetrahydrofuran (1,300 mL) were added to the reaction vessel and the reaction mixture was stirred for 10 minutes, and then potassium tert-butoxide (1M in THF, 300 mL) was slowly added dropwise thereto under the condition of 0° C. The temperature was raised slowly and the mixture was stirred at room temperature for 3 hours. Distilled water was added to the reaction solution to end the reaction, and the mixture was extracted with ethyl acetate. The extracted organic layer was dried over magnesium sulfate, and then the solvent was removed using a rotary evaporator. Next, it was purified by column chromatography to obtain compound 5-2 (71 g, yield: 90%).

3) Synthesis of Compound 5-3

Compound 5-2 (70 g, 238 mmol), Eaton's reagent (7 mL) and chlorobenzene (1.180 mL) were added to the reaction vessel and refluxed for 1 hour. After completion of the reaction, the mixture was cooled to room temperature and extracted with methylene chloride (MC). The extracted organic layer was dried over magnesium sulfate, and then the solvent was removed using a rotary evaporator. Next, it was purified by column chromatography to obtain compound 5-3 (60 g, yield: 96%).

4) Synthesis of Compound 5-4

Compound 5-3 (35 g, 133.2 mmol), bis(pinacolato)diborane (44 g, 173 mmol), tris(dibenzylideneacetone)dipalladium (6.1 g, 6.66 mmol), 2-dicyclohexylphosphino-2′,6′-dimethoxybiphenyl(s-phos) (5.5 g, 13.3 mmol), potassium acetate (39.2 g, 400 mmol) and 1,4-dioxane (666 mL) were added to the reaction vessel, and then stirred at 150° C. for 3 hours. After completion of the reaction, the mixture was cooled to room temperature and extracted with ethyl acetate. The extracted organic layer was dried over magnesium sulfate, and then the solvent was removed using a rotary evaporator. Next, it was purified by column chromatography to obtain compound 5-4 (38 g, yield: 81%).

5) Synthesis of Compound H2-29

Compound 5-4 (5 g, 14.1 mmol), 2-chloro-4-(dibenzo[b,d]furan-1-yl)-6-phenyl-1,3,5-triazine (6.6 g, 18.3 mmol), tetrakis(triphenylphosphine)palladium (0.8 g, 0.7 mmol), potassium carbonate (3.9 g, 28.2 mmol), toluene (42 mL), ethanol (10 mL) and distilled water (14 mL) were added to the reaction vessel, and then stirred at 140° C. for 8 hours. After the reaction was completed, the mixture was added dropwise to methanol, and the resulting solid was filtered. The resulting solid was purified by column chromatography to obtain compound H2-29 (6.8 g, yield: 88%).

| | MW | M.P. |
|---|---|---|
| H2-29 | 549.6 | 267° C. |

Hereinafter, the preparation method of an organic electroluminescent device comprising the organic electroluminescent compound and/or the plurality of host materials of the present disclosure, and the device property thereof will be explained in order to understand the present disclosure in detail.

### [Device Examples 1 and 2] Preparation of OLEDs Deposited with the Compound According to the Present Disclosure as a Host

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and compound HT-1 as a first hole transport compound was introduced into another cell. The two materials were evaporated at different rates and the first hole injection compound was deposited in a doping amount of 3 wt % based on the total amount of the first hole injection compound and the first hole transport compound to form a first hole injection layer having a thickness of 10 nm. Next, compound HT-1 was deposited as a first hole transport layer having a thickness of 80 nm on the first hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The host material described in the following Table 1 was introduced into a cell of the vacuum vapor deposition apparatus as a host, and compound D-39 was introduced into another cell as a dopant. The two materials were evaporated at different rates, and the dopant was deposited in a doping amount of 3 wt % based on the total amount of the host and dopant to form a light-emitting layer having a thickness of 40 nm on the second hole transport layer. Next, compounds ETL-1 and EIL-1 as materials for electron transport layer were deposited at a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 1] Preparation of an OLED Comprising the Conventional Compound as a Host

An OLED was produced in the same manner as in Device Example 1, except that compound CBP was used as the host of the light-emitting layer.

The driving voltage, the luminous efficiency, and the light-emitting color at a luminance of 1,000 nits, and the time taken for luminance to decrease from 100% to 95% at a luminance of 5,000 nits (lifespan; T95) of the OLEDs according to Device Examples 1 and 2 and Comparative Example 1 produced as described above, are measured, and the results thereof are shown in Table 1 below:

TABLE 1

| | | Driving | Luminous | Light- | |
|---|---|---|---|---|---|
| | Host | Voltage | Efficiency | Emitting | Lifespan |
| | Material | (V) | (cd/A) | Color | (T95, hr) |
| Device | C-32 | 3.2 | 27.2 | Red | 64.0 |
| Example 1 | | | | | |
| Device | C-53 | 3.3 | 27.9 | Red | 97.8 |
| Example 2 | | | | | |
| Device | CBP | 9.0 | 14.3 | Red | 0.31 |
| Comparative | | | | | |
| Example 1 | | | | | |

From Table 1 above, it can be seen that the organic electroluminescent device comprising the organic electroluminescent compound according to the present disclosure as a host material has a low driving voltage, high luminous efficiency and significantly improved lifespan characteristics, compared to an organic electroluminescent device using the conventional host material.

### [Device Example 3 and 4] Preparation of OLEDs Comprising a Plurality of Host Materials

OLEDs were produced in the same manner as in Device Example 1, except that the first host material and the second host material shown in Table 2 below as hosts were introduced into two cells of the vacuum vapor deposition apparatus, respectively, and compound D-39 as a dopant was introduced into another cell, and the two host materials were evaporated at a rate of 1:1, and the dopant material was evaporated at a different rate, simultaneously and deposited in a doping amount of 3 wt % based on the total amount of the hosts and dopant to form a light-emitting layer having a thickness of 40 nm on the hole transport layer.

The driving voltage, the luminous efficiency, and the light-emitting color at a luminance of 1,000 nits, and the time taken for luminance to decrease from 100% to 95% at a luminance of 5,000 nits (lifespan; T95) of the OLEDs according to Device Examples 3 and 4 produced as described above, were measured, and the results thereof are shown in Table 2 below:

TABLE 2

| | | | Driving | Luminous | | |
|---|---|---|---|---|---|---|
| | First Host | Second Host | Voltage | Efficiency | Light-Emitting | Lifespan |
| | Material | Material | (V) | (cd/A) | Color | (T95, hr) |
| Device | C-4 | H2-29 | 3.1 | 31.8 | Red | 1014 |
| Example 3 | | | | | | |
| Device | C-7 | H2-29 | 3.2 | 33.6 | Red | 470 |
| Example 4 | | | | | | |

From Table 2 above, it was confirmed that driving at low voltage, luminous efficiency and lifespan characteristics can improve by a specific combination of host materials according to the present disclosure.

The compounds used in Device Examples 1 to 4 and Device Comparative Example 1 above are specifically shown in the following Table 3.

TABLE 3

| | |
|---|---|
| Hole Injection Layer/ Hole Transport Layer | 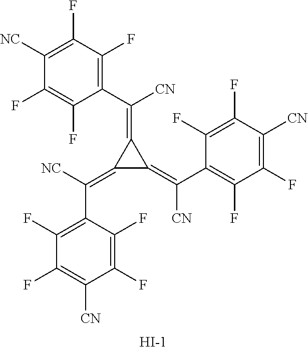 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 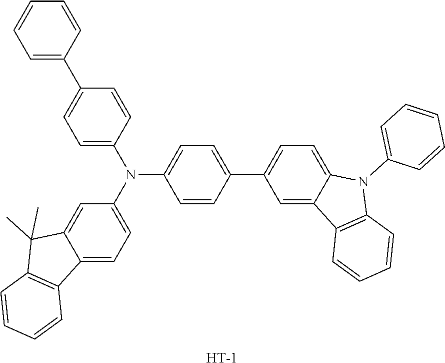 |
| | 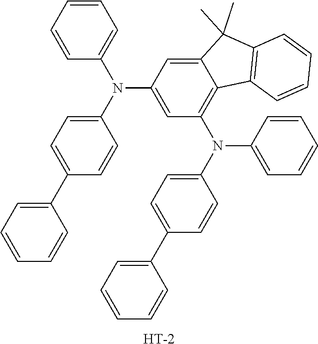 |
| Light-Emitting Layer | 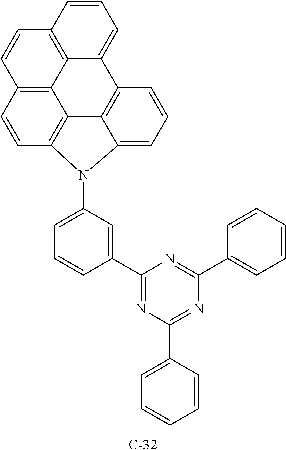 |
| | 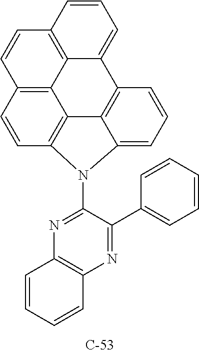 |
| | 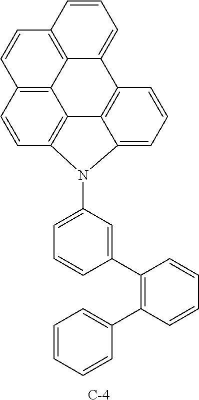 |
| | 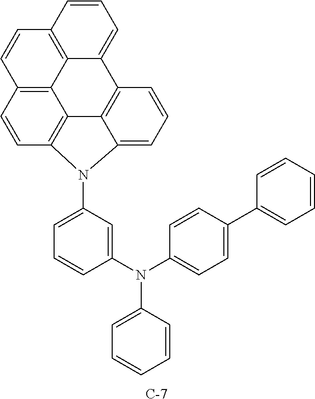 |
| | 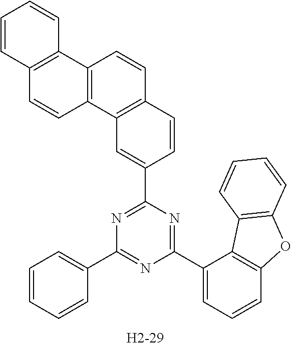 [SMILES: c1ccc(-c2nc(-c3ccc4ccc5c6ccccc6ccc5c4c3)nc(-c3cccc4oc5ccccc5c34)n2)cc1] |
| | 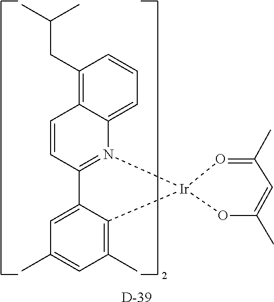 |
| | 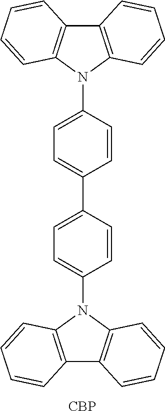 |
| Electron Transport Layer/ Electron Injection Layer | 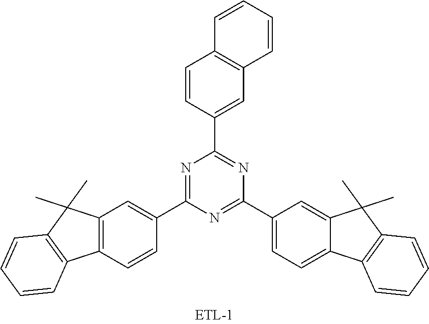 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 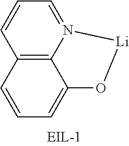 |

### [Device Example 5] Preparation of a Blue Light-Emitting OLED According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 as a hole injection compound was introduced into a cell of the vacuum vapor deposition apparatus, and hole transport compound HT-3 was introduced into another cell. The two materials were evaporated at different rates and the hole injection compound was deposited in a doping amount of 7 wt % based on the total amount of the hole injection compound and the hole transport compound to form a hole injection layer having a thickness of 10 nm. Next, compound HT-1 was deposited as a first hole transport layer having a thickness of 75 nm on the hole injection layer. Next, compound C-4 as a material for the second hole transport layer was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH was introduced into the two cells of the vacuum deposition apparatus as hosts, and compound BD was introduced into another cell as a dopant. The two materials were evaporated at different rates, and the dopant material was deposited in a doping amount of 2 wt % based on the total amount of the hosts and the dopant to form a light-emitting layer having a thickness of 20 nm on the second hole transport layer. Next, compound HBL as an electron buffer layer was deposited on the light-emitting layer to form an electron buffer layer having a thickness of 5 nm. Next, compounds ETL-1 and EIL-1 as electron transport layers were deposited at a ratio of 5:5 to form an electron transport layer having a thickness of 30 nm on the electron buffer layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### [Device Example 6] Preparation of a Blue Light-Emitting OLED According to the Present Disclosure

An OLED was produced in the same manner as in Device Example 5, except that compound C-7 was used as the second hole transport layer.

### [Device Comparative Example 2] Preparation of a Blue Light-Emitting OLED not According to the Present Disclosure

An OLED was produced in the same manner as in Device Example 5, except that the first hole transport layer was deposited to a thickness of 80 nm, without the second hole transport layer.

The driving voltage, the luminous efficiency, and the CIE color coordinates at a luminance of 1,000 nits of the OLEDs according to Device Examples 5 and 6 and Comparative Example 2 produced as described above, were measured, and the results thereof are shown in Table 4 below:

TABLE 4

| | Material for | Material for | | | | |
|---|---|---|---|---|---|---|
| | First Hole | Second Hole | Driving | Luminous | CIE(1931) | |
| | Transport | Transport | Voltage | Efficiency | Color Coordinates | |
| | Layer | Layer | (V) | (cd/A) | x | y |
| Device | HT-3 | C-4 | 3.6 | 6.8 | 0.132, | 0.080 |
| Example 5 | | | | | | |
| Device | HT-3 | C-7 | 3.7 | 6.9 | 0.132 | 0.079 |
| Example 6 | | | | | | |
| Device | HT-3 | — | 3.8 | 5.8 | 0.132 | 0.079 |
| Comparative | | | | | | |
| Example 2 | | | | | | |

From Table 4 above, it can be seen that the organic electroluminescent device comprising the organic electroluminescent compound according to the present disclosure as a hole transport material exhibits excellent efficiency characteristics.

The compounds used in Device Examples 5 and 6 and Device Comparative Example 2 are specifically shown in the following Table 5:

TABLE 5

| | |
|---|---|
| Hole Injection Layer/ Hole Transport Layer | 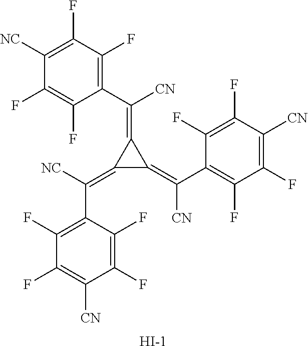 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 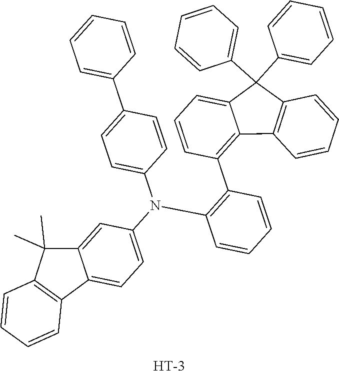 |
| | 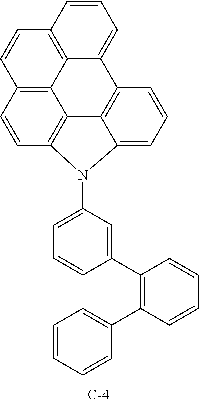 |
| | 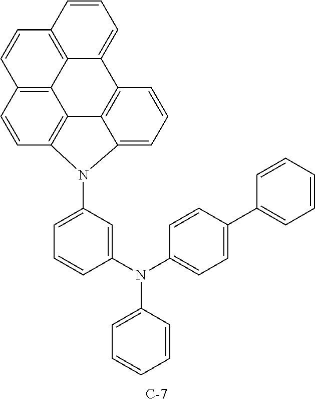 |
| Light-Emitting Layer | 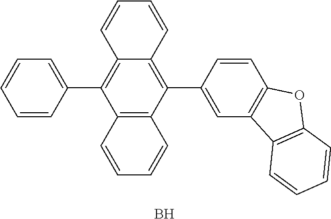 |
| | 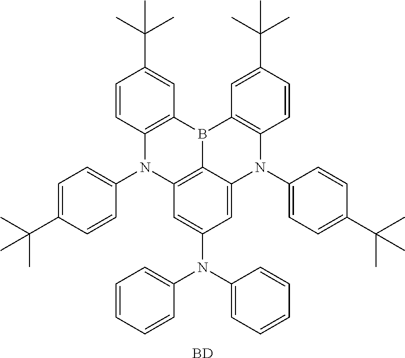 |
| Electron Buffer Layer | 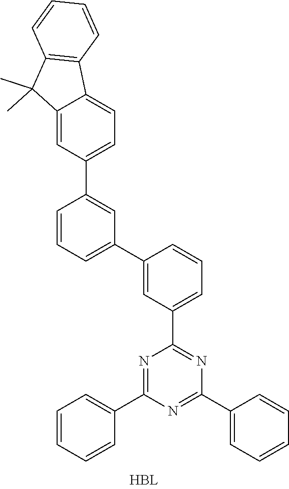 [SMILES: CC1(C)c2ccccc2-c2ccc(-c3cccc(-c4cccc(-c5nc(-c6ccccc6)nc(-c6ccccc6)n5)c4)c3)cc21] |
| Electron Transport Layer/ Electron Injection Layer | 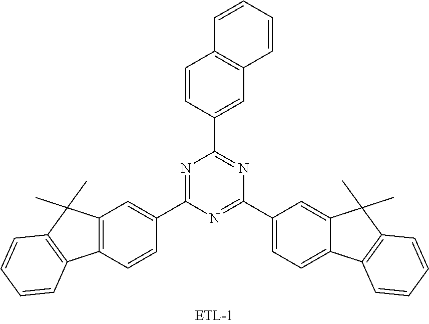 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 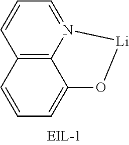 |

### [Device Comparative Example 3] Preparation of a Blue Light-Emitting OLED not Containing an Electron Buffer Layer

An OLED not according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with isopropyl alcohol, and thereafter was stored in isopropanol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and the first hole transport compound HT-4 was introduced into another cell. The two materials were evaporated at different rates and first hole injection compound was deposited in a doping amount of 3 wt % based on the total amount of the first hole injection compound and the first hole transport compound to form a first hole injection layer having a thickness of 10 nm. Next, compound HT-1 was deposited as a first hole transport layer having a thickness of 80 nm on the first hole injection layer. Next, compound HT-4 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH-1 was introduced into a cell of the vacuum vapor deposition apparatus as a host and compound BD-1 was introduced into another cell as a dopant. The two materials were evaporated at different rates, and the dopant material was deposited in a doping amount of 2 wt % based on the total amount of the host and dopant to form a light-emitting layer having a thickness of 20 nm on the second hole transport layer. Next, compounds ETL-1 and EIL-1 as electron transport materials were deposited at a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### [Device Example 7] Preparation of a Blue Light-Emitting OLED Comprising the Compound According to the Present Disclosure as an Electron Buffer Material

An OLED was produced in the same manner as in Comparative Example 3, except that the electron transport layer was deposited to a thickness of 30 nm, and compound C-32 was deposited between the light-emitting layer and the electron transport layer to form to an electron buffer layer having thickness of 5 nm.

The driving voltage, the luminous efficiency, the CIE Color Coordinates, and the external quantum efficiency at a luminance of 1,000 nits, and the time taken for luminance to decrease from 100% to 50% at luminance of constant current of 2,750 nits (lifespan; T50) of the OLEDs according to Device Comparative Example 3 and Device Example 7 produced as described above, were measured, and the results thereof are shown in Table 6 below:

TABLE 6

| | | | | | | External | |
|---|---|---|---|---|---|---|---|
| | Electron | Driving | Luminous | | | Quantum | |
| | Buffer | Voltage | Efficiency | Color Coordinates | | Efficiency | Lifespan |
| | Material | (V) | (cd/A) | x | y | (%) | (T50, hr) |
| Device | — | 3.6 | 8.6 | 0.133 | 0.118 | 8.8 | 681.4 |
| Comparative | | | | | | | |
| Example 3 | | | | | | | |
| Device | C-32 | 3.6 | 8.3 | 0.133 | 0.119 | 8.8 | 716.2 |
| Example 7 | | | | | | | |

From Table 6 above, it can be seen that, due to the fast electron current characteristics of the electron buffer material of the present disclosure, Device Example 7 has superior lifespan characteristics compared to Comparative Example 3 without the electron buffer material.

The compounds used in Device Comparative Example 3 and Device Example 7 are specifically shown in the following Table 7:

TABLE 7

| | |
|---|---|
| Hole Injection Layer/ Hole Transport Layer | 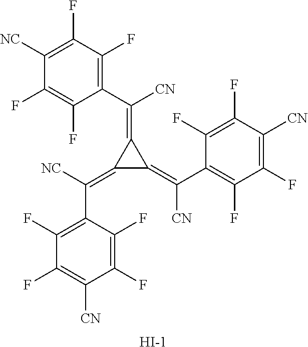 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 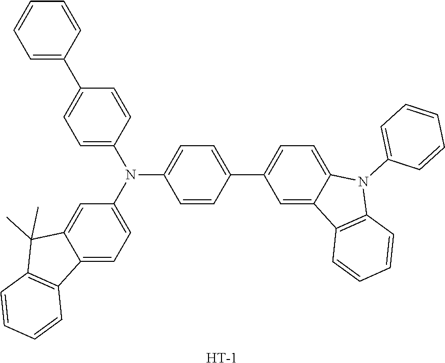 |
| | 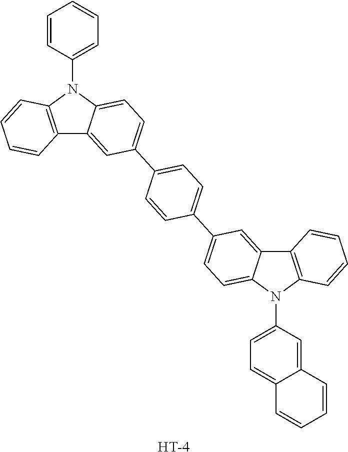 |
| Light-Emitting Layer | 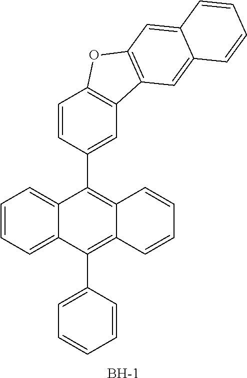 |
| | 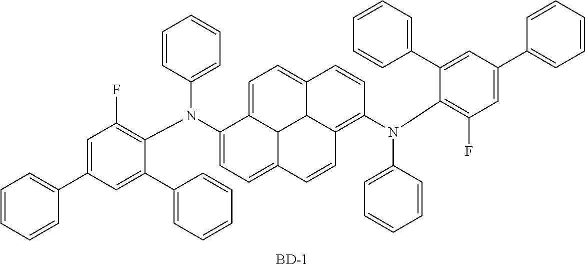 [SMILES: Fc1cc(-c2ccccc2)cc(-c2ccccc2)c1N(C1=CC=C2C=CC3=C(N(c4ccccc4)c4c(F)cc(-c5ccccc5)cc4-c4ccccc4)C=CC4=CC=C1C2C43)c1ccccc1 ; "BD-1"] |
| Electron Buffer Layer | 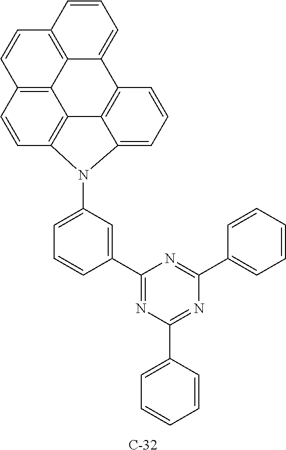 |
| Electron Transport Layer/ Electron Injection Layer | 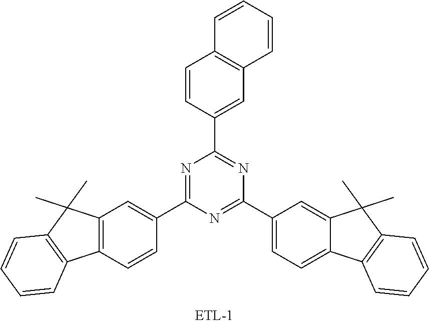 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 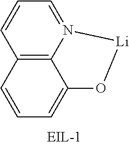 |

In the organic electroluminescent compound represented by formula 1 according to the present disclosure, the LUMO (lowest unoccupied molecular orbital) energy level, the HOMO (highest unoccupied molecular orbital) energy level, and triplet energy of the compounds where X represents N-L₁-Ar₁ (wherein L₁ represents a single bond, and Ar₁ represents phenyl), O, S, and CR₁R₂ (wherein all of R₁ and R₂ are methyl) were measured, respectively, and the results thereof are shown in Table 8 below.

TABLE 8

| | 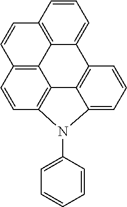 [SMILES: c1ccc(-n2c3cccc4c5cccc6ccc7ccc2c(c7c65)c43)cc1] | 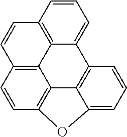 [SMILES: c1cc2ccc3ccc4oc5cccc6c(c1)c2c3c4c56] | 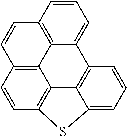 [SMILES: c1cc2ccc3ccc4sc5cccc6c(c1)c2c3c4c56] | 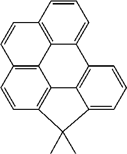 [SMILES: CC1(C)c2cccc3c4cccc5ccc6ccc1c(c23)c6c54] |
|---|---|---|---|---|
| LUMO (ev) | −1.309 | −1.492 | −1.515 | −1.330 |
| HOMO (ev) | −5.041 | −5.418 | −5.350 | −5.293 |
| Triplet (ev) | 2.271 | 2.338 | 2.302 | 2.326 |

* The structure was optimized by applying the hybrid density functionai theory (hybrid DFT) (B3LYP) and 6-31G(d) basis sets using Gaussian's quantum chemistry calculation program Gaussian16, and the triplet state was calculated by using TD-DFT (time dependent DFT).

Referring to Table 8 above, in the organic electroluminescent compound represented by formula 1 according to the present disclosure, when X represents N-L₁-Ar₁, O, S, and CR₁R₂, it can be confirmed that all the parent nuclei have similar energy levels. Through the above, even when O, S, or CR₁R₂ is introduced into the parent nucleus, instead of N-L₁-Ar₁, it is expected to have device characteristics similar to those of Device Examples 1 and 2 above.

1. An organic electroluminescent compound represented by the following formula 1: 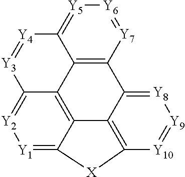
wherein,
X represents N-L₁-Ar₁, O, S, or CR₁R₂;
Y₁ to Y₁₀ each independently represent, CR₃ or N;
R₁ and R₂ each independently represent, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to each other to form a ring(s);
R₃ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or -L₂-Ar₂; or the adjacent R₃″s may be linked to each other to form a ring(s);
L₁ and L₂ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ and Ar₂ each independently represent, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted fused ring of a (C3-C30) aliphatic ring and a (C6-C30) aromatic ring, —Si—(R′₁)(R′₂) or —N—(R′₃)(R′₄); and
R′₁ to R′₄ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

2. The organic electroluminescent compound according to claim 1, wherein the substituent(s) of the substituted alkyl(ene), the substituted alkenyl, the substituted aryl(ene), the substituted heteroaryl(ene), the substituted cycloalkyl(ene), the substituted alkoxy, and the substituted fused ring of (C3-C30) aliphatic ring and (C6-C30) aromatic ring each independently represent at least one selected from the group consisting of deuterium, halogen, cyano, carboxyl, nitro, hydroxyl, phosphine oxide, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (3- to 30-membered)heteroaryl unsubstituted or substituted with at least one of deuterium and (C6-C30)aryl, (C6-C30)aryl unsubstituted or substituted with at least one of deuterium and (3- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, a fused ring of aliphatic ring and aromatic ring, amino, mono- or di-(C1-C30)alkylamino, mono- or di-(C2-C30)alkenylamino, (C1-C30)alkyl(C2-C30)alkenylamino, a substituted or unsubstituted mono- or di-(C6-C30)arylamino, (C1-C30)alkyl(C6-C30)arylamino, mono- or di-(3- to 30-membered)heteroarylamino, (C1-C30)alkyl(3- to 30-membered)heteroarylamino, (C2-C30)alkenyl(C6-C30)arylamino, (C2-C30)alkenyl(3- to 30-membered)heteroarylamino, (C6-C30)aryl(3- to 30-membered)heteroarylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl.

3. The organic electroluminescent compound according to claim 1, wherein the formula 1 is represented by any one of the following compounds 1-1 to 1-8: 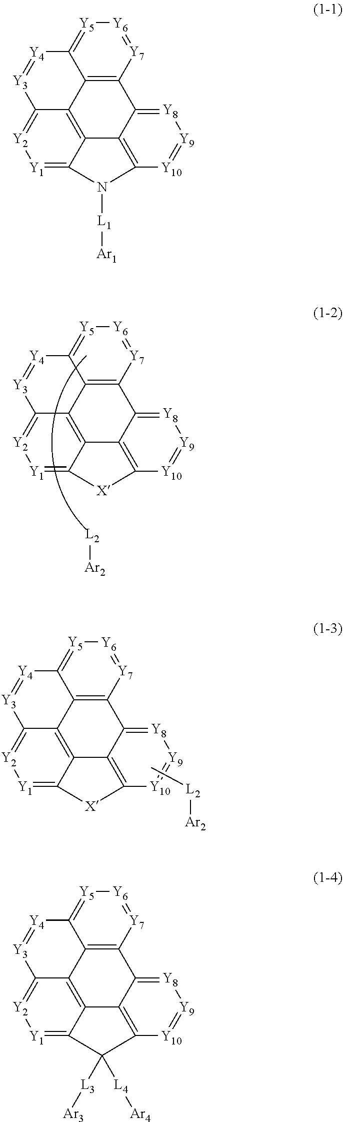 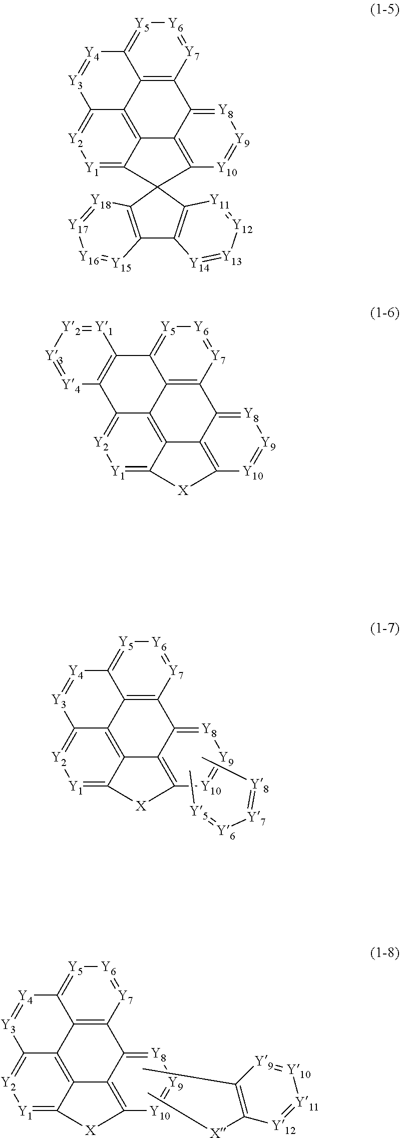
wherein,
X, Y₁ to Y₁₀, L₁, L₂, Ar₁ and Ar₂ are as defined in claim 1;
Y₁₁ to Y₁₈ and Y′₁ to Y′₁₂ are as defined as Y₁ in claim 1;
L₃ and L₄ are as defined as L₁ in claim 1;
Ar₃ and Ar₄ are as defined as Ar₁ in claim 1;
X′ represents O or S;
X″ represents O, S, CR₁₁R₁₂ or NR₁₃; and
R₁₁ to R₁₃ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to the adjacent substituent to form a ring(s).

4. The organic electroluminescent compound according to claim 1, wherein the compound represented by the formula 1 is selected from the following compounds:

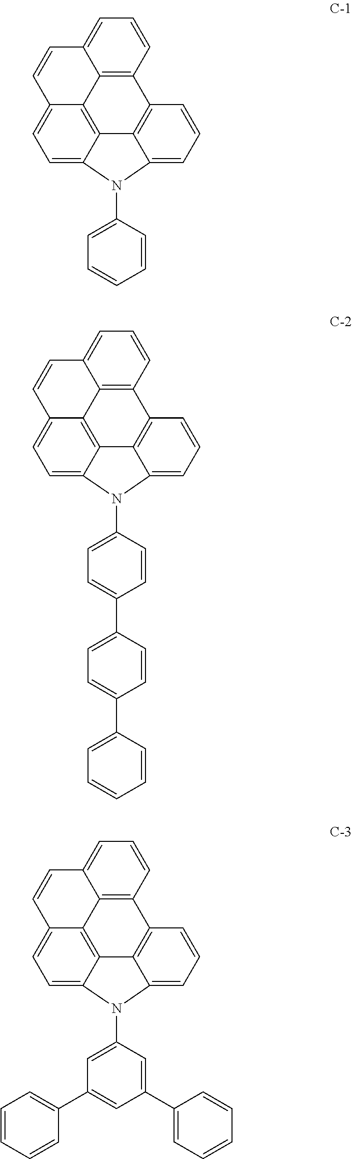

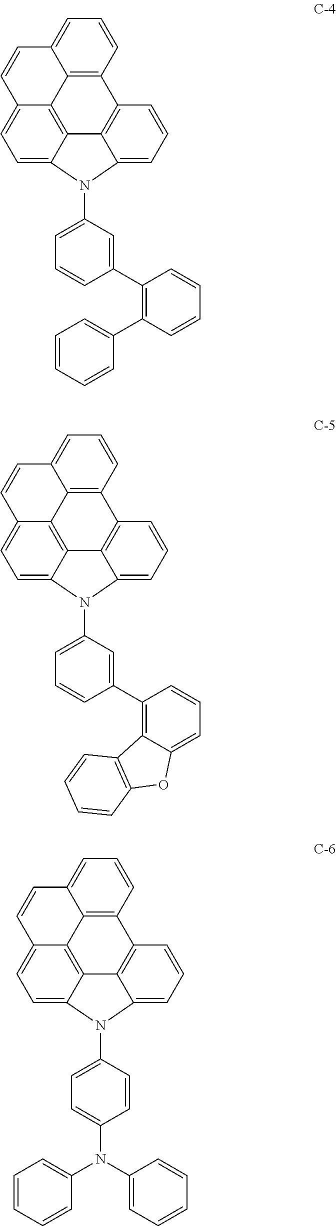

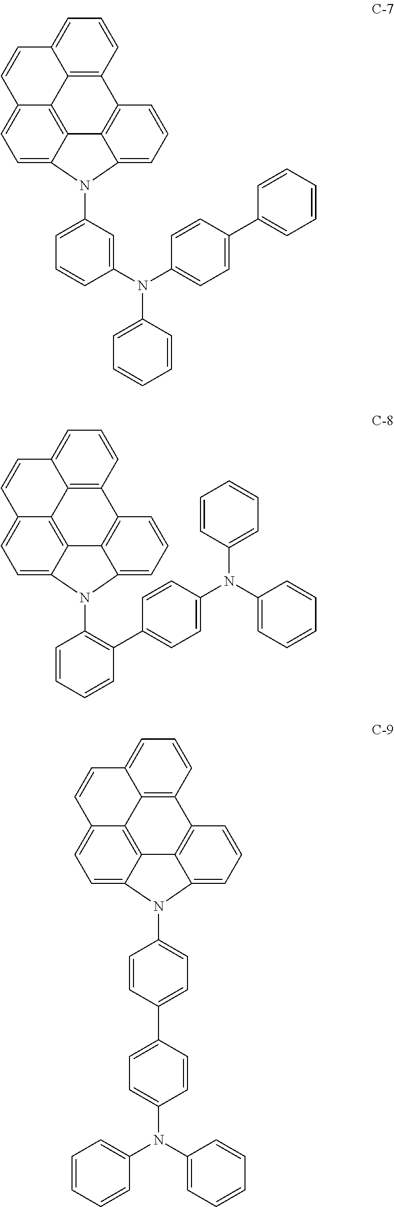 [SMILES: c1ccc(-c2ccc(N(c3ccccc3)c3cccc(-n4c5cccc6c7cccc8ccc9ccc4c(c9c87)c65)c3)cc2)cc1 ; c1ccc(N(c2ccccc2)c2ccc(-c3ccccc3-n3c4cccc5c6cccc7ccc8ccc3c(c8c76)c54)cc2)cc1 ; c1ccc(N(c2ccccc2)c2ccc(-c3ccc(-n4c5cccc6c7cccc8ccc9ccc4c(c9c87)c65)cc3)cc2)cc1]

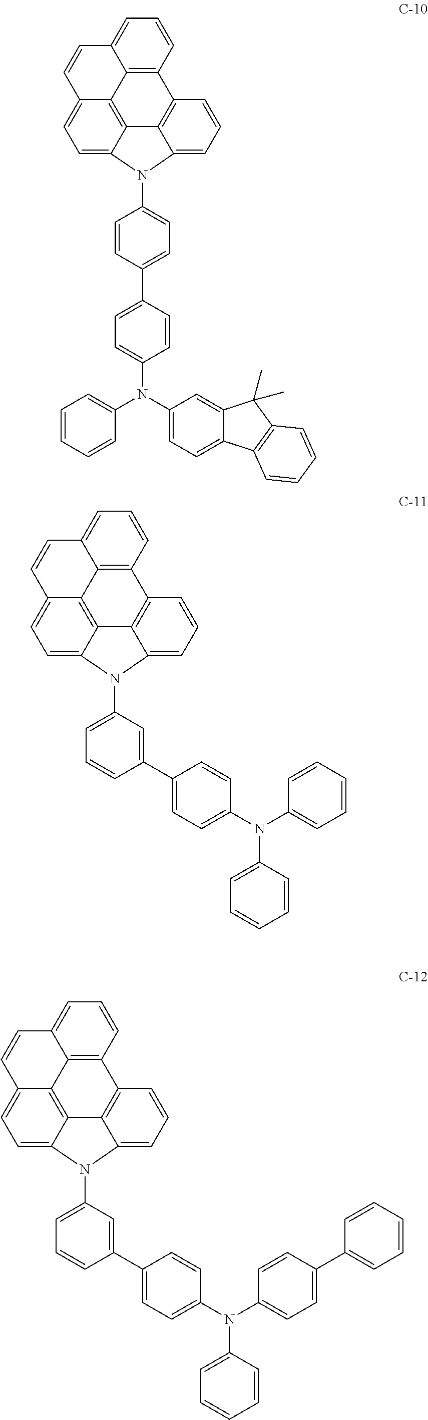 [SMILES: CC1(C)c2ccccc2-c2ccc(N(c3ccccc3)c3ccc(-c4ccc(-n5c6cccc7c8cccc9ccc%10ccc5c(c%10c98)c76)cc4)cc3)cc21 ; c1ccc(N(c2ccccc2)c2ccc(-c3cccc(-n4c5cccc6c7cccc8ccc9ccc4c(c9c87)c65)c3)cc2)cc1 ; c1ccc(-c2ccc(N(c3ccccc3)c3ccc(-c4cccc(-n5c6cccc7c8cccc9ccc%10ccc5c(c%10c98)c76)c4)cc3)cc2)cc1]

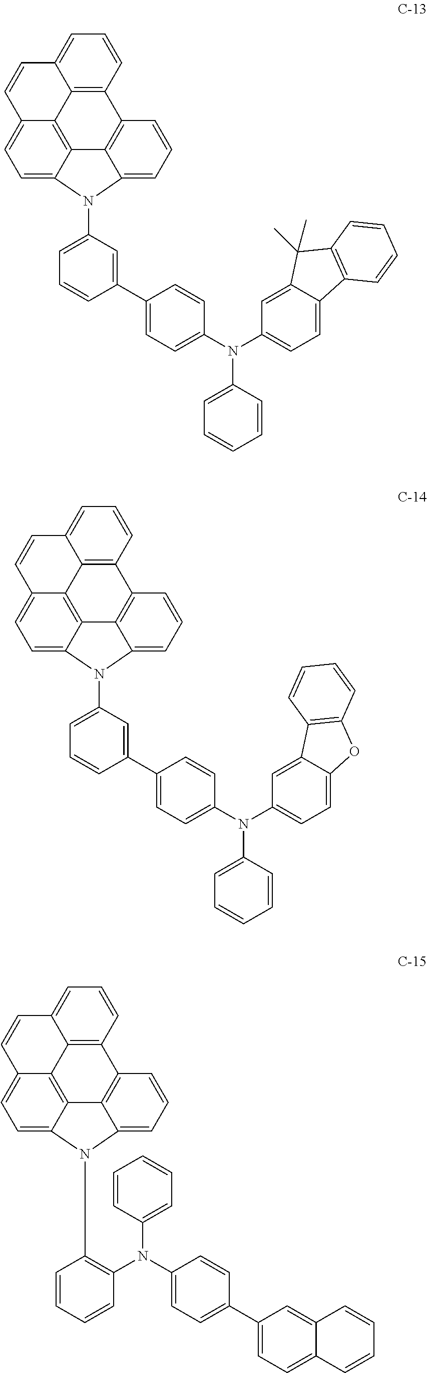 [SMILES: CC1(C)c2ccccc2-c2ccc(N(c3ccccc3)c3ccc(-c4cccc(-n5c6cccc7c8cccc9ccc%10ccc5c(c%10c98)c76)c4)cc3)cc21 ; c1ccc(N(c2ccc(-c3cccc(-n4c5cccc6c7cccc8ccc9ccc4c(c9c87)c65)c3)cc2)c2ccc3oc4ccccc4c3c2)cc1 ; c1ccc(N(c2ccc(-c3ccc4ccccc4c3)cc2)c2ccccc2-n2c3cccc4c5cccc6ccc7ccc2c(c7c65)c43)cc1]

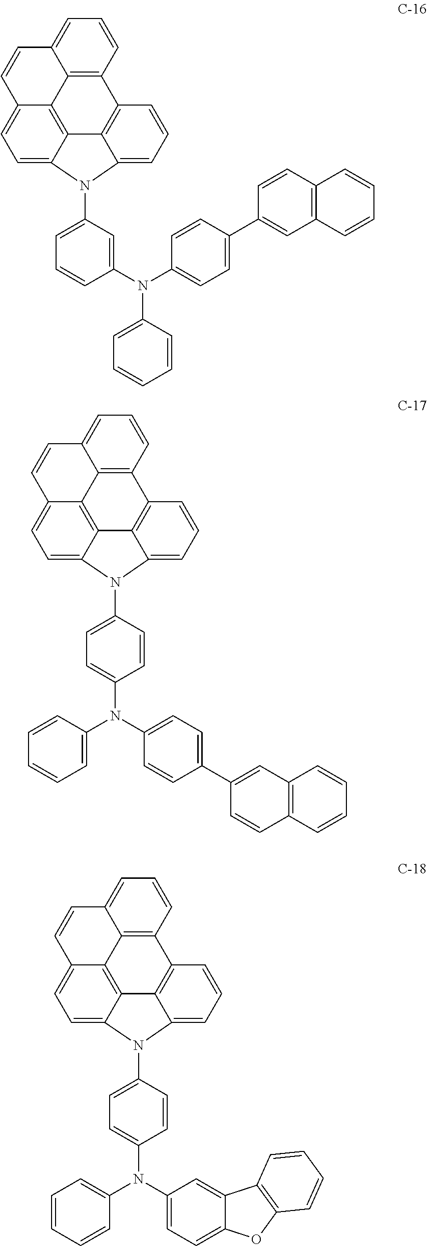

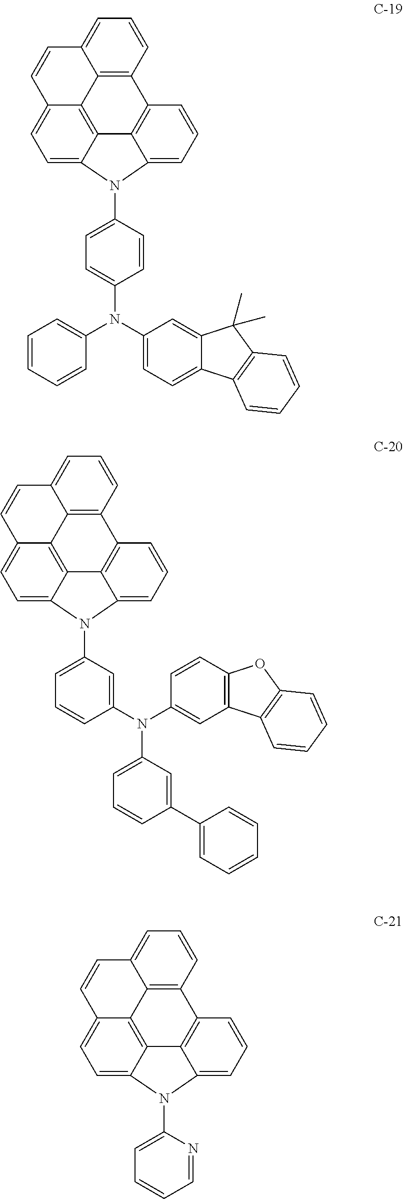 [SMILES: CC1(C)c2ccccc2-c2ccc(N(c3ccccc3)c3ccc(-n4c5cccc6c7cccc8ccc9ccc4c(c9c87)c65)cc3)cc21 ; c1ccc(-c2cccc(N(c3cccc(-n4c5cccc6c7cccc8ccc9ccc4c(c9c87)c65)c3)c3ccc4oc5ccccc5c4c3)c2)cc1 ; c1ccc(-n2c3cccc4c5cccc6ccc7ccc2c(c7c65)c43)nc1]

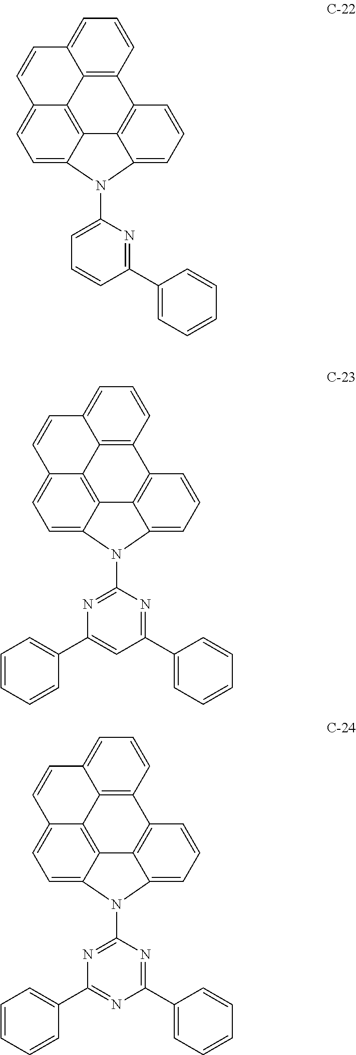 [SMILES: c1ccc(-c2cccc(-n3c4cccc5c6cccc7ccc8ccc3c(c8c76)c54)n2)cc1 ; c1ccc(-c2cc(-c3ccccc3)nc(-n3c4cccc5c6cccc7ccc8ccc3c(c8c76)c54)n2)cc1 ; c1ccc(-c2nc(-c3ccccc3)nc(-n3c4cccc5c6cccc7ccc8ccc3c(c8c76)c54)n2)cc1]

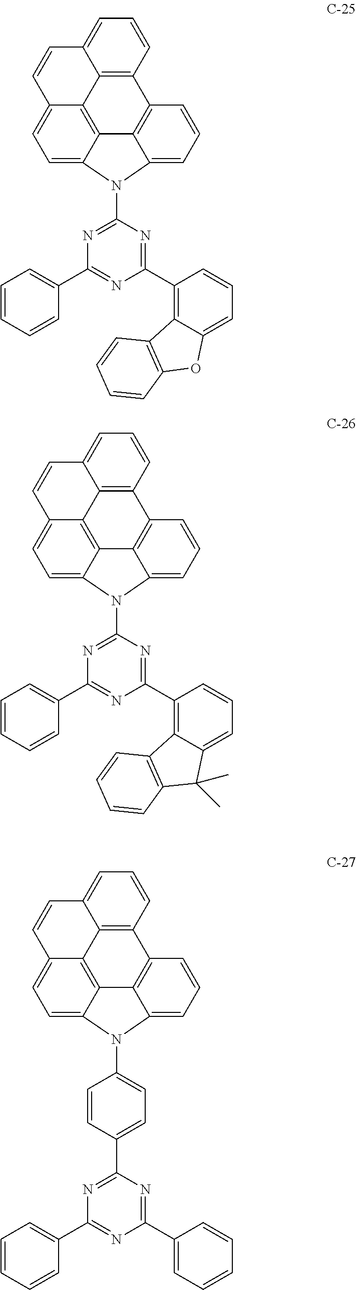

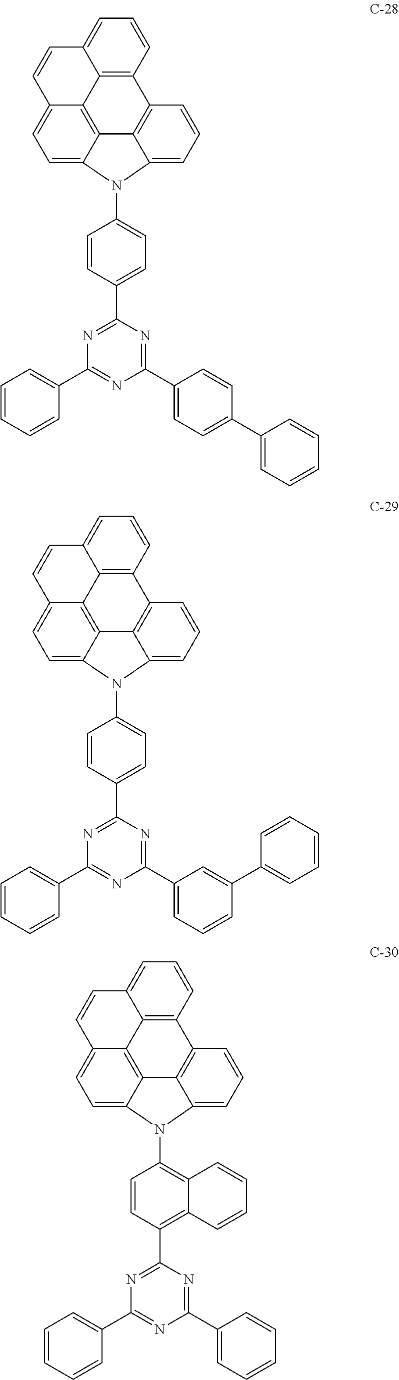

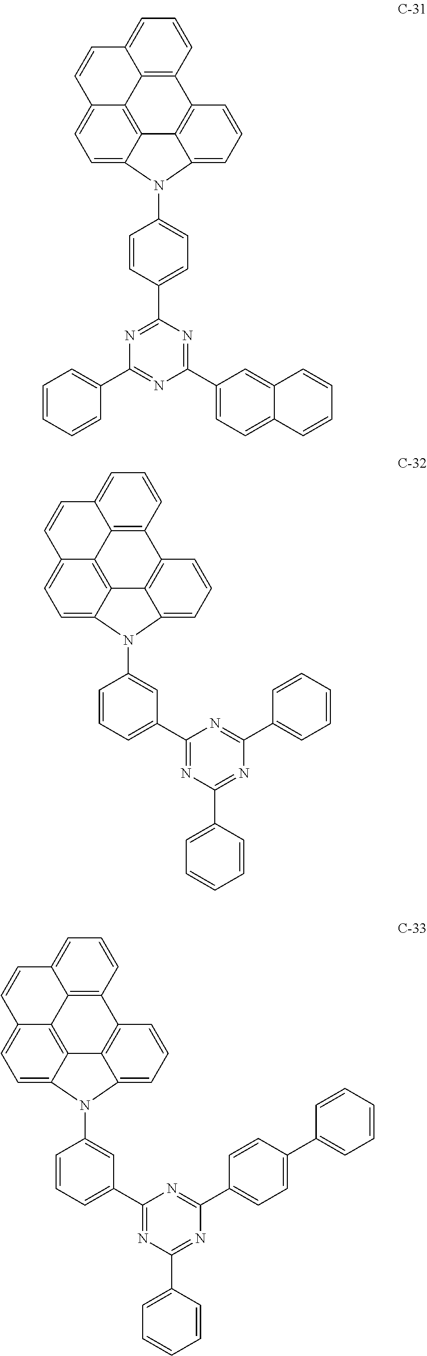 [SMILES: c1ccc(-c2nc(-c3ccc(-n4c5cccc6c7cccc8ccc9ccc4c(c9c87)c65)cc3)nc(-c3ccc4ccccc4c3)n2)cc1 ; c1ccc(-c2nc(-c3ccccc3)nc(-c3cccc(-n4c5cccc6c7cccc8ccc9ccc4c(c9c87)c65)c3)n2)cc1 ; c1ccc(-c2ccc(-c3nc(-c4ccccc4)nc(-c4cccc(-n5c6cccc7c8cccc9ccc%10ccc5c(c%10c98)c76)c4)n3)cc2)cc1]

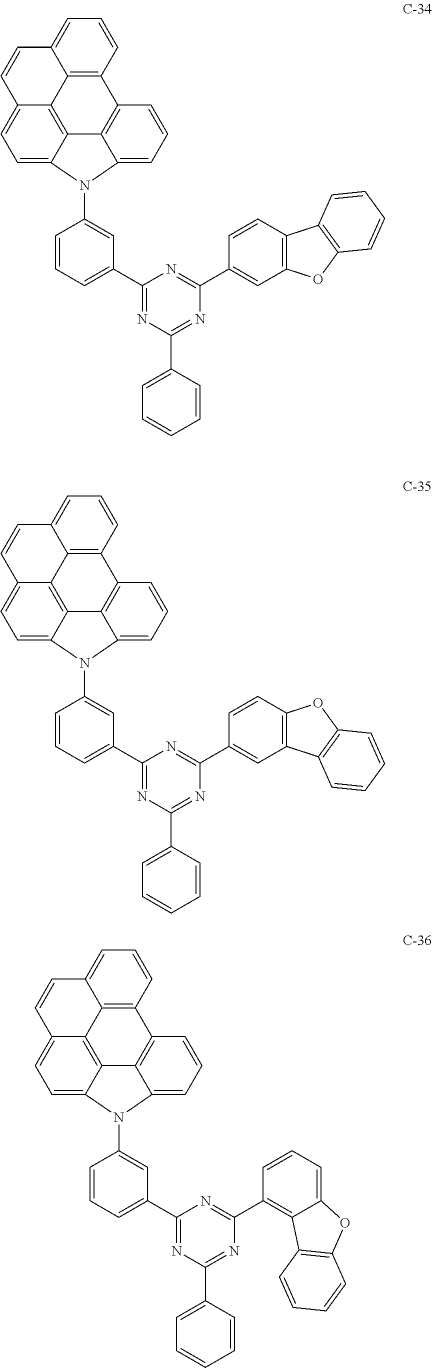 [SMILES: c1ccc(-c2nc(-c3cccc(-n4c5cccc6c7cccc8ccc9ccc4c(c9c87)c65)c3)nc(-c3ccc4c(c3)oc3ccccc34)n2)cc1 ; c1ccc(-c2nc(-c3cccc(-n4c5cccc6c7cccc8ccc9ccc4c(c9c87)c65)c3)nc(-c3ccc4oc5ccccc5c4c3)n2)cc1 ; c1ccc(-c2nc(-c3cccc(-n4c5cccc6c7cccc8ccc9ccc4c(c9c87)c65)c3)nc(-c3cccc4oc5ccccc5c34)n2)cc1]

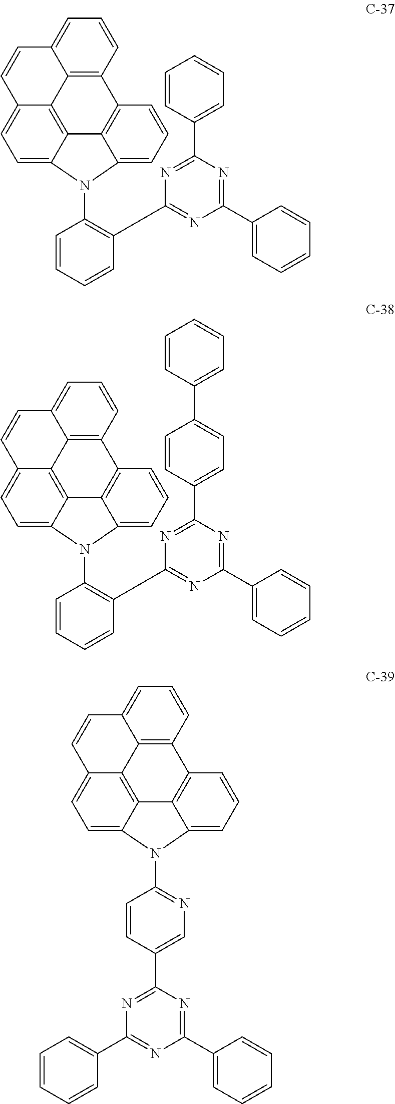 [SMILES: c1ccc(-c2nc(-c3ccccc3)nc(-c3ccccc3-n3c4cccc5c6cccc7ccc8ccc3c(c8c76)c54)n2)cc1 ; c1ccc(-c2ccc(-c3nc(-c4ccccc4)nc(-c4ccccc4-n4c5cccc6c7cccc8ccc9ccc4c(c9c87)c65)n3)cc2)cc1 ; c1ccc(-c2nc(-c3ccccc3)nc(-c3ccc(-n4c5cccc6c7cccc8ccc9ccc4c(c9c87)c65)nc3)n2)cc1]

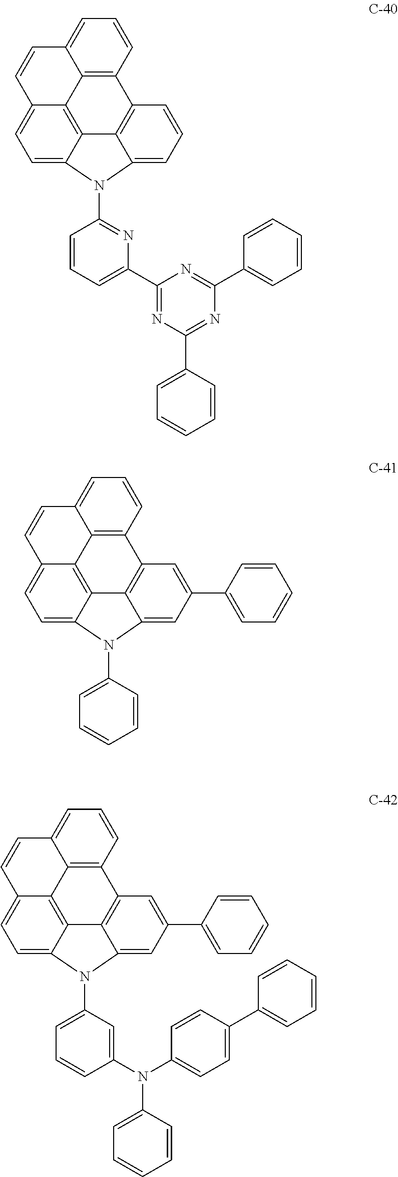 [SMILES: c1ccc(-c2nc(-c3ccccc3)nc(-c3cccc(-n4c5cccc6c7cccc8ccc9ccc4c(c9c87)c65)n3)n2)cc1 ; c1ccc(-c2cc3c4cccc5ccc6ccc7c(c6c54)c3c(c2)n7-c2ccccc2)cc1 ; c1ccc(-c2ccc(N(c3ccccc3)c3cccc(-n4c5cc(-c6ccccc6)cc6c7cccc8ccc9ccc4c(c9c87)c65)c3)cc2)cc1]

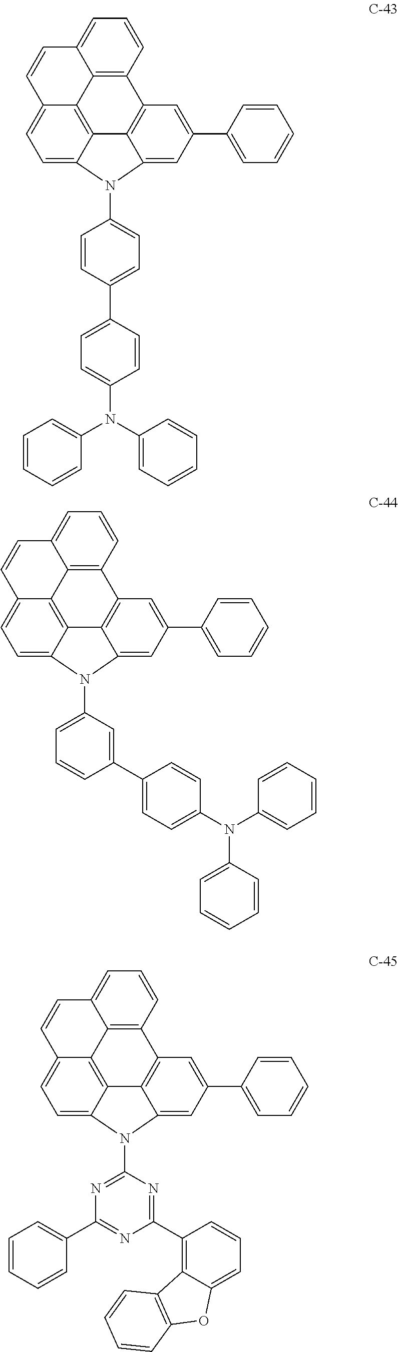

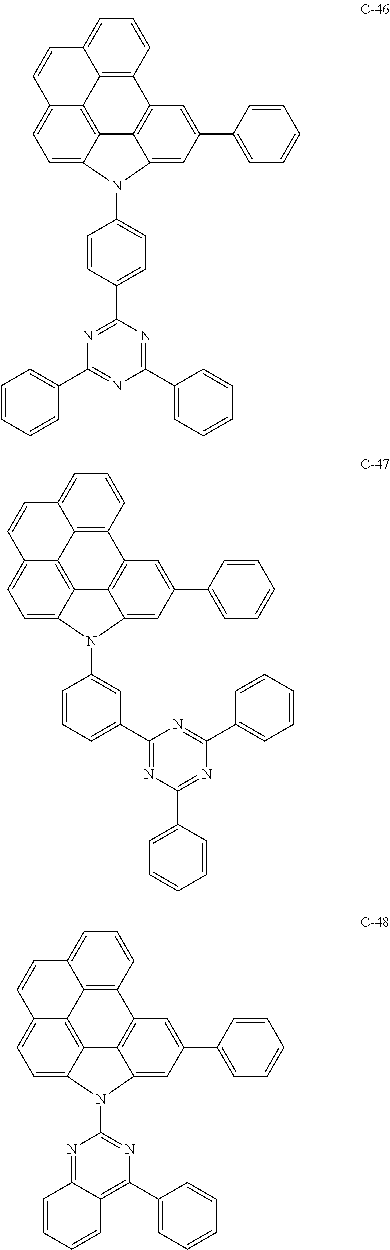 [SMILES: c1ccc(-c2cc3c4cccc5ccc6ccc7c(c6c54)c3c(c2)n7-c2ccc(-c3nc(-c4ccccc4)nc(-c4ccccc4)n3)cc2)cc1 ; c1ccc(-c2cc3c4cccc5ccc6ccc7c(c6c54)c3c(c2)n7-c2cccc(-c3nc(-c4ccccc4)nc(-c4ccccc4)n3)c2)cc1 ; c1ccc(-c2cc3c4cccc5ccc6ccc7c(c6c54)c3c(c2)n7-c2nc(-c3ccccc3)c3ccccc3n2)cc1]

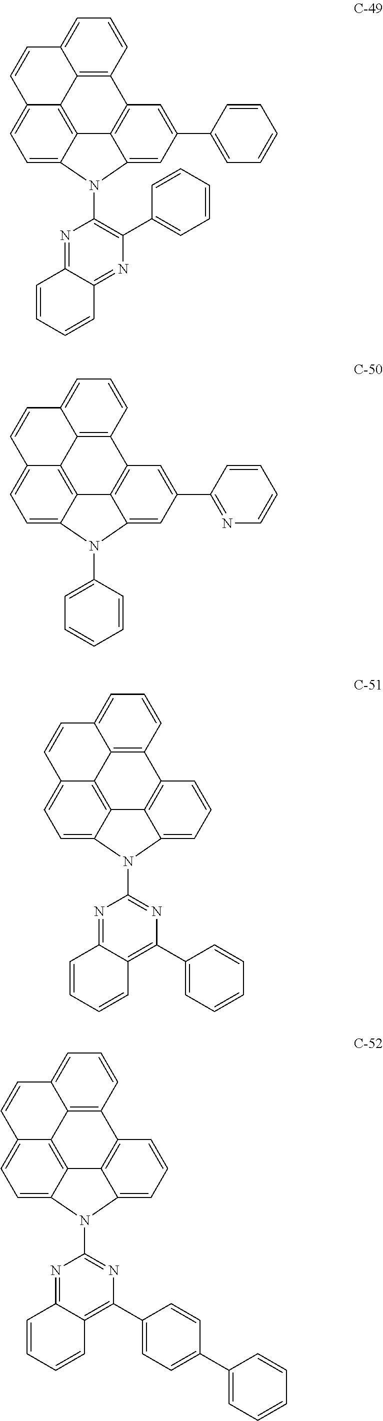

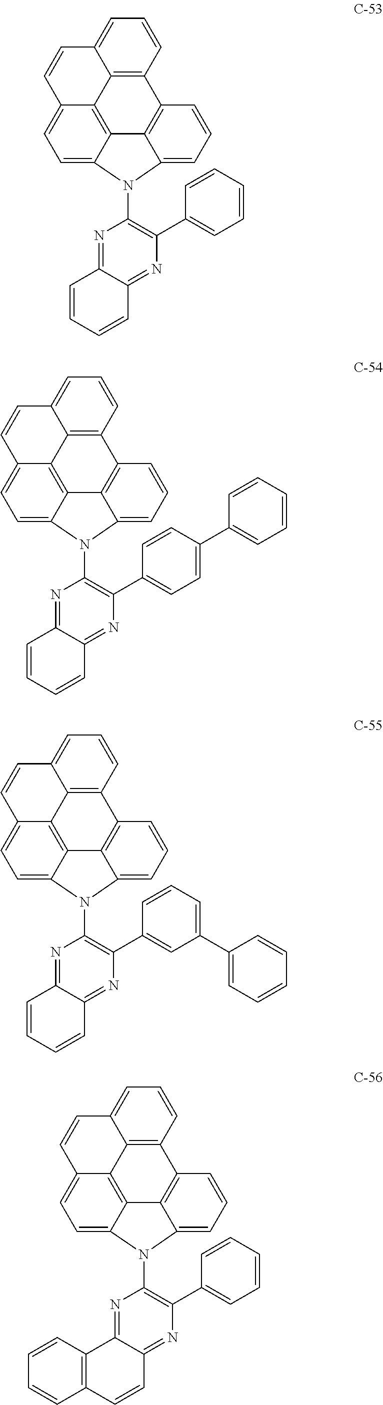

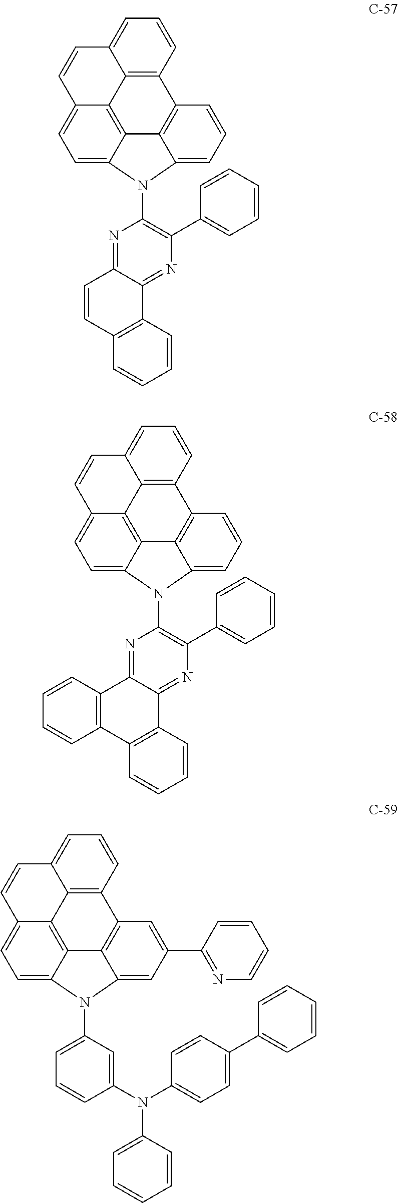

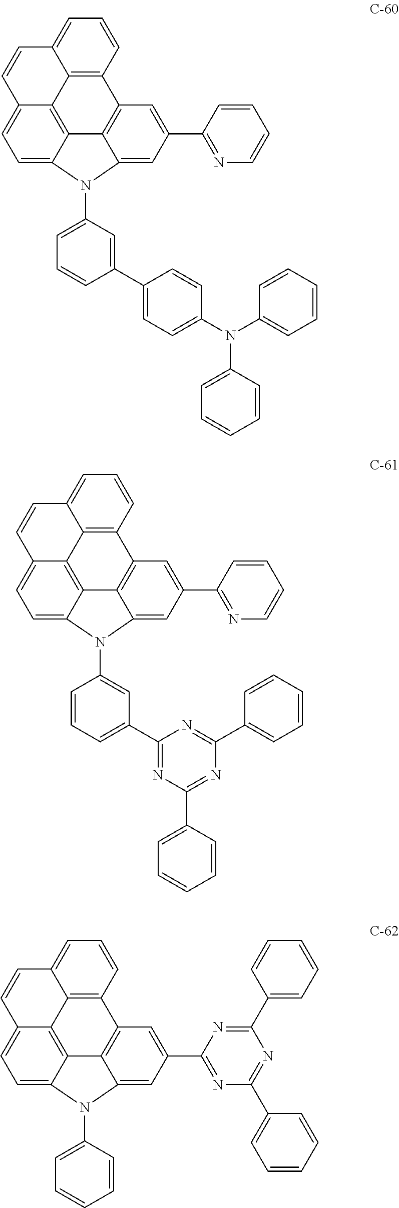 [SMILES: c1ccc(N(c2ccccc2)c2ccc(-c3cccc(-n4c5cc(-c6ccccn6)cc6c7cccc8ccc9ccc4c(c9c87)c65)c3)cc2)cc1 ; c1ccc(-c2nc(-c3ccccc3)nc(-c3cccc(-n4c5cc(-c6ccccn6)cc6c7cccc8ccc9ccc4c(c9c87)c65)c3)n2)cc1 ; c1ccc(-c2nc(-c3ccccc3)nc(-c3cc4c5cccc6ccc7ccc8c(c7c65)c4c(c3)n8-c3ccccc3)n2)cc1]

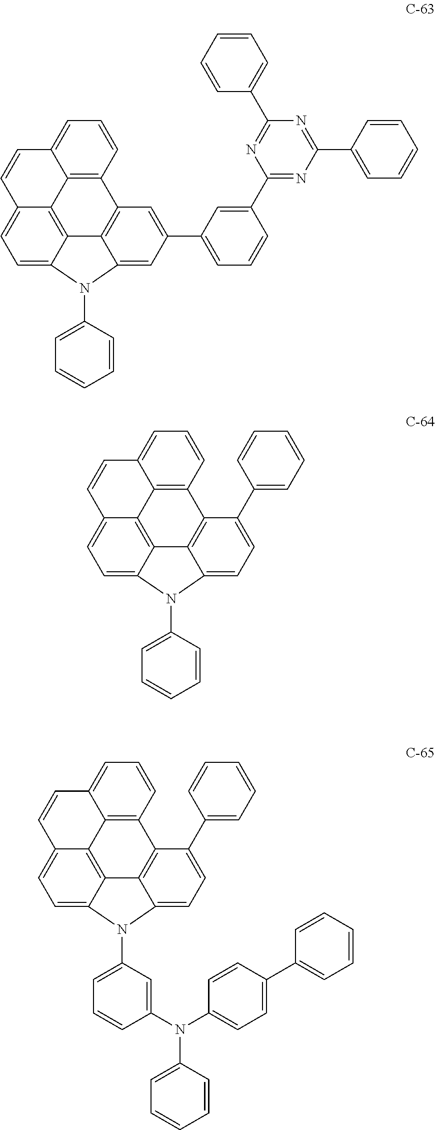 [SMILES: c1ccc(-c2nc(-c3ccccc3)nc(-c3cccc(-c4cc5c6cccc7ccc8ccc9c(c8c76)c5c(c4)n9-c4ccccc4)c3)n2)cc1 ; c1ccc(-c2ccc3c4c2c2cccc5ccc6ccc(c4c6c52)n3-c2ccccc2)cc1 ; c1ccc(-c2ccc(N(c3ccccc3)c3cccc(-n4c5ccc(-c6ccccc6)c6c7cccc8ccc9ccc4c(c9c87)c65)c3)cc2)cc1]

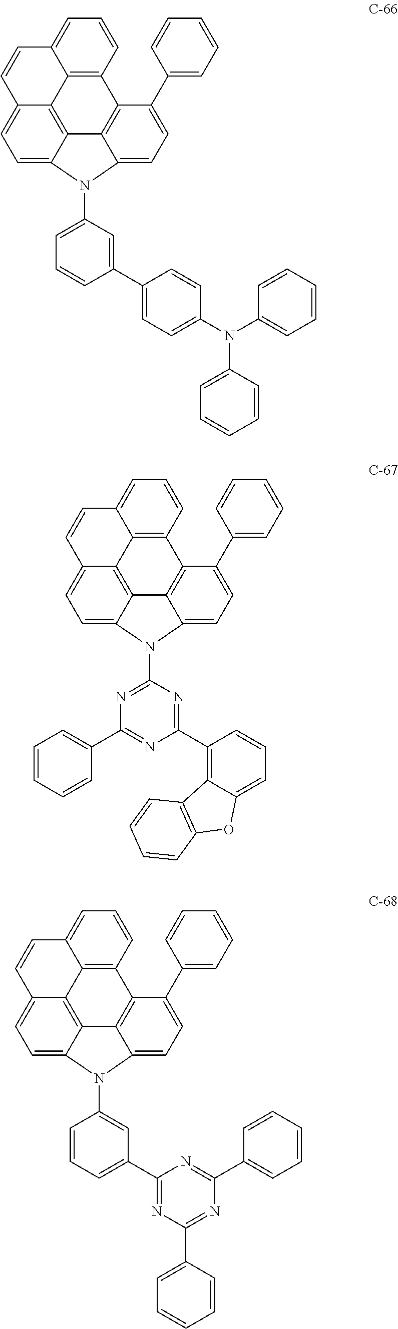

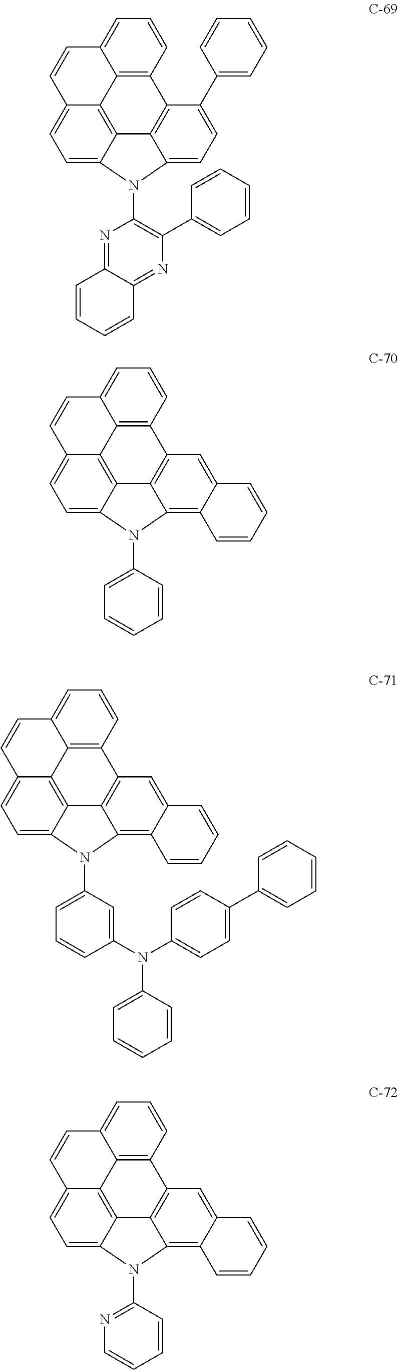

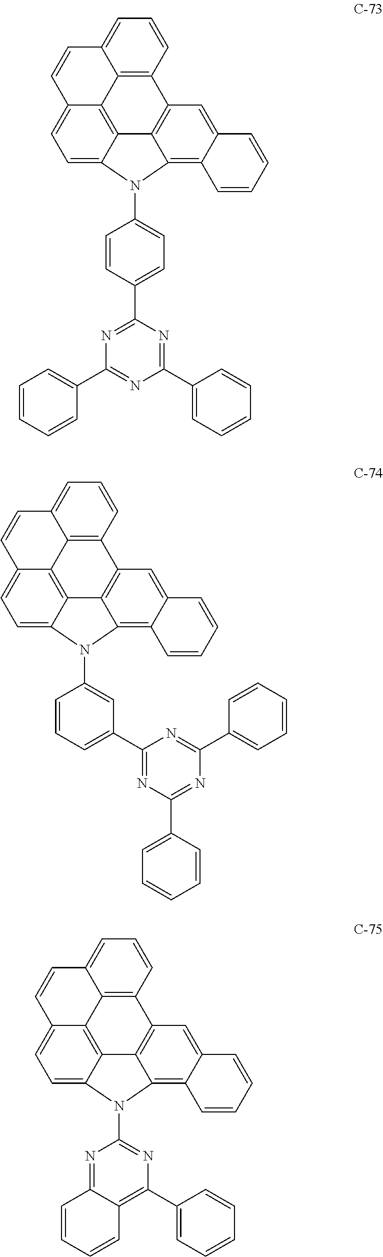 [SMILES: c1ccc(-c2nc(-c3ccccc3)nc(-c3ccc(-n4c5ccc6ccc7cccc8c9cc%10ccccc%10c4c9c5c6c78)cc3)n2)cc1 ; c1ccc(-c2nc(-c3ccccc3)nc(-c3cccc(-n4c5ccc6ccc7cccc8c9cc%10ccccc%10c4c9c5c6c78)c3)n2)cc1 ; c1ccc(-c2nc(-n3c4ccc5ccc6cccc7c8cc9ccccc9c3c8c4c5c67)nc3ccccc23)cc1]

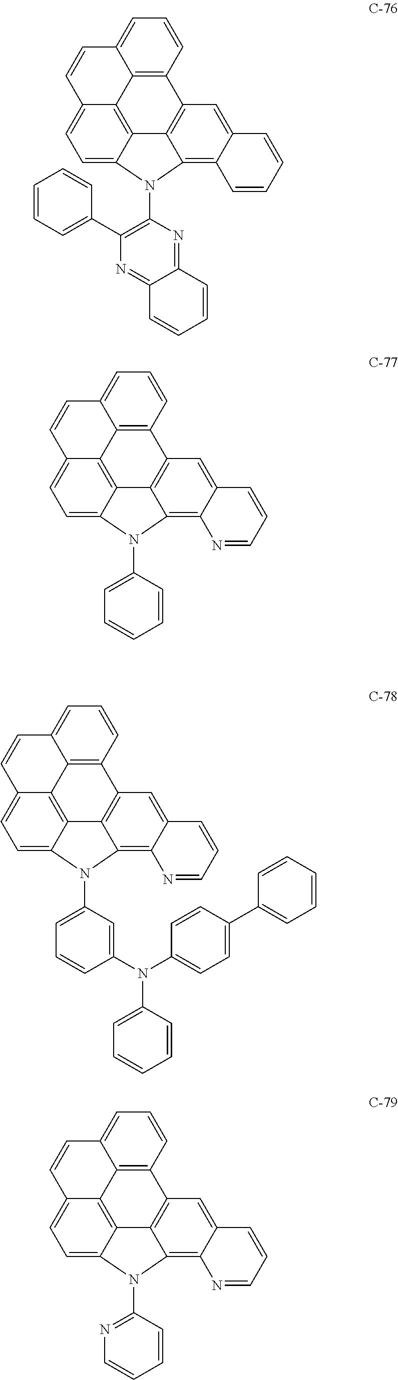

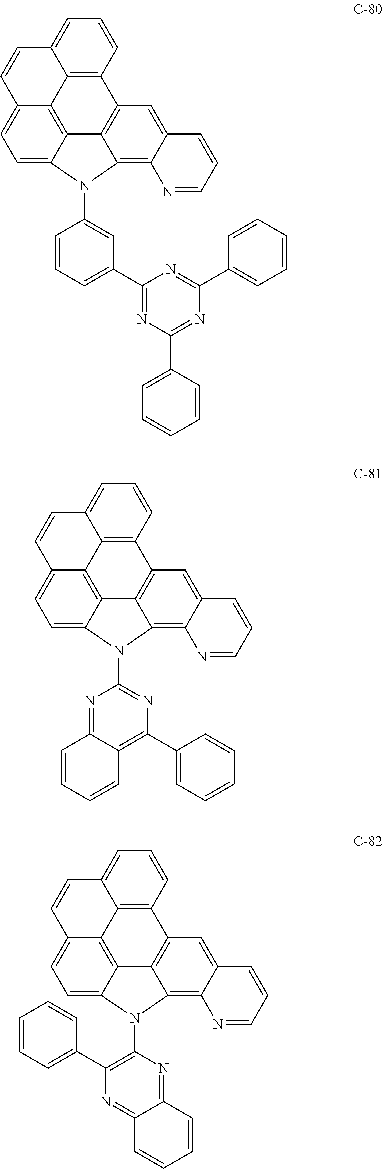 [SMILES: c1ccc(-c2nc(-c3ccccc3)nc(-c3cccc(-n4c5ccc6ccc7cccc8c9cc%10cccnc%10c4c9c5c6c78)c3)n2)cc1 ; c1ccc(-c2nc(-n3c4ccc5ccc6cccc7c8cc9cccnc9c3c8c4c5c67)nc3ccccc23)cc1 ; c1ccc(-c2nc3ccccc3nc2-n2c3ccc4ccc5cccc6c7cc8cccnc8c2c7c3c4c56)cc1]

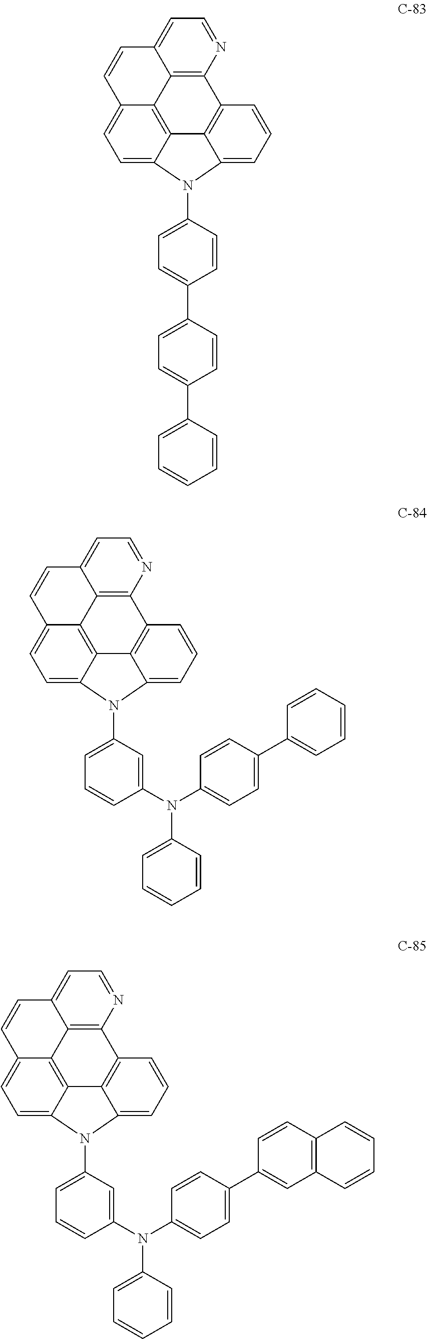 [SMILES: c1ccc(-c2ccc(-c3ccc(-n4c5cccc6c7nccc8ccc9ccc4c(c9c87)c65)cc3)cc2)cc1 ; c1ccc(-c2ccc(N(c3ccccc3)c3cccc(-n4c5cccc6c7nccc8ccc9ccc4c(c9c87)c65)c3)cc2)cc1 ; c1ccc(N(c2ccc(-c3ccc4ccccc4c3)cc2)c2cccc(-n3c4cccc5c6nccc7ccc8ccc3c(c8c76)c54)c2)cc1]

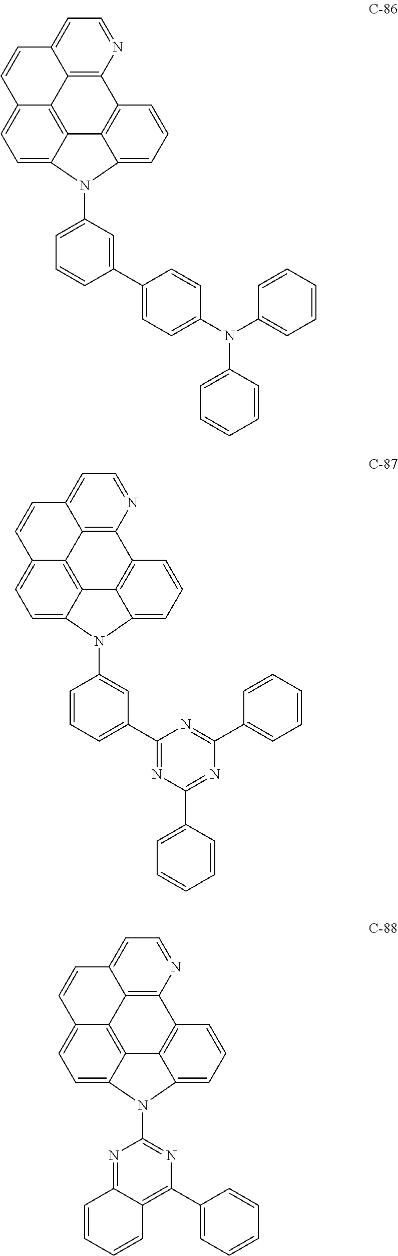 [SMILES: c1ccc(N(c2ccccc2)c2ccc(-c3cccc(-n4c5cccc6c7nccc8ccc9ccc4c(c9c87)c65)c3)cc2)cc1 ; c1ccc(-c2nc(-c3ccccc3)nc(-c3cccc(-n4c5cccc6c7nccc8ccc9ccc4c(c9c87)c65)c3)n2)cc1 ; c1ccc(-c2nc(-n3c4cccc5c6nccc7ccc8ccc3c(c8c76)c54)nc3ccccc23)cc1]

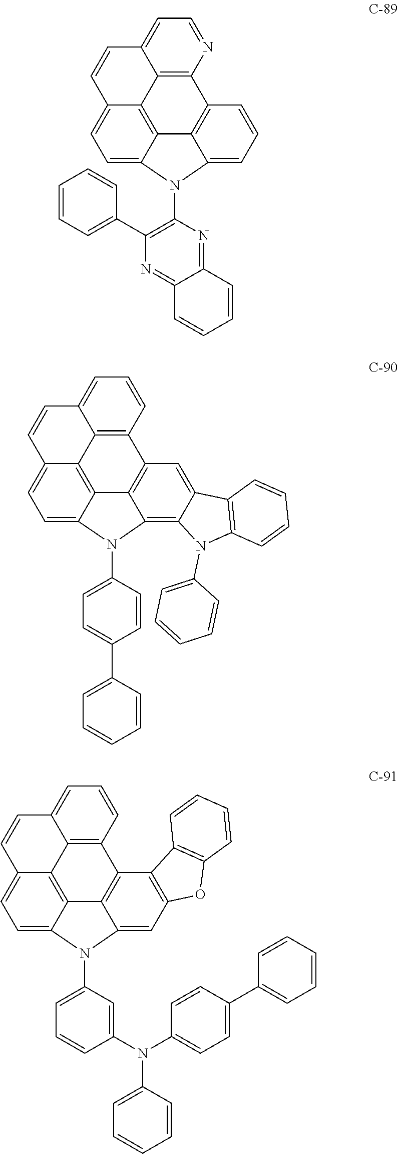

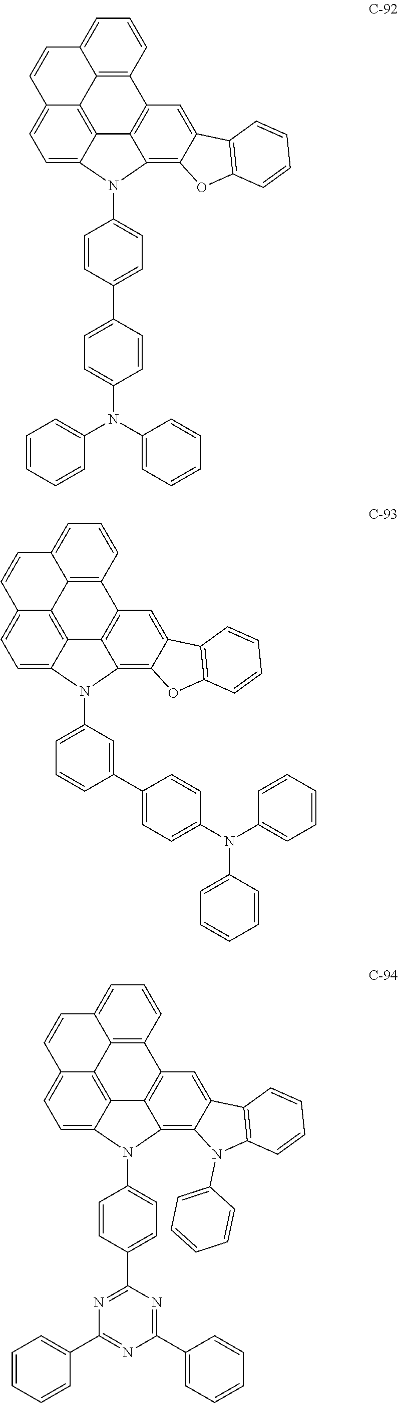

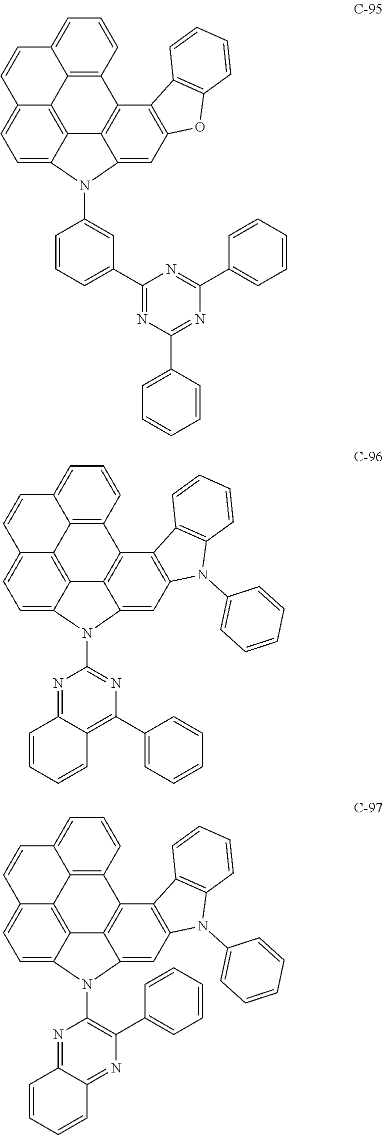

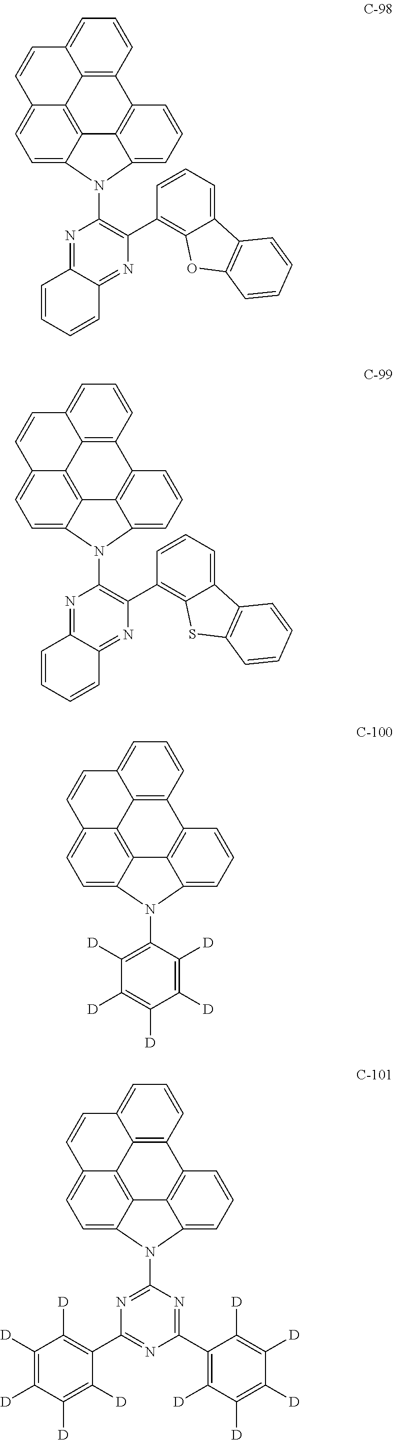

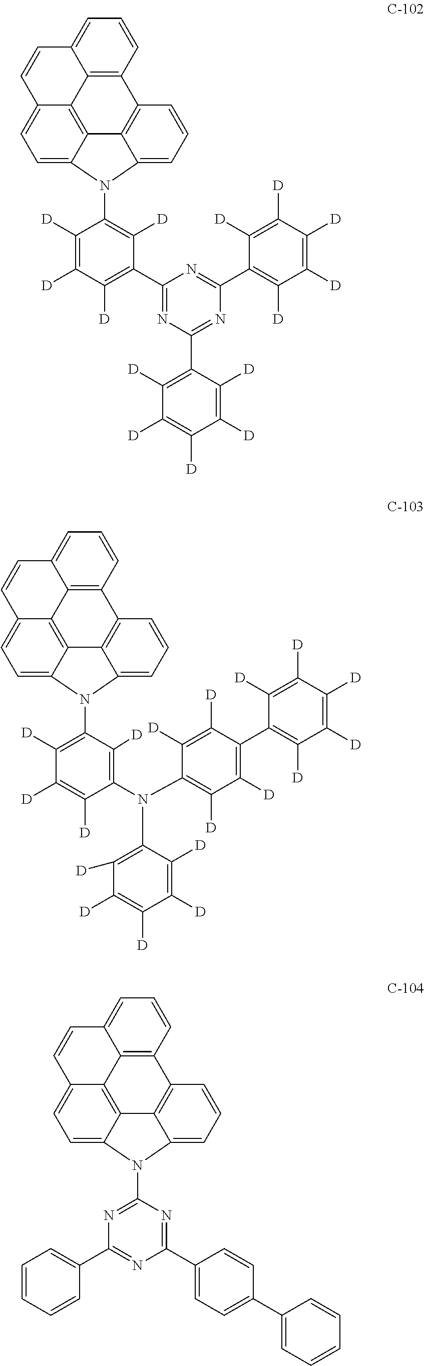 [SMILES: [2H]c1c([2H])c([2H])c(-c2nc(-c3c([2H])c([2H])c([2H])c([2H])c3[2H])nc(-c3c([2H])c([2H])c([2H])c(-n4c5cccc6c7cccc8ccc9ccc4c(c9c87)c65)c3[2H])n2)c([2H])c1[2H] ; [2H]c1c([2H])c([2H])c(-c2c([2H])c([2H])c(N(c3c([2H])c([2H])c([2H])c([2H])c3[2H])c3c([2H])c([2H])c([2H])c(-n4c5cccc6c7cccc8ccc9ccc4c(c9c87)c65)c3[2H])c([2H])c2[2H])c([2H])c1[2H] ; c1ccc(-c2ccc(-c3nc(-c4ccccc4)nc(-n4c5cccc6c7cccc8ccc9ccc4c(c9c87)c65)n3)cc2)cc1]

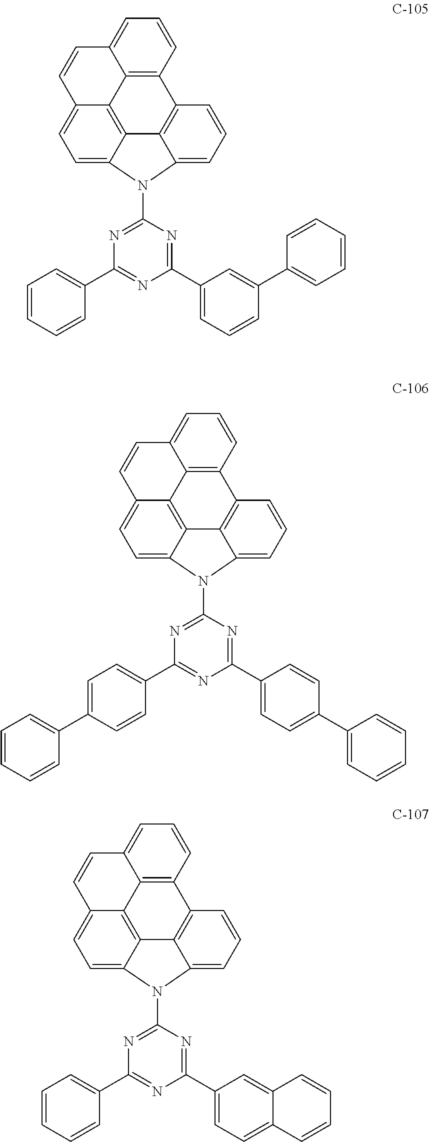 [SMILES: c1ccc(-c2cccc(-c3nc(-c4ccccc4)nc(-n4c5cccc6c7cccc8ccc9ccc4c(c9c87)c65)n3)c2)cc1 ; c1ccc(-c2ccc(-c3nc(-c4ccc(-c5ccccc5)cc4)nc(-n4c5cccc6c7cccc8ccc9ccc4c(c9c87)c65)n3)cc2)cc1 ; c1ccc(-c2nc(-c3ccc4ccccc4c3)nc(-n3c4cccc5c6cccc7ccc8ccc3c(c8c76)c54)n2)cc1]

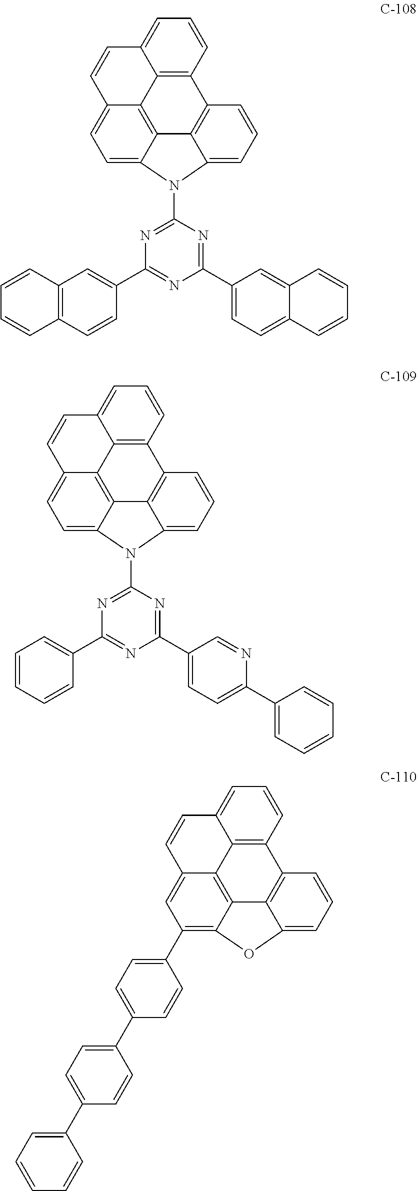 [SMILES: c1ccc2cc(-c3nc(-c4ccc5ccccc5c4)nc(-n4c5cccc6c7cccc8ccc9ccc4c(c9c87)c65)n3)ccc2c1 ; c1ccc(-c2ccc(-c3nc(-c4ccccc4)nc(-n4c5cccc6c7cccc8ccc9ccc4c(c9c87)c65)n3)cn2)cc1 ; c1ccc(-c2ccc(-c3ccc(-c4cc5ccc6cccc7c8cccc9oc4c(c98)c5c67)cc3)cc2)cc1]

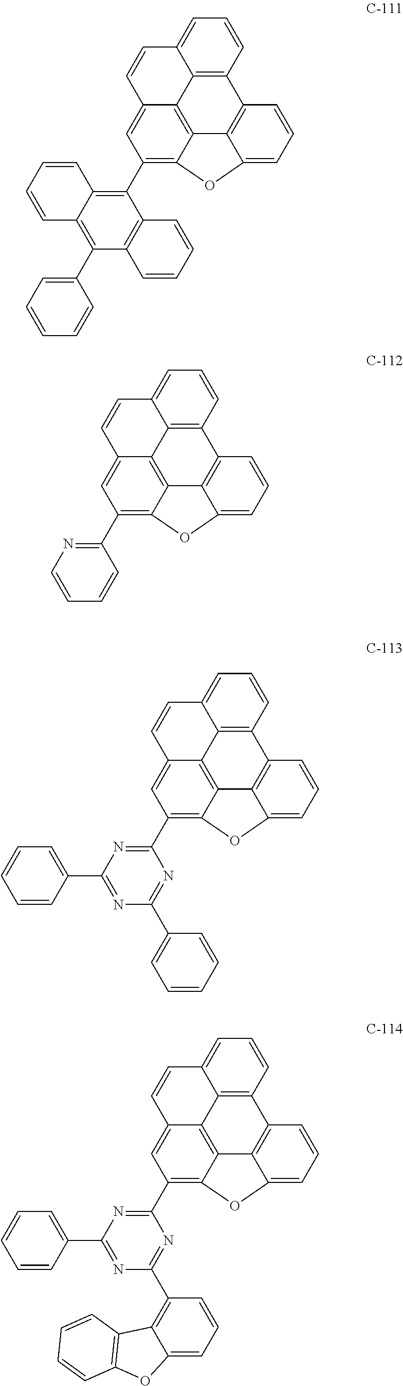 [SMILES: c1ccc(-c2c3ccccc3c(-c3cc4ccc5cccc6c7cccc8oc3c(c87)c4c56)c3ccccc23)cc1 ; c1ccc(-c2cc3ccc4cccc5c6cccc7oc2c(c76)c3c45)nc1 ; c1ccc(-c2nc(-c3ccccc3)nc(-c3cc4ccc5cccc6c7cccc8oc3c(c87)c4c56)n2)cc1 ; c1ccc(-c2nc(-c3cc4ccc5cccc6c7cccc8oc3c(c87)c4c56)nc(-c3cccc4oc5ccccc5c34)n2)cc1]

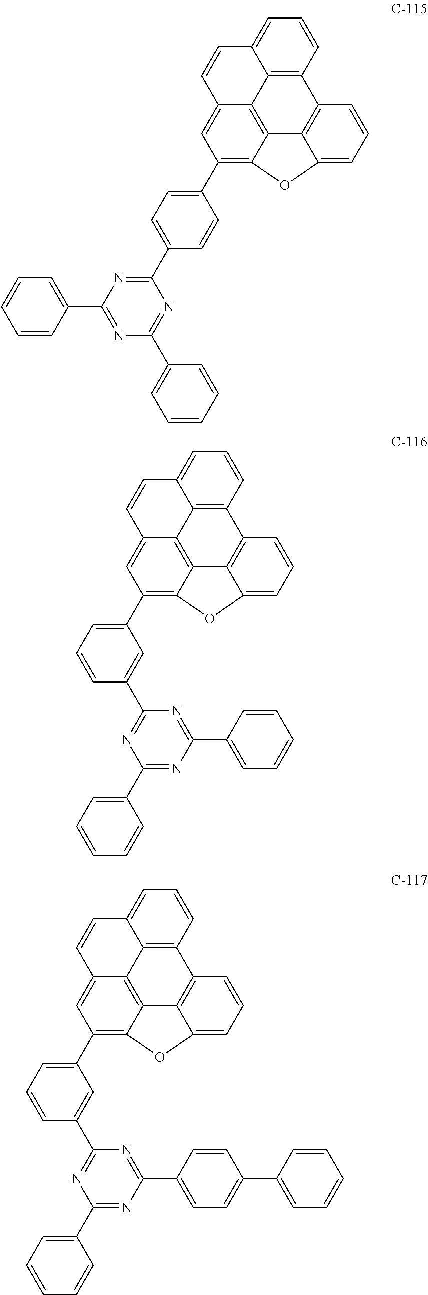

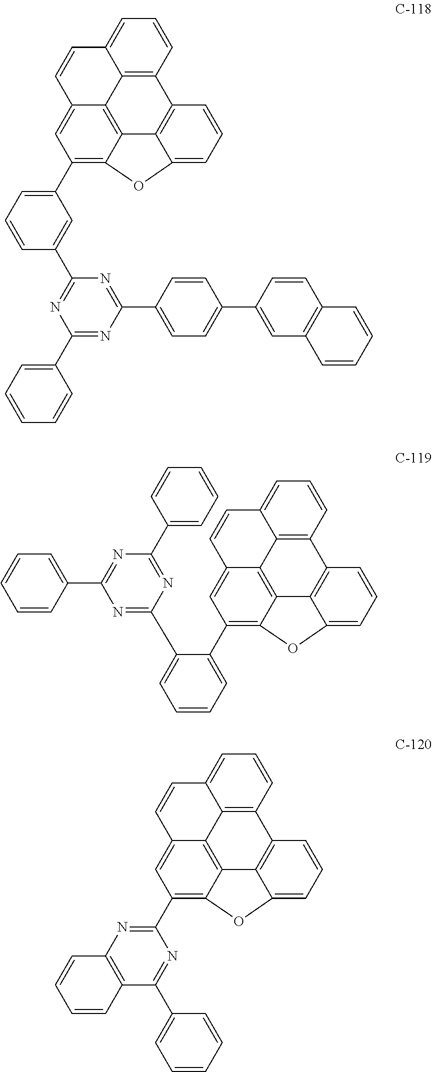

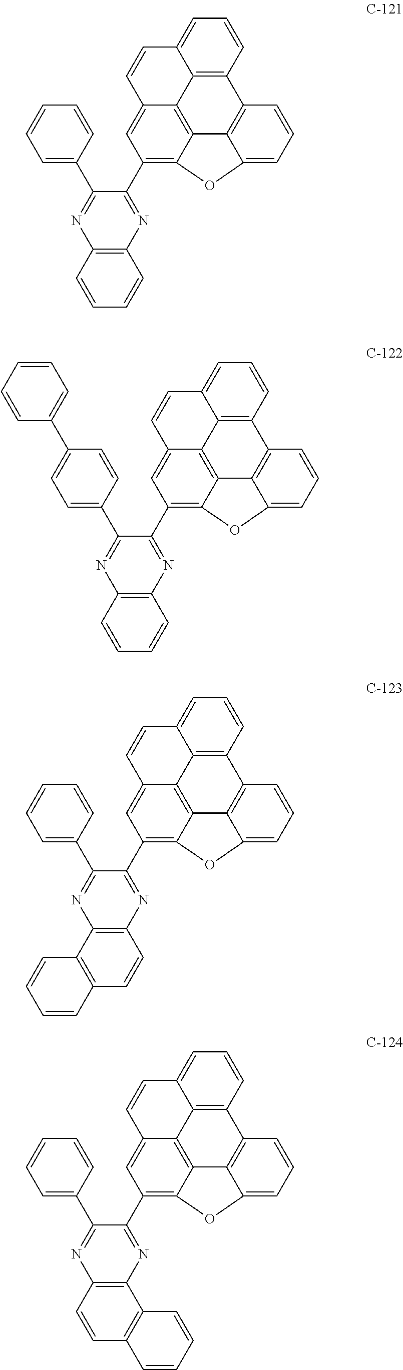 [SMILES: c1ccc(-c2nc3ccccc3nc2-c2cc3ccc4cccc5c6cccc7oc2c(c76)c3c45)cc1 ; c1ccc(-c2ccc(-c3nc4ccccc4nc3-c3cc4ccc5cccc6c7cccc8oc3c(c87)c4c56)cc2)cc1 ; c1ccc(-c2nc3c(ccc4ccccc43)nc2-c2cc3ccc4cccc5c6cccc7oc2c(c76)c3c45)cc1 ; c1ccc(-c2nc3ccc4ccccc4c3nc2-c2cc3ccc4cccc5c6cccc7oc2c(c76)c3c45)cc1]

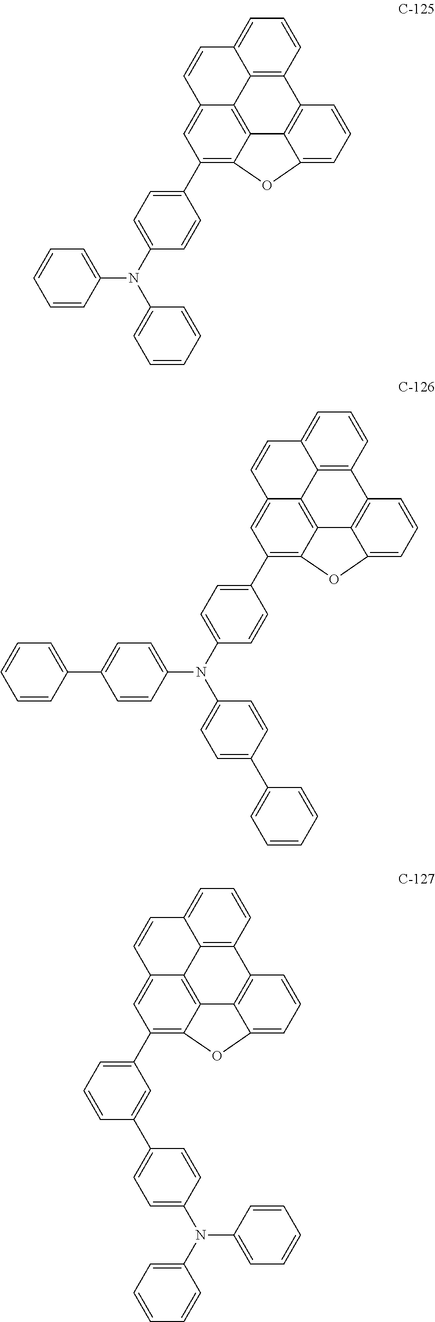 [SMILES: c1ccc(N(c2ccccc2)c2ccc(-c3cc4ccc5cccc6c7cccc8oc3c(c87)c4c56)cc2)cc1 ; c1ccc(-c2ccc(N(c3ccc(-c4ccccc4)cc3)c3ccc(-c4cc5ccc6cccc7c8cccc9oc4c(c98)c5c67)cc3)cc2)cc1 ; c1ccc(N(c2ccccc2)c2ccc(-c3cccc(-c4cc5ccc6cccc7c8cccc9oc4c(c98)c5c67)c3)cc2)cc1]

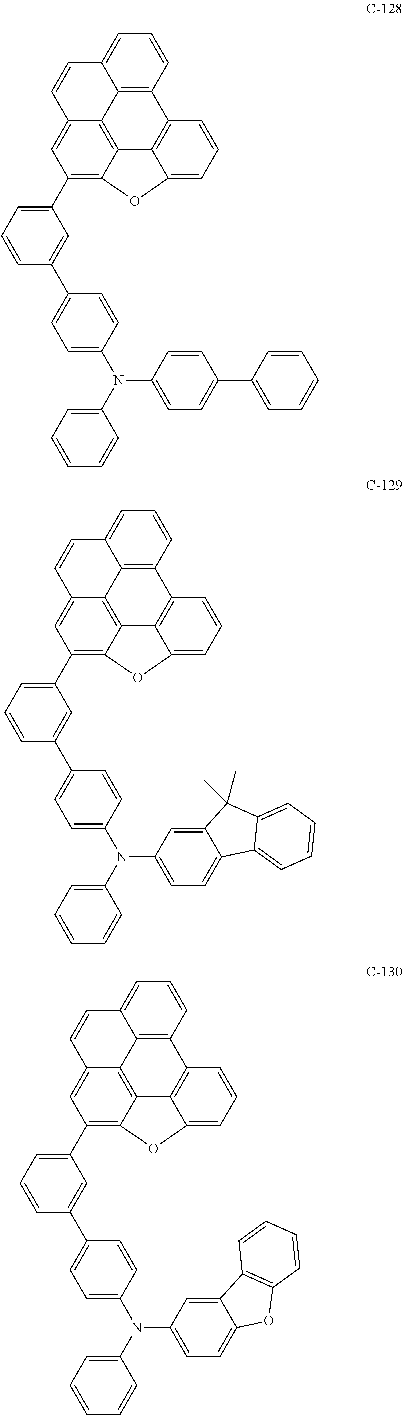 [SMILES: c1ccc(-c2ccc(N(c3ccccc3)c3ccc(-c4cccc(-c5cc6ccc7cccc8c9cccc%10oc5c(c%109)c6c78)c4)cc3)cc2)cc1 ; CC1(C)c2ccccc2-c2ccc(N(c3ccccc3)c3ccc(-c4cccc(-c5cc6ccc7cccc8c9cccc%10oc5c(c%109)c6c78)c4)cc3)cc21 ; c1ccc(N(c2ccc(-c3cccc(-c4cc5ccc6cccc7c8cccc9oc4c(c98)c5c67)c3)cc2)c2ccc3oc4ccccc4c3c2)cc1]

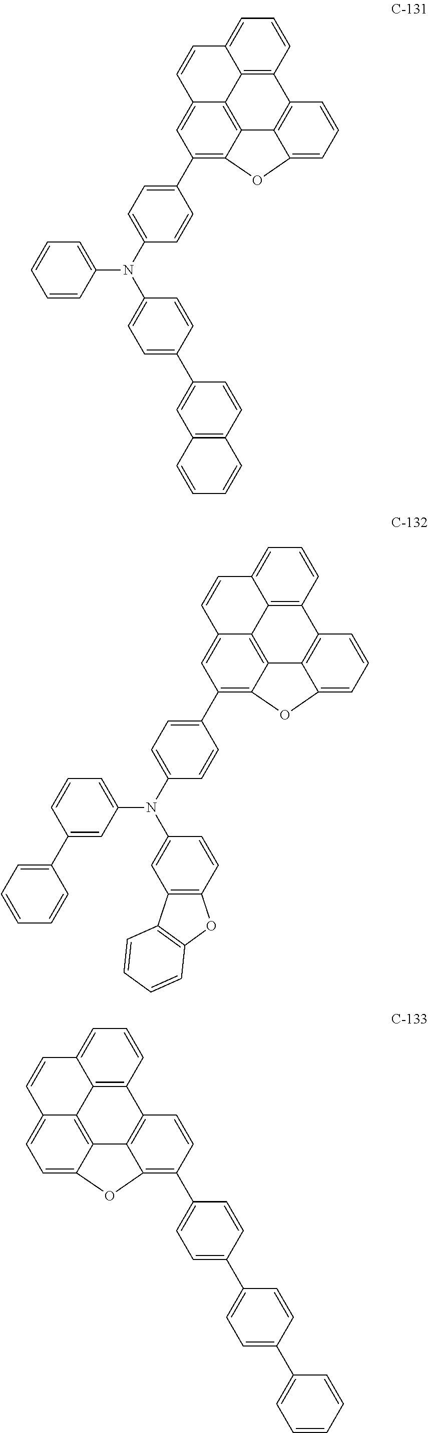

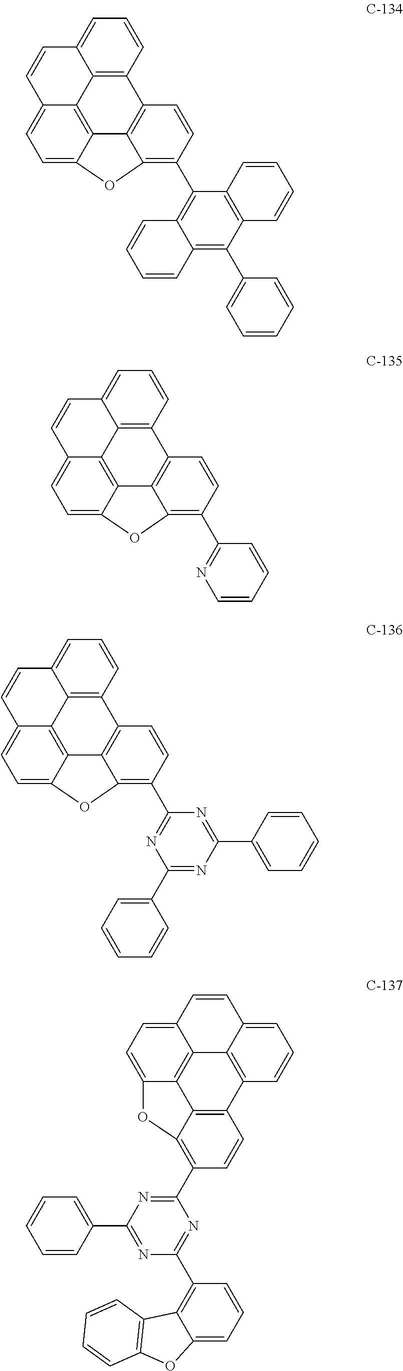 [SMILES: c1ccc(-c2c3ccccc3c(-c3ccc4c5cccc6ccc7ccc8oc3c4c8c7c65)c3ccccc23)cc1 ; c1ccc(-c2ccc3c4cccc5ccc6ccc7oc2c3c7c6c54)nc1 ; c1ccc(-c2nc(-c3ccccc3)nc(-c3ccc4c5cccc6ccc7ccc8oc3c4c8c7c65)n2)cc1 ; c1ccc(-c2nc(-c3ccc4c5cccc6ccc7ccc8oc3c4c8c7c65)nc(-c3cccc4oc5ccccc5c34)n2)cc1]

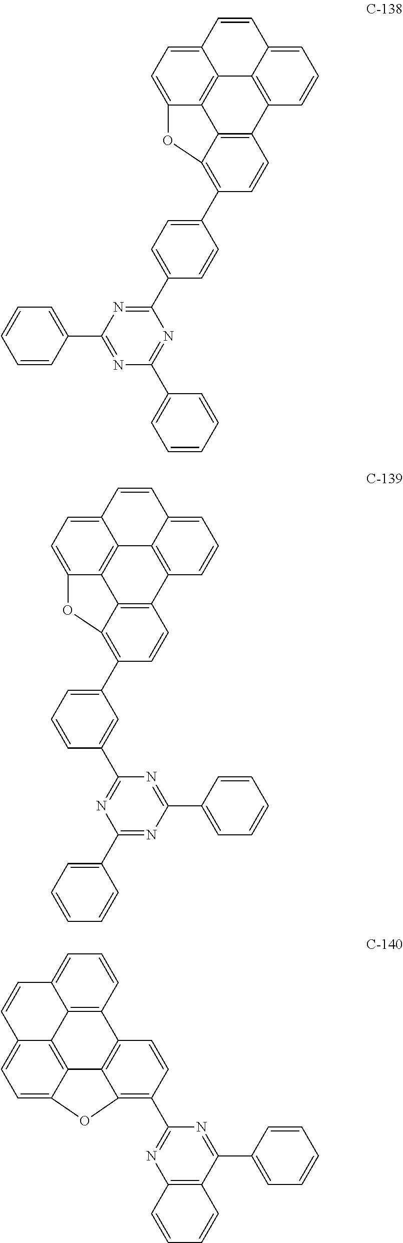

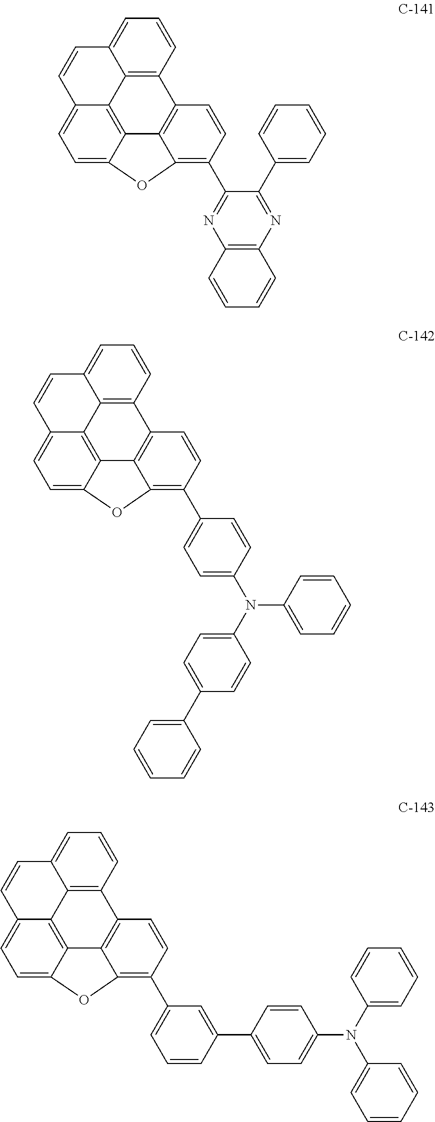 [SMILES: c1ccc(-c2nc3ccccc3nc2-c2ccc3c4cccc5ccc6ccc7oc2c3c7c6c54)cc1 ; c1ccc(-c2ccc(N(c3ccccc3)c3ccc(-c4ccc5c6cccc7ccc8ccc9oc4c5c9c8c76)cc3)cc2)cc1 ; c1ccc(N(c2ccccc2)c2ccc(-c3cccc(-c4ccc5c6cccc7ccc8ccc9oc4c5c9c8c76)c3)cc2)cc1]

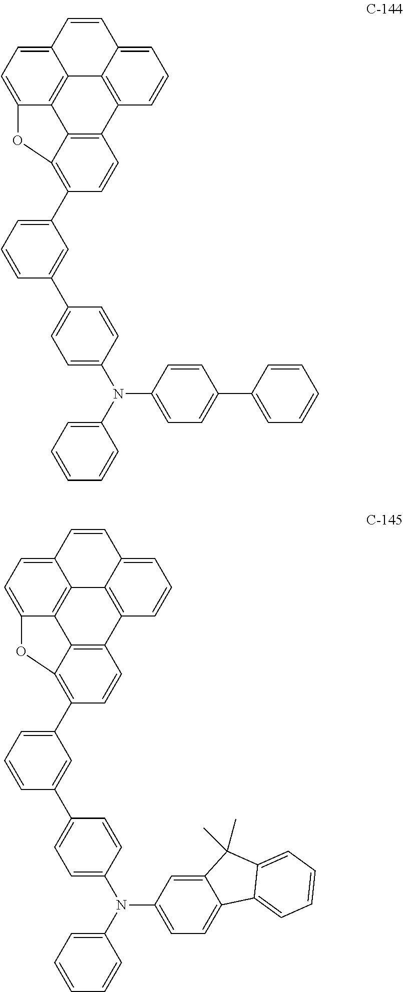 [SMILES: c1ccc(-c2ccc(N(c3ccccc3)c3ccc(-c4cccc(-c5ccc6c7cccc8ccc9ccc%10oc5c6c%10c9c87)c4)cc3)cc2)cc1 ; CC1(C)c2ccccc2-c2ccc(N(c3ccccc3)c3ccc(-c4cccc(-c5ccc6c7cccc8ccc9ccc%10oc5c6c%10c9c87)c4)cc3)cc21]

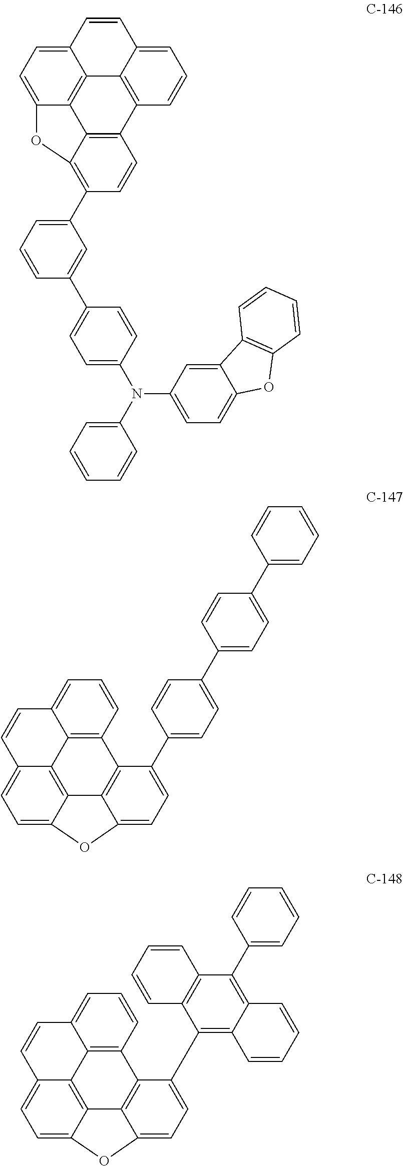 [SMILES: c1ccc(N(c2ccc(-c3cccc(-c4ccc5c6cccc7ccc8ccc9oc4c5c9c8c76)c3)cc2)c2ccc3oc4ccccc4c3c2)cc1 ; c1ccc(-c2ccc(-c3ccc(-c4ccc5oc6ccc7ccc8cccc9c4c5c6c7c89)cc3)cc2)cc1 ; c1ccc(-c2c3ccccc3c(-c3ccc4oc5ccc6ccc7cccc8c3c4c5c6c78)c3ccccc23)cc1]

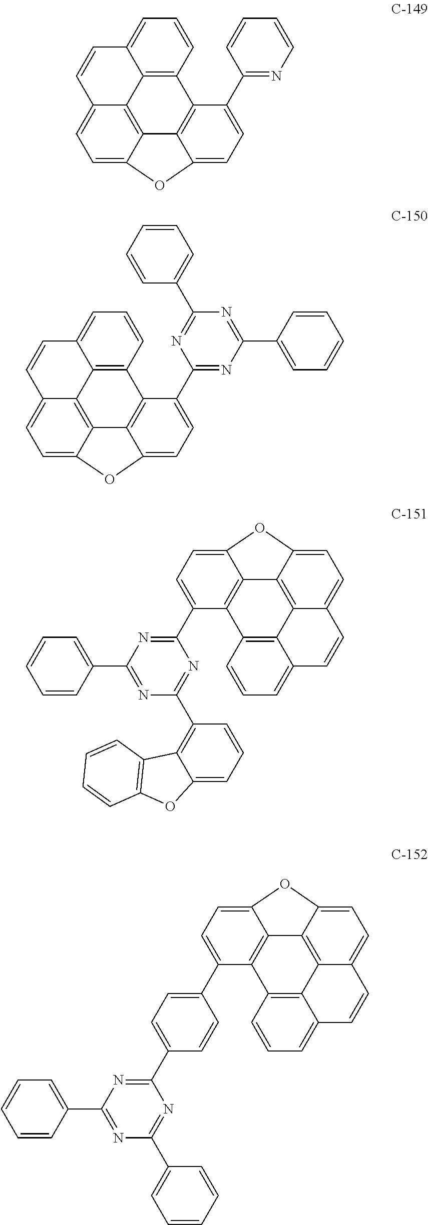

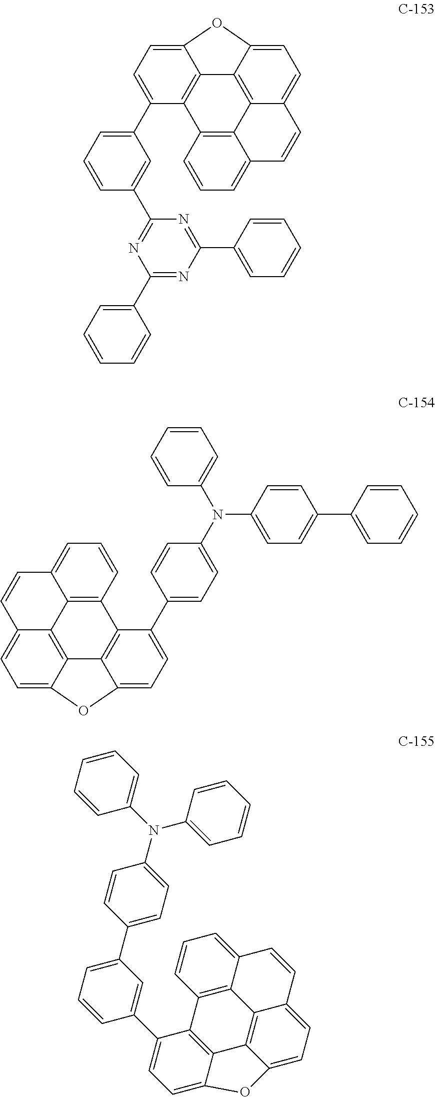 [SMILES: c1ccc(-c2nc(-c3ccccc3)nc(-c3cccc(-c4ccc5oc6ccc7ccc8cccc9c4c5c6c7c89)c3)n2)cc1 ; c1ccc(-c2ccc(N(c3ccccc3)c3ccc(-c4ccc5oc6ccc7ccc8cccc9c4c5c6c7c89)cc3)cc2)cc1 ; c1ccc(N(c2ccccc2)c2ccc(-c3cccc(-c4ccc5oc6ccc7ccc8cccc9c4c5c6c7c89)c3)cc2)cc1]

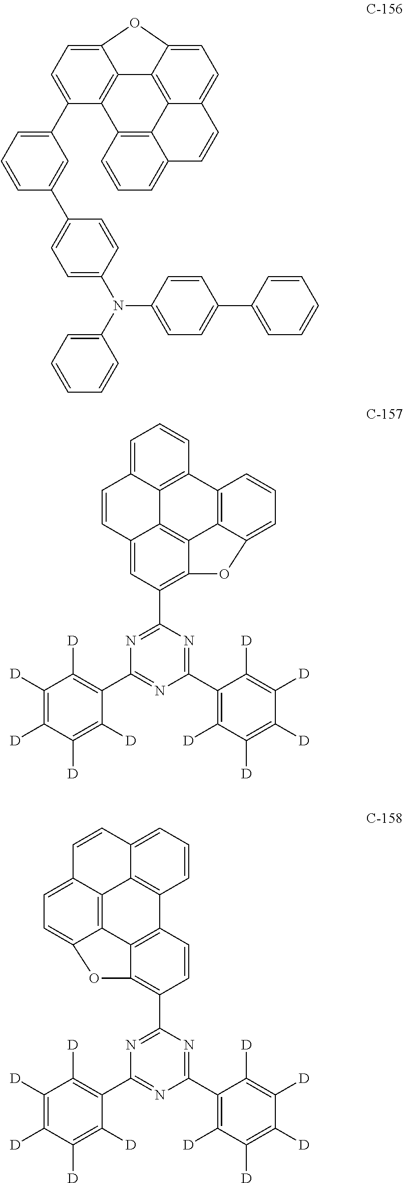 [SMILES: c1ccc(-c2ccc(N(c3ccccc3)c3ccc(-c4cccc(-c5ccc6oc7ccc8ccc9cccc%10c5c6c7c8c9%10)c4)cc3)cc2)cc1 ; [2H]c1c([2H])c([2H])c(-c2nc(-c3c([2H])c([2H])c([2H])c([2H])c3[2H])nc(-c3cc4ccc5cccc6c7cccc8oc3c(c87)c4c56)n2)c([2H])c1[2H] ; [2H]c1c([2H])c([2H])c(-c2nc(-c3c([2H])c([2H])c([2H])c([2H])c3[2H])nc(-c3ccc4c5cccc6ccc7ccc8oc3c4c8c7c65)n2)c([2H])c1[2H]]

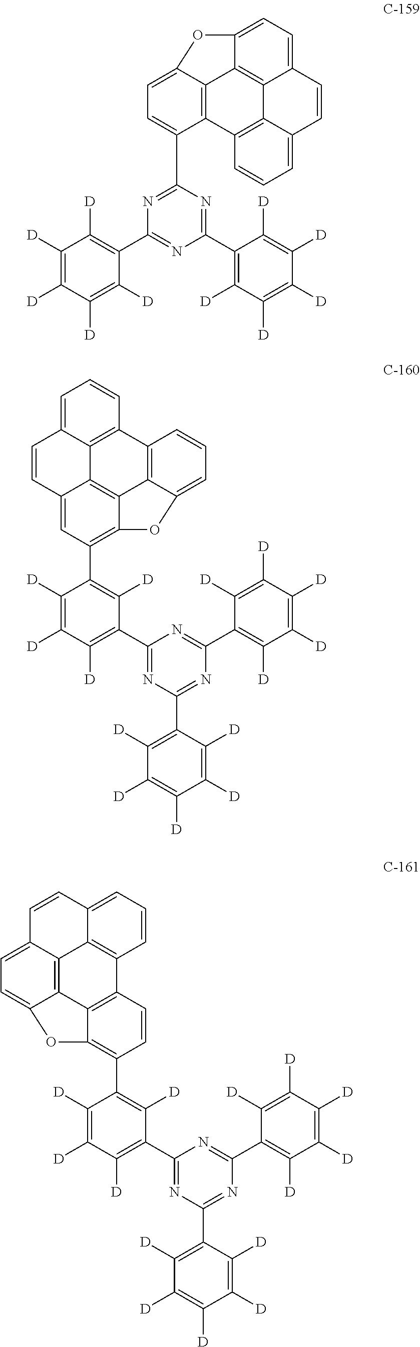

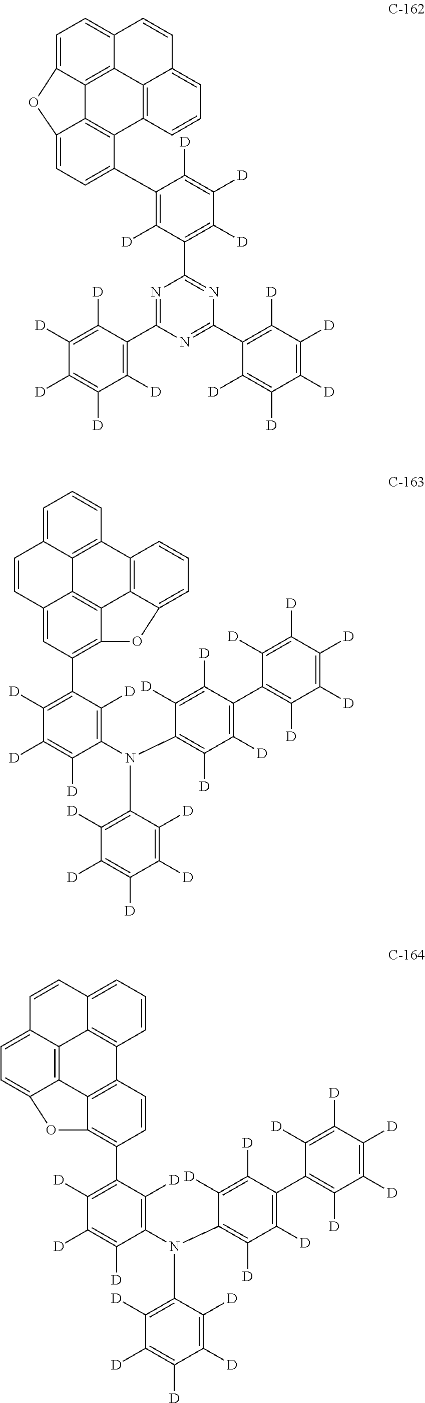

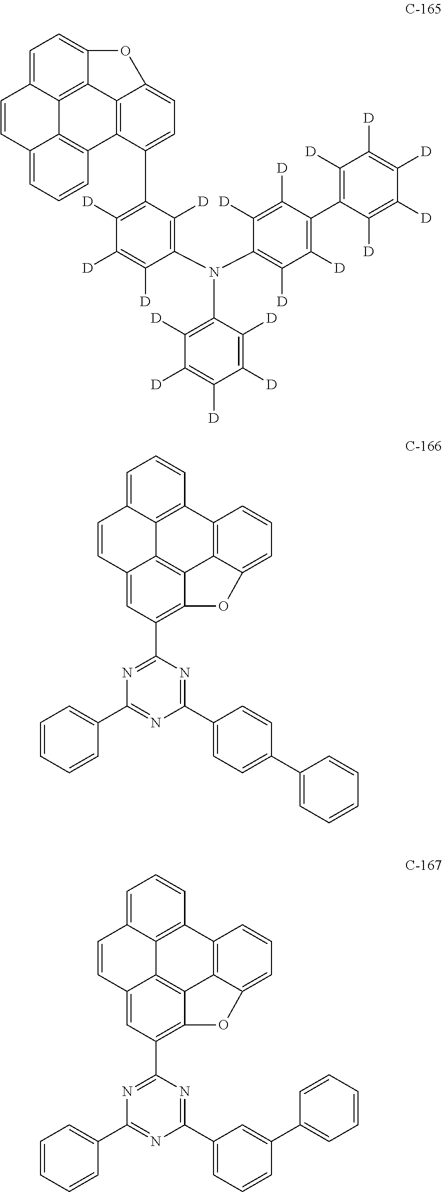 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4ccc5oc6ccc7ccc8cccc9c4c5c6c7c89)c3[2H])c([2H])c2[2H])c([2H])c1[2H] ; c1ccc(-c2ccc(-c3nc(-c4ccccc4)nc(-c4cc5ccc6cccc7c8cccc9oc4c(c98)c5c67)n3)cc2)cc1 ; c1ccc(-c2cccc(-c3nc(-c4ccccc4)nc(-c4cc5ccc6cccc7c8cccc9oc4c(c98)c5c67)n3)c2)cc1]

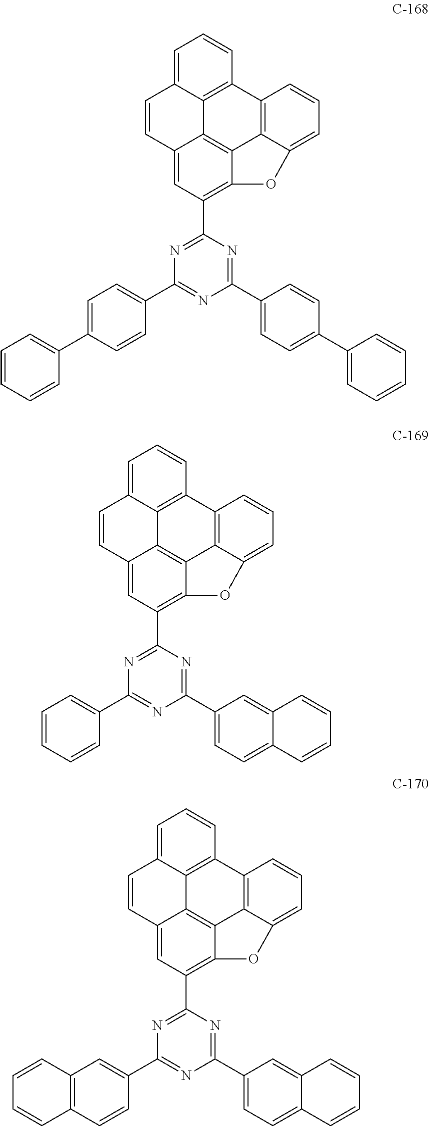 [SMILES: c1ccc(-c2ccc(-c3nc(-c4ccc(-c5ccccc5)cc4)nc(-c4cc5ccc6cccc7c8cccc9oc4c(c98)c5c67)n3)cc2)cc1 ; c1ccc(-c2nc(-c3ccc4ccccc4c3)nc(-c3cc4ccc5cccc6c7cccc8oc3c(c87)c4c56)n2)cc1 ; c1ccc2cc(-c3nc(-c4ccc5ccccc5c4)nc(-c4cc5ccc6cccc7c8cccc9oc4c(c98)c5c67)n3)ccc2c1]

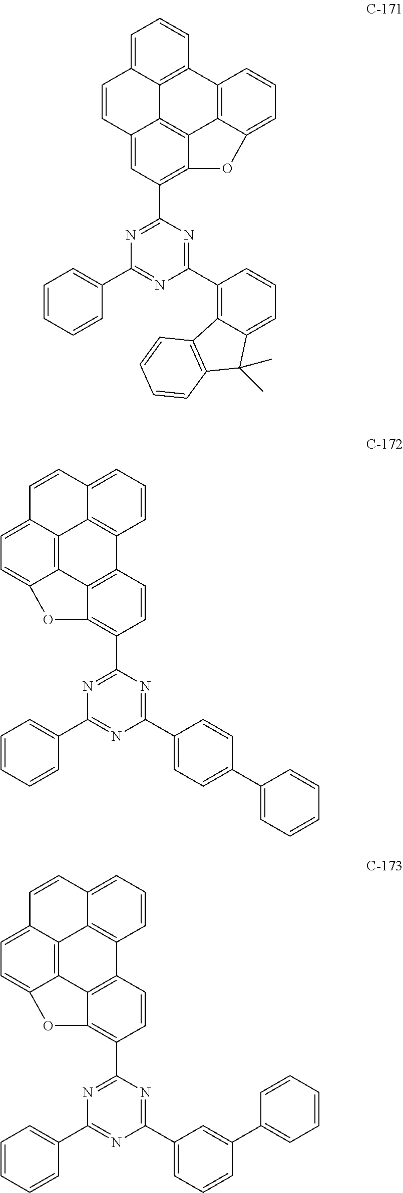 [SMILES: CC1(C)c2ccccc2-c2c(-c3nc(-c4ccccc4)nc(-c4cc5ccc6cccc7c8cccc9oc4c(c98)c5c67)n3)cccc21 ; c1ccc(-c2ccc(-c3nc(-c4ccccc4)nc(-c4ccc5c6cccc7ccc8ccc9oc4c5c9c8c76)n3)cc2)cc1 ; c1ccc(-c2cccc(-c3nc(-c4ccccc4)nc(-c4ccc5c6cccc7ccc8ccc9oc4c5c9c8c76)n3)c2)cc1]

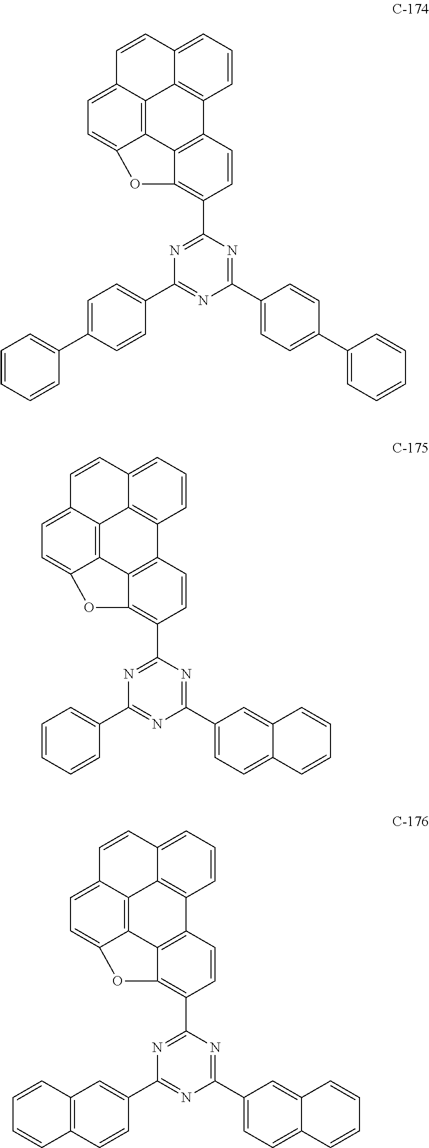 [SMILES: c1ccc(-c2ccc(-c3nc(-c4ccc(-c5ccccc5)cc4)nc(-c4ccc5c6cccc7ccc8ccc9oc4c5c9c8c76)n3)cc2)cc1 ; c1ccc(-c2nc(-c3ccc4ccccc4c3)nc(-c3ccc4c5cccc6ccc7ccc8oc3c4c8c7c65)n2)cc1 ; c1ccc2cc(-c3nc(-c4ccc5ccccc5c4)nc(-c4ccc5c6cccc7ccc8ccc9oc4c5c9c8c76)n3)ccc2c1]

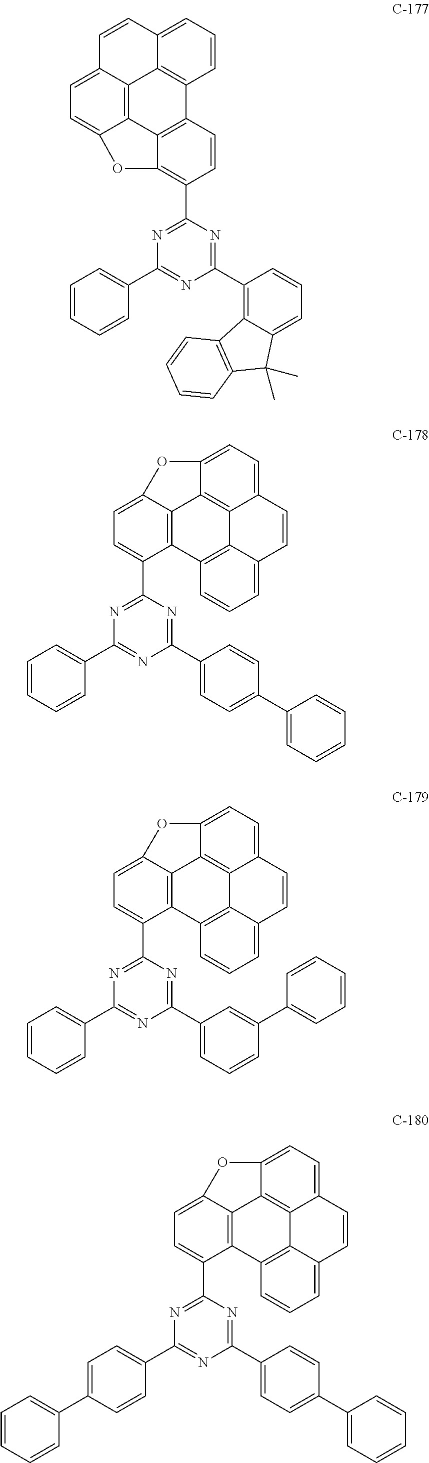 [SMILES: CC1(C)c2ccccc2-c2c(-c3nc(-c4ccccc4)nc(-c4ccc5c6cccc7ccc8ccc9oc4c5c9c8c76)n3)cccc21 ; c1ccc(-c2ccc(-c3nc(-c4ccccc4)nc(-c4ccc5oc6ccc7ccc8cccc9c4c5c6c7c89)n3)cc2)cc1 ; c1ccc(-c2cccc(-c3nc(-c4ccccc4)nc(-c4ccc5oc6ccc7ccc8cccc9c4c5c6c7c89)n3)c2)cc1 ; c1ccc(-c2ccc(-c3nc(-c4ccc(-c5ccccc5)cc4)nc(-c4ccc5oc6ccc7ccc8cccc9c4c5c6c7c89)n3)cc2)cc1]

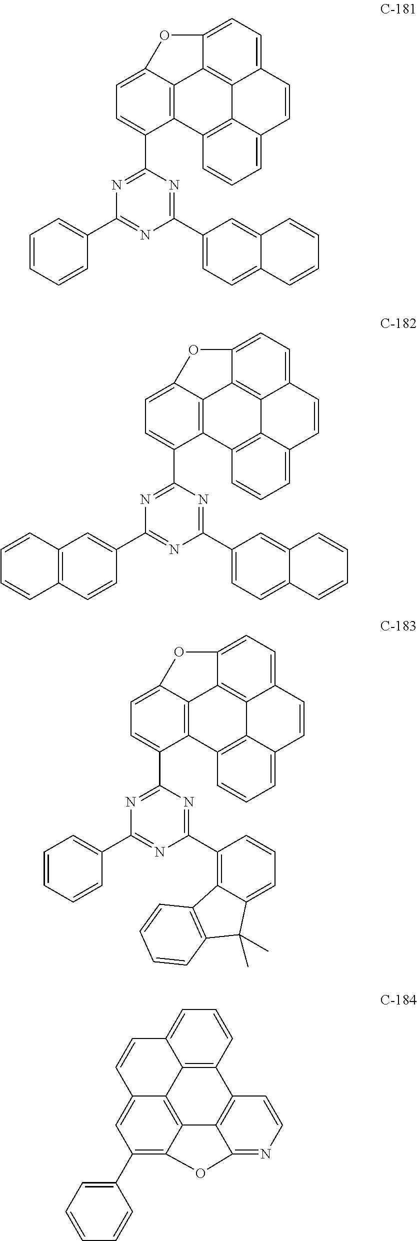

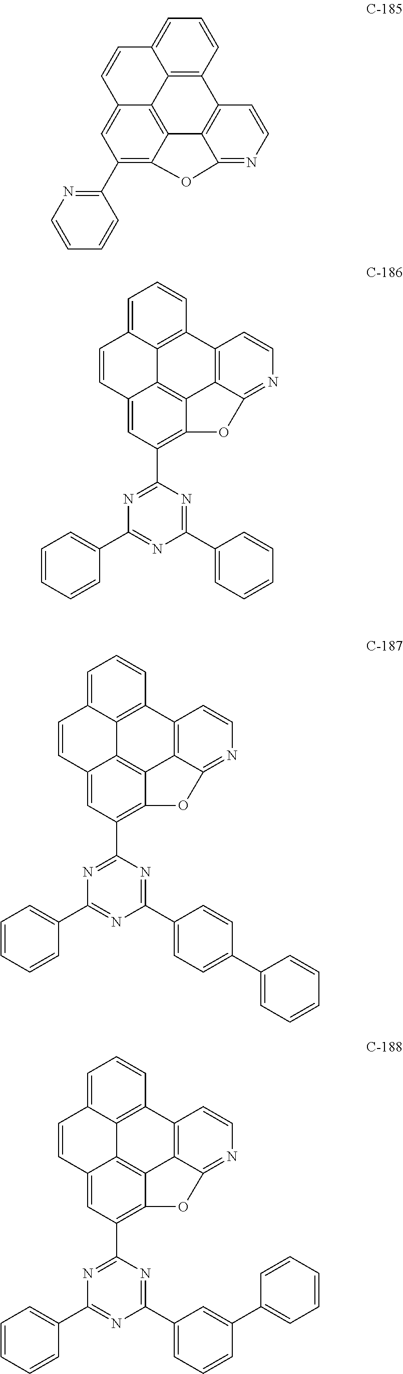 [SMILES: c1ccc(-c2cc3ccc4cccc5c6ccnc7oc2c(c76)c3c45)nc1 ; c1ccc(-c2nc(-c3ccccc3)nc(-c3cc4ccc5cccc6c7ccnc8oc3c(c87)c4c56)n2)cc1 ; c1ccc(-c2ccc(-c3nc(-c4ccccc4)nc(-c4cc5ccc6cccc7c8ccnc9oc4c(c98)c5c67)n3)cc2)cc1 ; c1ccc(-c2cccc(-c3nc(-c4ccccc4)nc(-c4cc5ccc6cccc7c8ccnc9oc4c(c98)c5c67)n3)c2)cc1]

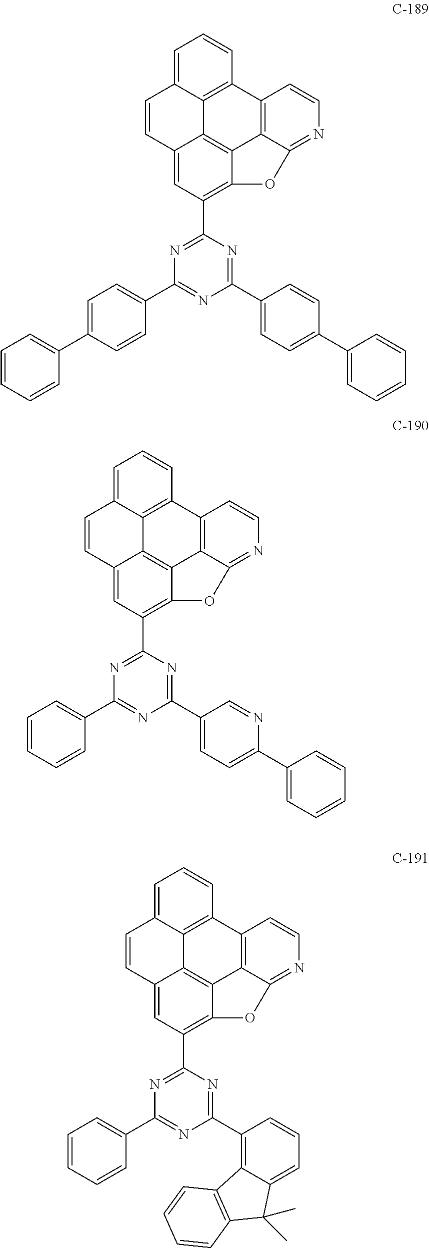 [SMILES: c1ccc(-c2ccc(-c3nc(-c4ccc(-c5ccccc5)cc4)nc(-c4cc5ccc6cccc7c8ccnc9oc4c(c98)c5c67)n3)cc2)cc1 ; c1ccc(-c2ccc(-c3nc(-c4ccccc4)nc(-c4cc5ccc6cccc7c8ccnc9oc4c(c98)c5c67)n3)cn2)cc1 ; CC1(C)c2ccccc2-c2c(-c3nc(-c4ccccc4)nc(-c4cc5ccc6cccc7c8ccnc9oc4c(c98)c5c67)n3)cccc21]

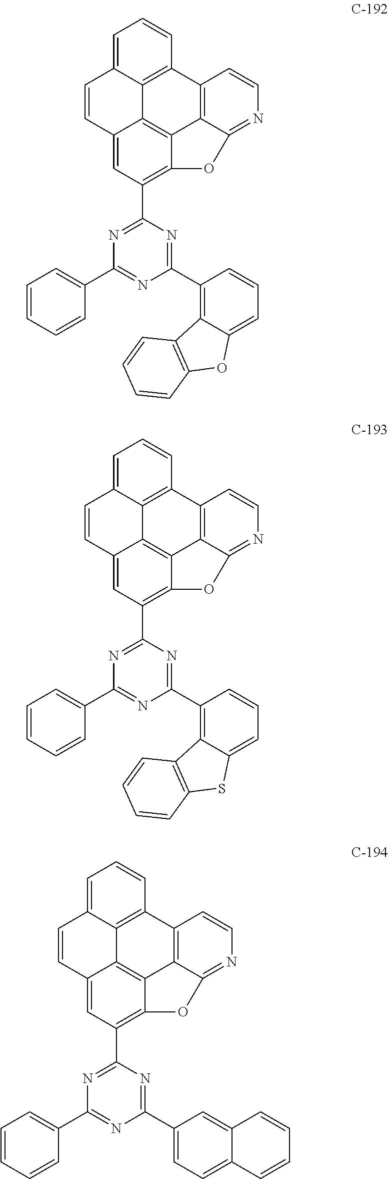

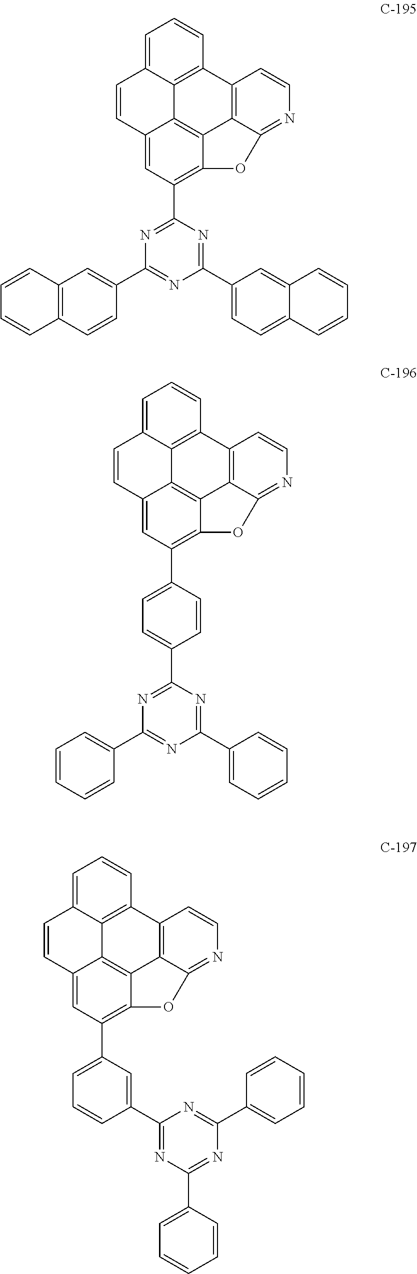 [SMILES: c1ccc2cc(-c3nc(-c4ccc5ccccc5c4)nc(-c4cc5ccc6cccc7c8ccnc9oc4c(c98)c5c67)n3)ccc2c1 ; c1ccc(-c2nc(-c3ccccc3)nc(-c3ccc(-c4cc5ccc6cccc7c8ccnc9oc4c(c98)c5c67)cc3)n2)cc1 ; c1ccc(-c2nc(-c3ccccc3)nc(-c3cccc(-c4cc5ccc6cccc7c8ccnc9oc4c(c98)c5c67)c3)n2)cc1]

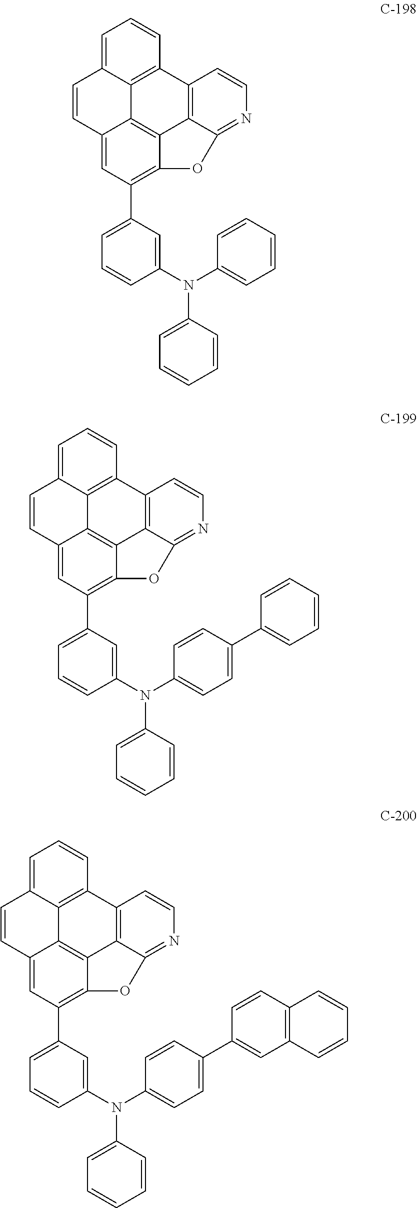 [SMILES: c1ccc(N(c2ccccc2)c2cccc(-c3cc4ccc5cccc6c7ccnc8oc3c(c87)c4c56)c2)cc1 ; c1ccc(-c2ccc(N(c3ccccc3)c3cccc(-c4cc5ccc6cccc7c8ccnc9oc4c(c98)c5c67)c3)cc2)cc1 ; c1ccc(N(c2ccc(-c3ccc4ccccc4c3)cc2)c2cccc(-c3cc4ccc5cccc6c7ccnc8oc3c(c87)c4c56)c2)cc1]

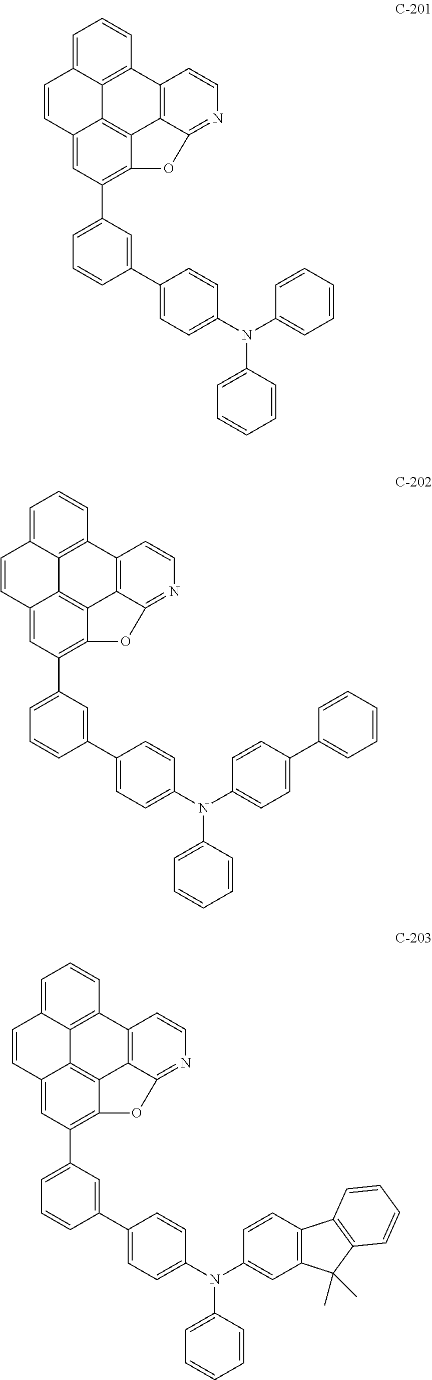 [SMILES: c1ccc(N(c2ccccc2)c2ccc(-c3cccc(-c4cc5ccc6cccc7c8ccnc9oc4c(c98)c5c67)c3)cc2)cc1 ; c1ccc(-c2ccc(N(c3ccccc3)c3ccc(-c4cccc(-c5cc6ccc7cccc8c9ccnc%10oc5c(c%109)c6c78)c4)cc3)cc2)cc1 ; CC1(C)c2ccccc2-c2ccc(N(c3ccccc3)c3ccc(-c4cccc(-c5cc6ccc7cccc8c9ccnc%10oc5c(c%109)c6c78)c4)cc3)cc21]

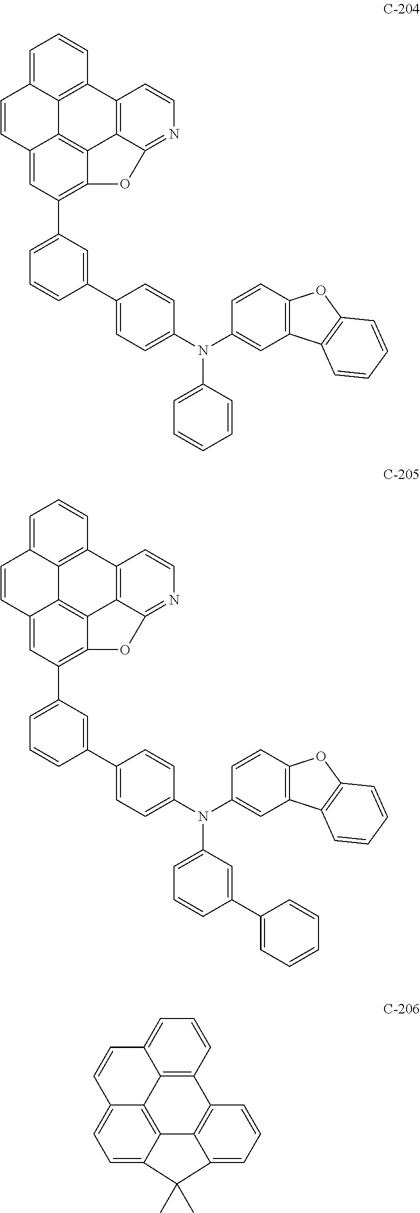

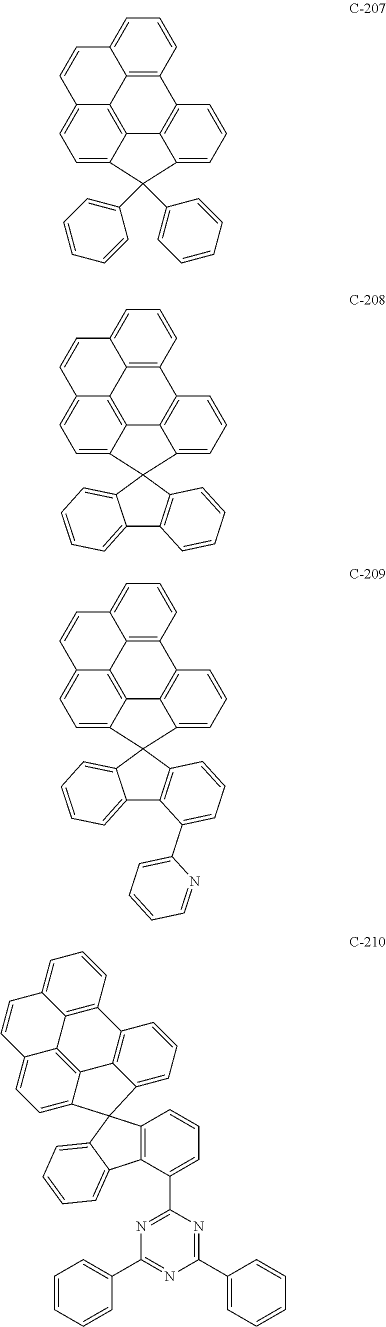

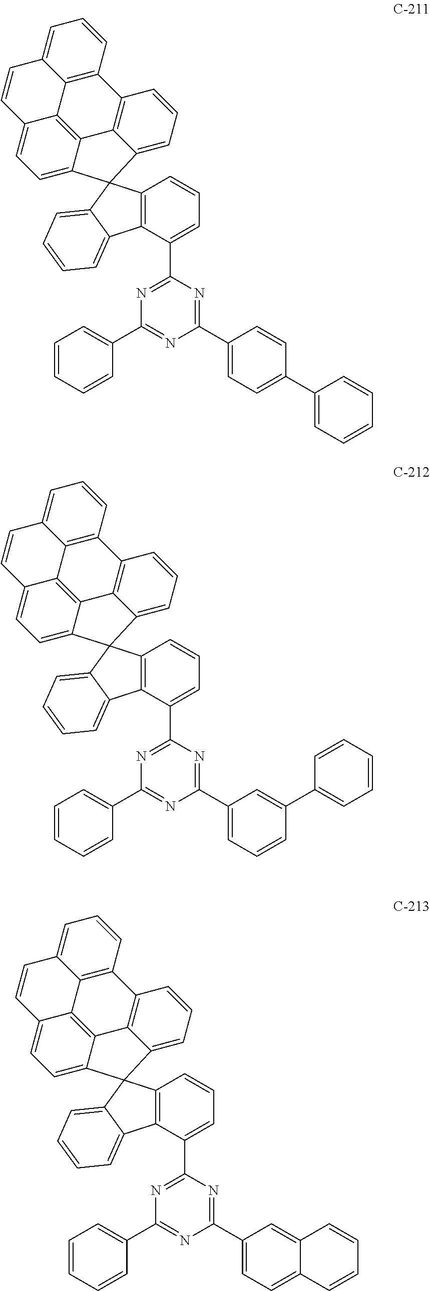 [SMILES: c1ccc(-c2ccc(-c3nc(-c4ccccc4)nc(-c4cccc5c4-c4ccccc4C54c5cccc6c7cccc8ccc9ccc4c(c56)c9c87)n3)cc2)cc1 ; c1ccc(-c2cccc(-c3nc(-c4ccccc4)nc(-c4cccc5c4-c4ccccc4C54c5cccc6c7cccc8ccc9ccc4c(c56)c9c87)n3)c2)cc1 ; c1ccc(-c2nc(-c3ccc4ccccc4c3)nc(-c3cccc4c3-c3ccccc3C43c4cccc5c6cccc7ccc8ccc3c(c45)c8c76)n2)cc1]

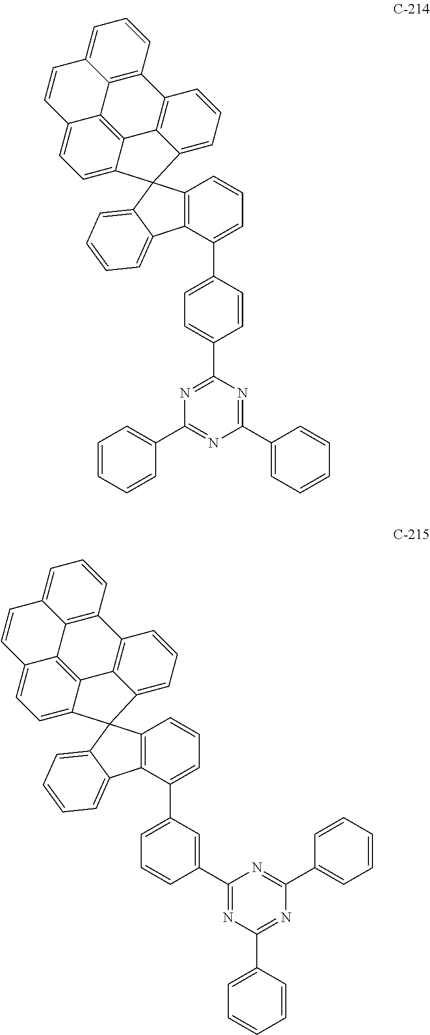 [SMILES: c1ccc(-c2nc(-c3ccccc3)nc(-c3ccc(-c4cccc5c4-c4ccccc4C54c5cccc6c7cccc8ccc9ccc4c(c56)c9c87)cc3)n2)cc1 ; c1ccc(-c2nc(-c3ccccc3)nc(-c3cccc(-c4cccc5c4-c4ccccc4C54c5cccc6c7cccc8ccc9ccc4c(c56)c9c87)c3)n2)cc1]

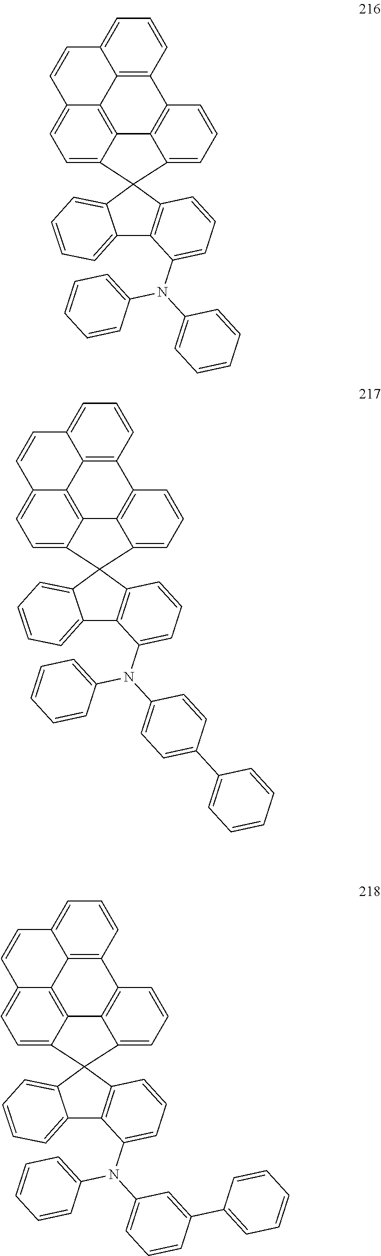 [SMILES: c1ccc(N(c2ccccc2)c2cccc3c2-c2ccccc2C32c3cccc4c5cccc6ccc7ccc2c(c34)c7c65)cc1 ; c1ccc(-c2ccc(N(c3ccccc3)c3cccc4c3-c3ccccc3C43c4cccc5c6cccc7ccc8ccc3c(c45)c8c76)cc2)cc1 ; c1ccc(-c2cccc(N(c3ccccc3)c3cccc4c3-c3ccccc3C43c4cccc5c6cccc7ccc8ccc3c(c45)c8c76)c2)cc1]

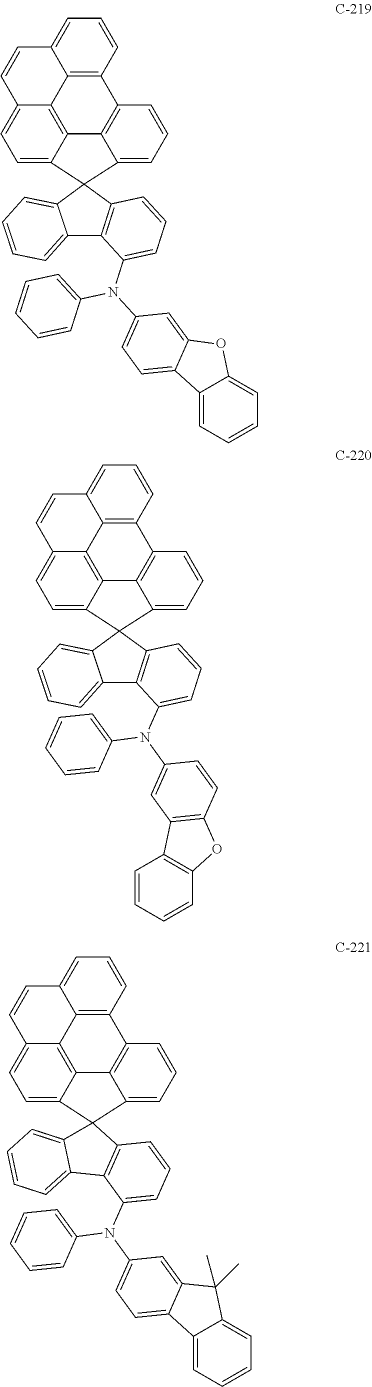 [SMILES: c1ccc(N(c2ccc3c(c2)oc2ccccc23)c2cccc3c2-c2ccccc2C32c3cccc4c5cccc6ccc7ccc2c(c34)c7c65)cc1 ; c1ccc(N(c2ccc3oc4ccccc4c3c2)c2cccc3c2-c2ccccc2C32c3cccc4c5cccc6ccc7ccc2c(c34)c7c65)cc1 ; CC1(C)c2ccccc2-c2ccc(N(c3ccccc3)c3cccc4c3-c3ccccc3C43c4cccc5c6cccc7ccc8ccc3c(c45)c8c76)cc21]

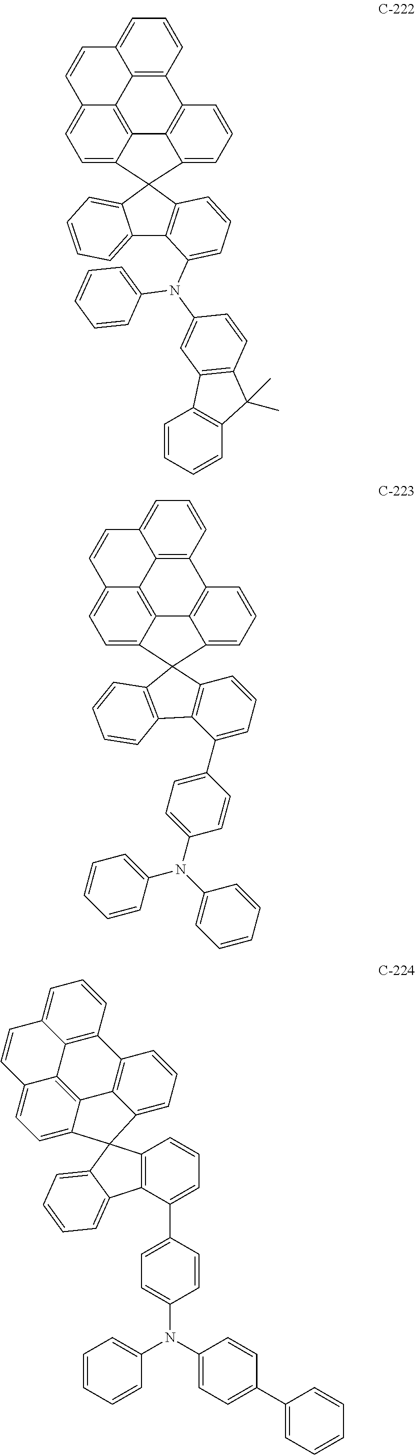

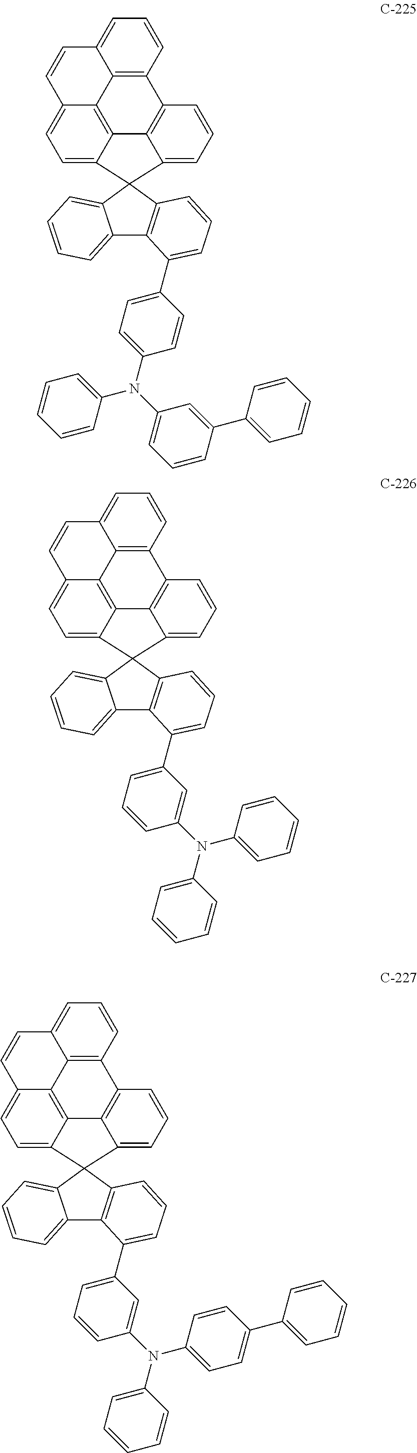

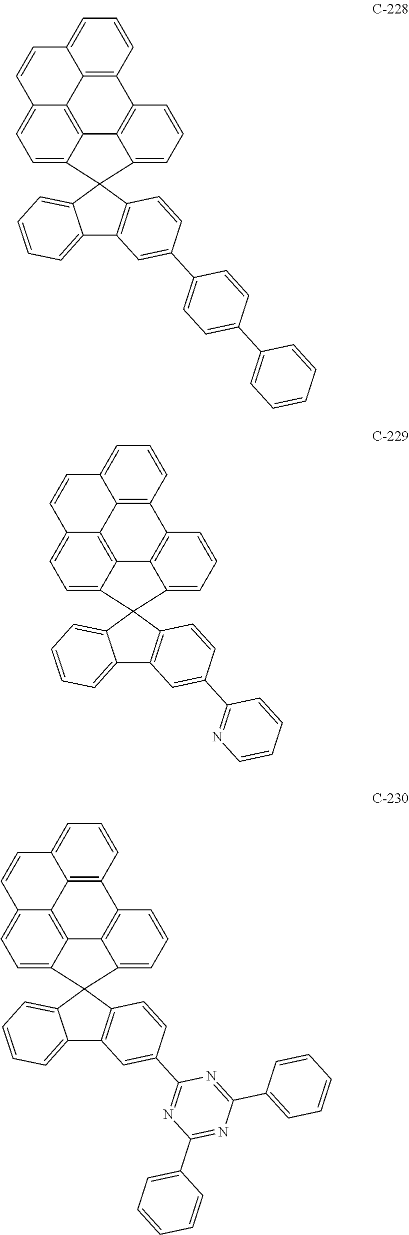

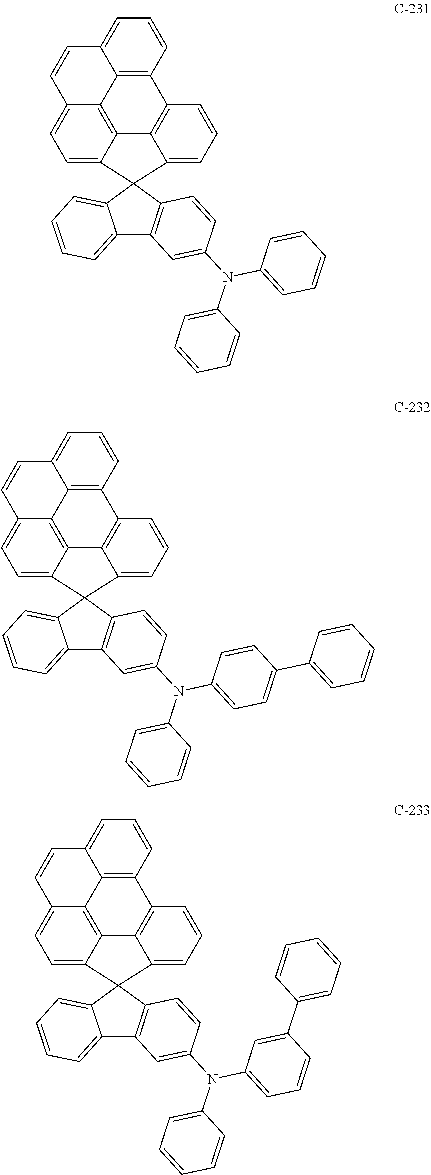

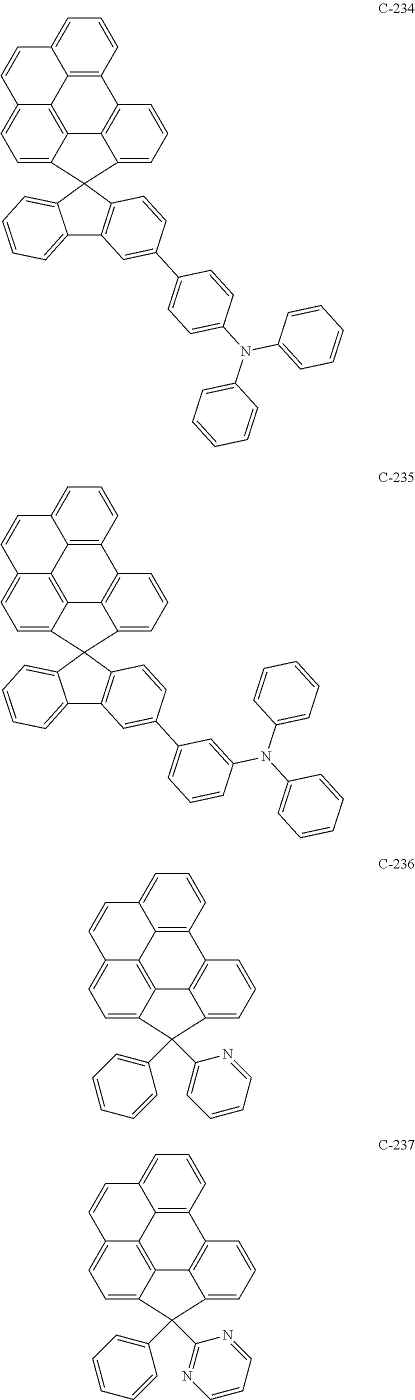

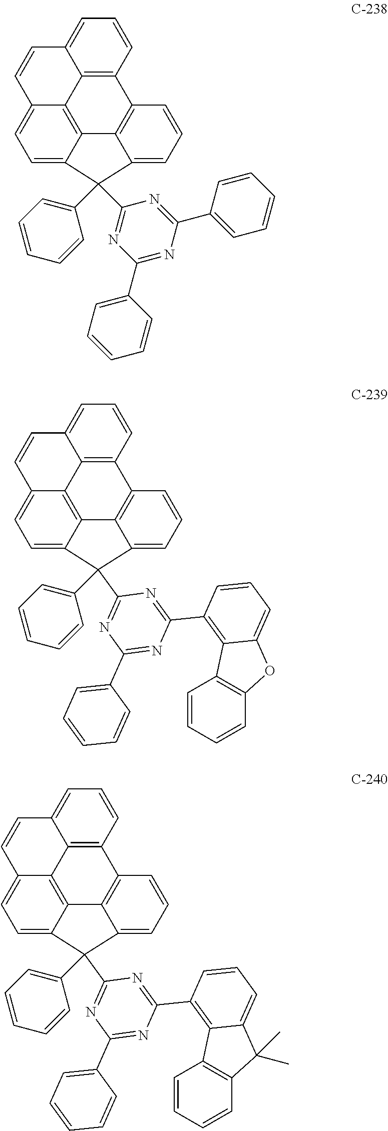

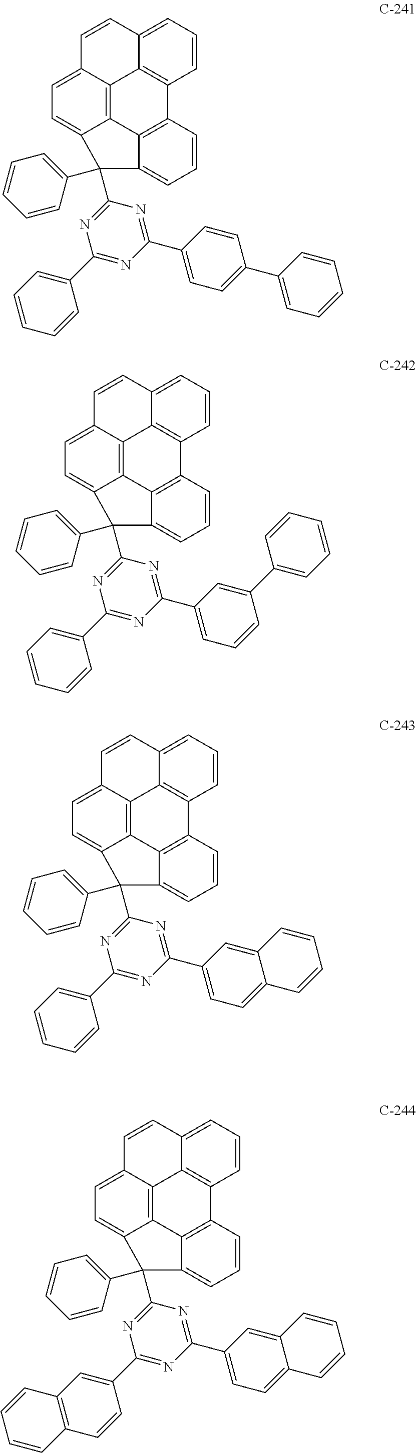 [SMILES: c1ccc(-c2ccc(-c3nc(-c4ccccc4)nc(C4(c5ccccc5)c5cccc6c7cccc8ccc9ccc4c(c56)c9c87)n3)cc2)cc1 ; c1ccc(-c2cccc(-c3nc(-c4ccccc4)nc(C4(c5ccccc5)c5cccc6c7cccc8ccc9ccc4c(c56)c9c87)n3)c2)cc1 ; c1ccc(-c2nc(-c3ccc4ccccc4c3)nc(C3(c4ccccc4)c4cccc5c6cccc7ccc8ccc3c(c45)c8c76)n2)cc1 ; c1ccc(C2(c3nc(-c4ccc5ccccc5c4)nc(-c4ccc5ccccc5c4)n3)c3cccc4c5cccc6ccc7ccc2c(c34)c7c65)cc1]

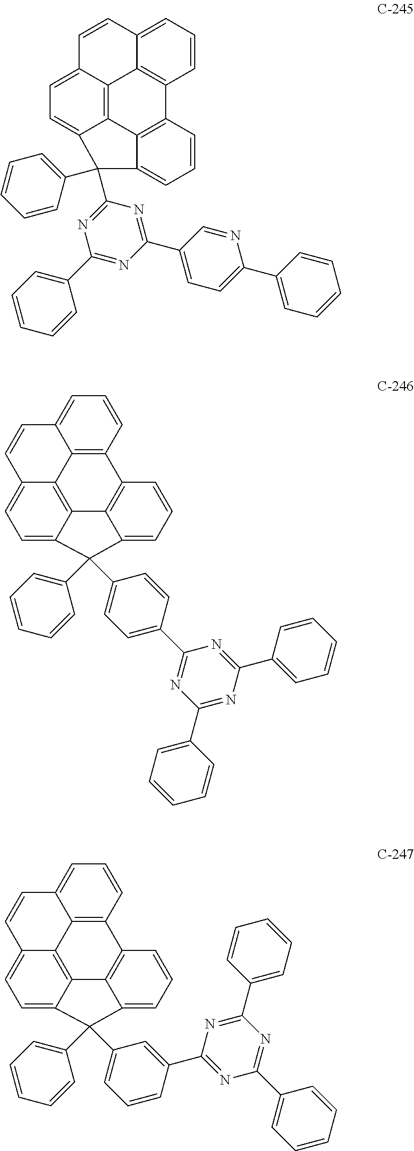 [SMILES: c1ccc(-c2ccc(-c3nc(-c4ccccc4)nc(C4(c5ccccc5)c5cccc6c7cccc8ccc9ccc4c(c56)c9c87)n3)cn2)cc1 ; c1ccc(-c2nc(-c3ccccc3)nc(-c3ccc(C4(c5ccccc5)c5cccc6c7cccc8ccc9ccc4c(c56)c9c87)cc3)n2)cc1 ; c1ccc(-c2nc(-c3ccccc3)nc(-c3cccc(C4(c5ccccc5)c5cccc6c7cccc8ccc9ccc4c(c56)c9c87)c3)n2)cc1]

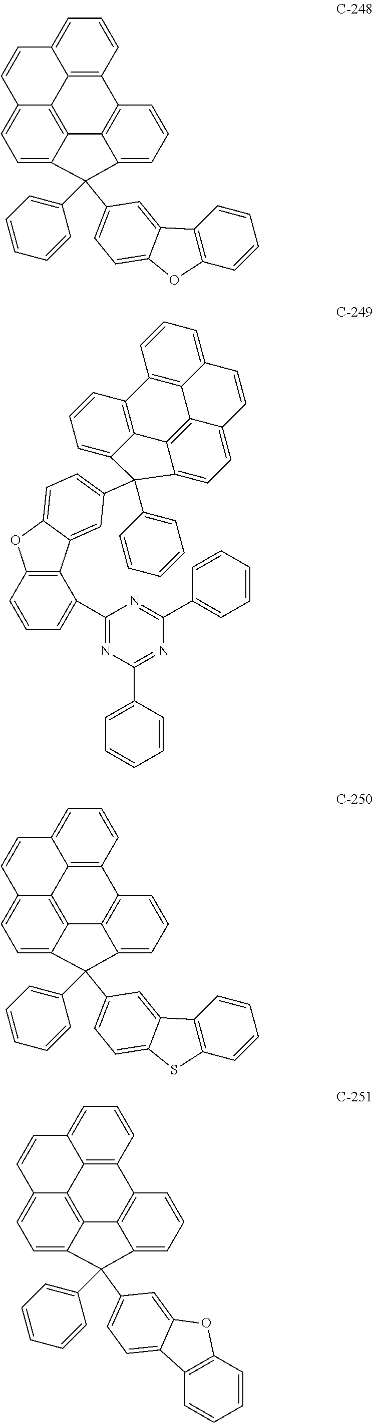

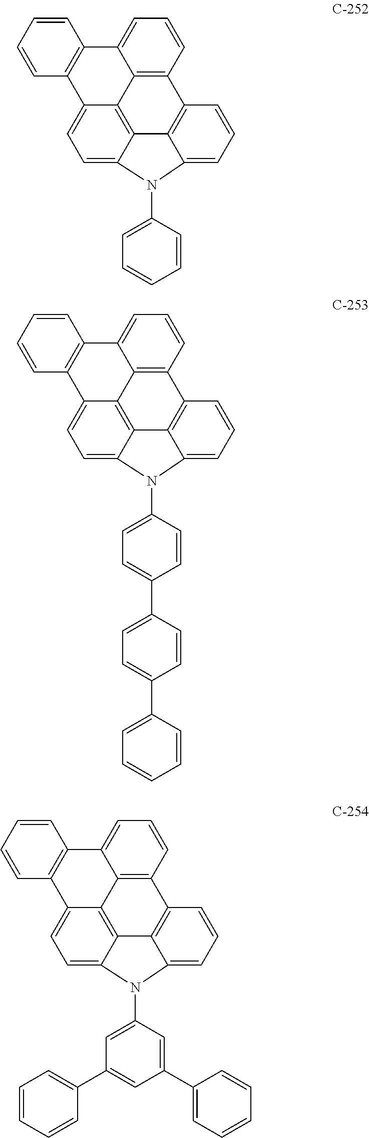

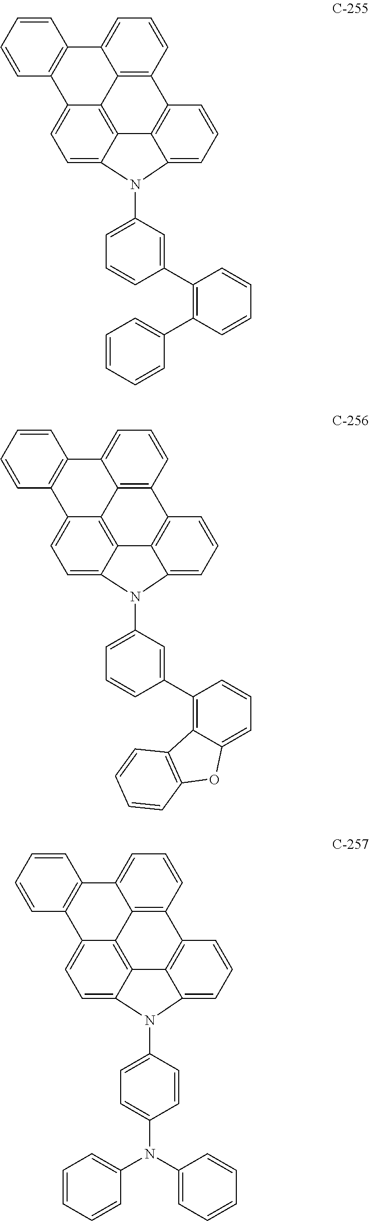

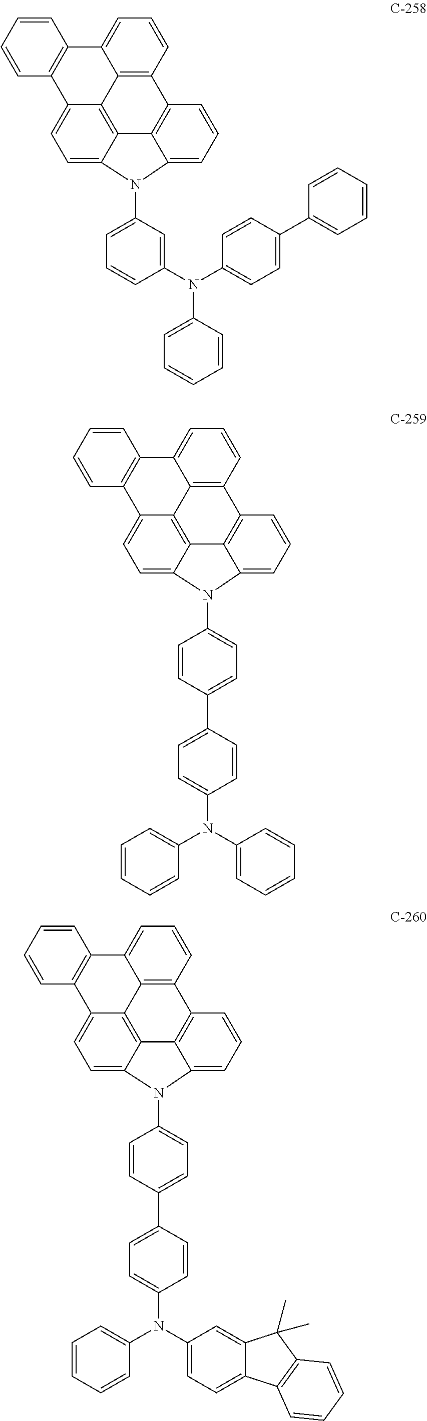

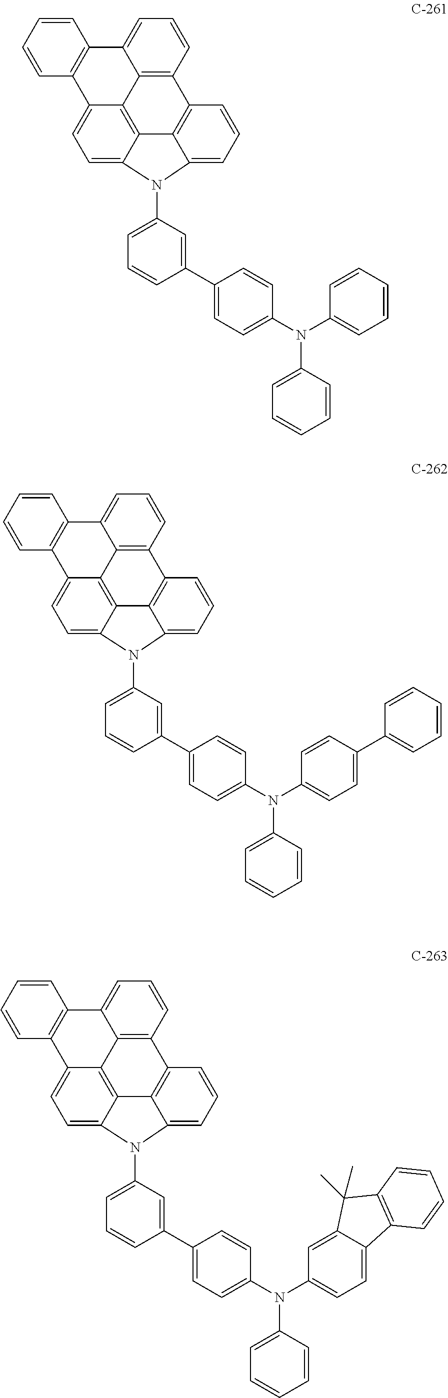 [SMILES: c1ccc(N(c2ccccc2)c2ccc(-c3cccc(-n4c5cccc6c7cccc8c9ccccc9c9ccc4c(c9c87)c65)c3)cc2)cc1 ; c1ccc(-c2ccc(N(c3ccccc3)c3ccc(-c4cccc(-n5c6cccc7c8cccc9c%10ccccc%10c%10ccc5c(c%10c98)c76)c4)cc3)cc2)cc1 ; CC1(C)c2ccccc2-c2ccc(N(c3ccccc3)c3ccc(-c4cccc(-n5c6cccc7c8cccc9c%10ccccc%10c%10ccc5c(c%10c98)c76)c4)cc3)cc21]

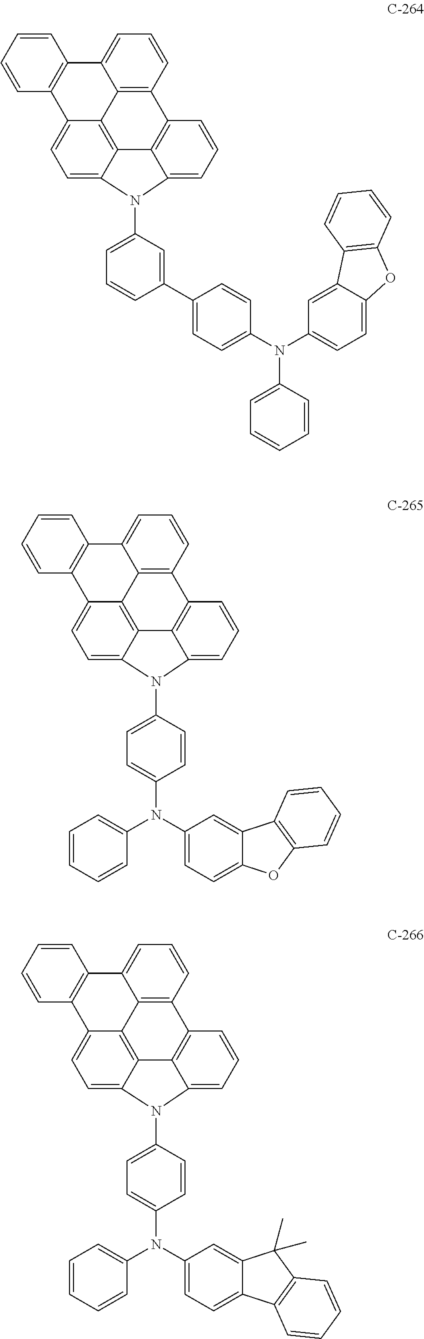 [SMILES: c1ccc(N(c2ccc(-c3cccc(-n4c5cccc6c7cccc8c9ccccc9c9ccc4c(c9c87)c65)c3)cc2)c2ccc3oc4ccccc4c3c2)cc1 ; c1ccc(N(c2ccc(-n3c4cccc5c6cccc7c8ccccc8c8ccc3c(c8c76)c54)cc2)c2ccc3oc4ccccc4c3c2)cc1 ; CC1(C)c2ccccc2-c2ccc(N(c3ccccc3)c3ccc(-n4c5cccc6c7cccc8c9ccccc9c9ccc4c(c9c87)c65)cc3)cc21]

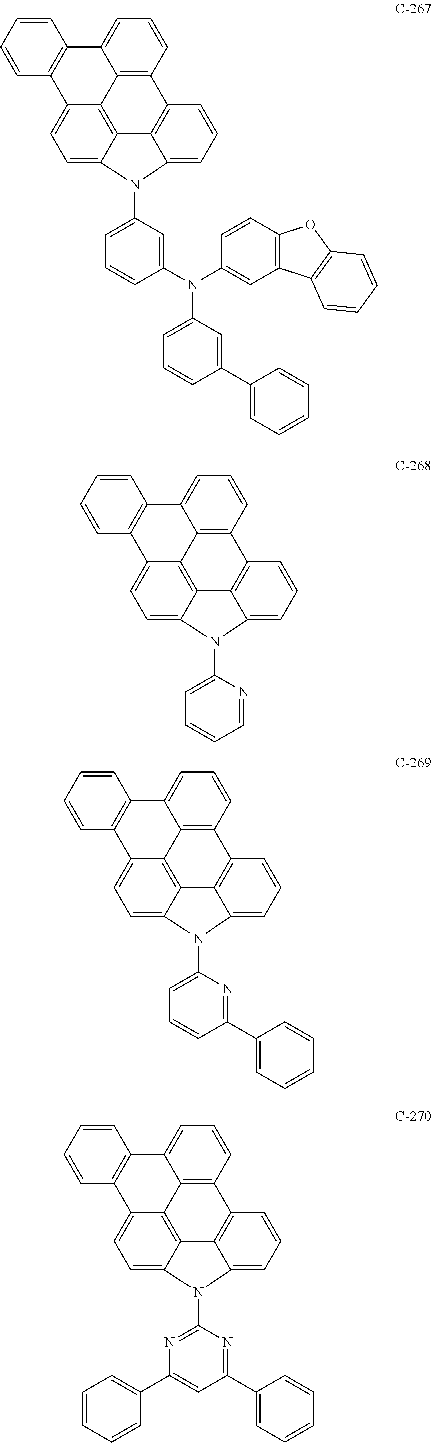

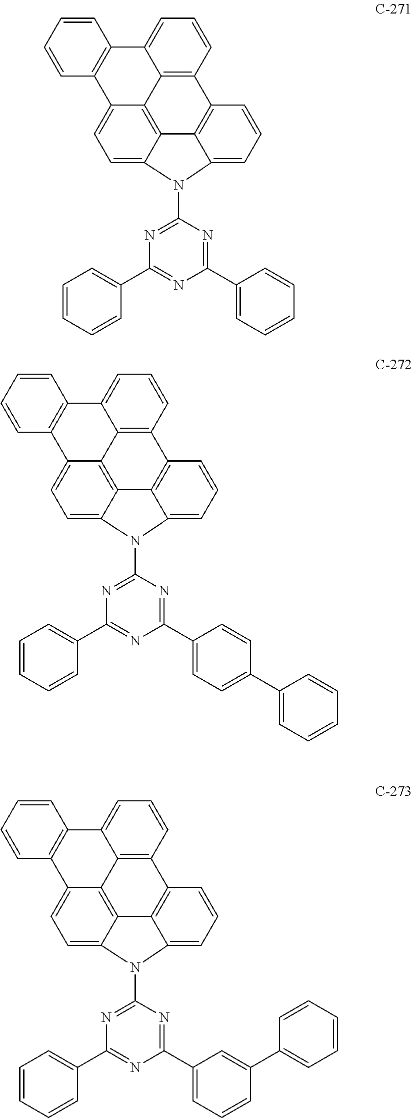 [SMILES: c1ccc(-c2nc(-c3ccccc3)nc(-n3c4cccc5c6cccc7c8ccccc8c8ccc3c(c8c76)c54)n2)cc1 ; c1ccc(-c2ccc(-c3nc(-c4ccccc4)nc(-n4c5cccc6c7cccc8c9ccccc9c9ccc4c(c9c87)c65)n3)cc2)cc1 ; c1ccc(-c2cccc(-c3nc(-c4ccccc4)nc(-n4c5cccc6c7cccc8c9ccccc9c9ccc4c(c9c87)c65)n3)c2)cc1]

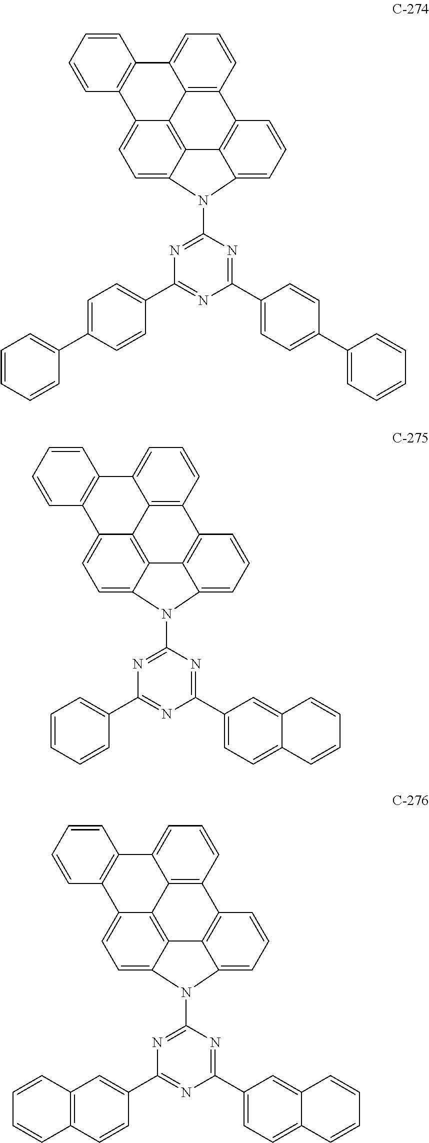 [SMILES: c1ccc(-c2ccc(-c3nc(-c4ccc(-c5ccccc5)cc4)nc(-n4c5cccc6c7cccc8c9ccccc9c9ccc4c(c9c87)c65)n3)cc2)cc1 ; c1ccc(-c2nc(-c3ccc4ccccc4c3)nc(-n3c4cccc5c6cccc7c8ccccc8c8ccc3c(c8c76)c54)n2)cc1 ; c1ccc2cc(-c3nc(-c4ccc5ccccc5c4)nc(-n4c5cccc6c7cccc8c9ccccc9c9ccc4c(c9c87)c65)n3)ccc2c1]

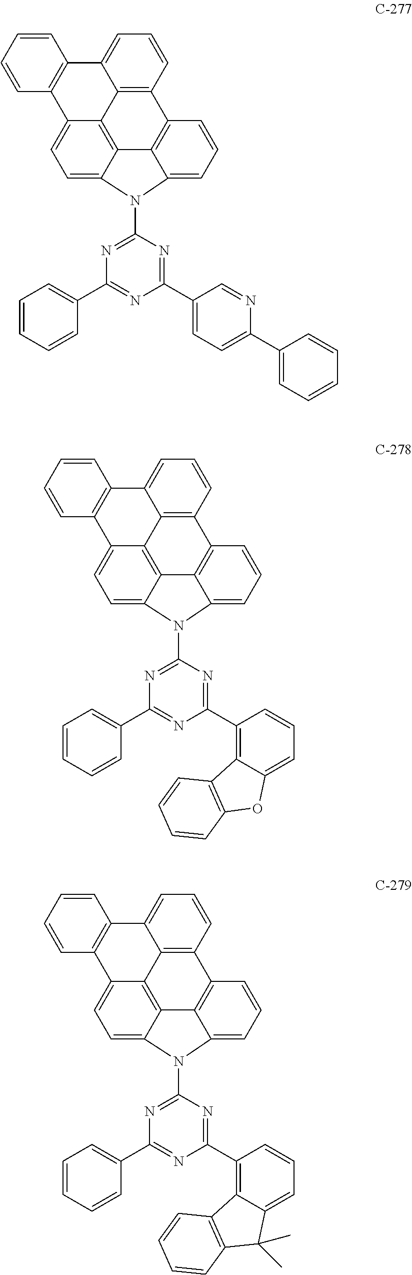 [SMILES: c1ccc(-c2ccc(-c3nc(-c4ccccc4)nc(-n4c5cccc6c7cccc8c9ccccc9c9ccc4c(c9c87)c65)n3)cn2)cc1 ; c1ccc(-c2nc(-c3cccc4oc5ccccc5c34)nc(-n3c4cccc5c6cccc7c8ccccc8c8ccc3c(c8c76)c54)n2)cc1 ; CC1(C)c2ccccc2-c2c(-c3nc(-c4ccccc4)nc(-n4c5cccc6c7cccc8c9ccccc9c9ccc4c(c9c87)c65)n3)cccc21]

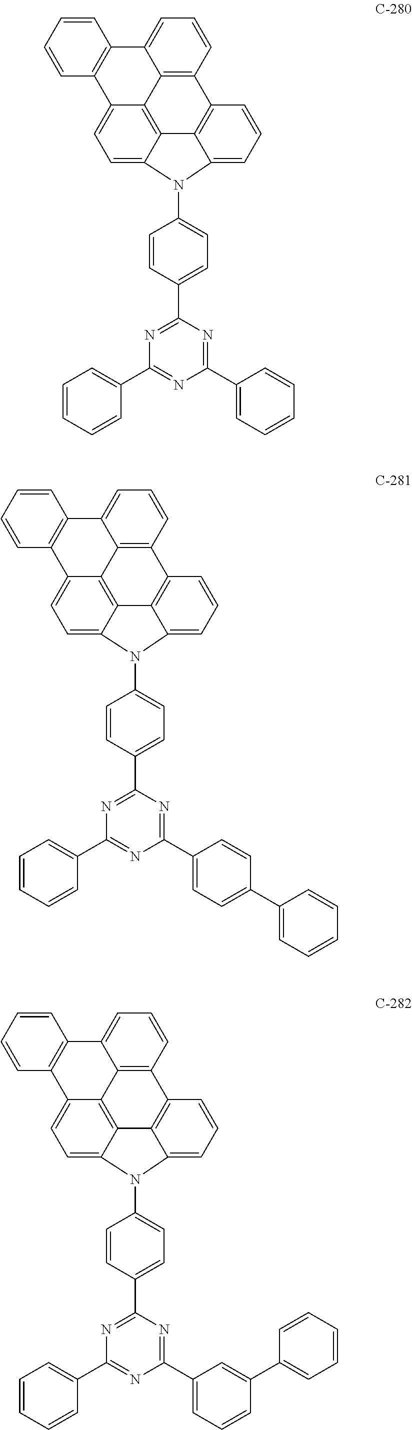 [SMILES: c1ccc(-c2nc(-c3ccccc3)nc(-c3ccc(-n4c5cccc6c7cccc8c9ccccc9c9ccc4c(c9c87)c65)cc3)n2)cc1 ; c1ccc(-c2ccc(-c3nc(-c4ccccc4)nc(-c4ccc(-n5c6cccc7c8cccc9c%10ccccc%10c%10ccc5c(c%10c98)c76)cc4)n3)cc2)cc1 ; c1ccc(-c2cccc(-c3nc(-c4ccccc4)nc(-c4ccc(-n5c6cccc7c8cccc9c%10ccccc%10c%10ccc5c(c%10c98)c76)cc4)n3)c2)cc1]

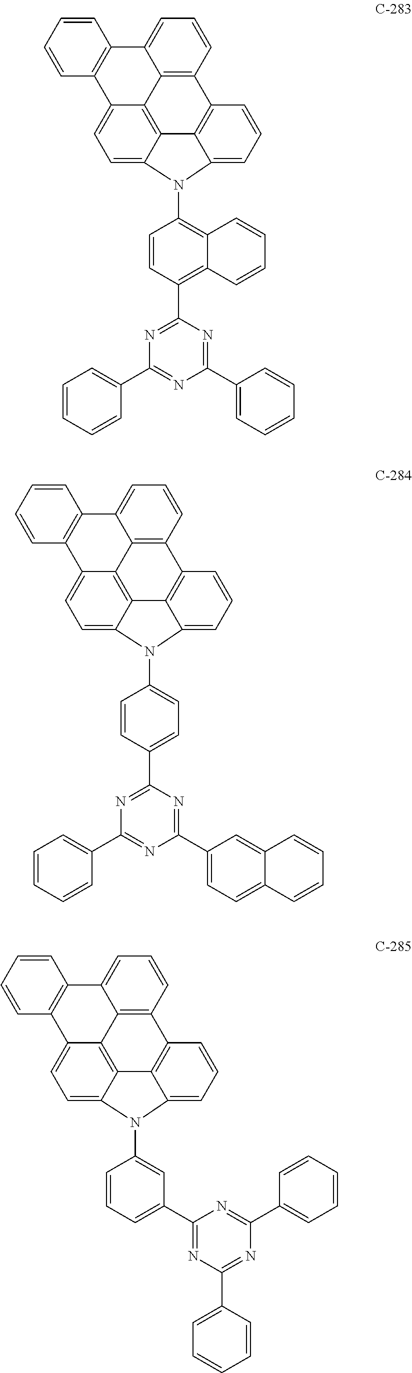 [SMILES: c1ccc(-c2nc(-c3ccccc3)nc(-c3ccc(-n4c5cccc6c7cccc8c9ccccc9c9ccc4c(c9c87)c65)c4ccccc34)n2)cc1 ; c1ccc(-c2nc(-c3ccc(-n4c5cccc6c7cccc8c9ccccc9c9ccc4c(c9c87)c65)cc3)nc(-c3ccc4ccccc4c3)n2)cc1 ; c1ccc(-c2nc(-c3ccccc3)nc(-c3cccc(-n4c5cccc6c7cccc8c9ccccc9c9ccc4c(c9c87)c65)c3)n2)cc1]

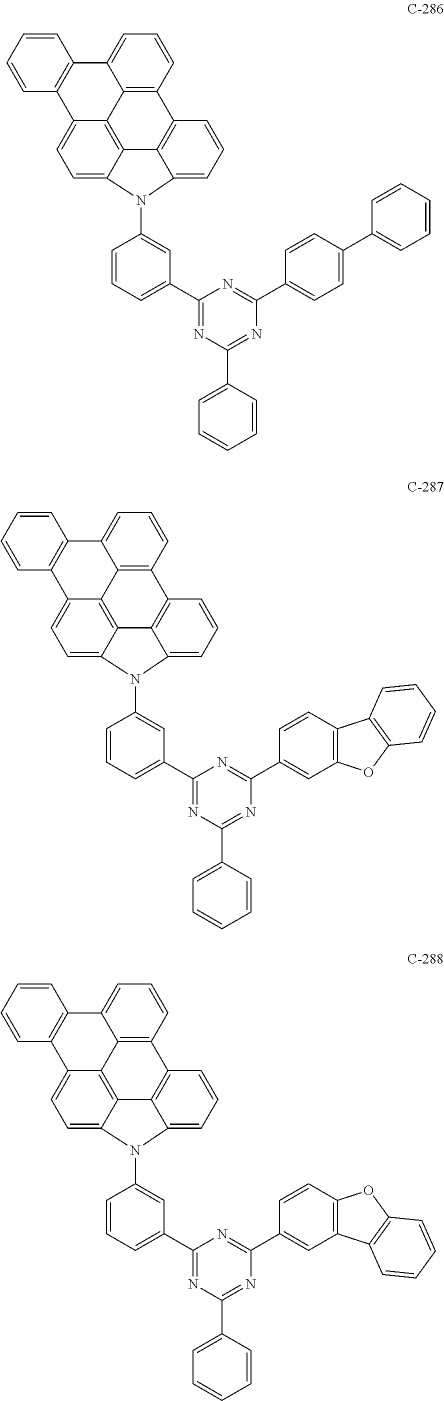 [SMILES: c1ccc(-c2ccc(-c3nc(-c4ccccc4)nc(-c4cccc(-n5c6cccc7c8cccc9c%10ccccc%10c%10ccc5c(c%10c98)c76)c4)n3)cc2)cc1 ; c1ccc(-c2nc(-c3cccc(-n4c5cccc6c7cccc8c9ccccc9c9ccc4c(c9c87)c65)c3)nc(-c3ccc4c(c3)oc3ccccc34)n2)cc1 ; c1ccc(-c2nc(-c3cccc(-n4c5cccc6c7cccc8c9ccccc9c9ccc4c(c9c87)c65)c3)nc(-c3ccc4oc5ccccc5c4c3)n2)cc1]

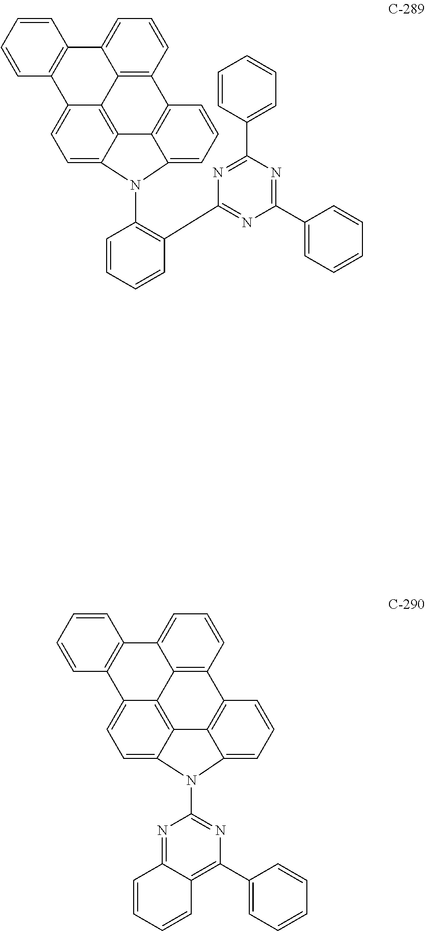 [SMILES: c1ccc(-c2nc(-c3ccccc3)nc(-c3ccccc3-n3c4cccc5c6cccc7c8ccccc8c8ccc3c(c8c76)c54)n2)cc1 ; c1ccc(-c2nc(-n3c4cccc5c6cccc7c8ccccc8c8ccc3c(c8c76)c54)nc3ccccc23)cc1]

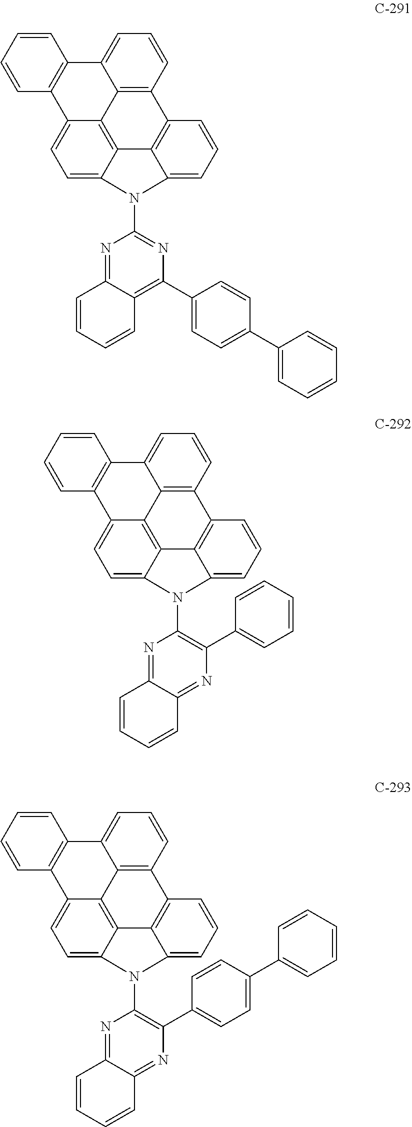 [SMILES: c1ccc(-c2ccc(-c3nc(-n4c5cccc6c7cccc8c9ccccc9c9ccc4c(c9c87)c65)nc4ccccc34)cc2)cc1 ; c1ccc(-c2nc3ccccc3nc2-n2c3cccc4c5cccc6c7ccccc7c7ccc2c(c7c65)c43)cc1 ; c1ccc(-c2ccc(-c3nc4ccccc4nc3-n3c4cccc5c6cccc7c8ccccc8c8ccc3c(c8c76)c54)cc2)cc1]

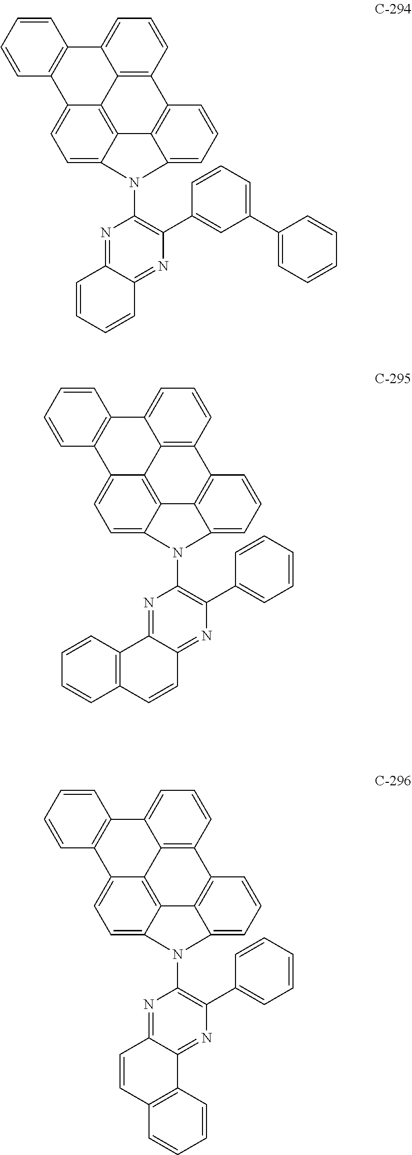 [SMILES: c1ccc(-c2cccc(-c3nc4ccccc4nc3-n3c4cccc5c6cccc7c8ccccc8c8ccc3c(c8c76)c54)c2)cc1 ; c1ccc(-c2nc3ccc4ccccc4c3nc2-n2c3cccc4c5cccc6c7ccccc7c7ccc2c(c7c65)c43)cc1 ; c1ccc(-c2nc3c(ccc4ccccc43)nc2-n2c3cccc4c5cccc6c7ccccc7c7ccc2c(c7c65)c43)cc1]

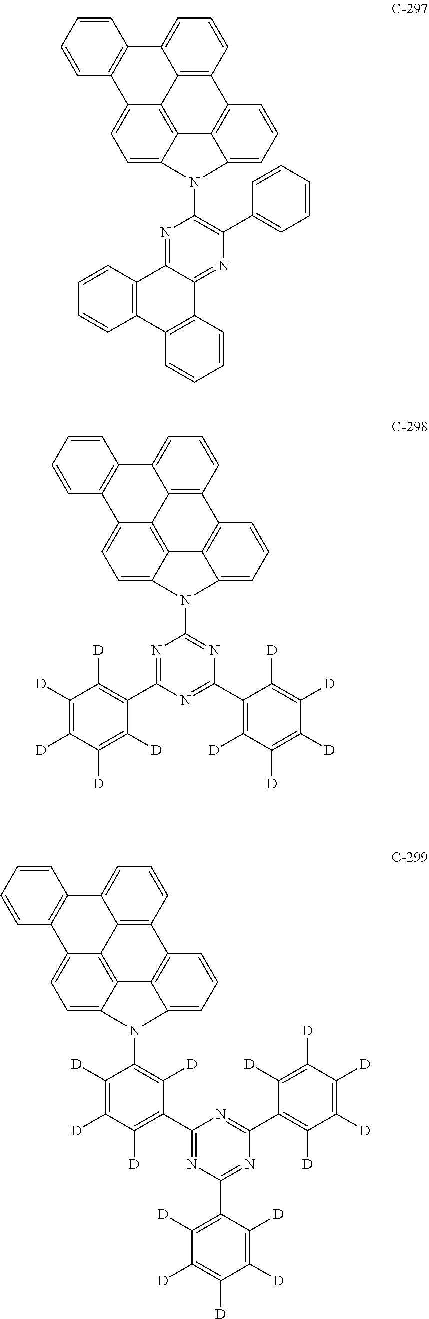 [SMILES: c1ccc(-c2nc3c4ccccc4c4ccccc4c3nc2-n2c3cccc4c5cccc6c7ccccc7c7ccc2c(c7c65)c43)cc1 ; [2H]c1c([2H])c([2H])c(-c2nc(-c3c([2H])c([2H])c([2H])c([2H])c3[2H])nc(-n3c4cccc5c6cccc7c8ccccc8c8ccc3c(c8c76)c54)n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c(-n4c5cccc6c7cccc8c9ccccc9c9ccc4c(c9c87)c65)c3[2H])n2)c([2H])c1[2H]]

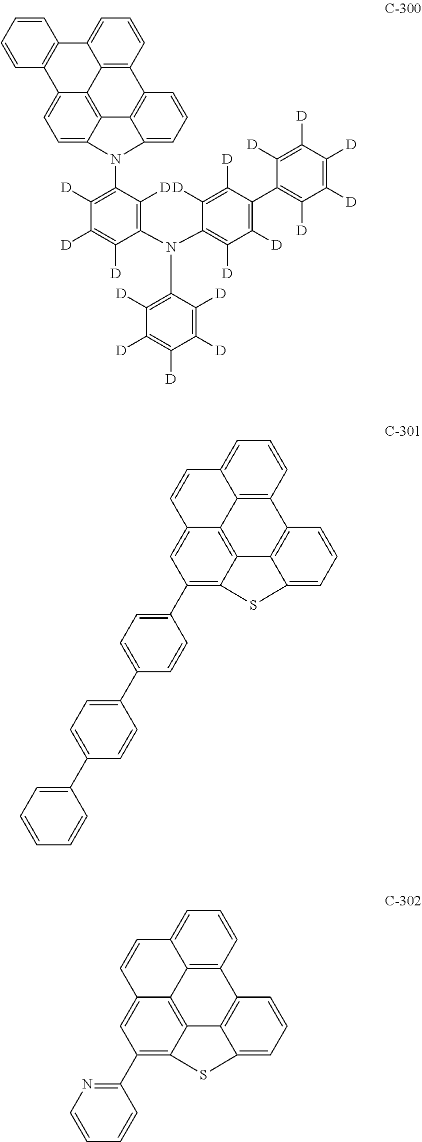 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-n4c5cccc6c7cccc8c9ccccc9c9ccc4c(c9c87)c65)c3[2H])c([2H])c2[2H])c([2H])c1[2H] ; c1ccc(-c2ccc(-c3ccc(-c4cc5ccc6cccc7c8cccc9sc4c(c98)c5c67)cc3)cc2)cc1 ; c1ccc(-c2cc3ccc4cccc5c6cccc7sc2c(c76)c3c45)nc1]

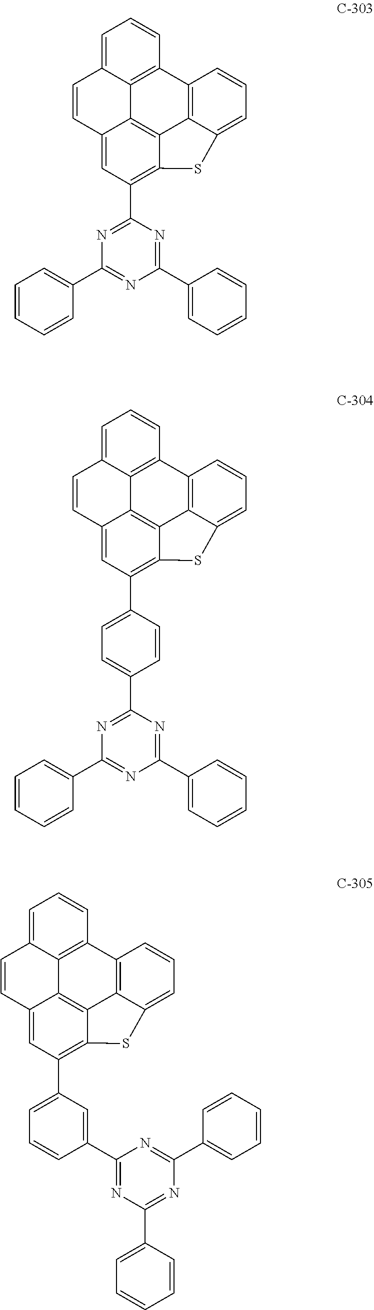 [SMILES: c1ccc(-c2nc(-c3ccccc3)nc(-c3cc4ccc5cccc6c7cccc8sc3c(c87)c4c56)n2)cc1 ; c1ccc(-c2nc(-c3ccccc3)nc(-c3ccc(-c4cc5ccc6cccc7c8cccc9sc4c(c98)c5c67)cc3)n2)cc1 ; c1ccc(-c2nc(-c3ccccc3)nc(-c3cccc(-c4cc5ccc6cccc7c8cccc9sc4c(c98)c5c67)c3)n2)cc1]

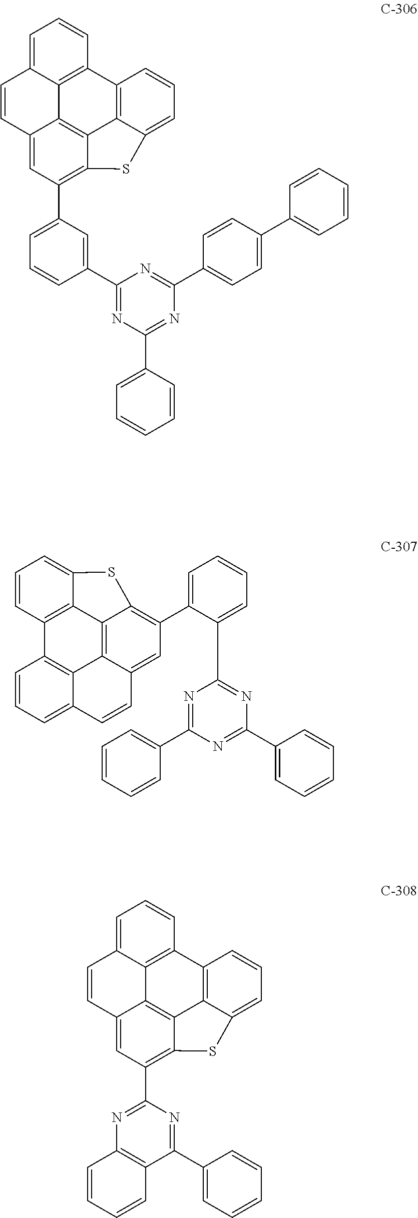 [SMILES: c1ccc(-c2ccc(-c3nc(-c4ccccc4)nc(-c4cccc(-c5cc6ccc7cccc8c9cccc%10sc5c(c%109)c6c78)c4)n3)cc2)cc1 ; c1ccc(-c2nc(-c3ccccc3)nc(-c3ccccc3-c3cc4ccc5cccc6c7cccc8sc3c(c87)c4c56)n2)cc1 ; c1ccc(-c2nc(-c3cc4ccc5cccc6c7cccc8sc3c(c87)c4c56)nc3ccccc23)cc1]

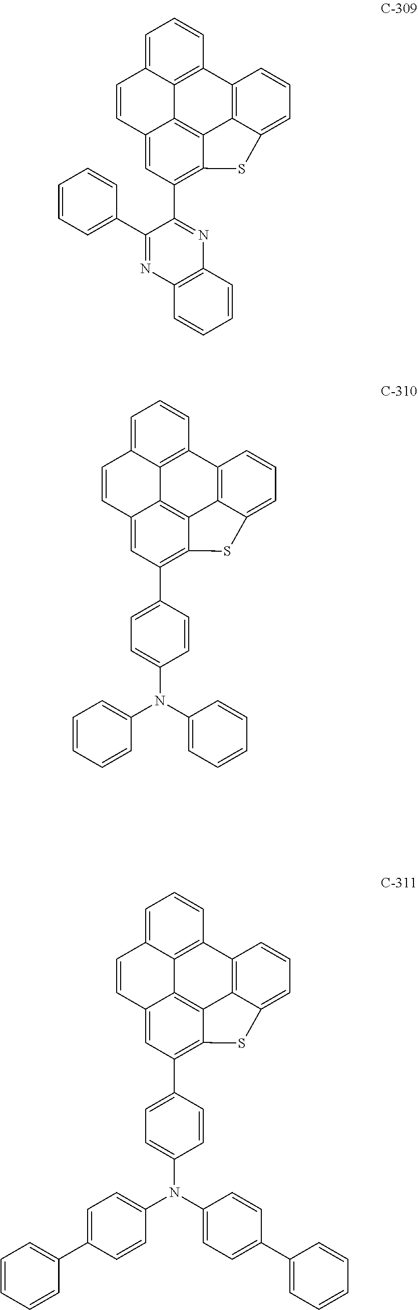 [SMILES: c1ccc(-c2nc3ccccc3nc2-c2cc3ccc4cccc5c6cccc7sc2c(c76)c3c45)cc1 ; c1ccc(N(c2ccccc2)c2ccc(-c3cc4ccc5cccc6c7cccc8sc3c(c87)c4c56)cc2)cc1 ; c1ccc(-c2ccc(N(c3ccc(-c4ccccc4)cc3)c3ccc(-c4cc5ccc6cccc7c8cccc9sc4c(c98)c5c67)cc3)cc2)cc1]

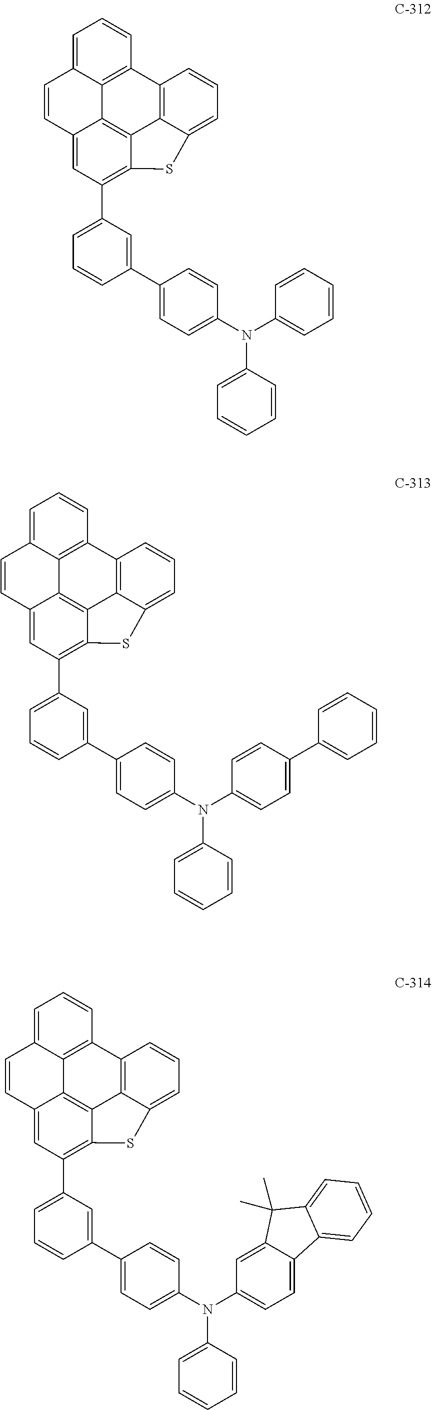 [SMILES: c1ccc(N(c2ccccc2)c2ccc(-c3cccc(-c4cc5ccc6cccc7c8cccc9sc4c(c98)c5c67)c3)cc2)cc1 ; c1ccc(-c2ccc(N(c3ccccc3)c3ccc(-c4cccc(-c5cc6ccc7cccc8c9cccc%10sc5c(c%109)c6c78)c4)cc3)cc2)cc1 ; CC1(C)c2ccccc2-c2ccc(N(c3ccccc3)c3ccc(-c4cccc(-c5cc6ccc7cccc8c9cccc%10sc5c(c%109)c6c78)c4)cc3)cc21]

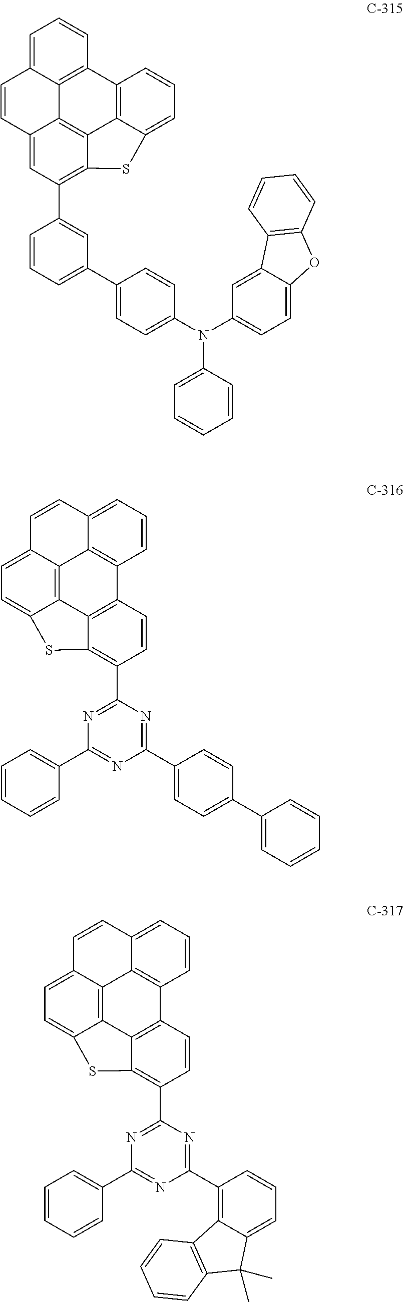 [SMILES: c1ccc(N(c2ccc(-c3cccc(-c4cc5ccc6cccc7c8cccc9sc4c(c98)c5c67)c3)cc2)c2ccc3oc4ccccc4c3c2)cc1 ; c1ccc(-c2ccc(-c3nc(-c4ccccc4)nc(-c4ccc5c6cccc7ccc8ccc9sc4c5c9c8c76)n3)cc2)cc1 ; CC1(C)c2ccccc2-c2c(-c3nc(-c4ccccc4)nc(-c4ccc5c6cccc7ccc8ccc9sc4c5c9c8c76)n3)cccc21]

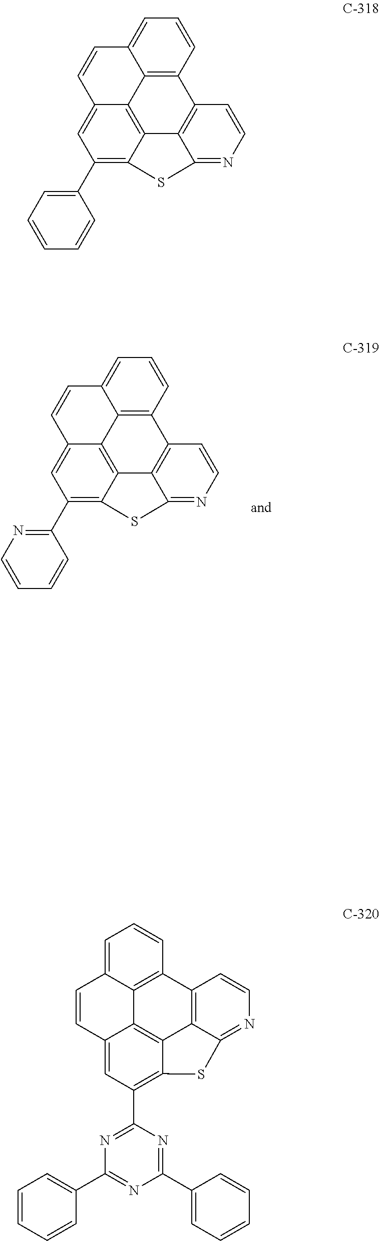 [SMILES: c1ccc(-c2cc3ccc4cccc5c6ccnc7sc2c(c76)c3c45)cc1 ; c1ccc(-c2cc3ccc4cccc5c6ccnc7sc2c(c76)c3c45)nc1 ; c1ccc(-c2nc(-c3ccccc3)nc(-c3cc4ccc5cccc6c7ccnc8sc3c(c87)c4c56)n2)cc1]

5. An organic electroluminescent material, comprising an organic electroluminescent compound according to claim 1.

6. An organic electroluminescent device comprising an organic electroluminescent compound according to claim 1.

7. The organic electroluminescent device according to claim 6, wherein the organic electroluminescent compound is included in at least one of a hole transport layer, a light-emitting layer, a buffer layer, and an electron transport layer.

8. A plurality of host materials comprising a first host material and a second host material, wherein the first host material comprises an organic electroluminescent compound according to claim 1 and the second host material comprises an organic electroluminescent compound represented by the following formula 2. 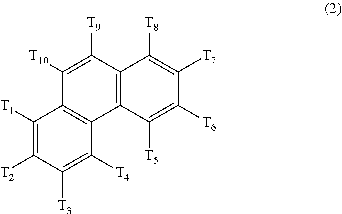
wherein,
T₅ and T₆ are linked to each other to form a ring of the following formula 3; or T₇ and T₈ are linked to each other to form a ring of the following formula 3; or T₅ and T₆ are linked to each other to form a ring of the following formula 3 and T₇ and T₈ are linked to each other to form a ring of the following formula 3; 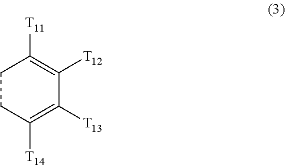
T₁ to T₄, T₉ to T₁₄, and T₅ to T₈, that do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂, provided that at least one of T₁ to T₁₄ is -L₂-Ar₂;
L₂ represents a single bond, a substituted or unsubstituted (C6-C30)arylene, or a substituted or unsubstituted (3- to 30-membered)heteroarylene;
Ar₂ represents a substituted or unsubstituted (C6-C30)aryl, or a substituted or unsubstituted (3- to 30-membered)heteroaryl; [symbol]
represents a site fused to the formula 2; and
the heteroaryl comprises at least one hetero atom selected from B, N, O, S, Si and P.

9. The plurality of host materials according to claim 8, wherein the compound represented by formula 2 is selected from the following compounds:

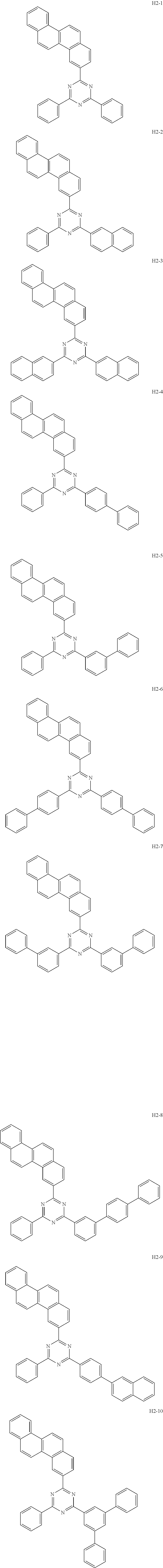

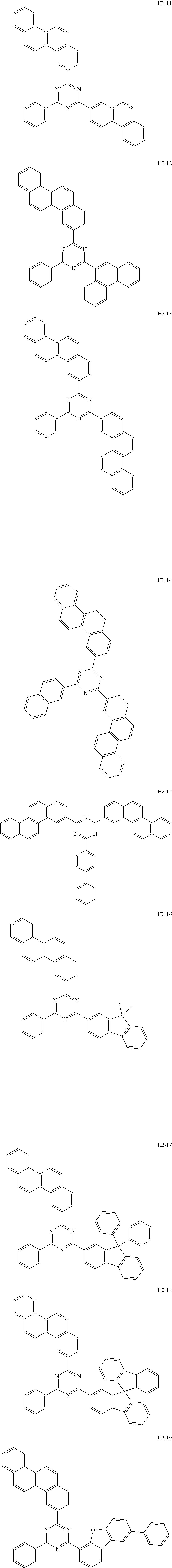

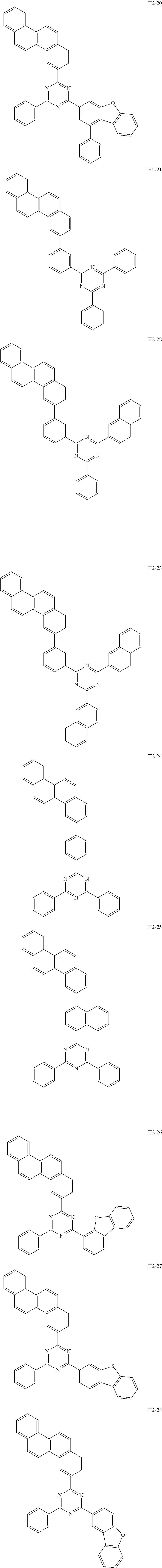

[chemical structure]

[chemical structure]

[chemical structure]

[chemical structure]

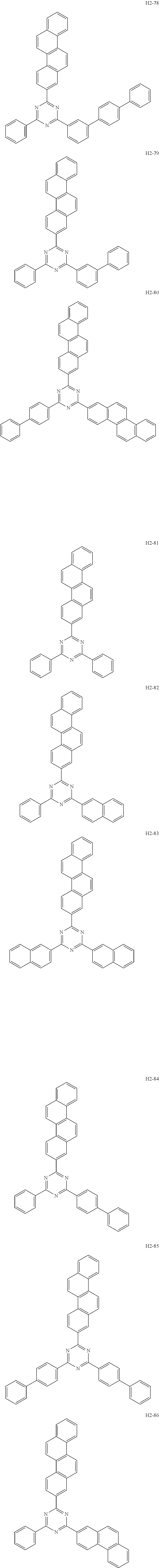

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

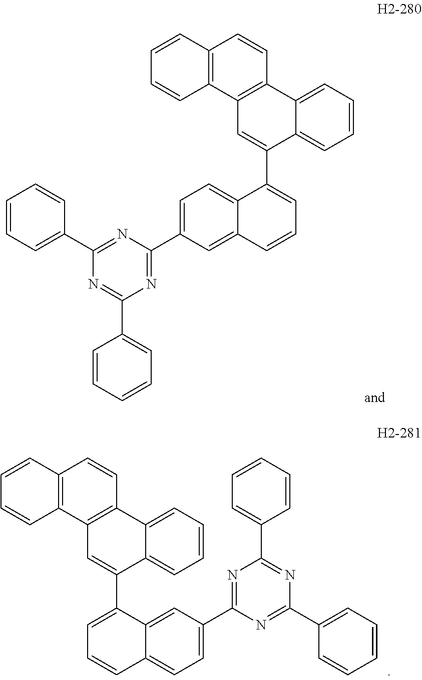 [SMILES: c1ccc(-c2nc(-c3ccccc3)nc(-c3ccc4c(-c5cc6c7ccccc7ccc6c6ccccc56)cccc4c3)n2)cc1 ; c1ccc(-c2nc(-c3ccccc3)nc(-c3ccc4cccc(-c5cc6c7ccccc7ccc6c6ccccc56)c4c3)n2)cc1]

10. An organic electroluminescent device comprising the plurality of host materials according to claim 8.
